(19) United States
(12) Patent Application Publication
(10) Pub. No.: US 2006/0078902 A1
(43) Pub. Date: Apr. 13, 2006

(54) Method and compositions for RNA interference

(76) Inventors: Michaeline Bunting, San Diego, CA (US); Knut R. Madden, Carlsbad, CA (US); Adam N. Harris, Oceanside, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/105,594

(22) Filed: Apr. 14, 2005

Related U.S. Application Data
(60) Provisional application No. 60/562,227, filed on Apr. 15, 2004.

(51) Int. Cl. C12Q 1/68

(52) U.S. Cl. 435/6

(57) ABSTRACT

The invention provides methods and compositions related to the field of gene expression regulation. In particular, methods and compositions of the invention can be used to identify RNAi cleavage sites along a target RNA molecule. Methods and compositions of the invention may also be used to knockdown expression of nucleic acid molecules which encode reporters.

### CROSS-REFERENCE TO RELATED APPLICATIONS

This application claims the benefit of U.S. Provisional Patent Application No. 60/562,227, filed Apr. 15, 2004, the content of which is relied upon and incorporated by reference in its entirety.

### BACKGROUND OF THE INVENTION

1. Field of the Invention

The invention provides methods and compositions related to the field of gene expression regulation. In particular, methods and compositions of the invention can be used to identify RNAi cleavage sites along a target RNA molecule. Methods and compositions of the invention may also be used to knock down expression of nucleic acid molecules which encode reporters.

2. Background

RNA interference (RNAi) is a phenomenon whereby double stranded RNA (dsRNA) molecules induce the sequence-specific cleavage of cognate mRNA in animal or plant cells. (Fire et al., Nature 391:806-811 (1998); Hutvagner et al., Curr. Opin. Genet. Dev. 12:225-232 (2002); Hannon, G. J., Nature 418:244-251 (2002); McManus and Sharp, Nature Reviews 3:737-747 (2002); Dykxhoorn et al., Nature Reviews 4 :457-466 (2003)). Gene silencing by RNAi involves cleavage of a dsRNA molecule into 21 to 25 nt RNA molecules. The 21 to 25 nt molecules are known as small interfering RNA (siRNA) molecules. Cleavage of dsRNA to produce siRNA molecules is mediated by the cellular RNase III enzyme Dicer. (Bernstein et al., Nature 409:363-366 (2001) and Ketting et al., Genes Dev. 15:2654-2659 (2001); Yang et al., Proc. Natl. Acad. Sci. USA 99:9942-9947 (2002)). Next, siRNA becomes associated with an RNA-inducing silencing complex (RISC) and its cognate mRNA, leading to cleavage of target mRNA, and consequently, silencing of the gene encoded by the RNA.

Gene silencing with dsRNA molecules can be used to investigate the functions of genes and gene products. An investigator can use dsRNA to target the destruction of a specific mRNA and observe the resulting phenotypic response. dsRNA-mediated gene silencing is useful in a variety of biological applications, including genetic screens, inhibiting infection by pathogenic agents (e.g., parasites, viruses, etc.), and gene therapy. (McManus and Sharp, Nature Reviews Genetics 3:737-747 (2002)).

Gene silencing can be accomplished by introducing siRNAs into cells. (Holen et al., Nucl. Acids Res. 30:1757-1766 (2002)). siRNAs can be produced by a variety of methods. For example, siRNAs can be obtained by chemical synthesis (Elbashir et al., Nature 411:494-498 (2001)), by in vitro transcription from short DNA templates (Yu et al., Proc. Natl. Acad. Sci. 99:6047-6052 (2002)), by in vivo transcription from transfected DNA constructs (Miyagishi and Taira, Nat. Biotechnol. 20:497-500 (2002)) and by in vitro cleavage of longer dsRNA molecules using an enzyme with RNase III activity. (Myers et al., Nat. Biotechnol. 21:324-328 (2003); Yang et al., Proc. Natl. Acad. Sci. USA 99:9942-9947 (2002); Kawasaki et al., Nucl. Acids Res. 31:981-987 (2003)).

Although siRNAs are powerful tools for gene silencing, there are certain considerations that must go into the design of siRNAs. The nucleotide sequence of an siRNA should correspond to a sequence found within the mRNA molecule that is targeted for cleavage. The sequence chosen should be relatively unique to the target mRNA to prevent unintended cleavage or translational repression of homologous mRNAs. (Doench et al., Genes Dev. 17:438-442 (2003)).

In addition, it has been observed that the efficacy of siRNAs is dependent on the target site to which the siRNAs correspond on the target mRNA. (Kawasaki et al., Nucl. Acids Res. 31:981-987 (2003); Holen et al., Nucl. Acids Res. 30:1757-1766 (2002)). siRNAs corresponding to certain regions along a target mRNA molecule may be able to mediate target mRNA cleavage to a greater or lesser extent as compared to siRNAs that correspond to other regions along the target mRNA. In some cases, siRNAs that differ in their target sites by only a few nucleotides have dramatically different gene silencing abilities. The susceptibility of a target sequence to siRNA mediated cleavage is believed to be determined by many factors including the secondary and tertiary structure of the target sequence, association with RNA binding proteins, and rate of translation. (Dykxhoorn et al., Nature Reviews 4:457-466 (2003). It has been suggested that, at least for some human genes, target sites that are susceptible to siRNA-mediated cleavage may be rare. (Holen et al., Nucl. Acids Res. 30:1757-1766 (2002)). Furthermore, the sequence contribution of the siRNA to the cleavage process has not been well defined and may influence the association with the RISC, affinity to the target sequence, target sequence cleavage, and disassociation following cleavage. (Dykxhoorn et al., Nature Reviews 4:457-466 (2003).

In order to design siRNA molecules that can effectively and efficiently mediate the cleavage of target mRNA molecules, it would be highly advantageous to be able to first identify the site (or sites) along a target mRNA molecule that are particularly susceptible to siRNA-mediated cleavage. It would also be advantageous to be able to compare various target sites along an RNA molecule in terms of their relative susceptibilities to siRNA-mediated cleavage. It would further be advantageous to have convenient markers which can be used to measure RNAi reactions. Accordingly, there is a need in the art for methods that can identify sites along target mRNA molecules that are susceptible to siRNA-mediated cleavage and for markers which allow for rapid and efficient measurement of RNAi reactions.

### SUMMARY OF THE INVENTION

The present invention fulfills the aforementioned need in the art by providing methods and compositions that can be used to identify RNAi cleavage sites along target RNA molecules and for measuring RNAi reactions. Thus, the invention provides methods and compositions for RNAi.

The invention is based, in part, on the concept of identifying RNAi cleavage sites along a target RNA molecule by first facilitating dsRNA-mediated cleavage of the target RNA molecule, and then analyzing the individual products of dsRNA-mediated cleavage in order to identify the sites of RNAi cleavage along the target RNA molecule. In methods of the invention, dsRNA-mediated cleavage of target RNA molecules may occur in vivo (e.g., in cells) or in vitro (e.g., under conditions where the target RNA molecules is not contained in a cell).

In certain embodiments, the invention utilizes multiple, non-identical dsRNA molecules which correspond to different segments along a selected target RNA molecule. In certain embodiments, dsRNA mixed populations are used. The dsRNA molecules are either introduced into a cell that comprises the target RNA molecule or are combined with a cell-free system that comprises the target RNA molecule and that allows for in vitro RNAi cleavage. In many cases, not all of the dsRNA molecules that are introduced into a cell, or are combined with a cell-free system, will correspond to segments of the target RNA molecule that are susceptible to efficient RNAi cleavage; some of the dsRNA molecules may correspond, for example, to segments that are highly susceptible to RNAi cleavage, and others may correspond, for example, to segments that are poorly susceptible or resistant to RNAi cleavage. Thus, an analysis of the products of dsRNA-mediated cleavage of the target RNA molecule (e.g., an analysis of the size or sequence of the cleavage products) will often reveal the sites along the target RNA molecule that are susceptible to RNAi cleavage. Further, in many instances, methods of the invention will result in the identification of RNAi cleavage sites and relative efficiency of RNAi mediated cleavage at these sites as compared to cleavage at other sites. In other words, methods of the invention will often lead to the identification of cleavage sites within a target RNA molecules that may be used for efficient knock-down of functional (e.g., translatable) forms of these target RNA molecules.

As an example, the invention includes the use of a mixed population of dsRNA molecules, substantially all of which share sequence identity with a target RNA molecule. The target RNA molecule is contacted with this mixed population of dsRNA molecules, either in vitro or in vivo, under condition which allow for RNAi processes to occur. This mixed population of dsRNA molecules may contain other nucleic acid molecules as well. For example, two mixed populations of dsRNA molecules may be mixed together prior to being contacted with a target RNA molecule. Further, the members of only one of the two mixed populations of dsRNA molecules may share sequence identity with the target RNA molecule.

After a suitable period of time, the cleavage sites in the target RNA molecule are identified. It may then be determined from the locations of the cleavage sites which of the dsRNA molecules mediated each cleavage reaction. Of course, with most target RNA molecules, multiple cleavage sites will be identified using the method described above. The relative number of cleavage products which correlate to particular cleavage sites may then be used to determine the relative effectiveness of individual dsRNA molecules present in the mixed population for mediating RNAi processes. In other words, not only do analyses of the invention lead to the identification of sites which are capable of being cleaved by RNAi, but it also allows the investigator to determine the relative susceptibilities of various sites to RNAi cleavage. The results of analyses such as these allow investigators to design specific dsRNA molecules that correspond to sites which are potentially highly susceptible to RNAi cleavage and, thus, are useful for efficient RNAi-mediated gene silencing. The invention also provides a basis for establishing a correlation between various primary, secondary, and tertiary RNA structures and their relative susceptibilities to RNAi cleavage. This is especially the case when the target RNA molecule is one which forms secondary and tertiary structures (e.g., tRNA molecules).

The invention therefore includes methods for identifying one or more RNAi cleavage sites along a target RNA molecule. According to certain embodiments, methods of the invention comprise: (a) introducing one or more double stranded RNA (dsRNA) molecules into a cell, or combining one or more dsRNA molecules in a cell-free system which allows for in vitro dsRNA-mediated cleavage of RNA molecules, wherein the cell or cell-free system comprises the target RNA molecule; (b) incubating the composition comprising the cell or cell-free system resulting from step (a) under conditions which allow for cleavage of the target RNA molecule, thereby producing two or more target RNA fragments; and (c) determining location(s) in which the target RNA molecule is cleaved.

In particular embodiments, cleaved target RNA molecules are isolated from the cell or cell free system prior to step (c). In other embodiments, cleavage sites in cleaved RNA molecules are determined by the sequence of all or part of individual cleaved target RNA molecules. Sequence data may be obtained by (a) determining the nucleotide sequence of: (i) one or more of the target RNA fragments, or (ii) one or more terminal portions of one or more of the target RNA fragments; and (b) comparing the sequences determined in (a) to the sequence of the intact target RNA molecule. The nucleotide sequence at the 5′ and/or 3′ end of the target RNA fragment, when compared to the nucleotide sequence of the intact target RNA molecule, may be used to identify positions of RNAi cleavage in the target RNA molecule.

According to certain embodiments of the invention, instead of, or in addition to determining the nucleotide sequence of the target RNA fragments or terminal portions thereof, methods of the invention comprise determining the sizes of cleavage products of the target RNA molecule. The sizes of these cleavage products may then be compared to the size of the intact target RNA molecule to determine the locations along the intact target RNA molecule that correspond to each of the target RNA fragments, thereby identifying the sites (or probable sites) of RNAi cleavage. This aspect of the invention is especially useful when (1) there are relatively few cleavage sites in the target RNA molecule, (2) the target RNA molecule is relatively long and the dsRNA molecules in the mixed population of dsRNA molecules share sequence identity to only one region at or near a terminus of the target RNA molecules, and/or (3) the number of different dsRNA molecules is small (e.g., less than two, three, five, seven, or ten). As an example of (2) above, if the target RNA molecule is 4 kb in length, the mixed population of dsRNA molecules may share sequence identity over a 1.5 kb stretch at the 3′ end. Typically, this 3′ stretch will not include a polyA tail portion, if present. Thus, cleavage sites may be identified by analysis of the cleaved target RNA molecules that are 2.5 kb or larger.

According to certain embodiments of the invention, a mixed population of dsRNA molecules is utilized. For example, the invention includes methods for identifying one or more RNAi cleavage sites along a target RNA molecule comprising introducing a mixed population of dsRNA molecules into a cell comprising the target RNA molecule. This mixed population may comprise two or more non-identical dsRNA molecules, wherein the non-identical dsRNA molecules correspond to different regions of the same target RNA molecule. Further, the mixed population of dsRNA may comprise two or more non-identical dsRNA molecules, wherein the non-identical dsRNA molecules correspond to different target RNA molecules (e.g. , two, three, five, seven, ten, etc.). Additionally, when individual members of a mixed population of dsRNA molecules correspond to different target RNA molecules, these dsRNA molecules may correspond to different regions of one or more of the target RNA molecules.

The invention also includes methods for producing mixed populations of dsRNA molecules. According to certain embodiments, methods of the invention comprise: (a) incubating a first intact dsRNA molecule with an enzyme having RNase activity (e.g., a dicer enzyme), thereby producing a first set of two or more dsRNA fragments; (b) incubating a second intact dsRNA molecule with an enzyme having RNase activity, thereby producing a second set of two or more dsRNA fragments; and (c) combining the first set of two or more dsRNA fragments with the second set of two or more dsRNA fragments, thereby producing a mixed population of dsRNA molecules. The first and second intact dsRNA molecules may share sequence identity with a single target RNA molecule or different target RNA molecules.

The invention further includes mixed populations of dsRNA molecules. The invention includes mixed populations produced by any method. Mixed populations of the invention, in certain embodiments, comprise at least one first dsRNA molecule and at least one second dsRNA molecule. The first dsRNA molecule corresponds to all or part of a first target RNA molecule and the second dsRNA molecule corresponds to all or part of the first target RNA molecule and/or all or part of a second target RNA molecule, wherein the first and second dsRNA molecule differ in sequence by at least one nucleotide. In particular embodiments, the first and second dsRNA molecules share no regions of nucleotide sequence identity which are greater than 6, 8, 10, 15 or 20 nucleotides in length.

The invention also includes nucleic acids which participate in RNAi processes and methods for using such nucleic acids in in vivo and in vitro RNAi-mediated knock-down target RNA molecule concentrations. Examples of such nucleic acids include those which may be used as controls for monitoring RNAi processes, such as nucleic acids encoding all or part of one or more lamin A/C and/or all or part of a reporter or other detectable tag (e.g., a β-lactamase, a β-galactosidase, a luciferase, Green Fluorescent Protein (GFP), Red Fluorescent Protein (RFP), Yellow Fluorescent Protein (YFP), a LUMIO™ tag (also referred to as a FlAsH tag), a FLAG tag, a myc tag, a V5 epitope tag, a His tag, a negative selection marker, etc.). In many instances, these nucleic acid molecules (e.g., dsRNA molecules) will be shorter than the full length of the nucleic acid to which they correspond.

Reporter and other detectable tags used with the invention can, for examples, fall into two categories: reporters or tags which are detectable (1) by themselves (e.g., luciferase, GFP, YFP, RFP, etc.) or (2) in conjunction with another compound (e.g., a substrate).

One example of a fluorescent protein which may be used with the invention is referred to as “Emerald”, which is described in U.S. Patent Publication No. 2004/0014128, the entire disclosure of which is incorporated herein by reference. Other examples of fluorescent proteins which may be used in the practice of the invention are described, for example, in U.S. Patent Appl. No. 60/508,142, filed Oct. 1, 2003, the entire disclosure of which is incorporated herein by reference.

The invention further includes nucleic acid fusions wherein all or part of a nucleic acid encoding one gene product is fused to all or part of a nucleic acid encoding another gene product. Such nucleic acid fusions may be used to monitor and/or measure RNAi processes.

In certain embodiments, nucleic acid molecules of the invention will comprise one or more (e.g., one, two, three, four, five, six, etc.) recombination sites (e.g., att sites, frt sites, dif sites, psi sites, cer sites, and lox sites or mutants, derivatives and variants thereof, as well as various combinations of these) and/or one or more topoisomerase recognition sites or bound topoisomerase molecules. In some instances, a recombination site will be present such that is allows for the generation of a nucleic acid encoding one gene product is fused to all or part of a nucleic acid encoding another gene product. An example of such a nucleic acid is shown in FIG. 4A-4B.

Additionally, a combination or recombination sites and topoisomerase recognition sites will be present in a configuration which allows the generation of a nucleic acid encoding one gene product is fused to all or part of a nucleic acid encoding another gene product. For example, topoisomerase mediated ligation of nucleic acid molecules may be used to position nucleic acid encoding one gene product next to a recombination site. In particular embodiments, one part of a fusion RNA transcription product may be on one side of the recombination site and the other part of the fusion RNA transcription product may be on the other side of the recombination site.

In particular embodiments, the invention includes nucleic acid encoding all or part of a lamin A/C or 3′ untranslated regions of gene such as bovine growth hormone or HSV thymidine kinase transcription termination sequences fused to nucleic acid encoding all or part of a β-lactamase or other reporter. In more specific embodiments, the invention includes nucleic acid which encodes (1) a β-lactamase fused to (2) nucleic acid which encodes all or part of (i) a lamin A/C, (ii) a β-galactosidase, or (iii) another polypeptide. The invention further includes vectors and cells which contain these fusion nucleic acids, as well as cells which contain these vectors.

In particular embodiments, the nucleic acid which encodes either the β-lactamase or other polypeptide may be replaced with nucleic acid which encodes an amino acid sequence that facilitates rapid protein turnover (e.g., a PEST sequence). As another option, the β-lactamase or other polypeptide encoding nucleic acid remains in place and the expression product additional contains the amino acid sequence which facilitates rapid protein turnover. Rapid turnover of protein expression products is desirable in some instances because it allows for their rapid degradation. Thus, systems may be designed to generate proteins with short half-lives so that protein levels quickly reflect the amount of translation of a particular mRNA and the level of that mRNA in the cell. In other words, the use of protein expression products with a short half-lives allows for a correlation between protein concentrations and the amount of translation which is occurring from the particular mRNA. Of course, one factor which will affect the amount of translation is the amount of mRNA present. Thus, under appropriate circumstances, protein concentration levels will approximate the amount of translatable mRNA present. Protein expression products may be designed to have a half-life of between about 2 minutes and about 60 minutes, about 5 minutes and about 60 minutes, about 10 minutes and about 60 minutes, about 20 minutes and about 60 minutes, about 2 minutes and about 180 minutes, about 5 minutes and about 180 minutes, about 10 minutes and about 180 minutes, about 30 minutes and about 180 minutes, etc.

In many instances, the concentration levels of the protein expression product will be measured by measuring an enzymatic activity of the protein. Further, the half-life will often be measured by the amount of time it takes for a 50% decrease in the enzymatic activity being measured.

In specific embodiment, fusion nucleic acids of the invention include those which comprise (1) nucleic acid which encodes a protein that, upon transcription and/or translation results in the production of a functional reporter (e.g., a dominant selectable marker such as HSV thymidine kinase, etc.) or tag (component 1) and (2) nucleic acid which is involved in RNAi mediated degradation (component 2). Of course, additional nucleic acid which encodes other components (e.g., a polyA tail, a linker between components (1) and (2), an internal ribosome entry site, etc.) may also be present. Further, when the target RNA molecule is an mRNA, components (1) and (2) may be part of the same open reading frame such that RNAi-mediated cleavage of the target RNA molecule in component 1 results in cleavage within the coding region. Thus, the invention provides, in part, nucleic acids which are “tagged” with segments which participate in RNAi processes. These segments which participate in RNAi processes may be identified by methods described, for example, elsewhere herein. Components (1) and (2), referred to immediately above, may be present in any orientation (e.g., 5′ to 3′ or 3′ to 5′).

In particular instances, component 2 above may be individual members of a library. The invention thus includes target RNA molecules that are fusion nucleic acids in which component 2 is a library. In particular, methods of the invention include the use of a positive selection marker and a negative selection marker to select for target RNA molecules which engage in RNAi mediated RNA degradation when contacted with particular dsRNA molecules. In particular instances, the negative selection marker is a dominant selection marker (e.g., HSV thymidine kinase). For example, component 2 may comprise individual members of a library and component 1 may encode a conditionally toxic protein such HSV thymidine kinase. Cells which contain such nucleic acids may then be contacted with one dsRNA molecule or multiple dsRNA molecules which correspond to one or more members of the library. When RNAi mediated degradation of a target RNA molecule which encodes a toxic protein or lead to a deleterious phenotype occurs, the toxic effects of the target RNA molecule are lessened or eliminated. As a result, such methods result in selection for cells which contain a positive selection maker (e.g., neomycin resistance, etc.) and wherein target RNA molecules which result in a deleterious phenotype is lessen or eliminated by RNAi mediated RNA degradation.

In one specific embodiment, cells are transfected with plasmids which contain a neomycin resistance marker and individual members of a library in which the library members are transcribed as part of a fusion RNA in which another portion of the fusion RNA encodes HSV thymidine kinase in a format that allows for transcription (i.e., the target RNA molecule). Thus, when cells are grown under suitable conditions in the presence of neomycin, a compound which is converted to a toxin in the presence of HSV thymidine kinase (e.g., acyclovir, ganciclovir, etc.), and a population of dsRNA molecules which correspond to one or more individual members of the library, selection will occur in favor of cells which contain plasmids and express fusion RNA molecules which are degraded by one or more members of the population of dsRNA molecules. The invention includes methods such as those described above and nucleic acid molecules used in such methods (e.g., libraries, dsRNA molecules, etc.).

In more specific embodiments of the invention, when nucleic acid molecules used in the practice of the invention encode a protein, transcription and/or translation of this nucleic acid results in the production of a functional reporter or tag, encodes a reporter protein with β-lactamase activity (e.g., a cytoplasmic form of a β-lactamase) and the nucleic acid which is involved in RNAi mediated degradation is a nucleic acid which encodes all or part of a lamin A/C. In other specific embodiments of the invention, a reporter may be produced which has β-galactosidase activity and the nucleic acid which is involved in RNAi mediated degradation is a nucleic acid which encodes all or part of a β-lactamase.

Component (2) referred to above (i.e., nucleic acid which is involved in RNAi mediated degradation), may be of any length sufficient to allow for it to participate in RNAi processes. In many instances, component (2) will be from about 19 to about 200, from about 19 to about 150, from about 19 to about 100, from about 19 to about 75, from about 19 to about 50, from about 25 to about 100, from about 20 to about 50, from about 50 to about 200, from about 75 to about 300, from about 100 to about 600, from about 200 to about 500, from about 100 to about 5000, from about 50 to about 600, etc. nucleotides in length.

In particular embodiments, fusion nucleic acids of the invention are introduced into cells in an expressible format (e.g., as DNA vector) which can result in the constitutive or inducible production of RNA molecules corresponding thereto. Thus, the invention includes nucleic acids which encode fusion target RNA molecules operably linked to constitutive or regulatable promoters.

Fusion nucleic acids of the invention need not encode fusion proteins. For example, when a fusion nucleic acid molecule of the invention contains all or part of two different protein coding regions, this fusion nucleic acid may be structured such that (1) all or part of only one protein may be translated, (2) all or part of both proteins may be translated as separate proteins (e.g., one or more internal ribosome entry sites may be present), (3) all or part of both proteins may be translated as a fusion protein, or (4) neither protein is produced.

In particular embodiments, the invention includes expressing RNA in a cell and then contacting that RNA with nucleic acid molecules which result in cleavage of the RNA. Thus, in specific embodiments, the invention includes (1) introducing an expression vector into a cell under conditions which allow for transcription of an RNA, and (2) contacting the transcribed RNA with double-stranded nucleic acid (e.g., RNA or DNA) which is capable of mediating RNA interference based degradation of the transcribed RNA.

The invention further includes methods for monitoring and/or measuring RNAi processes which involve (1) introducing one or more fusion nucleic acids (e.g., DNA or RNA) referred to above into a cell; (2) exposing the fusion nucleic acids introduced into the cell in (1) or transcription products thereof to one or more double stranded nucleic acids which participate in RNAi processes and results in the degradation of the fusion nucleic acids or transcription products thereof; and (3) monitoring and/or measuring the progression, if any, of the RNAi processes.

The invention further comprises individual RNA molecules (e.g., dsRNA molecules) which correspond to particular target RNA molecules. One example of such a target RNA molecule is a mRNA molecule which encodes β-lactamase.

The invention also includes methods which employ control nucleic acid molecules (e.g., vectors). For example, when a vector which encodes a fusion transcript as described elsewhere herein is introduced into a cell, it may not always be possible to determine whether a particular level of signal associated with a reporter or other detectable tag results from RNAi mediate transcript degradation or low levels of transfection. Thus, methods of the invention also employ the introduction into cells of nucleic acid molecules which result in the expression of two or more reporters and/or other detectable tags. These reporters and/or other detectable tags may be encoded by the same nucleic acid molecule or different nucleic acid molecules which are co-transfected into cells. In many instances, the signals which are generated by these reporters and/or other detectable tags will be distinguishable so that it is readily determinable which signal is being generated by which reporters or other detectable tags. Thus, the invention provides ratio metric means for measuring RNAi mediated degradation of nucleic acid molecules. This ratio metric means for monitoring RNAi mediated degradation may be performed by comparing the change in the signal level of a reporter or other detectable tag which is the subject of or is expected to be the subject of RNAi mediated degradation with the signal level of a reporter or other detectable tag which not the subject of RNAi mediated degradation. This will often be done over a time course (e.g., 10 minutes to 3 hours, 30 minutes to 2 hours, 1 hour to 3 hours, etc.).

In particular instances, the reporters used in the above systems are two different forms of fluorescent proteins (e.g., GFP, YFP, RFP, etc.). These reporter may be selected such that they are excited by different wavelengths of light or are excited by the same wavelengths of light but emit wavelengths of light which are sufficiently distinct that it allows for differential identification.

The invention also includes kits and compositions comprising one or more dsRNA molecules. For example, the invention includes kits and compositions comprising a mixed population of dsRNA molecules.

Kits of the invention may comprise one or more additional components selected from the group consisting of, but not limited to, (1) one or more cells; (2) one or more reagents for introducing nucleic acid molecules into cells (e.g., LIPOFECTAMINE 2000™); (3) one or more enzymes having RNase activity (e.g., a dicer enzyme); (4) one or more enzymes having RNA polymerase activity; (5) one or more enzymes having DNA polymerase activity; (6) one or more restriction endonucleases; (7) one or more nucleotides; (8) one or more enzymes having DNase activity; (9) one or more buffers; (10) one or more RNA purification columns; (11) a poly A affinity resin (e.g., an oligo dT resin); (12) one or more RNA ligases; (13) one or more reagents (e.g., an enzyme) having reverse transcriptase activity; (14) one or more reagents which inhibit RNAse activity; (15) one or more RNA oligonucleotides; (16) one or more dsRNA molecules or one or more mixed populations of such molecules; (17) one or more DNA oligonucleotides; and (18) one or more sets of instructions for performing methods of the invention and/or using compositions of the invention.

Compositions of the invention (e.g., reaction mixtures, kits, etc.) may comprise one or more additional components selected from the group consisting of: (1) a reagent for introducing nucleic acid molecules into cells; (2) one or more cells; (3) one or more enzymes having RNase activity; (4) one or more enzymes having RNA polymerase activity; (5) one or more enzymes having DNA polymerase activity; (6) one or more restriction endonucleases; (7) one or more nucleotides; (8) one or more enzymes having DNase activity; (9) one or more buffers; one or more reagents have ligase activity; (10) one or more sets of instructions for performing methods of the invention and/or using compositions of the invention; and (11) one or more lysates (or extracts) obtained from one or more cells.

### BRIEF DESCRIPTION OF THE DRAWINGS/FIGURES

FIG. 1 shows a schematic representation of particular aspects of the invention. “UTR” refers to the 5′ and 3′ untranslated regions of the target RNA molecule. The ORF refers to an open reading frame which is present in the target RNA molecule.

FIG. 2 shows a schematic representation of additional aspects of the invention. This schematic shows three separate reaction tubes in which target RNA molecules are contacted with mixed populations of dsRNA molecules (represented by the “=” symbols). “UTR” refers to the 5′ and 3′ untranslated regions of the target RNA molecule. “GOI” refers to the gene of interest.

FIG. 3 shows a schematic representation of methods of the invention. The label “CAATC CGCTAT” indicates a cut site in the mRNA shown at the top of the figure. The label “OH” refers to hydroxyl groups located at the 3′ ends of nucleic acid molecules. The label “P” refers to phosphate groups located at the 5′ ends of nucleic acid molecules. “GSP” is an abbreviation for gene specific primer and “ASP” is an abbreviation for adapter specific primer. The process shown in this schematic presentation is described in Example 1.

FIG. 4A-4G shows particular features of the pSCREEN-iT™/lacZ-DEST destination vector (A) and the vector sequence (B-G) (SEQ ID NO: 1).

FIG. 5A-5G shows particular features of the pSCREEN-iT™/lacZ-GW/CDK2 destination vector (A) and the vector sequence (B-G) (SEQ ID NO: 2). In this instance CDK2 functions as the target gene.

FIG. 6A-6H shows results from lacZ screening vectors and their correlation with qRT-PCR data. SiRNAs from which qRT-PCR data had been previously generated at Sequitur were tested in cotransfections with a luc reporter and lacZ-ULTIMATE™ ORF screening vector fusions with or without a stop codon after lacZ. For each transfection, the reporters were also transfected alone (Rep. only) or in combination with a lacZ siRNA (lacZ-67) positive control or β-lactamase siRNA (β-lac18) negative control. (A-F) Normalized qRT-PCR and mean β-gal RFU/luc RLU± the standard error for (A) CDK2, (B) IKBKG, (C) PEN2, (D) PTP4A1, and (E-F) two independent MAP2K3 experiments. SiRNAs with mismatches to the MAP2K3 ULTIMATE™ ORF are indicated by asterisks. (G-H) Scatter plots comparing without stop (G) or with stop (H) screening vectors to qRT-PCR data (mismatched siRNAs were excluded).

FIG. 7 shows position effect in RNA-only fusion screening vectors. The ORF of human -actin was placed downstream of lacZ with or without a stop codon between the coding regions. GRIPTITE™ 293 cells were cotransfected with the screening vector and a luc reporter alone (Rep. only), or in conjunction with siRNAs targeting the positions indicated in the coding region of -actin. lacZ siRNA and β-lac18 siRNAs were used as controls as in FIG. 6. Activities are reported as mean ratios of β-galactosidase RFU to luc RLU± standard error.

FIG. 8A-8B shows data derived from a 200 base pair amplicon from β-lactamase was cloned into pCR®8/GW/TOPO® TA and recombined into pSCREEN-iT™/lacZ-DEST. The amplicon is out of frame with lacZ and terminates early in the β-lactamase sequence. (A) In separate experiments, GRIPTITE™ 293 MSR cells were transfected with the 200 base pair fusion clone or full length β-lactamase screening vectors. These plasmids were transfected alone (Rep. only) or with siRNAs targeting sites within the β-lactamase amplicon. (B) CHO cells stably expressing β-lac were transfected with lipid only (mock) or with 5 pmol siRNAs. Activities are reported as normalized mean β-galactosidase RFU or β-lactamase blue/green ratios± standard error.

FIG. 9 shows data derived using positive and negative STEALTH™ controls. GRIPTITE™ 293 cells were cotransfected with pSCREEN-iT™/lacZ-GW/CDK2 and the RNAi reagents indicated as previously described. Activities are reported as normalized mean β-galactosidase RFU± standard error.

FIG. 10 shows the amino acid sequence (SEQ ID NO: 3) of an example of an altered polypeptide having β-lactamase activity that is retained in the cytosol of prokaryotic and/or eukaryotic cells, and the nucleotide sequence (SEQ ID NO: 4) that endodes the amino acid sequence.

FIG. 11 shows the results of a Dicer reaction buffer optimization experiment.

FIG. 12 shows Dicer activity in the presenceof inhibitory reagents.

FIG. 13 shows an outline of a possible dicer stimulatory reagent screen.

FIG. 14A-14B shows a schematic of the process of siRNA target sequence identification. The top nucleotide sequence shown in 14B is SEQ ID NO: 5, the middle nucleotide sequence shown in 14B is SEQ ID NO: 6, and the bottom nucleotide sequence shown in 14B is SEQ ID NO: 7.

FIG. 15A-15B shows the results of an experiment involving the amplification of RISC cleavage fragments following transfection of a Luciferase-specific siRNA.

FIG. 16A-16C shows the results of an experiment involving amplification of RISC cleavage fragments following transfection of mixed populations of siRNAs.

FIG. 17 shows the RISC cleavage sites in Luciferase (SEQ ID NO: 8) following transfection of siRNAs.

FIG. 18 shows the RISC cleavage sites in Luciferase (SEQ ID NO: 8) following transfection of d-siRNA.

FIG. 19A-19C illustrates the efficient knockdown of Luciferase expression by emperically identified siRNA. The top sequence in 19A “ID1” is SEQ ID NO: 9; The bottom sequence in 19A “ID1” is SEQ ID NO: 10; The top sequence in 19A “ID2” is SEQ ID NO: 11; The bottom sequence in 19A “ID2” is SEQ ID NO: 12; The top sequence in 19A “ID3” is SEQ ID NO: 13; The bottom sequence in 19A “ID3” is SEQ ID NO: 14.

FIG. 20 shows the RISC cleavage sites in Luciferase (SEQ ID NO: 8) following transfection of a mixture of effective siRNAs.

FIG. 21 shows the RISC cleavage sites in LacZ (SEQ ID NO: 15) following transfection of i-siRNA.

FIG. 22 illustrates the efficient knockdown of LacZ expression by emperically identified sirRNAs.

FIG. 23 shows the cleavage site mapping from synthetic hairpins (GL2=SEQ ID NO: 16; GL2-22-AS=SEQ ID NO: 17) using target ID.

FIG. 24 is a schematic representation of exemplary RNAi screening vectors.

FIG. 25 is a schematic representation of the use of pScreen-iT/lacZ-GW/DEST or pScreen-iT/lacZ-GW/DT in a Gateway or Topo cloning reaction.

FIG. 26 shows an outline of an exemplary process for high throughput screening of a vector clone collection.

FIG. 27 shows the results of an exemplary clone quantitation and validation experiment.

FIG. 28 is a shematic showing how the BLOCK-iT™ RNAi target screening system works.

FIG. 29 shows the nucleotide sequence of the recombination region of an expression clone resulting from a pScreen-iT/lacZ-DEST x entry clone reaction (SEQ ID NO: 18). The amino acid sequence encoded by a portion of this region is also shown. (SEQ ID NO: 19)

FIG. 30 shows an example of expected results in which several syntheic siRNAs are screened targeting the human CDK2 gene.

FIG. 31 shows a sample β-galactosidase standard curve.

FIG. 32 shows a summary of the features of the pENTR-gus vector.

### DETAILED DESCRIPTION OF THE INVENTION

The present invention includes methods for identifying one or more RNAi cleavage sites along a target RNA molecule. Also included are mixed populations of double-stranded RNA (dsRNA) that are useful for identifying RNAi cleavage sites, methods for producing dsRNA mixed populations, kits for identifying RNAi cleavage sites, and compositions comprising dsRNA molecules.

Methods for Identifying RNAi Cleavage Sites

The term “RNAi cleavage site”, as used herein, refers to a position along a target RNA molecule at which the target RNA molecule is cleaved following the introduction of a dsRNA molecule into a cell or cell-free system containing the target RNA molecule, wherein the dsRNA molecule has a nucleotide sequence that corresponds to at least a portion of the target RNA molecule. (See, e.g., Kawasaki et al., Nucl. Acids Res. 31:981-987 (2003); Tuschl, Chembiochem 2:239-245 (2001)).

As used herein, a “portion” of a nucleic acid molecule, e.g., a “portion of the target RNA molecule,” is intended to mean any part of the nucleic acid molecule that has at least one less nucleotide than the entire nucleic acid molecule and that is at least 10 nucleotides in length. A “portion” may be expressed as a fraction of the nucleic acid molecule; e.g., a “portion” of a nucleic acid molecule may be one half, one third, one quarter, one fifth, one sixth, one seventh, one eighth, one tenth, one twelfth, one sixteenth, one twentieth, one thirtieth, one fiftieth, one one hundredth, one two hundredth, one five hundredth, one one thousandth, one two thousandth, etc. of the nucleic acid molecule.

The term “target RNA molecule”, as used herein, refers to any RNA molecule which is chosen for cleavage or degradation. For example, when an investigator is interested in examining RNAi-mediated silencing of a particular gene of interest, the messenger RNA (mRNA) molecule that is transcribed from the gene of interest may be selected by the investigator as the target RNA molecule. The target RNA molecule can be an RNA molecule that is found naturally within a cell or cell-free system, or it can be an RNA molecule that is not naturally found within a cell or cell-free system. The target RNA molecule can be encoded by and/or transcribed from DNA or RNA. The target RNA molecule may be double-stranded, single-stranded, or may be partially double-stranded and partially single-stranded. In many embodiments of the invention, the target RNA molecule is single-stranded. The target RNA can be encoded by a chromosome, by a plasmid, or by any other nucleic acid-containing molecule. Also, the target RNA molecules may be essentially any RNA molecule, for example, a mRNA molecule a ribozyme, a tRNA molecule, a small nuclear RNA molecule, a microRNA molecule, a small nucleolar RNA molecule, etc. In many instances, the nucleotide sequence of the target RNA molecule and/or the sequence of a nucleic acid molecule that encodes the target RNA molecule is known prior to the practice of methods of the invention.

Target RNA molecules may represent, for example, transcription products of genomic DNA, expressed sequence tags, cDNAs, etc.

The term “intact dsRNA molecule” refers to a dsRNA molecule which has not been process into smaller fragments. For example, a blunt ended dsRNA molecule which is 900 nucleotides in length and is formed by annealing two separate single-stranded RNA molecules, each of which are also 900 nucleotides in length, is an intact dsRNA molecule. This molecule may be used in methods of the invention directly or may be processed first using, for example, an enzyme with RNase activity to generate fragments which may then be used in methods of the invention. In particular instances, a “Dicer” enzyme may be used to process intact dsRNA molecules, resulting in the production of dsRNA molecules which are 21 to 23 nucleotides in length. When, for example, RNA molecules are synthesized chemically and then annealed to each other to form dsRNA molecules which are 21 to 23 nucleotides in length, these dsRNA molecules are “intact dsRNA molecule”. These dsRNA molecules may then be used in methods of the invention without prior processing, for example, by an enzyme with RNase activity. In particular instances, intact dsRNA molecule which are longer than 23 nucleotides in length may be used in methods of the invention. For example, cells of organisms such as C. elegans do not undergo apoptosis when exposed to dsRNA molecules which are over about 30 nucleotides in length. Thus, in vivo methods for mapping dsRNA-mediated cleavage of target RNA molecules, for example, in such cells need not involve the introduction of dsRNA which are 21 to 23 nucleotides in length.

As used herein, the term “dsRNA molecule” is intended to mean a double-stranded RNA molecule comprising two strands that interact with one another through base-pair interactions. The two strands may be referred to as, e.g., a “top strand” and a “bottom strand,” or a “sense strand” and an “antisense strand.” The two strands may be connected to one another or they may be separate. Thus, both siRNA (short interfering RNA) molecules and shRNA (short hairpin RNA) molecules are both considered to be dsRNA molecules. For sake of clarity siRNA molecules are composed of two separate strands and shRNA molecules are formed by intramolecular hybridization. In many instances, the dsRNA molecules of the invention will not possess any mismatched base pairs (a mismatched base pair occurs, e.g., when an A is not paired with a U, or vice versa; or when a G is not paired with a C, or vice versa); however, the invention includes the use of dsRNA molecules having 1, 2, 3, 4, 5, 6, 7, 8, 9, 10, or more mismatched base pairs. The term “dsRNA molecule” includes dsRNA molecules having any number of nucleotides. dsRNA molecules can comprise one or more modified nucleotides (e.g., 2′-aminouridine, 2′-deoxythymidine, 5′-iodouridine, 2′-O-methyl, etc.). In other words, one or more of the nucleotides present in dsRNA molecules used in methods and compositions of the invention may be nucleotides other than the four nucleotides commonly found in RNA.

dsRNA molecules that are included within or used in the practice of the invention may comprise a single RNA molecule, e.g., a single RNA molecule in a hairpin conformation (thereby being double stranded). Alternatively, dsRNA molecules of the invention may comprise multiple (one, two, three, four, etc.) individual RNA molecules. Typically, when dsRNA molecules comprise multiple RNA molecules, they will comprise two RNA molecules: a sense strand and an antisense strand.

The term “dsRNA molecule” as used herein, includes siRNA molecules. The term “siRNA molecule” is intended to mean a dsRNA molecule with a length of between 15 and 30 nucleotides. Typically, siRNA molecules are between 21 and 23 nucleotides in length. (McManus and Sharp, Nature Reviews 3:737-747 (2002)). In instances where there is a two nucleotide overhang at each end of the dsRNA molecule and the total length of the dsRNA molecule is between 21 and 23 nucleotides, the length of each of the individual strands of the molecule siRNA molecules will be between 19 and 21 nucleotides. As a specific example, if the dsRNA molecule is 23 nucleotides in length and there are 3′ overhangs on each end of two nucleotides each, then the individual strands of the dsRNA molecule will each be 21 nucleotides in length and they will share 19 nucleotides of sequence complementarity. siRNA molecules may be used or included in any embodiments of the invention that use, include, or make reference to “dsRNA molecules.” The term “dsRNA molecules” also includes short-hairpin RNA (shRNA) molecules. shRNA molecules will typically have double-stranded regions of between 15 and 30 nucleotides and a loop which connects the stands which form this double-stranded region. This interconnecting loop will often be four, five, six, seven, eight, nine, ten, eleven, twelve, thirteen, fourteen or fifteen nucleotides in length. In some instances, the loops may be between 10 and 25, 10 and 30, 10 and 40, 10 and 20, 20 and 40, or 15 and 25 nucleotides in length.

The two strands of dsRNA molecules may not have fully complementary nucleotide sequences. For example, there may be at least 1, 2, 3, or 4 mismatches between the strands. These mismatches, when present, may be localized internal in the dsRNA molecule, may be at a terminus, or may be interspersed within the ds RNA molecule. When the mismatches are localized at a terminus, they may be localized at either the 5′ or 3′ terminus of the antisense strand in the double-stranded RNA molecules. Further, in some instances, the antisense strand of the dsRNA molecule will correspond more to the target RNA molecule's sequence than the sense strand of the dsRNA molecule. For example, the antisense strand of the dsRNA molecule may be 100% identical to the to sequence of the target RNA molecule and the sense strand may be less than 100% identical to the to sequence of the target RNA molecule. In many such instances, the dsRNA molecule will contain mismatches between the antisense and sense strands.

In particular embodiment, double-stranded nucleic acid molecules composed of one strand which is DNA and the other strand which is RNA, and related nucleic acids containing modified nucleotides, may be used in methods and compositions of the invention instead of dsRNA molecules. Thus, the invention includes the use of DNA/RNA hybrids in methods and compositions of the invention.

dsRNA molecules that are included within or used in the practice of the invention will often have nucleic acid sequences that correspond to all or a portion of the target RNA molecule.

dsRNA molecules used in the practice of the invention may contain chemical modifications, for example, as described below.

As used herein, a dsRNA molecule is regarded as “corresponding” to all or a portion of a target RNA molecule (or to a sequence encoded by a DNA molecule) if the nucleotide sequence of at least one of the strands of the dsRNA molecule is at least 90% identical to a sequence found within the target RNA molecule or complement thereof (or sequence encoded by a DNA molecule). Typically, the region(s) of dsRNA molecules of the invention which correspond to that of a target RNA molecule will be the double-stranded region and, in particular instances, overhangs. Thus, in many instances, nucleotides present in the loop, for example, of a shRNA molecule will not correspond to the target RNA molecule.

As used herein, the term “isolated”, when used in reference to a nucleic acid, means that the nucleic acid has been removed from its native environment. For example, recombinant DNA molecules contained in a vector are considered isolated for the purposes of this invention. Isolated RNA molecules include in vivo or in vitro RNA transcripts of recombinant DNA molecules. Isolated nucleic acid molecules according to the present invention further include such molecules produced synthetically.

For example, a dsRNA molecule will “correspond” to a portion of a target RNA molecule if the nucleotide sequence of at least one of the strands of the dsRNA molecule is at least 90% to 100% (e.g., 90%, 91%, 92%, 93%, 94%, 95%, 96%, 97%, 98%, 99%, or 100%) identical to a nucleotide sequence found within the target RNA molecule. Typically, the corresponding portions which are compared will be at least 18 nucleotides in length. In many instances, the nucleotide sequence of one of the strands of the dsRNA molecules is identical (i.e., 100% identical) to a nucleotide sequence found within the target RNA molecule.

By a dsRNA molecule having a nucleotide sequence at least, for example, 90% “identical” to a reference nucleotide sequence (e.g., a nucleotide sequence found within the target RNA molecule), it is intended that the nucleotide sequence of at least one of the strands of the dsRNA molecule is identical to the reference sequence except that the nucleotide sequence may include up to 10 nucleotide alterations per each 100 nucleotides of the nucleotide sequence of the reference nucleic acid molecule. In other words, to obtain a dsRNA molecule having a nucleotide sequence at least 90% identical to a reference nucleotide sequence, up to 10% of the nucleotides in the reference sequence may be deleted or substituted with another nucleotide, or a number of nucleotides, up to 10% of the total nucleotides in the reference sequence, may be inserted into the reference sequence. These alterations of the reference sequence may occur, e.g., at the 5′ or 3′ ends of the reference nucleotide sequence and/or anywhere between those terminal positions, interspersed either individually among nucleotides in the reference sequence and/or in one or more contiguous groups within the reference sequence.

As a practical matter, whether any particular nucleic acid molecule is at least 90%, 91%, 92%, 93%, 94%, 95%, 96%, 97%, 98% or 99% identical to a nucleotide sequence found within the target RNA molecule can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The best overall match between a query sequence (a sequence of a strand of a dsRNA molecule) and a subject sequence, also referred to as a global sequence alignment, can be determined using, for example, the FASTDB computer program based on the algorithm of Brutlag et al., Comp. Appl. Biosci. 6:237-245 (1990). In a sequence alignment, the query and subject sequences are both DNA sequences. An RNA sequence can be compared by converting U's to T's. The result of the global sequence alignment is in percent identity. Suitable parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty=0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by the results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence are calculated for the purposes of manually adjusting the percent identity score.

For example, a 90 base subject sequence is aligned to a 100 base query sequence to determine percent identity. The deletions occur at the 5′ end of the subject sequence and, therefore, the FASTDB alignment does not show a match/alignment of the first 10 bases at the 5′ end. The 10 unpaired bases represent 10% of the sequence (number of bases at the 5′ and 3′ ends not matched/total number of bases in the query sequence), so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal, so that there are no bases on the 5′ or 3′ ends of the subject sequence which are not matched/aligned with the query. In this case, the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

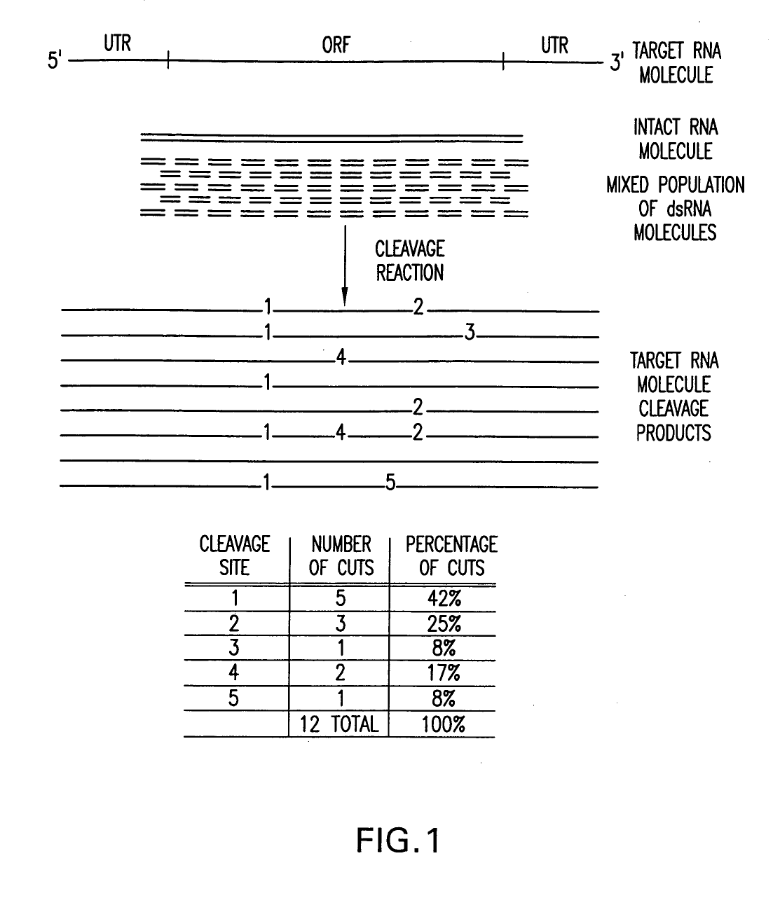

As used herein, the phrase “recombination site” refers to a recognition sequence on a nucleic acid molecule which participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP which is a 34 base pair sequence comprised of two 13 base pair inverted repeats (serving as the recombinase binding sites) flanking an 8 base pair core sequence. (See FIG. 1 of Sauer, B., Curr. Opin. Biotech. 5:521-527 (1994).) Other examples of recognition sequences include the attB, attP, attL, and attR sequences described herein, and mutants, fragments, variants and derivatives thereof, which are recognized by the recombination protein λ Int and by the auxiliary proteins integration host factor (IHF), FIS and excisionase (Xis). (See Landy, Curr. Opin. Biotech. 3:699-707 (1993).)

Recombination sites may be added to molecules by any number of known methods. For example, recombination sites can be added to nucleic acid molecules by blunt end ligation, PCR performed with fully or partially random primers, or inserting the nucleic acid molecules into an vector using a restriction site which flanked by recombination sites.

As used herein, the phrase “recombinational cloning” refers to methods, such as that described in U.S. Pat. Nos. 5,888,732 and 6,143,557 (the contents of which are fully incorporated herein by reference), whereby segments of nucleic acid molecules or populations of such molecules are exchanged, inserted, replaced, substituted or modified, in vitro or in vivo. Such cloning method will often be in vitro methods.

As used herein, the term “topoisomerase recognition site” or “topoisomerase site” means a defined nucleotide sequence that is recognized and bound by a site specific topoisomerase. For example, the nucleotide sequence 5′ -(C/T)CCTT-3′ is a topoisomerase recognition site that is bound specifically by most poxvirus topoisomerases, including vaccinia virus DNA topoisomerase I, which then can cleave the strand after the 3′-most thymidine of the recognition site to produce a nucleotide sequence comprising 5′-(C/T)CCTT-PO₄₋TOPO, i.e., a complex of the topoisomerase covalently bound to the 3′ phosphate through a tyrosine residue in the topoisomerase (see Shuman, J. Biol. Chem. 266:11372-11379, 1991; Sekiguchi and Shuman, Nucl. Acids Res. 22:5360-5365, 1994; each of which is incorporated herein by reference; see, also, U.S. Pat. No. 5,766,891; PCT/US95/16099; and PCT/US98/12372 also incorporated herein by reference). In comparison, the nucleotide sequence 5′-GCAACTT-3′ is the topoisomerase recognition site for type IA E. coli topoisomerase III.

As used herein, the term “library” refers to a collection of nucleic acid molecules (circular or linear). In one embodiment, a library may comprise a plurality of nucleic acid molecules (e.g., two, three, four, five, seven, ten, twelve, fifteen, twenty, thirty, fifty, one hundred, two hundred, five hundred one thousand, five thousand, or more), which may or may not be from a common source organism, organ, tissue, or cell. In another embodiment, a library is representative of all or a portion or a significant portion of the nucleic acid content of an organism (a “genomic” library), or a set of nucleic acid molecules representative of all or a portion or a significant portion of the expressed nucleic acid molecules (a cDNA library or segments derived therefrom) in a cell, tissue, organ or organism. A library may also comprise nucleic acid molecules having random sequences made by de novo synthesis, mutagenesis of one or more nucleic acid molecules, and the like. Such libraries may or may not be contained in one or more vectors (e.g., two, three, four, five, seven, ten, twelve, fifteen, twenty, thirty, fifty, etc.).

The schematic representation set out in FIG. 1 illustrates various aspects of the invention. As described in FIG. 1, a target RNA molecule which contains an open reading frame and 5′ and 3′ untranslated regions, is contacted with either an intact RNA molecule or a mixed population of dsRNA molecules under conditions which allow for RNAi mediate cleavage of the target RNA molecule. In both instances, the sequences of the intact RNA molecule or the individual members of the mixed population of dsRNA molecules correspond to the target RNA molecules. The result is a series of cleavage products of the target RNA molecule. As one skilled in the art would recognize, not all of the individual target RNA molecules are cleaved at the same location(s). The locations of the cleavage sites in the individual, cleaved target RNA molecules (also referred to herein as “target RNA fragments”) may then be determined by any number of means, including those described elsewhere herein.

As discussed in more detail below, methods for identifying cleavage sites may be based upon methods which identify or isolate fragments based upon termini of undigested target RNA molecules. In such instances, internal cleavage sites may be under represented when the data is generated. Using the schematic shown in FIG. 1 for purposes of illustration, cleavage site 4 may be under represented due to it residing between two other cleavage sites in the target RNA molecule.

A number of things may be done to lessen or prevent the above under representation of data. One rectification involves the use of cleavage site detection methods which employ terminal portions of the undigested target RNA molecules in the cleavage site identification process (e.g., amplification employing primers which hybridize to sequences at or near one or both termini) conditions under which a substantial majority (e.g., greater than 95%) of the target RNA molecules are cleaved either once or twice.

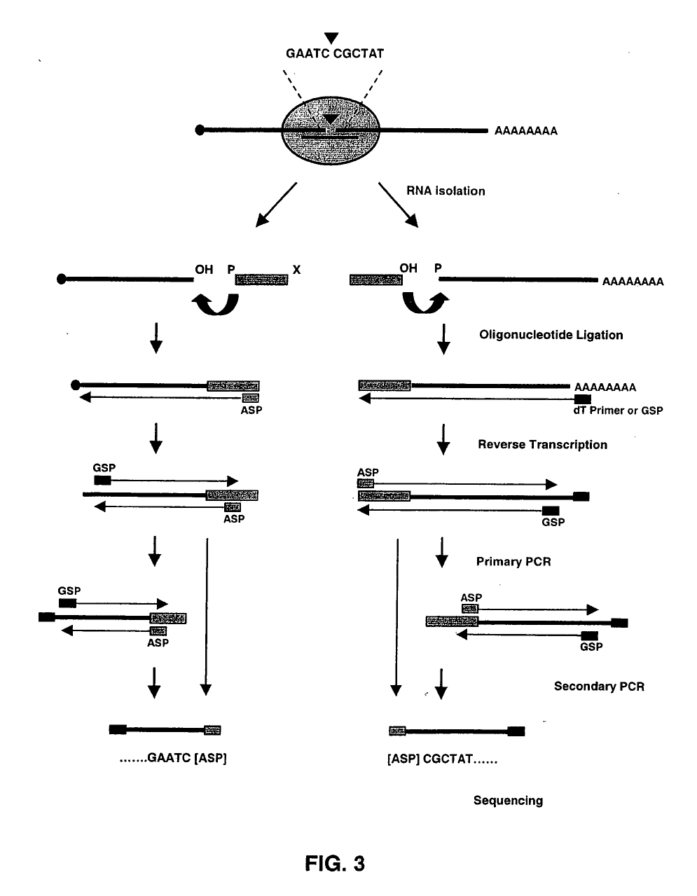

Another rectification involves the use of cleavage site identification methods which do not rely upon terminal portions of the undigested target RNA molecules as part of the cleavage site identification process. For example, mixed populations of primers (e.g., at least 3, 4, 5, 6, 7, 8, 9, 10, 11, 12, etc primer which differ in nucleotide sequences) designed to hybridize at various locations within the target RNA molecules may be used for reverse transcription, then cleavage sites may be identified using these reverse transcripts. For example, using the schematic shown in FIG. 3 for purposes of illustration, multiple gene specific primers (GSPs) may be employed in similar processes.

In one aspect, the invention therefore includes methods for identifying one or more RNAi cleavage sites along a target RNA molecule. According to certain embodiments, methods of the invention comprise: (a) introducing one or more double-stranded RNA (dsRNA) molecules into a cell, or combining one or more dsRNA molecules in a cell-free system which allows for in vitro dsRNA-mediated cleavage of RNA molecules; (b) incubating the composition comprising the cell or cell-free system resulting from step (a) under conditions which allow for cleavage of a target RNA molecule which corresponds to some or all of the dsRNA molecules produced in step (a), thereby producing two or more target RNA fragments; and (c) determining the location(s) in which the target RNA molecules is cleaved. In some instances, methods for identifying one or more RNAi cleavage sites along a target RNA molecule will involve the use of compositions to which purified RISC complexes are added.

In some embodiments, the cleaved, target RNA molecule is isolated from the cell or cell free system prior to step (c). In particular embodiments, cleavage sites in the cleaved, target RNA molecule are determined by the sequence of all or part of one or more target RNA fragments. Sequence data may be obtained by (a) determining the nucleotide sequence of: (i) one or more of the target RNA fragments, or (ii) one or more terminal portions of one or more of the target RNA fragments; and (b) comparing the sequences determined in (a) to the sequence of the uncleaved target RNA molecule. The nucleotide sequence at the 5′ and/or 3′ end of a target RNA fragment, when compared to the nucleotide sequence of the target RNA molecule, may be used to identify the positions of RNAi cleavage in the target RNA molecule. Methods for performing the above and identifying dsRNA molecules which mediate cleavage at specific locations in particular target RNA molecules are described in more detail below.

Any cell in which dsRNA-mediated cleavage of RNA molecules can occur can be used in the context of the invention. Exemplary cells include mammalian cells (e.g., mouse cells, human cells, etc), insect cells (e.g., Drosophila melanogaster cell), yeast cells (e.g., Schizosaccharomyces pombe cells), protozoan cells (e.g., T. brucei cells), Caenorhabditis elegans cells, and plant cells (e.g., A. thaliana cells). In most instances, cells used in the practice of methods of the invention will express an endogenous Dicer protein. Also, in most instances, cells used in the practice of methods of the invention will contain all of the components necessary to form RNA-initiated silencing complexes (RISC). One example of such cells are Drosophila S2 cells. See, e.g., Liu et al., Science 301:1921-1925 (2003), the entire disclosure of which is incorporated herein by reference.

Exemplary mammalian cells that can be used in the context of the invention include, e.g., somatic cells, including blood cells (erythrocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells). Mammalian germ line cells (spermatocytes and oocytes) may also be used, as may the progenitors, precursors and stem cells that give rise to the above-described somatic and germ cells. The cells can be immortalized cells.

The type of cell chosen for the practice of methods of the invention will vary with the system that the user employs and the particular application. The type of dsRNA molecule used will vary with the particular application. For example, when dsRNA molecules of greater than about 30 nucleotides in length are introduced into mammalian cells, these cells may undergo apoptosis. However, the same is not true of cells of Caenorhabditis elegans or Drosophila melanogaster. Thus, characteristics of the dsRNA molecules used will vary with the cell type employed. In addition, when mammalian cells are used in the practice of the invention, in most instances, the dsRNA molecules will be less than 30 nucleotides in length to limit the amount of apoptotic cell death in the cell population.

Cells used in the practice of the invention may be cultured cells. Exemplary cultured cells for use in the context of the invention include: CHO, HEK, HeLa, 3T3, rat FB, Caco2, HL-5, 293, T cells, Cos, HaCaT, MEF, U-2 OS, H1299, C6, Daoy, DBTRG-05MG, DI-TNC1, HCN-1A, Neuro-2a, PC-12, SK-N-MC, SVG p12, and C-33A cells (see McManus and Sharp, Nature Reviews 3:737-747 (2002) and references cited therein).

dsRNA molecules can be introduced into cells by any method that will transfer nucleic acid molecules to the intracellular confines of the cell. Exemplary methods include the use of lipophilic agents (e.g., OLIGOFECTAMINE, LIPOFECTAMINE, LIPOFECTAMINE PLUS, LIPOFECTAMINE-2000 (Invitrogen Corporation, Carlsbad, Calif.)), non-cationic lipid based carriers (TRANSIT-TKO (Mirus Corporation, Madison, Wis.)), and electroporation. Certain cells are capable of taking up dsRNA molecules simply by soaking the cells in a solution containing the dsRNA molecules, or by feeding organism (e.g., worms) that comprise the dsRNA molecules (Timmons and Fire, Nature 395:854 (1998); Tabara et al., Science 282:430-431 (1998)).

Cell-free systems which allow for in vitro dsRNA-mediated cleavage of RNA molecules include systems which comprise a mixture of one or more components that facilitate the cleavage of RNA molecules through the interaction of dsRNA molecules with RNA molecules. Such cell-free systems can be a synthetic combination of the necessary components to carry out dsRNA-mediated cleavage of RNA molecules. Alternatively, at least some of the components of the cell-free system can be obtained from cells. For example, a cell-free system may comprise or consist of a cell extract or lysate. Exemplary cell-free systems include cell extracts from D. melanogaster embryos (Zamore et al., Cell 101:25-33 (2000); Tuschl et al., Genes Dev. 13:3191-3197 (1999)), extracts from D. melanogaster S2 cells (Bernstein et al., Nature 409:363-366 (2001); Hammond et al., Nature 404:293-296 (2000)), extracts from C. elegans cells (Elbashir et al., Genes Dev. 15:188-200 (2001); Ketting et al., Genes Dev. 15:2654-2659 (2001)) and extracts of HeLa cells (Yang et al., Proc. Natl. Acad. Sci. USA 99 :9942-9947 (2002)). Other cell-free systems include immunoprecipitates from cell extracts (e.g., D. melanogaster cell extracts, C. elegans cell extracts) that contain one or more enzymes having RNase activity (e.g., one or more RNase III activities such as that of a Dicer enzyme) (Nykanen et al., Cell 107:309-321 (2001)).

dsRNA molecules used in the practice of the invention typically have strands that are from about 15 nucleotides in length to about 3,000 nucleotides in length. For example, one or both of the strands of the dsRNA molecules may be 15, 16, 17, 18, 19, 20, 21, 22, 23, 24, 25, 26, 27, 28, 29, 30, 32, 34, 36, 38, 40, 45, 50, 100, 200, 300, 500, 700, 850, 950, 1,100, 1,200, 1,400, 1,600, 1,800, 2,000, 2,300, 2,500, 2,750 or 2,900 nucleotides in length. As additional examples, one or both of the strands of the dsRNA molecules may be between 19 and 23, 18 and 25, 19 and 28, 21 and 28, 19 and 50, 25 and 50, 30 and 60, 40 and 90, 50 and 100, 75 and 125, 100 and 200, or 150 and 300 nucleotides in length. Further, these dsRNA molecules may be siRNA molecules short-hairpin RNA molecules (shRNA molecules), or long-hairpin RNA molecules (lhRNA molecules). When the dsRNA molecules used are shRNA molecules, the above numbers will typically refer to the double-stranded regions of the shRNA molecules or lhRNA molecules. Wherever, the terms “shRNA molecules” or “ilhRNA molecules” are employed, the other may optionally be used.

As used herein, the term shRNA molecules means that the double-stranded region of the RNA molecules is less than about 50 nucleotides in length. Further, the term lhRNA molecules means that the double-stranded region of the RNA molecules is greater than a bout 50 nucleotides in length.

The invention thus includes methods for identifying shRNA molecules and/or lhRNA molecules which function efficiently in RNAi mediated cleavage processes. Generally, these shRNA molecules and/or lhRNA molecules will be present in a mixed population of shRNA molecules and/or lhRNA molecules.

The invention further includes the use of libraries of nucleic acid molecules which are designed to express shRNA molecules, lhRNA molecules, and dsRNA molecules. In many instances, these libraries will be composed of DNA molecules (e.g., DNA vectors). The invention further includes the libraries of nucleic acid molecules referred to above, as well as individual members of these libraries.

Libraries of nucleic acid molecules designed to express shRNA molecules, lhRNA molecules, and dsRNA molecules may be formed by any number of means. For example, one method for forming vectors which express shRNA molecules involves (1) fragmentation of a nucleic acid molecule (e.g., by sonication, digestion with one or more restriction endonucleases, etc.), (2) ligating nucleic acid fragments resulting from step (1) to an oligonucleotide which forms a loop and contain a recognition site for a type IIs restriction endonuclease (e.g., MmeI) positioned so that a cut occurs 18, 19, 20, 21, 22, 23, 24, or 25 nucleotides into the ligated nucleic acid fragments, (3) digesting the ligation product with the particular type IIs restriction endonuclease, (4) ligating a second oligonucleotide which forms a loop to the cut end, (5) amplifying the resulting nucleic acids molecules to generate closed circular double-stranded molecules which encode shRNA molecules corresponding to sequences of the nucleic acid fragments, (6) removing the loops by, for example, restriction endonuclease digestion (e.g., using endonucleases which recognize sites in the oligonucleotides), and (7) operably connecting the cleaved nucleic acid molecules obtained by step 6 to a promoter. In many instances, step (7) will be performed by inserting the cleaved nucleic acid molecules obtained by step 6 into a vector. In many instances, the resulting vectors will be designed such that transcription of the inserts is driven by an RNA polymerase III promoter. A system similar to that described above is set out in Sen et al., Nature Genetics, 36:183-189 (2004). In brief, Sen et al. describe a method for producing siRNA constructs using individual genes or pool of genes. Libraries used in the practice of the invention may be generated using one gene (e.g., a single ORF) or multiple genes (e.g., 2, 3, 4, 5, 6, 7, 8, 9, 10, etc. ORFs). The invention includes the use of such libraries, as well as the libraries themselves.

A method which may be used to prepare libraries of nucleic acid molecules designed to express shRNA molecules, lhRNA molecules, and dsRNA molecules involves the insertion of double-stranded nucleic acid segments between opposing promoters. For example, one or more (e.g., 1, 2, 3, 4, 5, 6, 7, 8, 9, 10, etc.) different double-stranded nucleic acid segments may be generated which correspond to a particular target RNA molecule and then positioned between RNA polymerase III promoters. The opposing promoters may then be used to produce sense and antisense strands of the double-stranded nucleic acid segments. The resulting transcripts may then hybridize to form dsRNA molecules. One systems which has been designed to produce nucleic acid molecules such as those described above is set out in Zheng et al., Proc. Natl. Acad. Sci. (USA) 101:135-140 (2004). Zheng et al. describes the positioning of gene specific oligonucleotides between opposing U6 and H1 promoters in a vector. The gene specific oligonucleotides are generated such that they have different four nucleotide overhangs on each end. This allows for directional insertion into the vector. The inserted gene specific oligonucleotides have five regions from left to right: (1) an overhang, (2) a strand specific TTTTT terminator sequence which allows for the termination of transcription driven by the promoter which will ultimately be positioned to the right of the oligonucleotide, (3) a sequence which corresponds to a target RNA molecule, (4) a strand specific TTTTT terminator sequence which allows for the termination of transcription driven by the promoter which will ultimately be positioned to the left of the oligonucleotide, and (5) an overhang. Of course, the U6 and H1 promoters may be present on separate nucleic acid molecules and ligated to the gene specific oligonucleotides to generate a linear construct in which the promoters flank the oligonucleotides.

While section (3) of each gene specific oligonucleotide will typically correspond to only one target RNA molecule, section (3) of different gene specific oligonucleotides may correspond d to one or more target RNA molecules. Further, section (3) may be 18, 19, 20, 21, 22, 23, 24, 25, 26, 27, 28, 29, or 30 nucleotides in length. In addition, section (3) may be a randomly generated sequence. Methods which may be used to generate randomly generated sequences are known in the art. One such method is referred to as “dirty bottle” synthesis. In dirty bottle synthesis, more than one nucleoside is present in the synthesis reaction and incorporated in oligonucleotides being formed at one or more locations.

Dirty bottle synthesis, for example, may be used to generate single-stranded oligonucleotides in which section (3) is a random sequence (e.g., A, T, C, or G at each location). Sections (1) and (5) of the oligonucleotide may be flanked by (a) primer binding sites and (b) restriction endonuclease recognition sites which generate the overhangs of sections (1) and (5). The primer binding sites, in conjunction with primers and one or more polymerases, may then be used to convert the above single-stranded oligonucleotides to double-stranded form. The double-stranded oligonucleotides may then be digested with appropriate restriction endonucleases to generate suitable overhangs in sections (1) and (5). These oligonucleotides may then be positioned between opposing promoters, for example, as described above. Using such methods, it should be possible to generate fully random libraries which express dsRNA molecules. These libraries may then be screened using methods of the invention to identify nucleic acid molecules (e.g., vectors) which participate in RNAi-mediated degradation of particular transcripts. The invention includes the use of libraries such as those described above, as well as the libraries themselves.

Similarly, synthetic single-stranded nucleic acid molecules may also be positioned between opposing promoters (e.g., inserted into a vector), such as the opposing RNA polymerase III promoters described above, and/or cloned. Such positioning of the single-stranded synthetic oligonucleotides may be done, for example, by a number of means known by those skilled in the art and allows for the incorporation of randomly synthesized oligonucleotides without prior generation of complimentary strands such as by PCR amplification. One method of positioning single-stranded oligonucleotides between opposing promoters and/or cloning single-stranded oligonucleotides employs methods utilizing topoisomerases to join the ends of DNA (or RNA). Topoisomerase mediated DNA or RNA end-joining are described, for examples, in U.S. Patent Publication No. 2004/0058417 and in U.S. Pat. Nos. 6,548,277 and 6,653,106, the entire disclosures of which are incorporated herein be reference. An example of how topoisomerase end joining may be used to insert synthetic-single stranded DNA oligonucleotide into a vector with opposing RNA polymerase III promoters is as follows. Vaccinia topoisomerase is covalently attached to the 3′ end of one strand of one end of the double stranded vector. The 5′ end of the complimentary strand contains a single-stranded overhang of 1 or more bases, extending past the 3′ end of the base covalently attached to the topoisomerase molecule. The synthetic oligonucleotide to be inserted into the vector contains a 5′ hydroxyl group and one or more 5′ nucleotides which are complementary to the 5′ overhang at the topoisomerase adapted end of the vector. The 3′ end of the oligonucleotide is joined to the vector by ligase utilizing a 5′ phosphate group from the vector. In specific instances, the result is a circular vector results which contains a single-stranded region corresponding to most of the oligonucleotide. The single-stranded region may then be converted to double-stranded form by, for examples, (1) treatment with a polymerase or (2) by nucleic acid repair mechanisms after transformation into a cell (e.g., E. coli). In other instances, the single-stranded oligonucleotide attached to the vector by topoisomerase at one end is converted to double-stranded form prior to the joining of the second set of ends to create a circular double-stranded DNA molecule.

Additionally, libraries may be generated, as an invention in this application, by positioning fragmented DNA of the appropriate size between opposing promoter (e.g., insertion into a vector), such as that described above with opposing RNA polymerase III promoters. In such cases, the fragmented DNA may be transcribed by opposing promoters and, thus, does not have to be “duplicated” in a DNA fragment prior to cloning, as is often necessary when a single RNA polymerase III promoter is used to generate short hairpin (shRNA) transcripts.

Mixed populations of shRNA molecules and/or lhRNA molecules may be formed by any number of methods. For example, DNA molecules (e.g., vectors) which encode shRNA molecules and/or lhRNA molecules may be introduced into cells which contain target RNA molecules. After expression of the encoded shRNA molecules and/or lhRNA molecules, cleavage sites in the target RNA molecules may then be identified. The locations of these cleavage sites may then be used to identify shRNA molecules and/or lhRNA molecules involved in the cleavage reactions. As another example, shRNA molecules and/or lhRNA molecules may be generated by in vitro transcription. The transcripts may then be introduced either into a cell or a cell free reaction mixture which contains a target RNA molecule. Again, RNAi mediated cleavage sites may be then be identified and used to identify the shRNA molecules and/or lhRNA molecules involved in the cleavage reaction.

The two strands of the dsRNA molecules may have the same length as each other, or they may have different lengths. The dsRNA molecules may have overhangs on one end or both ends. These overhangs may be 1, 2, 3, 4, 5, 6, 7, 8, etc. nucleotides in length. Further, the dsRNA molecules may one blunt end or two blunt ends. Thus, dsRNA molecules used in the practice of the invention may be 23 nucleotides in length and composed of two RNA strands one of which is 21 nucleotides in length and the other one of which is 23 nucleotides in length. In such a case, there may be a two nucleotide overhang on one end and the other end may be blunt.

dsRNA molecules used in the practice of the invention may be produced by any number of methods, including synthetically or enzymatically. Methods for synthetically producing dsRNA molecules are known in the art. Commercial suppliers of synthetic dsRNA molecules include Invitrogen Corporation (Carlsbad, Calif.), Proligo (Hamburg, Germany), Ambion Inc. (Austin, Tex.), Qiagen (Valencia, Calif.), Dharmacon Research (Lafayette, Colo.), Pierce Chemical (Rockford, Ill.), Glen Research (Sterling, Va.), ChemGenes (Ashland, Mass.), Cruachem (Glasgow, UK), and others.

dsRNA molecules may be produced from DNA vectors. (Lee et al., Nature Biotechnol. 20:500-505 (2002); Sui et al., Proc. Natl. Acad. Sci. USA 99:5515-5520 (2002)). Thus, the invention includes methods for identifying RNAi cleavage sites comprising introducing one or more DNA vectors (e.g., a mixed population of DNA vectors) into a cell or cell-free system, wherein the vectors encode one or more dsRNA molecules. One example of a vector system which may be used to produce shRNA molecules, for example, is the BLOCK-IT™ Lentiviral RNAi Expression System (Invitrogen Corporation, Carlsbad, Calif., cat. nos. K4943-00 and K4944-00). In many instances, the dsRNA will be transcribed using a RNA polymerase III promoter such as a U6 or H1 promoter. As with other vectors of the invention or used in methods of the invention, these vectors may comprise one or more recombination sites.

dsRNA molecules used in the methods and compositions of the invention may also be produced by cleaving longer “intact” dsRNA molecules with an enzyme having RNase activity. The expression “enzyme having RNase activity” is intended to mean a substance (e.g., a substance comprising a protein or nucleic acid molecule) that, when combined with an RNA molecule (either a double stranded or a single stranded RNA molecule), catalyzes the hydrolysis of one or more of the chemical bonds between adjacent nucleotides or nucleotide base pairs. Exemplary enzymes having RNase activity include “Dicer,” e.g., Dicer from nematodes, fruit flies, fission yeast, flowering plants, and mammals, including mouse and human (Wilson et al., Nature 368:32-38 (1994); Rotondo and Frendewey, Nucl. Acids Res. 24:2377-2386 (1996); Jacobsen et al., Development 126:5231-5243 (1999); Kawasaki et al., Nucl. Acids Res. 31:981-987 (2003)). Other enzymes having RNase activity that can be used to produce dsRNA molecules for use with the present invention include prokaryotic RNase III enzymes (Yang et al., Proc. Natl. Acad. Sci. USA 99:9942-9947 (2002)). Products which may be used to generate dsRNA molecules suitable for use in the practice of the invention include the BLOCK-IT™ RNAi TOPO® Transcription Kit and the BLOCK-IT™ Dicer RNAi Transfection Kit (Invitrogen Corp. Carlsbad, Calif., see, e.g., cat. nos. K3500-01, K3600-01, and K3650-01). These products allow one to attach T7 promoters to the 5′ and 3′ termini of a DNA molecule, followed by the production of RNA using these promoters. The resulting single-stranded RNA molecules may then be annealed to each other to form what is referred to herein as an intact dsRNA molecule and then either used directly or processed to a smaller size. As noted in the literature associated with the above products, the single-stranded RNA molecule may be purified prior to annealing and the dsRNA molecules may be purified prior to used in RNAi processes.

Intact dsRNA molecules that can be cleaved by enzymes having RNase activity (to produce smaller dsRNA molecules for use with the methods according to this aspect of the invention) can be synthesized, or they can be produced by transcription from DNA or RNA templates. (U.S. Pat. No. 3,597,318; U.S. Pat. No. 3,582,469; U.S. Pat. No. 5,795,715; Bhattacharyya, Nature 343:484 (1990); Milligan, Nucl. Acids Res. 21:8783 (1987); Provost et al., EMBO J. 21:5864-5874 (2002); Yang et al., Proc. Natl. Acad. Sci. USA 99:9942-9947 (2002)). Intact dsRNA molecules can also be extracted from biological material, e.g., from viruses (Dulieu et al., J. Virol. Meth. 24:77-84 (1989)) and yeasts (Fried et al., Proc. Natl. Acad. Sci. USA 75:4225 (1978)).

In certain embodiments, methods of the invention comprise the use of a mixed population of dsRNA molecules. The expression “mixed population of dsRNA molecules” is intended to mean a composition comprising two or more non-identical dsRNA molecules. Two dsRNA molecules are regarded as “non-identical dsRNA molecules” if the nucleotide sequence of at least one of the strands of the first dsRNA molecule differs from both strands of the second dsRNA molecule by at least one nucleotide. Non-identical dsRNA molecules will often have nucleotide sequences that correspond to different portions of the same target RNA molecule.

A mixed population of dsRNA molecules may comprise any number (greater than one) of non-identical dsRNA molecules. In certain embodiments, the mixed population of dsRNA molecules comprises between 2 and 1000, between 2 and 500, between 2 and 200, between 5 and 1000, between 5 and 500, between 5 and 400, between 5 and 300, between 5 and 200, between 5 and 100, between 5 and 50, between 10 and 1000, between 10 and 500, between 10 and 400, between 10 and 300, between 10 and 200, between 10 and 100, between 10 and 80, between 10 and 60, between 10 and 40, or between 10 and 20 non-identical dsRNA molecules.

In certain embodiments, non-identical dsRNA molecules of the mixed population may each correspond to different segments of the same target RNA molecule. In some cases, non-identical dsRNA molecules of the mixed population will all correspond to different nucleotide sequences within the same portion of the target RNA molecule. For example, non-identical dsRNA molecules of the mixed population may correspond to different nucleotide sequences found within the same one half, one third, one quarter, one fifth, one sixth, one seventh, one eighth, one tenth, one twelfth, one sixteenth, one twentieth, one thirtieth, one fiftieth, one one hundredth, etc., of the target RNA molecule. Using the schematic in FIG. 1 for purposes of illustration, the mixed population of dsRNA molecules shown therein correspond to the ORF and the 5′ end of the 3′ UTR of the target RNA molecule.

In other embodiments, at least some of the non-identical dsRNA molecules of the mixed population of dsRNA molecules will correspond to different nucleotide sequences from different target RNA molecules. For example, a mixed population of dsRNA molecules can comprise at least one dsRNA molecule corresponding to a specific portion of a first target RNA molecule, and at least one dsRNA molecule corresponding to a specific portion of a second target RNA molecule. The first and second target RNA molecules may be, for example, mRNA molecules which are (1) transcribed from DNAs which encode two distinct polypeptides which do not share substantial regions of homology or (2) splice variants of the same transcription product.

In at least certain embodiments of the invention, after dsRNA molecules (e.g., a mixed population of dsRNA molecules) are introduced into a cell or are combined with a cell-free system, the cell or the cell-free system containing the dsRNA molecules is incubated under conditions sufficient to allow cleavage of a target RNA molecule. The conditions sufficient to allow cleavage of a target RNA molecule are known by persons of ordinary skill in the art and include, e.g., incubation for about 30 seconds to about 96 hours at a temperature of about 16 C to about 60 C. The exact times and temperatures of incubation will depend on the types of cells/cell-free systems used and the characteristics of the target RNA molecule and of the dsRNA molecules used. In many instances, the temperature will be the optimal growth temperature of the cell type used. Exemplary conditions include incubation temperature of about 16° C., 25° C., 27° C., 37° C., or 42° C, as well as ranges of from about 16° C. to about 37° C., from about 22° C. to about 37° C., from about 25° C. to about 37° C., from about 25° C. to about 42° C., or from about 27° C. to about 37° C. Incubation times may vary from 30 seconds, 1 minute, 5 minutes, 30 minutes, 1 hour, 2 hours, 3 hours, 5 hours, 10 hours, 12 hours, 14 hours, 16 hours, 18 hours, 20 hours, 24 hours or 36 hours, as well as ranges of from about 1 minute to about 336 hours, from about 10 minute to about 336 hours, from about 30 minute to about 336 hours, from about 1 hour to about 336 hours, from about 1 minute to about 72 hours, from about 1 hour to about 72 hours, from about 6 hours to about 72 hours, from about 10 hours to about 72 hours, from about 24 hours to about 72 hours, from about 1 hour to about 24 hours, from about 2 hour to about 24 hours, from about 4 hours to about 24 hours, from about 6 hours to about 24 hours, from about 8 hours to about 24 hours, from about 10 hours to about 24 hours, etc.

In certain instances, it may be advantageous to use a range of times and/or temperatures of incubation. By adjusting the time and/or the temperature of incubation, the extent of dsRNA-mediated cleavage may be controlled.

The cleavage of the target RNA molecule will produce two or more target RNA fragments. The expression “target RNA fragment” is intended to mean an RNA molecules, produced by cleavage of a target RNA molecule. The number of target RNA fragments produced from each target RNA molecule depends upon the number of times the target RNA molecule is cleaved. For example, if a target RNA molecule is cleaved only once, then two target RNA fragments are produced. If the target RNA molecule is cleaved twice, then three target RNA fragments are produced, etc.

The number of times the target RNA molecule is cleaved depends upon numerous factors including (1) the incubation conditions (referred to above), (2) the dsRNA molecules used, and (3) the degree of susceptibility of the target RNA molecule to dsRNA-mediated cleavage by the dsRNA molecules used. While not wishing to be bound by theory, points (2) and (3) above are believed to be inter-related.

According to certain embodiments of the invention, after cells containing the dsRNA molecules are incubat ed under conditions that allow for cleavage of the target RNA molecule into two or more target RNA fragments, RNA is released from the cells. As used herein, the term “released” means removing RNA from the cell so that it is accessible to reagents (e.g., nucleotide molecules, enzymes, etc.). Released RNA comprises target RNA fragments as well as possibly other RNA species. In certain embodiments, released RNA will be the total RNA from the cell.

In certain embodiments, RNA is released from cells by treating the cells in a manner that disrupts the integrity of the cell membrane. For example, the cells can be treated with one or more reagents that disrupt the cell membrane. One example of such a reagent is water, which can be used to induce osmotic shock. The cells can also be subjected to physical disruption of the cell membrane to release RNA from the cells (e.g., sonication, etc.). Any known manner of disrupting cell membranes can be used to release RNA from cells.

It is not necessary in the context of the present invention for the RNA to be isolated or purified from the cells or cell-free systems; however, according to some embodiments, the invention includes methods which comprise isolating and/or purifying RNA from the cells or cell-free systems. Methods for isolating and/or purifying RNA are known in the art, including methods involving hybridization of RNA to a probe to form a hybrid molecule, and separating the hybrid molecule from the remaining components (e.g., by immobilizing the probe to a bead or other surface or substrate). In certain embodiments, methods of the invention comprise isolating total RNA from cells or cell-free systems. An exemplary method for isolating total RNA is the guanidine isothiocyanate/acid-phenol method. (Chomczynsk i and Sacchi, Anal. Biochem. 162:156 (1987)). An improvement of the Chomczynski and Sacchi method is the TRIzol Reagent method (Invitrogen Corporation, Carlsbad, Calif., see, e.g., cat. nos. 15596-018 and 15596-026). (Chomczynski, Biotechniques 15:532 (1993)). Other products which may be used to purify RNA is the S.N.A.P. Total RNA Isolation Kit (Invitrogen Corporation, Carlsbad, Calif., see, e.g., K1950-01 and K1950-05), Concert 96 RNA Purification System (Invitrogen Corporation, Carlsbad, Calif., see, e.g., 12173-011), or RNA Catcher kits (Sequitur Corp, Natick, Mass., an Invitrogen company, see, e.g., 7001).

In certain instances, a DNase enzyme may be used in the process of RNA release, isolation or purification to remove or reduce DNA contamination. It may also be advantageous to include RNase inhibitors, proteases, and/or protease inhibitors.

Once target RNA molecules have undergone dsRNA-mediate cleavage and are purified, if necessary, the location(s) of the cleavage sites are determined. In many instances, it can be determined from these cleavage sites which dsRNA molecules mediated cleavage of the target RNA molecule. For example, in higher eukaryotic cells, when cleavage of a target RNA molecule is mediated by dsRNA molecules as part of a RNA-induced silencing complex, the target RNA molecule is cleaved at a location which corresponds to the position between the 10th and 11th nucleotide of the antisense guide strand of the particular dsRNA molecule involved in the cleavage reaction (Elbashir et al., EMBO Jour. 20(23):6877-6888 (2001)). Thus, identification of a cleavage site in a target RNA molecules, in effect, results in the identification of the dsRNA molecule which mediated the cleavage reaction.

The identification of cleavage sites in target RNA molecules may be done by any number of means. One methods for identifying cleavage sites is by determining the sequence of all or part of the target RNA fragments. In many instances, the target RNA fragments will be reverse transcribed into DNA prior to determination of their sequences. Also, when the nucleotide sequence of the target RNA molecule is known, identification of a cleavage site will generally not require that the entire sequence of the target RNA fragment be determined. Thus, in particular embodiments, following the incubation of cells or a cell-free system under conditions sufficient to allow cleavage of a target RNA molecule, and after releasing and/or isolating RNA from the cells or cell-free system (if appropriate), the nucleotide sequence of: (i) one or more of the target RNA fragments; or (ii) one or more terminal portions of one or more of the target RNA fragments may be determined. The expression “terminal portion” of a target RNA fragment is intended to mean part of the target RNA fragment having a length that is at least one nucleotide less than the length of the entire target RNA fragment but that includes at least four (e.g., four, five, six, seven, eight, nine, ten, etc.) nucleotides at the 5′ or 3′ ends of the target RNA fragment. As noted above, the sequence of the target RNA fragments and/or the sequence of the terminal portion(s) of the target RNA fragments will generally reveal the nucleotide sequence at the position of RNAi cleavage.

Determining the nucleotide sequence of one or more target RNA fragments and/or one or more terminal portions of one or more target RNA fragments can be accomplished by a variety of methods known to those of ordinary skill in the art. Exemplary methods are discussed elsewhere herein.

Following the incubation of the cells or cell-free systems under conditions sufficient to allow cleavage of the target RNA molecule, and following the release of the RNA from the cell (unless a cell-free system is used), the target RNA fragments will generally be found within a mixture of different RNA molecules (e.g., dsRNA molecules, tRNA, rRNAs, mRNAs, etc.). For instance, when RNA is released from a cell or when total RNA is isolated, the released and/or isolated RNA will generally include the target RNA fragments, target RNA molecules that were not cleaved, and other RNA molecules. Likewise, when a cell-free system is used, the cell-free system may comprise, in addition to target RNA fragments, other RNA molecules that were included in the cell free system cell, as well as target RNA molecules that were not cleaved. Therefore, it may be advantageous to distinguish or separate one or more of the target RNA fragments from other RNA molecules prior to determining their sequence. One methods of separating a nucleic acid which corresponds to the sequence of a single target RNA fragment from nucleic acids corresponding to other fragments is by introducing DNA which corresponds to the target RNA fragment into a vector and then amplifying the vector either in vitro or in vivo.

Other methods involve the physical separation of RNA molecules from each. In many instances, this separation will occur prior to reverse transcription of one or more of the separated RNA molecules. Physical separation may occur by connecting a purification entity such as biotin or digoxigenin. Such a purification entity may be connected to the RNA prior or subsequent to RNAi mediated cleavage.

Purification entities may be introduced into target RNA molecules or target RNA fragments by any number of means. For example, the RNA may be synthesized in the present of one or more nucleotides which contain the purification entity. Further, a purification entity may be added to the 3′ end, 5′ end or 3′ and 5′ ends as part of an oligonucleotide (e.g., DNA, RNA, etc.) which is connected to the RNA. Methods for addition purification entities to RNA are described for example in U.S. Patent Publication Nos. 2003/0044822 and 2003/0104467, the entire disclosures of which are incorporated herein by reference. The invention thus also includes methods for purifying RNA which contains one or more purification entities. These methods will often include binding of the purification entity to another entity to separate the RNA using ligand/anti-ligand association. For example, one anti-ligand for biotin is avidin.

Additionally, or alternatively, it may be advantageous to synthesize nucleic acid molecules (DNA or RNA), e.g., nucleic acid molecules, that are complementary to one or more of the target RNA fragments or terminal portions thereof. The complementary nucleic acid molecules can be distinguished or separated from other nucleic acid molecules using a variety of methods, and then their sequences can be determined. The complementary nucleic acid molecules may be labeled. From the sequence of the complementary nucleic acid molecules, the sequence of the target RNA fragments or terminal portions thereof can be easily ascertained.

dsRNA molecules used in the practice of the invention may contain chemical modifications. Typically such chemical modifications will be (1) on the bases, (2) between in the linkages between the ribose or deoxyribose sugars of one or both strands (e.g., substitute linkages), or (3) on the ribose or deoxyribose sugars of one or both strands.

As used herein, the term “linkage” includes a naturally occurring, unmodified phosphodiester moiety (—O—(PO₂⁻)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47).

Modifications of the ribose or deoxyribose sugars include modifications at the 2′ position. Typically this will involve replacement of the 2′ OH group. Modified dsRNA molecules used herein may contain one or more 2′-fluoro, 2′-O-methyl, 2′-O-ethyl, and/or 2′-O-propyl groups.

As one skilled in the art would understand dsRNA molecules are composed of either a single molecules which engages in intramolecular hybridization or two separate molecules which associate with each other. Accordingly, for a given first oligonucleotide strand, a number of complementary second oligonucleotide strands are permitted according to the invention. For example, in the tables set out below, a targeted and a non-targeted oligonucleotide are illustrated with several possible complementary oligonucleotides. The individual nucleotides may be 2′-OH RNA nucleotides (R) or the corresponding 2′-O-methyl nucleotides (M), and the oligonucleotides themselves may contain mismatched nucleotides (lower case letters).

(i) Targeted Oligonucleotide:

TABLE 1

| | | |
|---|---|---|
| First Strand: | CCCUUCUGUCUUGAACAUGAG | (SEQ ID NO: 20) |
| Second | CTgATGTTCAAGACAGAAcGG | (SEQ ID NO: 21) |
| Strand: | MMMMMMMMMMMMMMMMMMMMM | |
| (methyl | CTgATGTTCAAGACAGAAcGG | (SEQ ID NO: 21) |
| groups:) | RRRRRRRRRRRRRRRRRRRDD | |
| | CTCAUGUUCAAGACAGAAGGG | (SEQ ID NO: 22) |
| | RRRRRRMMMMMMMMMRRRRRR | |
| | CTCAUGUUCAAGACAGAAGGG | (SEQ ID NO: 22) |
| | MMMMMMRRRRRRRRRMMMMMM | |
| | CTCAUGUUCAAGACAGAAGGG | (SEQ ID NO: 22) |
| | RMRMRMRMRMRMRMRMRMRMR | |

(ii) Non-Targeted Oligonucleotide:

TABLE 2

| | | |
|---|---|---|
| First Strand: | GAGTACAAGTTCTGTCTTCCC | (SEQ ID NO: 23) |
| Second | GGcAAGACAGAACTTGTAgTC | (SEQ ID NO: 24) |
| Strand: | MMMMMMMMMMMMMMMMMMMMM | |
| (methyl | GGGAAGACAGAACTTGTACTC | (SEQ ID NO: 25) |
| groups:) | RRRRRRMMMMMMMMMRRRRRR | |
| | GGGAAGACAGAACTTGTACTC | (SEQ ID NO: 25) |
| | MMMMMMRRRRRRRRRMMMMMM | |
| | GGGAAGACAGAACTTGTACTC | (SEQ ID NO: 25) |
| | RMRMRMRMRMRMRMRMRMRMR | |

Another example of further modifications that may be used in conjunction with 2′-O-methyl nucleomonomers are modification of the sugar residues themselves, for example alternating modified and unmodified sugars, particularly in the sense strand.

The invention further includes double stranded nucleic acid molecules (e.g., RNA molecules) which have structures defined by the following formula:

TABLE 3

| | |
|---|---|
| First Strand | X₁₅₋₃₀ |
| Second Strand | A₀₋₂₅X₀₋₂₅B₀₋₂₅ |

In the formula set out above, X, A, and B are nucleotides (e.g., A, G, C, U, etc.). Also, either of the first strand or the second strand may be a sense strand. As a results, either of the first strand or the second strand may be an antisense strand. Further, X is typically a nucleotide which has no modifications on the base or sugar. Further, A and/or B are nucleotides which may independently contain one or more base or sugar modifications. These modifications may be any modifications known in the art or described elsewhere herein. Examples of sugar modifications include ribose modifications at the 2′ position such as 2′-O-propyl (P), 2′-O-methyl (M), 2′-O-ethyl (E), and 2′-fluoro (F). Generic examples of nucleic acid molecules of the invention include those with the following:

TABLE 4

XXXXXXXXXXXXXXXXXXXX
AXXXXXXXXXXXXXXXXXXB
XXXXXXXXXXXXXXXXXXXX
AAXXXXXXXXXXXXXXXXBB
XXXXXXXXXXXXXXXXXXXX
AAAXXXXXXXXXXXXXXBBB
XXXXXXXXXXXXXXXXXXXX
AAAAXXXXXXXXXXXXBBBB
XXXXXXXXXXXXXXXXXXXX
AAAAXXXXXXXXXXXXXXBB
XXXXXXXXXXXXXXXXXXXX
AAXXXXXXXXXXXXXBBBBB
XXXXXXXXXXXXXXXXXXXX
AAAAAAAAAAAAAAAAAAAA
XXXXXXXXXXXXXXXXXXXX
AAAAAAAXXXBBBBBBBBBB

Examples of nucleic acid molecules of the invention (e.g., dsRNA molecules) which contain specific modifications include those with the following modifications, in which X represents an unmodified nucleotide, P represents 2′-O-propyl, M represents 2′-O-methyl, E represents 2′-O-ethyl, and F represents 2′-fluoro:

TABLE 5

XXXXXXXXXXXXXXXXXXXXXXXXX
PPMMXXXXXXXXXXXXXXXXEEMMM
XXXXXXXXXXXXXXXXXXXXXXXXX
EEEEXXXXXXXXXXXXXXXXEEMMM
XXXXXXXXXXXXXXXXXXXXXXXXX
PPEEXXXXXXXXYXXXXXXXEEMMM
XXXXXXXXXXXXXXXXXXXXXXXXX
EEEEEXXXXXXXXXXXXXXXEEEEE
XXXXXXXXXXXXXXXXXXXXXXXXX
PPPPPPPXXXXXXXXXXXPPPPPPP
XXXXXXXXXXXXXXXXXXXXXXXXX
FFPPPXXXXXXXXXXXXXXXPPPFF
XXXXXXXXXXXXXXXXXXXXXXXXX
MPPPPPPPPPPPPPPPPXXXPPPPM
XXXXXXXXXXXXXXXXXXXXXXXXX
FFFFFXXXXXXXXXXXXXXXFFFFF
XXXXXXXXXXXXXXXXXXXXXXXXX
PEEPEEMPXXXXXXXXXPMEEPEEP
XXXXXXXXXXXXXXXXXXXXXXXXX
MEXXXXXXXXXXXXXXMMMMMMMMM
XXXXXXXXXXXXXXXXXXXXXXXXX
MXXXXXXXXXXXXXXXMMMMMMMMM
XXXXXXXXXXXXXXXXXXXXXXXXX
EEXXXXXXXXXXXXXXXEEEEEEEE

In some embodiments, the length of the sense strand can be 29, 28, 27, 26, 25, 24, 23, 22, 21, 20, 19, or 18 nucleotides. Similarly, the length of the antisense strand can be 29, 28, 27, 26, 25, 24, 23, 22, 21, 20, 19, or 18 nucleotides. Further, when a double-stranded nucleic acid molecule (e.g., a dsRNA molecule) is formed from such sense and antisense molecules, the resulting duplex may have blunt ends or overhangs of 0, 1, 2, 3, 4, 5, 6, 7, 8, 9, 10, 11, 12, 13, or 14 nucleotides on one end or independently on each end. Further, double stranded nucleic acid molecules of the invention may be composed of a sense strand and an antisense strand wherein these strands are of lengths described above, and are of the same or different lengths, but share only 10, 11, 12, 13, 14, 15, 16, 17, 18, 19, or 20 nucleotides of sequence complementarity. By way of illustration, in a situation where the sense strand is 20 nucleotides in length and the antisense is 25 nucleotides in length and the two strands share only 15 nucleotides of sequence complementarity, a double stranded nucleic acid molecule may be formed with a 10 nucleotide overhang on one end and a 5 nucleotide overhang on the other end.

Double-stranded oligonucleotides (e.g., dsRNA molecules) of the invention include STEALTH™ RNAs which may be obtained from either Sequitur Inc. (Natick, Mass.), recently acquired by Invitrogen Corporation (Carlsbad, Calif.) or Invitrogen Corporation directly. STEALTH™ RNAs are described in U.S. Provisional Application No. 60/540,552, filed on Feb. 2, 2004 and entitled “DOUBLE-STRANDED OLIGONUCLEOTIDES”.

According to certain embodiments of the invention, the nucleotide sequence of (i) one or more of the target RNA fragments or (ii) one or more terminal portions of one or more of the target RNA fragments may be determined by a method comprising: (a) synthesizing one or more DNA molecules complementary to one or more of the target RNA fragments or to a terminal portion of one or more of the target RNA fragments, thereby producing one or more complementary DNA molecules; and (b) sequencing the complementary DNA molecules. The complementary nucleic acid molecules may be cloned into a vector prior to sequencing. The sequence of the complementary DNA molecules will be the complement of the sequence of the target RNA fragments or a terminal portion thereof.

The complementary DNA molecules may be labeled, e.g., by adding one or more labeled nucleotides to the DNA synthesis reaction. The synthesis of the complementary DNA molecules can be accomplished, for example, by providing a mixture comprising: (1) a nucleic acid primer that hybridizes to one or more portions of the target RNA fragments, (2) nucleotides, and (3) an enzyme that is capable of producing a DNA molecule from an RNA molecule template. Exemplary enzymes that can be used in this regard include, e.g., reverse transcriptases. The complementary DNA molecules can subsequently be amplified using known methods of DNA amplification (e.g., polymerase chain reaction (PCR)).

In another aspect of the invention, the process of determining the sequence of the target RNA fragments or terminal portions thereof may, in certain instances, involve the addition of one or more “linker” nucleic acid molecules to one or both of the ends of the target RNA fragments or to a nucleic acid molecule complementary thereto. Often, the nucleic acid sequence of the linker will be known so that one or more primers complementary to the linker (or portion thereof) can be used to amplify the target RNA fragments or to create a nucleic acid molecule that is complementary to the target RNA fragments. The linker may also be used to isolate the target RNA fragments, e.g., by using a nucleic acid probe having a nucleic acid sequence complementary to the linker (or portion thereof). After hybridizing to the probe to the linker, the hybridized molecule can be isolated, e.g., by immobilizing the probe to a bead or other substrate or surface. 138 Methods for determining the nucleotide sequence of (i) one or more of the target RNA fragments or (ii) one or more terminal portions of one or more of the target RNA fragments may comprise the process known as “RACE” (rapid amplification of cDNA ends). (Frohman et al., Proc. Natl. Acad. Sci. USA 85:8998 (1988); Ohara et al., Proc. Natl. Acad. Sci. USA 86:5673 (1989); Loh et al., Science 243:217 (1989)). Either 5′ RACE or 3′ RACE can be used in the context of the present invention to produce and amplify one or more complementary DNA molecules from the target RNA fragments or terminal portions thereof. The complementary DNA molecules can then be sequenced to determine the nucleotide sequence of one or more target RNA fragments or terminal portions thereof.

According to certain other embodiments of the invention, the nucleotide sequence of (i) one or more of the target RNA fragments or (ii) one or more terminal portions of one or more of the target RNA\fragments is determined by using a nuclease protection assay to identify target RNA\fragments or to identify a nucleic acid molecule that is complementary thereto. Methods according to this aspect of the invention may comprise: (a) hybridizing one or more of the target RNA fragments to at least a portion of a labeled single stranded nucleic acid molecule, wherein the labeled single stranded nucleic acid molecule comprises a nucleotide sequence that is complementary to one or more of the target RNA fragments; (b) digesting any portion of the labeled single-stranded nucleic acid molecule that is not bound to one or more of the target RNA fragments through base-pair interactions (i.e., the single stranded portion of the labeled nucleic acid molecule), thereby producing a labeled complementary nucleic acid molecule having a nucleotide sequence complementary to one or more of the target RNA fragments; and (c) sequencing the labeled complementary nucleic acid molecule, or a portion thereof. The sequence of the complementary nucleic acid molecule or portion thereof will be the complement of the sequence of the target RNA fragments or terminal portion thereof.

Single-stranded nucleic acid molecules used in the nuclease protection assay can be either DNA or RNA. These single-stranded nucleic acid molecules may correspond to all or a portion of the target RNA molecule, or the complement thereof. The hybridizing can be carried out using nucleic acid hybridization methods that are known in the art. Digesting the portion of the single-stranded nucleic acid molecule that is not bound to one or more of the target RNA fragments through base-pair interactions can be accomplished using an enzyme that specifically hydrolyzes or cleaves single-stranded nucleic acid molecules. When the single-stranded nucleic acid molecule is an RNA molecule, an RNase enzyme (e.g., Ribonuclease A, Ribonuclease T1, or a combination of the two) can be used to digest the un-hybridized portion of the molecule. For example, a number of products which may be used to measure RNAse protection are sold by Ambion Corporation (cat. nos. 1415, 1420, and 1412, Austin, Tex.).

When nucleic acid molecules complementary to the target RNA fragments are synthesized or are otherwise obtained according to the methods of the invention, the complementary nucleic acid molecules can be separated by size, e.g., by chromatography or by gel electrophoresis (e.g., agarose gel electrophoresis, HPLC, or polyacrylamide gel electrophoresis). The separation of the molecules may facilitate their isolation, concentration, and/or purification prior to nucleic acid sequencing.

Any method for sequencing nucleic acid molecules can be used in the context of the present invention (Barrell, FASEB J. 5:40-45 (1991); Trainor, Anal. Chem. 62:418-26 (1990); Maxam and Gilbert, Methods Enzymol. 65:499-560 (1980); Sanger et al., Proc. Natl. Acad. Sci. USA 74:5463-67 (1977); U.S. Pat. No. 6,238,871 (and references cited therein)).

In related embodiments, adapter linkers may be used to place particular sequences on the 5′ or 3′ ends of target RNA fragments. Target RNA fragments generated from a target RNA molecule which was a mRNA are used for purposes of illustration. mRNA molecules generally contain a cap at the 5′ end and a poly(A) tail at the 3′ end. In most instances, it will be desirable to identify cleavage location which are not at the cap or in the poly(A) tail. Further, in order to identify the cleavage location in a target RNA molecule, it is only necessary to determine the nucleotide sequence of the new terminus of only one of the two target RNA fragments. One example of a method which may be used to identify cleavage sites in a target RNA molecule is shown in FIG. 3. This process set out in this figure is described in Example 1 below. In some embodiments, serial analysis of gene expression (SAGE) is used to identify cleavage points in the target RNA molecule. One example of a commercially available product which may be used for SAGE is Invitrogen Corporation's I-SAGE™ kits (see, e.g., Invitrogen Corporation, Carlsbad, Calif., T5000-01 and T5001-01). In brief, methods performed by users of these kits are as follows. First, mRNA in a sample is bound to magnetic beads containing oligo dT. This mRNA is then reverse transcribed to form cDNA. The cDNA is then digested with NlaIII restriction endonuclease to generate “sticky” ends. The digested cDNA is then split into two different aliquots and the cDNA in each of the aliquots which remains bound to the oligo dT is ligated to different adapters (i.e., adapters A and B). These adapters contain recognitions sites for BsmFI; a Type IIs restriction enzyme which cleaves nucleic acid 10-14 nucleotides away from its recognition sequence. Further, the restriction enzyme recognition sites are positions such that 10-14 nucleotides of cDNA is linked to the adapter after cleavage with BsmFI. Upon completion of digestion with BsmFI restriction endonuclease, two populations of molecules are formed: (1) a population in which 10-14 nucleotides of cDNA is linked to adapter A and (2) a population in which 10-14 nucleotides of cDNA is linked to adapter B. All of the nucleic acid molecules of these two populations share a compatible sticky end. Next, the two populations are mixed together under conditions which allow for molecules of the populations to be joined via their sticky ends. Nucleic acid molecules which contain adapter A and adapter B sequences near their termini are then amplified by PCR, resulting in nucleic acid molecules which contain short cDNA segments connected to each other in opposite orientation.

Adapter nucleic acid is remove by digestion with NlaIII restriction enzyme, to form what are referred to as “ditags”. These ditags are separated from adapter nucleic acid by gel electrophoresis (i.e., the ditags are “isolated”) and then ligated to each other to form concatamers. These concatamers are then inserted into pZERO-1 vectors and sequenced. mRNA molecules which were present in the original sample are identified by analysis of the sequence data.

A more detailed description of the I-SAGE™ methods is set out in the manual for I-SAGE™ products, which is available on Invitrogen Corporation's web page.

Once the nucleotide sequence of one or more of the target RNA fragments or a terminal portion thereof is determined, the positions of RNAi cleavage can be determined. For example, the nucleotide sequence of one or more of the target RNA fragments or a terminal portion thereof can be compared to the nucleotide sequence of the intact target RNA molecule. The nucleotide sequence of the intact target RNA molecule that corresponds to the nucleotide sequence at the 5′ or 3′ ends of the target RNA fragments will identify the location(s) of RNAi cleavage.

In certain instances, for example, when the sequences of multiple target RNA fragments are determined, it will be possible to align the sequences of the target RNA fragments (or terminal portions thereof) with the corresponding sequence of the intact target RNA molecule. The termini of the target RNA fragments, or the junction of two adjacent target RNA fragments, will correspond to the position(s) of RNAi cleavage.

If the sequence of only one target RNA fragment is determined, the position(s) of RNAi cleavage can be determined by aligning the sequence of the target RNA fragment (or terminal portions thereof) with the corresponding sequence of the intact target RNA molecule. The termini of the target RNA fragments (unless they correspond to the 5′ or 3′ end of the intact target RNA molecule) are positions of RNAi cleavage.

As an alternative to, or in addition to, determining the nucleic acid sequence of one or more of the target RNA fragments or terminal portions thereof, the invention also includes methods for identifying one or more RNAi cleavage sites along a target RNA molecule comprising: (a) determining the size of the target RNA fragments; and (b) comparing the size of the target RNA fragments to one another and to the intact target RNA molecule to determine the RNAi cleavage sites (or probable sites of RNAi cleavage). The size of the target RNA fragments can be determined by synthesizing nucleic acid molecules (e.g., labeled nucleic acid molecules) complementary to the target RNA fragments, and separating the complementary nucleic acid molecules according to size, e.g., using chromatographic and/or electrophoretic methods. Alternatively, the size of the target RNA fragments themselves can be determined using methods that are known in the art. The relative size(s) of the target RNA fragments, when compared to the size of the intact target RNA molecule, can be used to help deduce the positions of RNA cleavage.

Methods for Producing Mixed Populations of dsRNA Molecules

The invention also includes methods for producing dsRNA mixed populations. Methods according to this aspect of the invention comprise: (a) incubating a first intact dsRNA molecule with an enzyme having RNase activity, thereby producing a first set of two or more dsRNA fragments; (b) incubating a second intact dsRNA molecule with an enzyme having RNase activity, thereby producing a second set of two or more dsRNA fragments; and (c) combining the first set of two or more dsRNA fragments with the second set of two or more dsRNA fragments, thereby producing a mixed population of dsRNA molecules. The first and second intact RNA molecules may correspond to the same target RNA molecule or different target RNA molecules.

Methods according to this aspect of the invention may further comprise incubating a third, fourth, fifth, sixth, seventh, eighth, ninth, tenth, eleventh, twelfth, thirteenth, fourteenth, fifteenth, sixteenth, seventeenth, eighteenth, nineteenth and/or twentieth (or more) intact dsRNA molecule with an enzyme having RNase activity, thereby producing a third, fourth, fifth, sixth, seventh, eighth, ninth, tenth, eleventh, twelfth, thirteenth, fourteenth, fifteenth, sixteenth, seventeenth, eighteenth, nineteenth and/or twentieth (or more) set of two or more dsRNA fragments; and combining the first, second, third, fourth, fifth, sixth, seventh, eighth, ninth, tenth, eleventh, twelfth, thirteenth, fourteenth, fifteenth, sixteenth, seventeenth, eighteenth, nineteenth and/or twentieth (or more) sets of two or more dsRNA fragments, thereby producing a mixed population of dsRNA fragments. As above, intact RNA molecules used to generate the above mixed populations of dsRNA molecules may correspond to the same or different target RNA molecules.

The expression “enzyme having RNase activity” is intended to mean a substance (e.g., a substance comprising a protein or nucleic acid molecule) that, when combined with an RNA molecule (either a double stranded or a single stranded RNA molecule), catalyzes the hydrolysis of one or more of the chemical bonds between adjacent nucleotides or nucleotide base pairs. Exemplary enzymes having RNase activity include “Dicer,” e.g., Dicer from nematodes, fruit flies, fission yeast, flowering plants, and mammals, including mouse and human (Wilson et al., Nature 368:32-38 (1994); Rotondo and Frendewey, Nucl. Acids Res. 24:2377-2386 (1996); Jacobsen et al., Development 126:5231-5243 (1999); Kawasaki et al., Nucl. Acids Res. 31:981-987 (2003)). Other enzymes having RNase activity that can be used to produce dsRNA molecules for use with the present invention include prokaryotic RNase III enzymes (Yang et al., Proc. Natl. Acad. Sci. USA 99:9942-9947 (2002)). A Dicer enzyme may also be obtained from Invitrogen Corporation, Carlsbad, Calif. (see e.g., cat. nos. K3600-01 and K3650-01).

As indicated above, enzymes having RNase activity can be obtained from commercial sources. Enzymes having RNase activity can also be obtained from cells that express RNases using classical protein purification techniques. Alternatively, RNases can be obtained from recombinant sources. For example, a gene encoding an RNase can be cloned into an expression vector and the RNase can be produced by expressing the cloned gene in an appropriate host cell or in an in vitro system (Kawasaki et al., Nucl. Acids Res. 31:981-987 (2003); Myers et al., Nat. Biotechnol. 21:324-328 (2003); Provost et al., EMBO J. 21:5864-5874 (2002); Zhang et al., EMBO J. 21:5875-5885 (2002); Yang et al., Proc. Natl. Acad. Sci. USA 99:9942-9947 (2002)).

The term “incubating” refers to allowing the combination comprising the intact dsRNA molecule(s) and the enzyme having RNase activity to interact with one another under conditions sufficient for the RNase enzyme to cleave the intact dsRNA molecule(s) at least once. The conditions sufficient for the RNase enzyme to cleave the intact dsRNA molecule will depend on the nature of the RNase enzyme used and/or on the nature of the dsRNA molecule(s) included in the reaction. Such conditions are known in the art (Kawasaki et al., Nucl. Acids Res. 31:981-987 (2003); Myers et al., Nat. Biotechnol. 21:324-328 (2003); Provost et al., EMBO J. 21:5864-5874 (2002); Zhang et al., EMBO J. 21:5875-5885 (2002); Yang et al., Proc. Natl. Acad. Sci. USA 99:9942-9947 (2002)).

According to this aspect of the invention, each of the first, second, third, fourth, fifth, sixth, seventh, eighth, ninth, tenth, eleventh, twelfth, thirteenth, fourteenth, fifteenth, sixteenth, seventeenth, eighteenth, nineteenth and/or twentieth (or more) intact dsRNA molecules may be non-identical.

The intact dsRNA molecules that can be used in the creation of a dsRNA mixed population can be synthesized, or they can be produced by transcription from DNA or RNA templates. (U.S. Pat No. 3,597,318; U.S. Pat. No. 3,582,469; U.S. Pat. No. 5,795,715; Bhattacharyya, Nature 343:484 (1990); Milligan, Nucl. Acids Res. 21:8783 (1987); Provost et al., EMBO J. 21:5864-5874 (2002); Yang et al., Proc. Natl. Acad. Sci. USA 99:9942-9947 (2002)). The intact dsRNA molecules can also be extracted from biological material, e.g., from viruses (Dulieu et al., J. Virol. Meth. 24:77-84 (1989)) and yeasts (Fried et al., Proc. Natl. Acad. Sci. USA 75:4225 (1978)).

The term “combining” is intended to mean introducing into the same container or vessel the first, second, third, fourth, fifth, sixth, seventh, eighth, ninth, tenth, eleventh, twelfth, thirteenth, fourteenth, fifteenth, sixteenth, seventeenth, eighteenth, nineteenth and/or twentieth (or more) sets of two or more dsRNA fragments. The container or vessel can be any container or vessel, including but not limited to a test tube, vial, petri dish, centrifuge tube, micro-centrifuge tube (e.g., EPPENDORF®-style tube), jar, flask, pouch, etc.

For particular applications, it may be desirable to use mixed populations of dsRNA molecules which (1) correspond to the same target RNA molecule and (2) vary in their start and stop points. Put another way, it may be desirable to use a mixed population of dsRNA molecules which correspond to a target RNA molecule but do not represent the same 21-23 nucleotide sequences generated when Dicer digests a homogenous collection of intact RNA molecules. With this as a backdrop, Dicer is believed to associate with the end of an intact RNA molecule and then cleave this molecule about 21 nucleotides away. This generates a new terminus which forms the basis for the next cut another 21 nucleotides into the RNA molecules (Carmell and Hannon, Nature Structural & Molecular Biology 11:214-218(2004)). By generating a mixed population of dsRNA molecules using an enzyme such as Dicer from a starting population of RNA molecules which (1) correspond to the target RNA molecule and (2) are longer than 21 nucleotides, it is possible to generate a highly heterogeneous mixed population of dsRNA molecules, all of which correspond to the target RNA molecule. One method for doing this is to generate an intact RNA molecule and then shear it using, for example, mechanical force, so that the majority (e.g., 60-80%) of the intact RNA molecules are broken at least once. The resulting population of RNA molecules may then be digested with an enzyme with RNase activity (e.g., a Dicer enzyme) to generate a mixed population of dsRNA molecules which may then be used in methods of the invention.

Further, mixed populations of dsRNA molecules may be generated by shearing intact RNA molecules to particular average size. Depending on the sizes of these molecules and the particular application, they may either be used directly or may be separated from other dsRNA molecules which are of sizes that are not desired. For example, an intact RNA molecule of 900 bps may be sheared using physical force (e.g., vortexing, sonication, etc.) or otherwise broken by, for example, enzymatic (e.g., RNAse) digestion or chemical hydrolysis, or a combination of these, to an average length of 30 nucleotides, in which greater than 90% of the dsRNA molecules are between 20 and 40 nucleotides in length. The dsRNA molecules which are 30 nucleotides and less in length may be separated from those which are greater than 30 nucleotides in length using any number of methods. One example of such a method is gel electrophoresis. Another example is column purification using glass fiber filters and alcohol step gradients. Products which can be used for the purification of short dsRNA molecules include those associated with Invitrogen Corporation's manual entitled “BLOCK-iT Dicer RNAi Kits”, (cat. nos. K3600-01 and K3650-01).

Mixed populations of dsRNA molecules may also be produced by transcription of nucleic acid molecules which encode them. For example, a population of DNA vectors which encode two or more different shRNA molecules may be transcribed either in vitro or in vivo to generate a mixed population of dsRNA molecules. This mixed population may then be used in methods of the invention. Methods for preparing vectors which could be used in this aspect of the invention are known in the art (see, e.g., PCT Publications WO 03/006477 and WO 03/022052). One example of a commercial product which may be used to produce such vectors is BLOCK-IT™ U6 RNAi Entry Vector Kit (cat. nos. K4944-00 and K4945-00) and BLOCK-IT™ Lentiviral RNAi Expression System (cat. nos. K4943-00, K4944-00), available from Invitrogen Corp., Carlsbad, Calif.), which allows for the production of vectors which encode shRNA molecules that may be transcribed using an RNA polymerase III promoter.

Mixed populations of dsRNA molecules in which the majority of the individual members of the population are between 21 and 23 nucleotides in length may also be generated by “dicing” a population of intact dsRNA molecules which share substantial sequence similarity but vary in terms of their termini. One method for producing such mixed populations of dsRNA molecules takes advantage of the property of dicer enzymes to cleave intact dsRNA molecules 21-23 nucleotides in from the ends. Thus, if a population of intact dsRNA molecules is generated in which the individual members of the population vary in one or both termini, then dicer mediated cleavage will result in the generation of a population of dsRNA molecules which differ in nucleotide sequence based upon different cleavage points. For example, if in vitro transcription (e.g., using a T7 promoter based in vitro transcription system) is used to generate both strands of intact dsRNA molecules, the original DNA which is transcribed may be designed so that a mixed population of intact dsRNA molecules is subjected to “dicing”. For example, DNA molecules subjected to in vitro transcription may be designed such that transcription begins at a particular nucleotide in the sequence. Other DNA molecules in the same in vitro transcription reaction mixture may be designed to begin transcription at the −1 position, the −2 position, the −3 position and so on until the −21, −22, or −23 position is reached. This may be done to produce both strands of intact ds RNA molecules. When the two strands are hybridized to each other and then subjected to “dicing” the result is a mixed population of dsRNA molecules which vary in sequence but correspond to the intact dsRNA molecule.

In methods related to those described above, the individual DNA molecules are prepared in and transcribed in separate tube, wells or other containers, instead of one container, to prevent single-stranded RNA molecules which do not share full sequence complementarity from hybridizing to each other. The intact RNA molecules in each of these tube, wells or other containers may also be “diced” and then mixed to form the final population which is contacted with cells.

Mixed Populations of dsRNA Molecules

The invention also includes mixed populations of dsRNA. The mixed populations of dsRNA of the invention include dsRNA mixed populations produced by any of the methods for producing a mixed population of dsRNA molecules that are included within the invention. As well as mixtures of nucleic acid molecules (e.g., DNA molecules) which encode these mixed populations.

The invention includes a mixed population of dsRNA molecules comprising at least one first dsRNA molecule and at least one second dsRNA molecule, wherein the nucleotide sequence of at least one of the strands of the first dsRNA molecule is at least 90% (e.g., 90%, 91%, 92%, 93%, 94%, 95%, 96%, 97%, 98%, 99% or 100%) identical to the nucleotide sequence of a first target RNA molecule or a portion thereof, as well as the complements thereof, and wherein the nucleotide sequence of at least one of the strands of the second dsRNA molecule is at least 90% (e.g., 90%, 91%, 92%, 93%, 94%, 95%, 96%, 97%, 98%, 99% or 100%) identical to the nucleotide sequence of a second target RNA molecule or a portion thereof, and wherein the first and the second dsRNA molecules are non-identical. In many instances, the first and second target RNA molecules will have no regions of sequence identity, with the exception of a poly(A) tail, which are longer than 10, 15, 20, 30, 40, or 50 nucleotides.

Mixed populations of dsRNA molecules may comprise any number (greater than one) of non-identical dsRNA molecules, the nucleotide sequences of which correspond to different target RNA molecules or portions thereof. Mixed populations of dsRNA molecules may further comprise one or more additional non-identical dsRNA molecules, wherein the nucleotide sequence of at least one of the strands of the additional dsRNA molecules is at least 90% (e.g., 90%, 91%, 92%, 93%, 94%, 95%, 96%, 97%, 98%, 99% or 100%) identical to the nucleotide sequence of the first or second target RNA molecules, or a portion thereof or to the nucleotide sequence of a third target RNA molecule or a portion thereof. For example, mixed populations of the invention include mixed populations comprising two or more (e.g., 2, 3, 4, 5, 6, 7, 8, 9, 10, 20, 30, 50, 100, 150, 300, 500, 800, 1000, etc.) non-identical dsRNA molecules, each corresponding to a nucleotide sequence of a different target RNA molecule or portion thereof. In particular embodiments, mixed populations of RNA molecules of the invention comprise dsRNA molecules which correspond to two or more (e.g., 2, 3, 4, 5, 6, 7, 8, 9, 10, 20, 30, 50, 100, 150, 300, 500, 800, 1000, or more) different target RNA molecules. 166 Mixed populations of dsRNA molecules included within the invention may be produced by a variety of methods, for example, by combining: (i) one or more first dsRNA molecules that correspond to the nucleotide sequence of a first target RNA molecule or a portion thereof, and (ii) one or more second dsRNA molecules that correspond to the nucleotide sequence of a second target RNA molecule or a portion thereof.

As indicated above, the invention also includes mixed populations of nucleic acid molecules which encode mixed populations of dsRNA molecules. Such nucleic acid molecules may encode individual single-stranded RNA molecules or both complementary strands of double-stranded RNA molecules. When nucleic acid molecules encode both strands of dsRNA molecules, these strands may be separate or connected. In other words, the RNA molecules may be siRNA molecules or shRNA molecules. Exemplary nucleic acid molecules of this aspect of the invention include DNA expression vectors.

The invention further includes the use of mixed populations of nucleic acid molecules which encode mixed populations of dsRNA molecules in methods of the invention. In other words, instead of directly using mixed populations of dsRNA molecules in methods of the invention, mixed populations of nucleic acid molecules which encode mixed populations of dsRNA molecules may be used. Typically, in such methods, some or all of these nucleic acid molecules will be transcribed either in vitro or in vivo to produce a mixed population of dsRNA molecules. This aspect of the invention provides the flexibility of expressing sub-portions of the mixed population of nucleic acid molecules at different times. For example, nucleic acid molecules which encode different dsRNA molecules may be operably connected to different promoters. As an example, nucleic acid molecules which encode dsRNA molecules that correspond to a first target RNA molecule may be operably linked to a constitutive promoter and nucleic acid molecules which encode dsRNA molecules that correspond to a second target RNA molecule may be operably linked to a inducible promoter. Such a mixed population of nucleic acid molecules may then be introduced into a cell, for example. If the constitutive promoter activates transcription in the cell, knock-down of the first target RNA molecule would be expected to occur shortly thereafter but knock-down of the second target RNA molecule would be expected to occur only after induction of transcription.

Fusion RNA Molecules

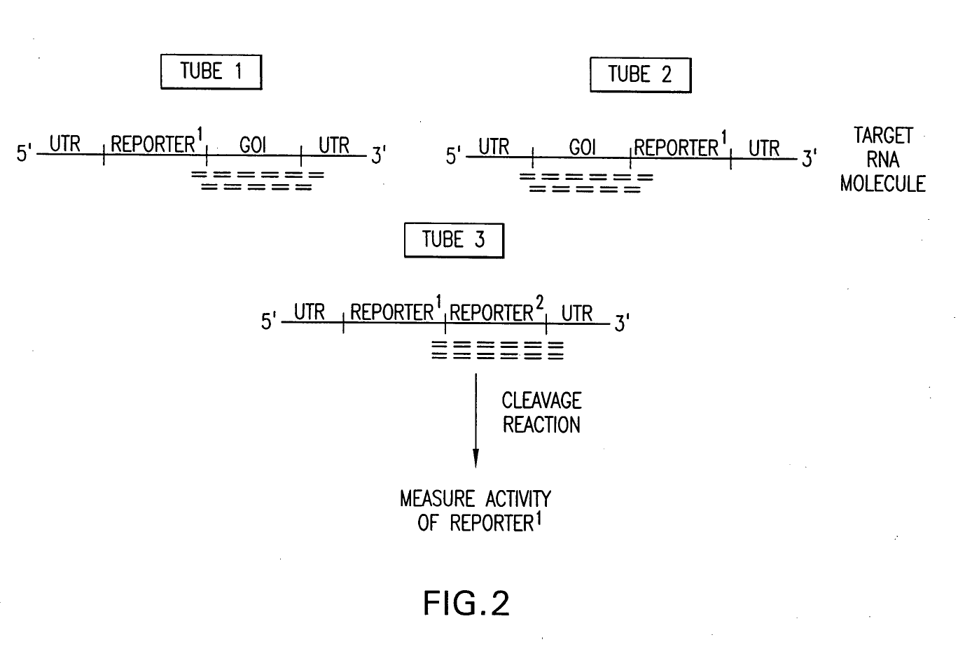

The invention further includes RNA molecules which contain at least two components. Typically, these two components comprise nucleic acid segments which are not normally associated with each other. These two components may become associated with each other as the results of, for example, molecular cloning. FIG. 2 illustrates three different variants of this aspect of the invention. More specifically, three different target RNA molecules are shown. Each of these target RNA molecules comprises two UTRs and two additional segments comprising nucleic acid which encodes a reporter or a gene of interest. In tube 1, the target RNA molecules comprises reporter 1 located near the 5′ end of the transcript and nucleic acid corresponding to a gene of interest (e.g., all or part of an open reading frame) near the 3′ end. In tube 2, the target RNA molecules comprises reporter 1 located near the 3′ end of the transcript and nucleic acid corresponding to a gene of interest (e.g., all or part of an open reading frame) near the 5′ end. In tube 3, the target RNA molecules comprises reporter 1 located near the 5′ end of the transcript and nucleic acid corresponding to reporter 2 near the 3′ end. In each instance, a mixed population of dsRNA molecules is shown which corresponds to one sub-portion of the target RNA molecule. The target RNA molecules and the mixed populations of dsRNA molecules are contacted with each other under conditions which allow for RNAi mediated cleavage reactions to occur. After either a particular length of time or while the cleavage reaction is occurring, reporter activity is measured. In particular embodiments, the report shown schematically in FIG. 2 may be replaced by a non-reporter tag.

Non-reporter tags may be used in any number of ways to monitor RNAi mediated degradation. Examples of such non-reporter tag include resistance markers (e.g., nucleic acids which encode polypeptides which confer resistance to hygromycin, Zeocin, or agent such as metal ions), “negative” selection markers (e.g. , HSV thymidine kinase), proteins which become localized to the surface of cells (e.g., cell surface markers), and fluorescent tags such as LUMIO™ tags described elsewhere herein. As explained in more detail below, these tags, as well as reporter tags, may be expressed using either a constitutive or regulatable promoter.

Resistance markers include antibiotic resistance markers and markers which encode proteins such as metallothioneins. In many instances, these tags will be used in conjunctions with agents to which the particular resistance marker(s) confers resistance. As an example, a cell which expresses a fusion target RNA that encode a metallothionein, may be contacted with dsRNA molecules which are designed to degrade the target RNA. Test samples of these cells may then be taken and contacted with varying concentrations of a heavy metal ion (e.g., copper, cadmium, mercury, etc.). After a certain period of time, the number of viable cells remaining in the population may be compared to control samples which were not contacted with the dsRNA molecules. The number of viable cells present in the various test samples, as compared to the control samples to determine the level of RNA degradation mediated by the dsRNA. Further, in specific methods, the fusion target RNA may be transcribed using a regulatable promoter. For example, the invention includes methods wherein, the cells are contacted with the dsRNA molecules either before or simultaneous with production of fusion target RNA, followed by contacting of the cells with the heavy metal ions. In such instances, the cells used assay methods described above will generally start of with little or no metallothionein at the time they are contacted with the dsRNA molecules. Thus, in most instances, continued viability of the cells will depend upon the translation of metallothionein in the presence of dsRNA molecules. Similar methods may be used for resistance markers which confer resistance to agents other than heavy metal ions (e.g., antibiotics).

As noted above non-reporter tags also include cell surface proteins. For example, the fusion RNA molecules may encode proteins which become localized to the surface of the cells in which they are expressed. Thus, in many instances, the expressed polypeptide will contain a signal peptide. After the protein is localized to the cell surface, cell surface localization may be detected using, for example a fluorescently labeled antibody. Further, if the protein present on the surface of the cells contains a tag which may be detected using a fluorescent agent other than an antibody, then detection of the protein on the cells surface may be done by other means. Examples of such tags are the Lumio™ tags described elsewhere herein. These tags form fluorescent complexes when bound to particular compounds (e.g., biarsenical compounds).

The schematic in FIG. 2 shows mixed populations of dsRNA molecules being used in the process set out therein. Once a suitable dsRNA molecule has been identified which efficiently mediates RNAi processes, one or a small number of such dsRNA molecules may then be used. Of course, when one purpose of performing a cleavage reaction such as that set out in FIG. 2 is to identify dsRNA molecules which efficiently mediate RNAi processes, then it will typically be advantageous to use mixed populations of dsRNA molecules as set out in this figure.

The dsRNA molecules used in methods such as those shown in FIG. 2 need not correspond to just coding regions of the target RNA molecule. dsRNA molecules may also correspond to the 5′ and 3′ untranslated regions or, in appropriate circumstances, intervening regions located between the two open reading frames. In instances where dsRNA molecules corresponds to the intervening regions between the open reading frames, there will often be a non-coding region positioned in between. In other words, using the target RNA molecule represented in FIG. 2, Tube 1 for purposes of illustration, an additional nucleic acid segment may be located between “Reporter 1” and “GOI” and dsRNA molecules may correspond to this nucleic acid segment. The invention further includes nucleic acid molecules (e.g., the target RNA molecules and DNA molecules which encode them) which contain such additional nucleic acid segments, as well as cells and reaction mixtures which contain these nucleic acid molecules.

In particular embodiments, target RNA molecules used in methods and present in compositions of the invention comprise RNA corresponding to two components, wherein the two components are (1) all or part of a gene of interest and (2) a reporter or other tag. In additional embodiments, neither of these components are present in a format which allows them to be translated either in the cell or reaction mixture in which they are located. In further embodiments, either one or both of these components are present in a format which allows them to be translated either in the cell or reaction mixture in which they are located. Thus, the invention includes methods for monitoring the progression of RNAi mediated cleavage of target RNA molecules. In many instances, these methods will involve detecting the expression level of a reporter or other tag (e.g., β-lactamase, luciferase, etc.) by measuring the activity of a translation product of RNA encoding the reporter or other reporter.

Reporters which may be used in the practice of methods of the invention include β-galactosidase, alkaline phosphatase, green fluorescent protein, yellow fluorescent protein, red fluorescent protein, cyanin fluorescent protein, β-lactamase, luciferase, and dominant selectable markers such as HSV thymidine kinase and HPRT. Tags which may be used in the practice of methods of the invention include peptides which may be detected due to their affinity for one or more chemical agents (e.g., a peptide that binds LUMIO™, a His Tag, etc.) or antibody (e.g., a V5 epitope tag, a FLAG tag, a T7 tag, a myc tag, etc.). Tags also include proteins and peptides which are capable of mediating negative selection (e.g., Diphtheria toxin, thymidine kinase, HPRT, etc.).

When a tag is use which is inherently toxic (e.g., Diphtheria toxin) to cells, it will often be advantageous to express this tag using a regulatable promoter. Thus, regulation of expression of such toxic tags may be controlled by regulating tag expression.

In specific embodiments, at least one of the reporters used is β-lactamase. Methods for measuring β-lactamase activity in cells and in cell free systems are known in the art (see, e.g., U.S. Pat. Nos. 5,741,657, 5,955,604, 6,291,162, and 6,472,205, the entire disclosures of which are incorporated herein by reference). Methods of the invention include those where β-lactamase activity is measured by detection of products that are generated by the reaction of enzymatic substrates which become fluorescent after reaction with a β-lactamase. Examples of such substrates are CCF2 and CCF4 (Invitrogen Corp., Carlsbad, Calif., cat. nos. 12578-126, 12578-134, 12578-019 and 12578-027; U.S. Patent Appl. No. 60/487,301, filed on Jul. 16, 2003, the entire disclosure of which is incorporated herein by reference).

Often, when progression of RNAi mediated cleavage of a target RNA molecule comprising a reporter is used in a cell free system, this cell free system will allow for translation of the target RNA molecule. Thus, a translation product of RNA encoding the reporter can be monitored.

In a specific embodiment of the invention, nucleic acid molecules of the invention may comprise a nucleic acid sequence encoding a polypeptide having an enzymatic activity (e.g., β-lactamase activity). In some embodiments, nucleic acid molecules of the invention may comprise a nucleic acid sequence encoding a polypeptide having a detectable β-lactamase activity. Assays for β-lactamase activity are known in the art. U.S. Pat. No. 5,955,604, issued to Tsien, et al. Sep. 21, 1999, U.S. Pat. No. 5,741,657 issued to Tsien, et al., Apr. 21, 1998, U.S. Pat. No. 6,031,094, issued to Tsien, et al., Feb. 29, 2000, U.S. Pat. No. 6,291,162, issued to Tsien, et al., Sep. 18, 2001, and U.S. Pat. No. 6,472,205, issued to Tsien, et al. Oct. 29, 2002, disclose the use of β-lactamase as a reporter gene and fluorogenic substrates for use in detecting β-lactamase activity and are specifically incorporated herein by reference. In one embodiment of the invention, a nucleic acid sequence encoding a polypeptide having a detectable activity may be a nucleic acid sequence encoding a polypeptide having β-lactamase activity and desired host cells may be identified by assaying the host cells for β-lactamase activity.

A β-lactamase catalyzes the hydrolysis of a β-lactam ring. Those skilled in the art will appreciate that the sequences of a number of polypeptides having β-lactamase activity are known. In addition to the specific β-lactamases disclosed in the Tsien, et al. patents listed above, any polypeptide having β-lactamase activity is suitable for use in the present invention.

β-lactamases are classified based on amino acid and nucleotide sequence (Ambler, R. P., Phil. Trans. R. Soc. Lond. [Ser.B.] 289: 321-331 (1980)) into classes A-D. Class A β-lactamases possess a serine in the active site and have an approximate weight of 29 kd. This class contains the plasmid-mediated TEM β-lactamases such as the RTEM enzyme of pBR322. Class B β-lactamases have an active-site zinc bound to a cysteine residue. Class C enzymes have an active site serine and a molecular weight of approximately 39 kd, but have no amino acid homology to the class A enzymes. Class D enzymes also contain an active site serine. Representative examples of each class are provided below with the accession number at which the sequence of the enzyme may be obtained in the indicated database. The sequences of the enzymes in the following lists are specifically incorporated herein by reference.

TABLE 6

| | Accession | |
|---|---|---|
| Class A β-lactamases | No. | Data Bank |
| Bacteroides fragilis CS30 | L13472 | GenBank |
| Bacteroides uniformis WAL-7088 | P30898 | SWISS-PROT |
| PER-1, P. aeruginosa RNL-1 | P37321 | SWISS-PROT |
| Bacteroides vulgatus CLA341 | P30899 | SWISS-PROT |
| OHIO-1, Enterobacter cloacae | P18251 | SWISS-PROT |
| SHV-1, K. pneumoniae | P23982 | SWISS-PROT |
| LEN-1, K. pneumoniae LEN-1 | P05192 | SWISS-PROT |
| TEM-1, E. coli | P00810 | SWISS-PROT |
| Proteus mirabilis GN179 | P30897 | SWISS-PROT |
| PSE-4, P. aeruginosa Dalgleish | P16897 | SWISS-PROT |
| Rhodopseudomonas capsulatus SP108 | P14171 | SWISS-PROT |
| NMC, E. cloacae NOR-1 | P52663 | SWISS-PROT |
| Sme-1, Serratia marcescens S6 | P52682 | SWISS-PROT |
| OXY-2, Klebsiella oxytoca D488 | P23954 | SWISS-PROT |
| K. oxytoca E23004/SL781/SL7811 | P22391 | SWISS-PROT |
| S. typhimurium CAS-5 | X92507 | GenBank |
| MEN-1, E. coli MEN | P28585 | SWISS-PROT |
| Serratia fonticola CUV | P80545 | SWISS-PROT |
| Citrobacter diversus ULA27 | P22390 | SWISS-PROT |
| Proteus vulgaris 5E78-1 | P52664 | SWISS-PROT |
| Burkholderia cepacia 249 | U85041 | GenBank |
| Yersinia enterocolitica serotype O: 3/Y-56 | Q01166 | SWISS-PROT |
| M. tuberculosis H37RV | Q10670 | SWISS-PROT |
| S. clavuligerus NRRL 3585 | Z54190 | GenBank |
| III, Bacillus cereus 569/H | P06548 | SWISS-PROT |
| B. licheniformis 749/C | P00808 | SWISS-PROT |
| I, Bacillus mycoides NI10R | P28018 | SWISS-PROT |
| I, B. cereus 569/H/9 | P00809 | SWISS-PROT |
| I, B. cereus 5/B | P10424 | SWISS-PROT |
| B. subtilis 168/6GM | P39824 | SWISS-PROT |
| 2, Streptomyces cacaoi DSM40057 | P14560 | SWISS-PROT |
| Streptomyces badius DSM40139 | P35391 | SWISS-PROT |
| Actinomadura sp. strain R39 | X53650 | GenBank |
| Nocardia lactamdurans LC411 | Q06316 | SWISS-PROT |
| S. cacaoi KCC S0352 | Q03680 | SWISS-PROT |
| ROB-1, H. influenzae F990/LNPB51/ | P33949 | SWISS-PROT |
| serotype A1 | | |
| Streptomyces fradiae DSM40063 | P35392 | SWISS-PROT |
| Streptomyces lavendulae DSM2014 | P35393 | SWISS-PROT |
| Streptomyces albus G | P14559 | SWISS-PROT |
| S. lavendulae KCCS0263 | D12693 | GenBank |
| Streptomyces aureofaciens | P10509 | SWISS-PROT |
| Streptomyces cellulosae KCCS0127 | Q06650 | SWISS-PROT |
| Mycobacterium fortuitum | L25634 | GenBank |
| S. aureus PC1/SK456/NCTC9789 | P00807 | SWISS-PROT |
| BRO-1, Moraxella catarrhalis ATCC 53879 | Z54181 | GenBank; |
| | Q59514 | SWISS-PROT |

TABLE 7

| Class B β-lactamases | Accession No. | Data Bank |
|---|---|---|
| II, B. cereus 569/H | P04190 | SWISS-PROT |
| II, Bacillus sp. 170 | P10425 | SWISS-PROT |
| II, B. cereus 5/B/6 | P14488 | SWISS-PROT |
| Chryseobacterium meningosepticum | X96858 | GenBank |
| CCUG4310 | | |
| IMP-1, S. marcescens AK9373/TN9106 | P52699 | SWISS-PROT |
| B. fragilis TAL3636/TAL2480 | P25910 | SWISS-PROT |
| Aeromonas hydrophila AE036 | P26918 | SWISS-PROT |
| L1, Xanthomonas maltophilia IID 1275 | P52700 | SWISS-PROT |

TABLE 8

| Class C β-lactamases | Accession No. | Data Bank |
|---|---|---|
| Citrobacter freundii OS60/GN346 | P05193 | SWISS-PROT |
| E. coli K-12/MG1655 | P00811 | SWISS-PROT |
| P99, E. cloacae P99/Q908R/MHN1 | P05364 | SWISS-PROT |
| Y. enterocolitica IP97/serotype O: 5B | P45460 | SWISS-PROT |
| Morganella morganii SLM01 | Y10283 | GenBank |
| A. sobria 163a | X80277 | GenBank |
| FOX-3, K. oxytoca 1731 | Y11068 | GenBank |
| K. pneumoniae NU2936 | D13304 | GenBank |
| P. aeruginosa PAO1 | P24735 | SWISS-PROT |
| S. marcescens SR50 | P18539 | SWISS-PROT |
| Psychrobacter immobilis A5 | X83586 | GenBank |

TABLE 9

| | Accession | |
|---|---|---|
| Class D β-lactamases | No. | Data Bank |
| OXA-18, Pseudomonas aeruginosa Mus | U85514 | GenBank |
| OXA-9, Klebsiella pneumoniae | P22070 | SWISS-PROT |
| Aeromonas sobria AER 14 | X80276 | GenBank |
| OXA-1, Escherichia coli K10-35 | P13661 | SWISS-PROT |
| OXA-7, E. coli 7181 | P35695 | SWISS-PROT |
| OXA-11, P. aeruginosa ABD | Q06778 | SWISS-PROT |
| OXA-5, P. aeruginosa 76072601 | Q00982 | SWISS-PROT |
| LCR-1, P. aeruginosa 2293E | Q00983 | SWISS-PROT |
| OXA-2, Salmonella typhimurium type 1A | P05191 | SWISS-PROT |

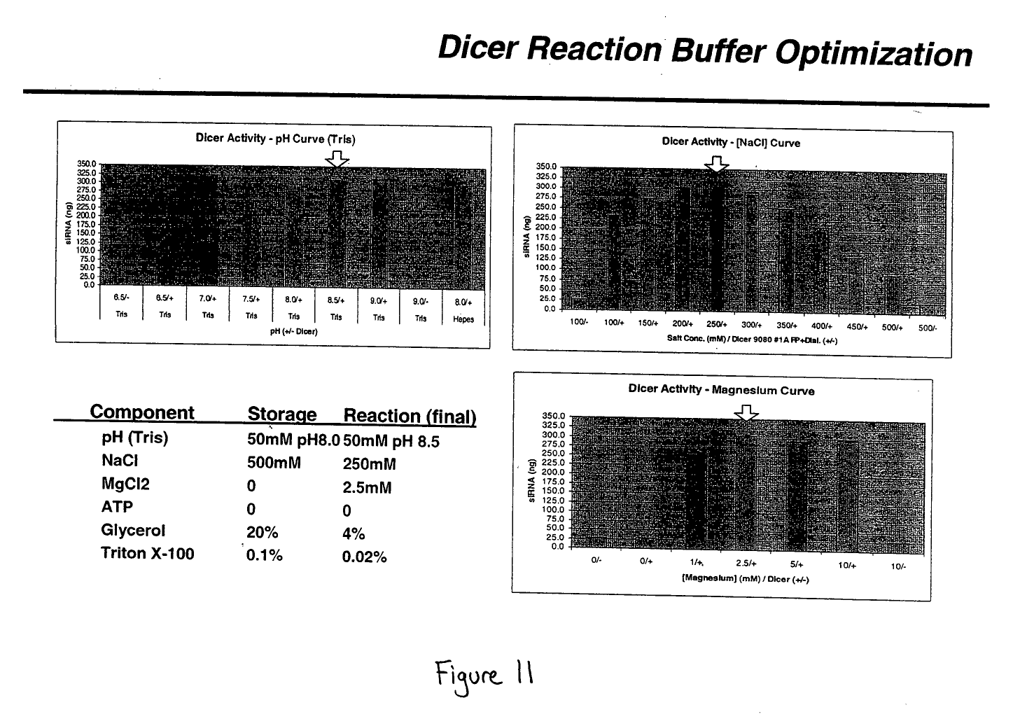

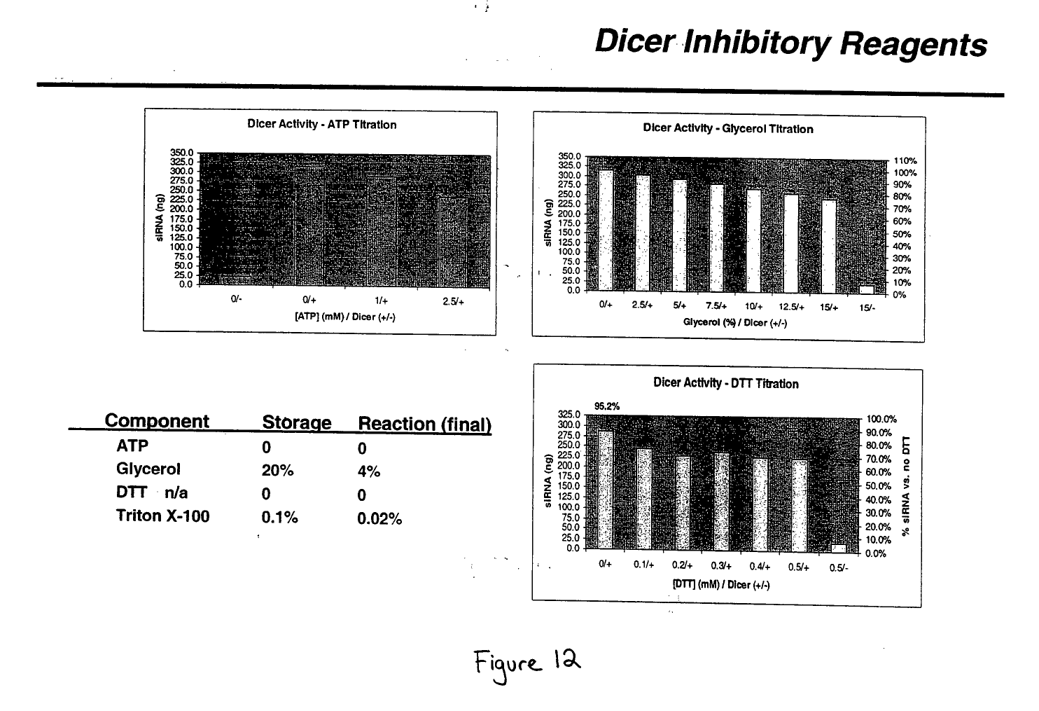

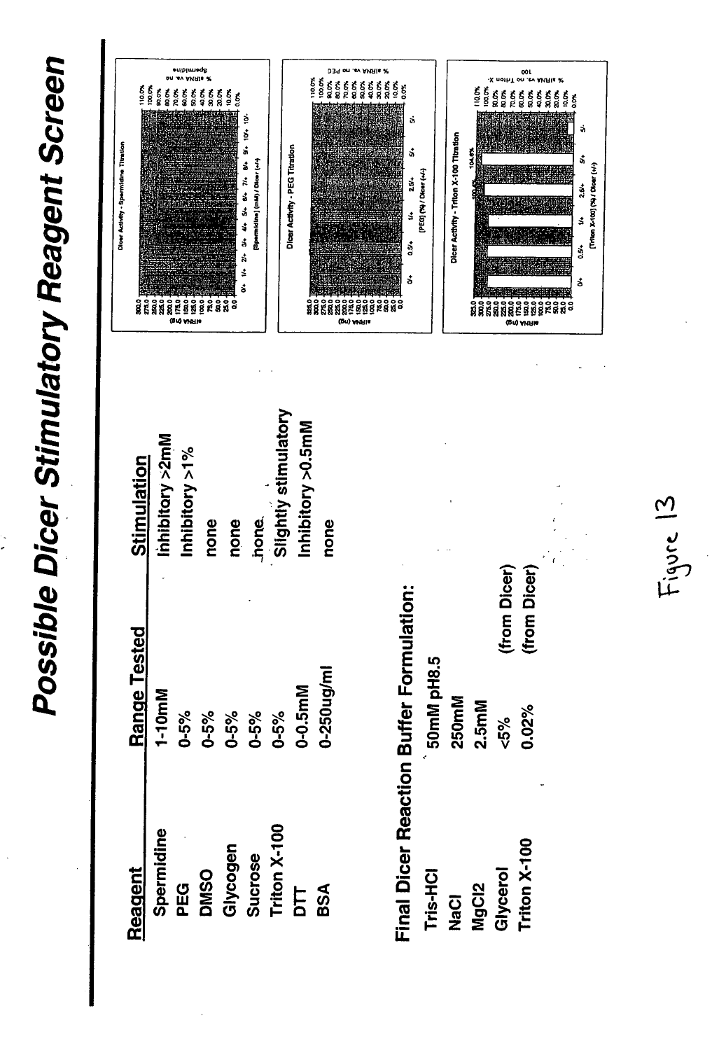

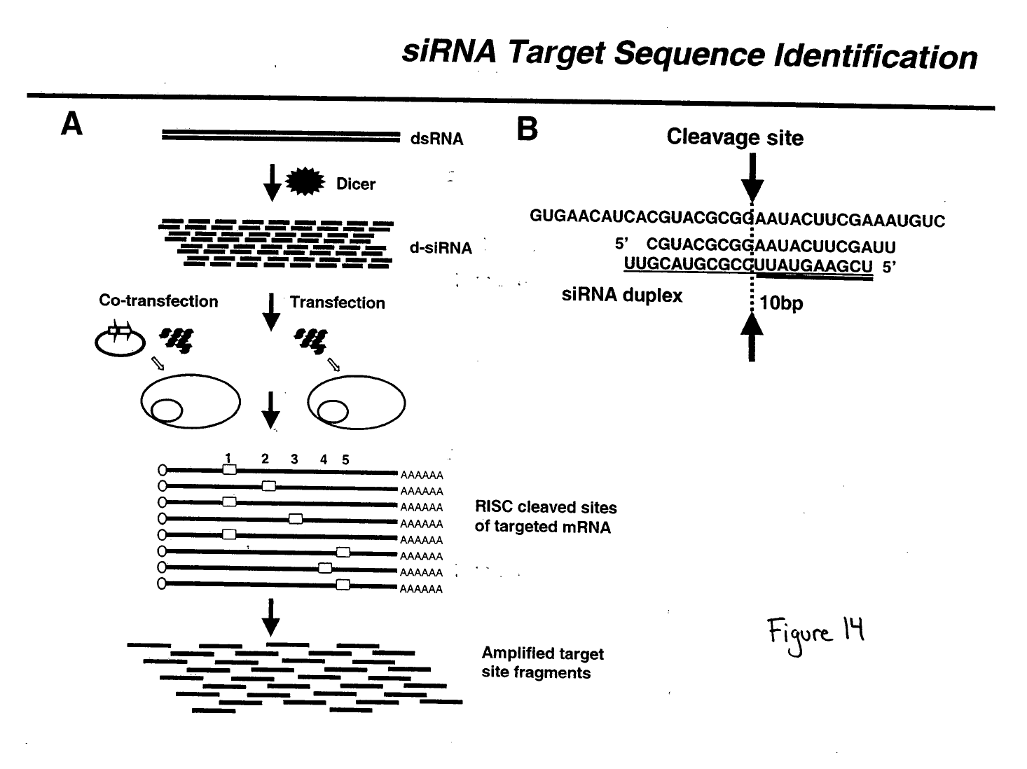

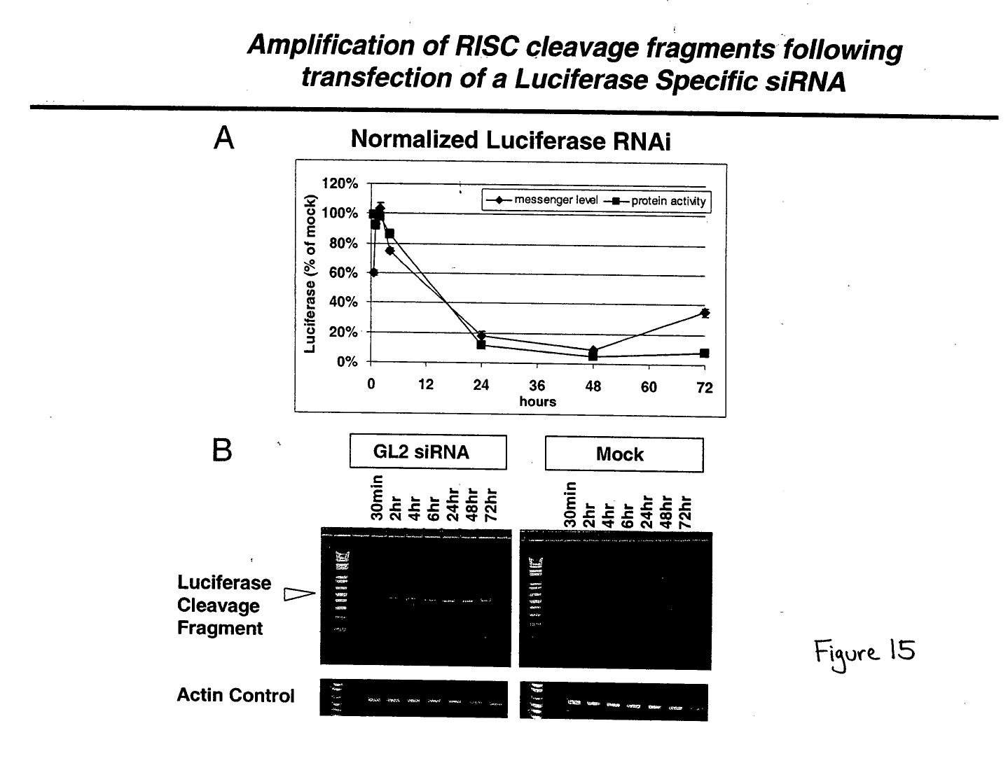

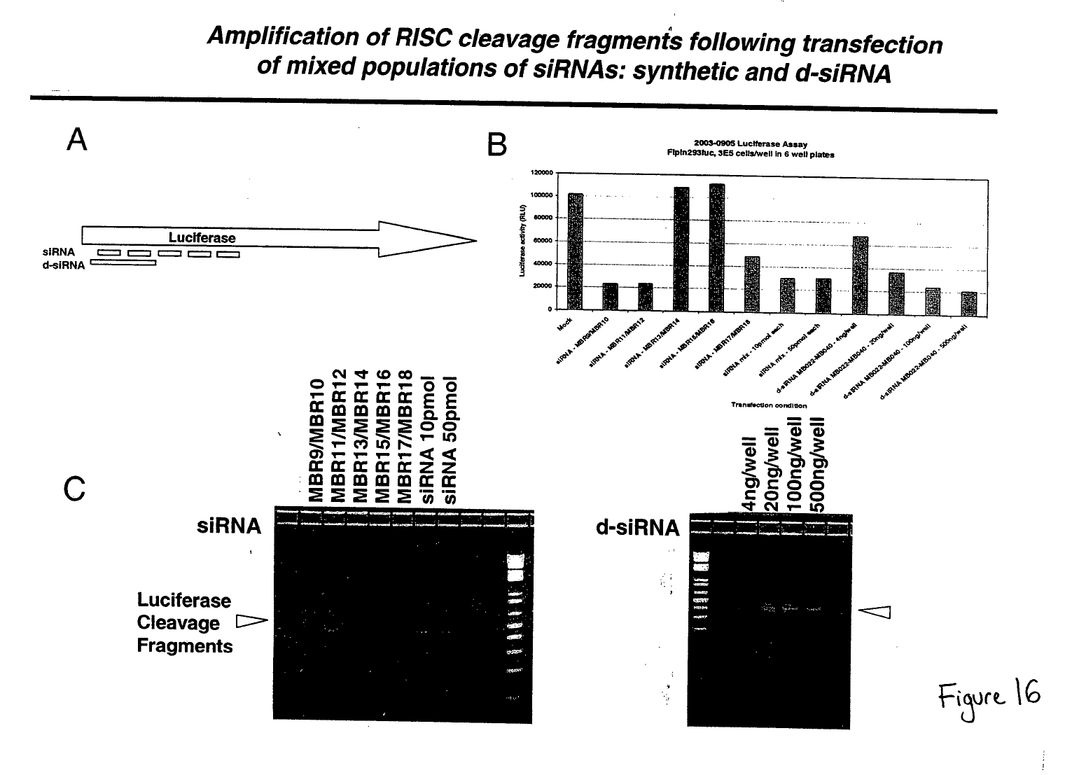

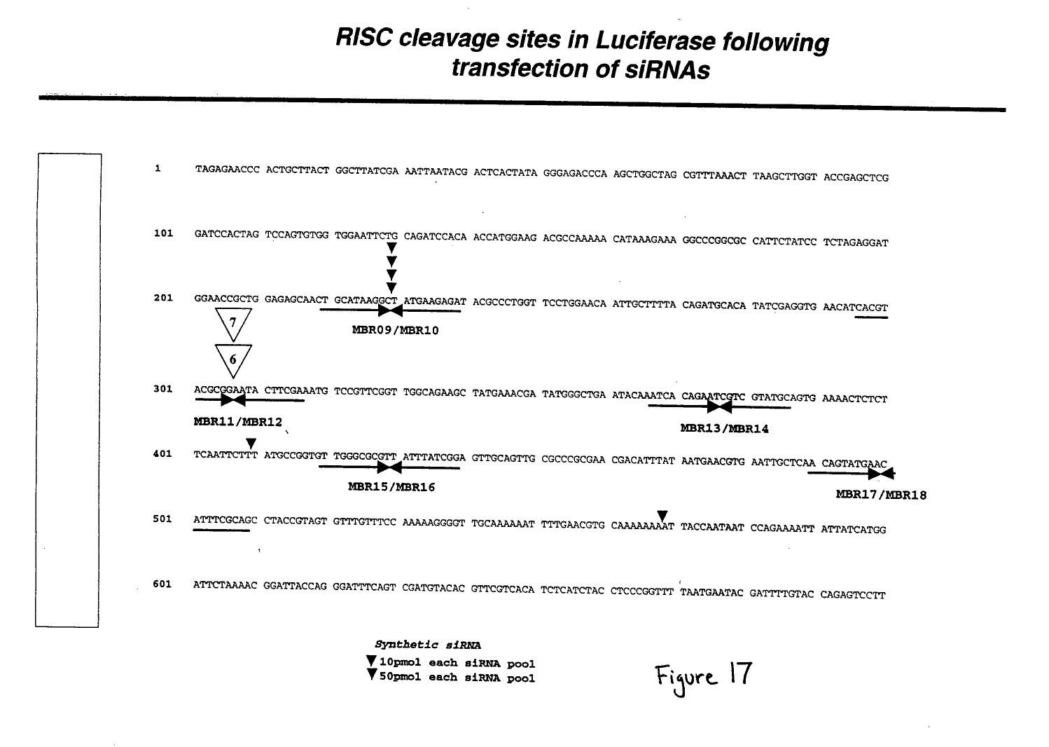

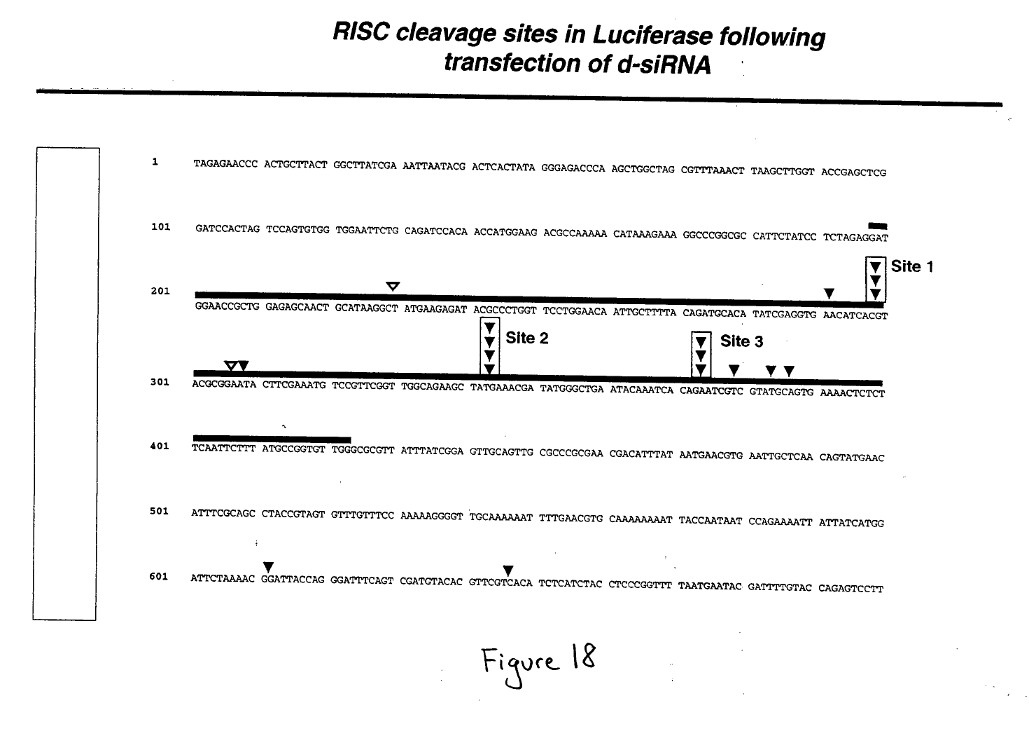

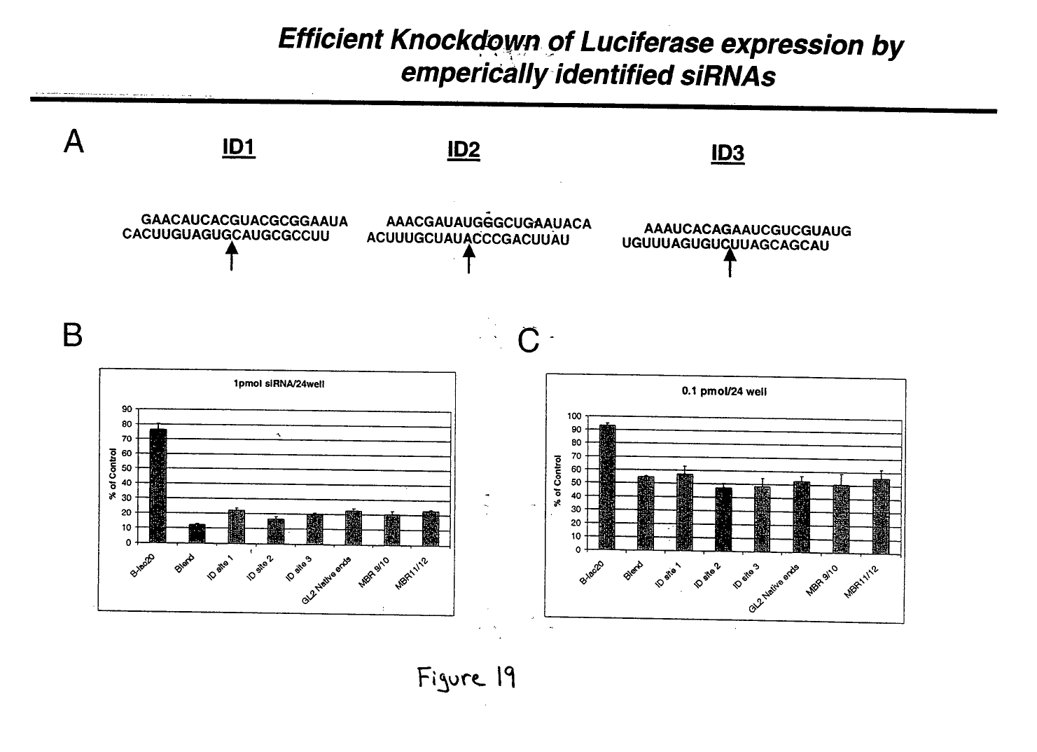

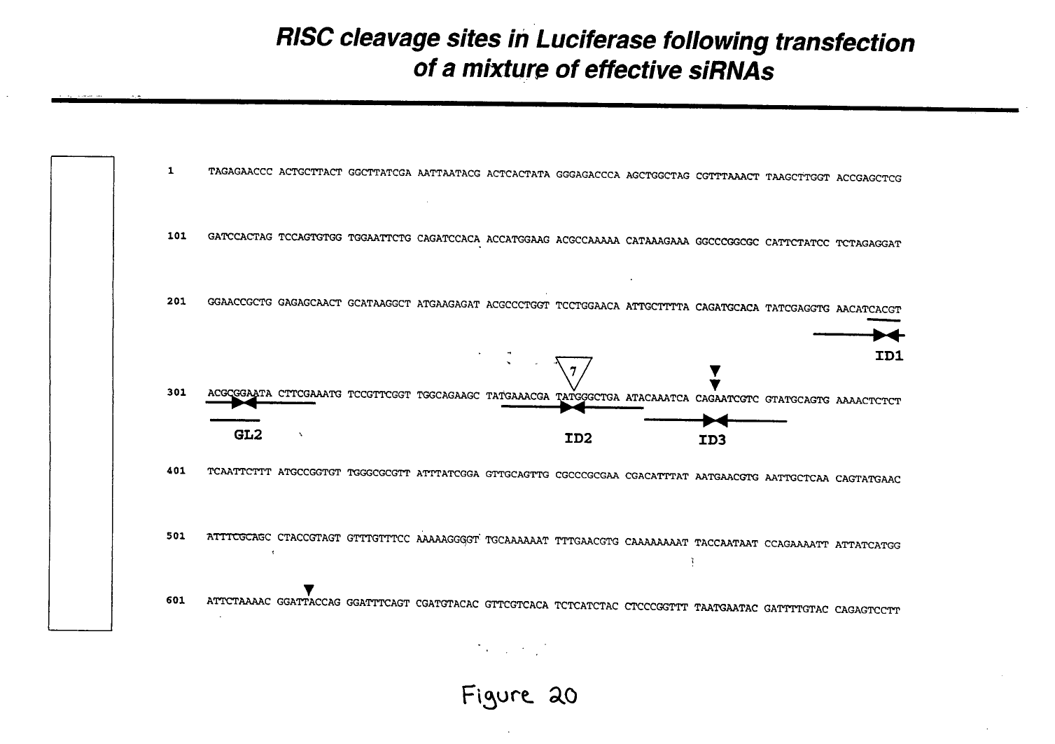

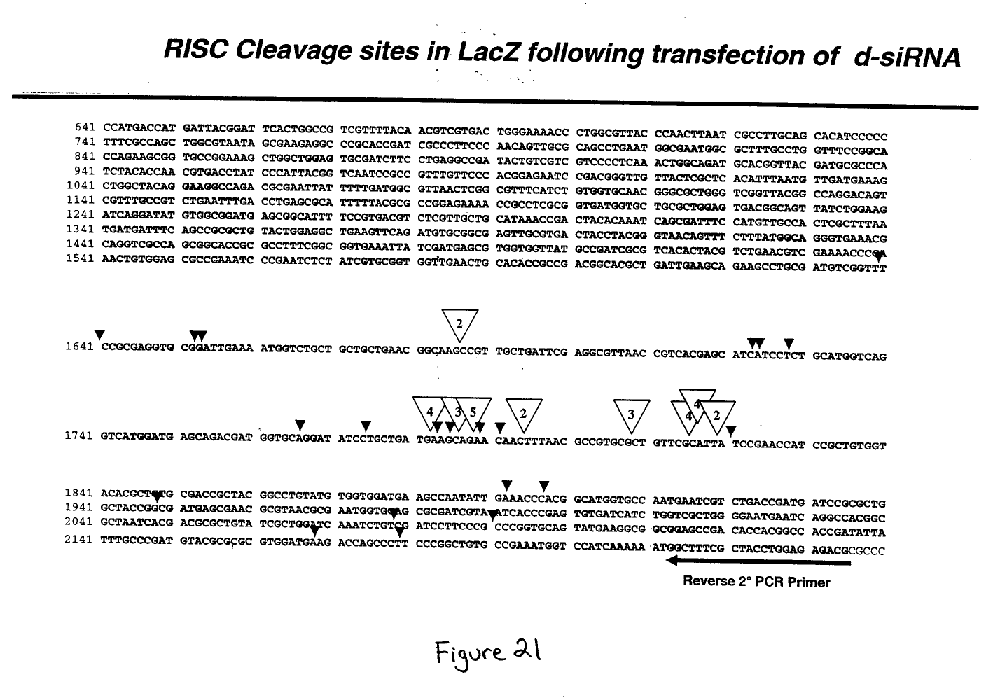

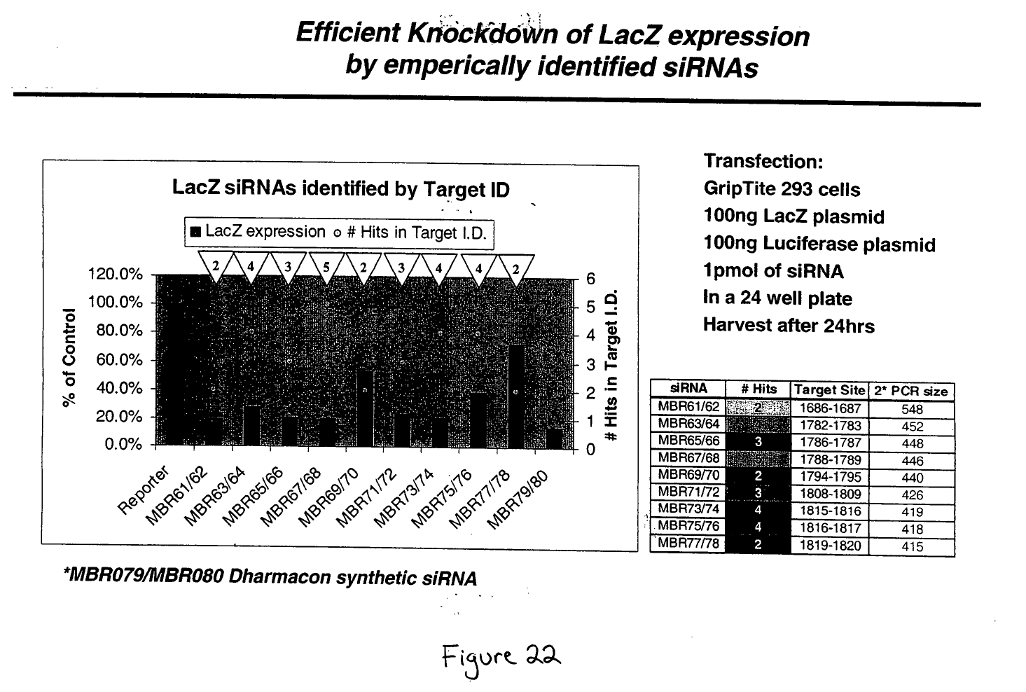

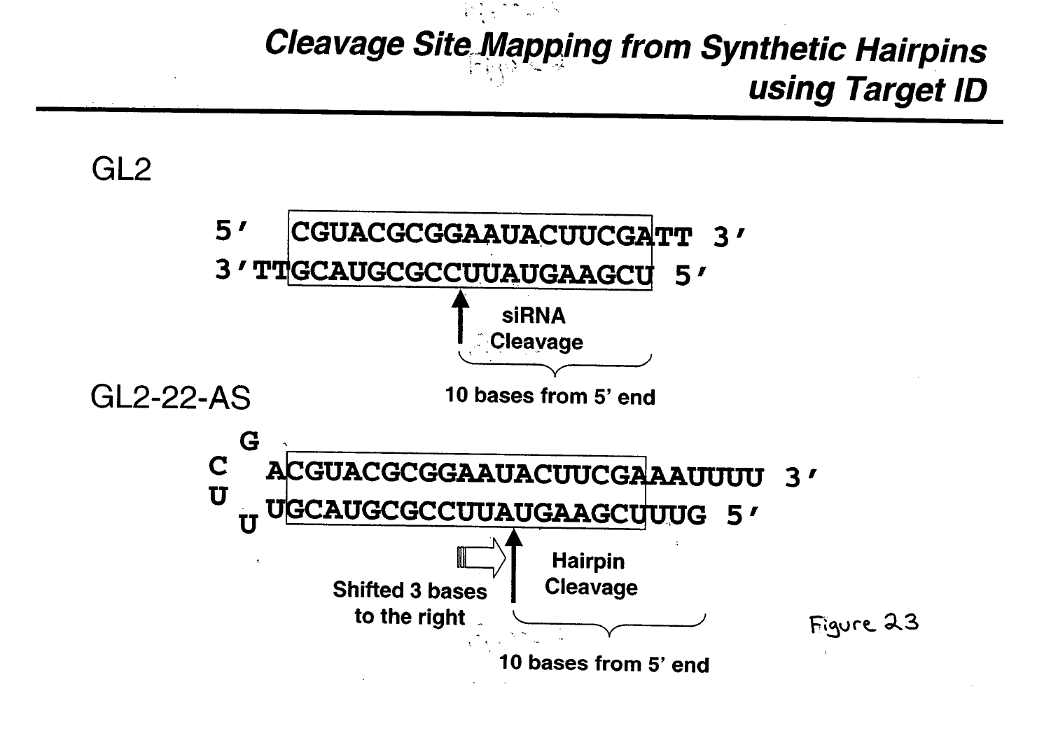

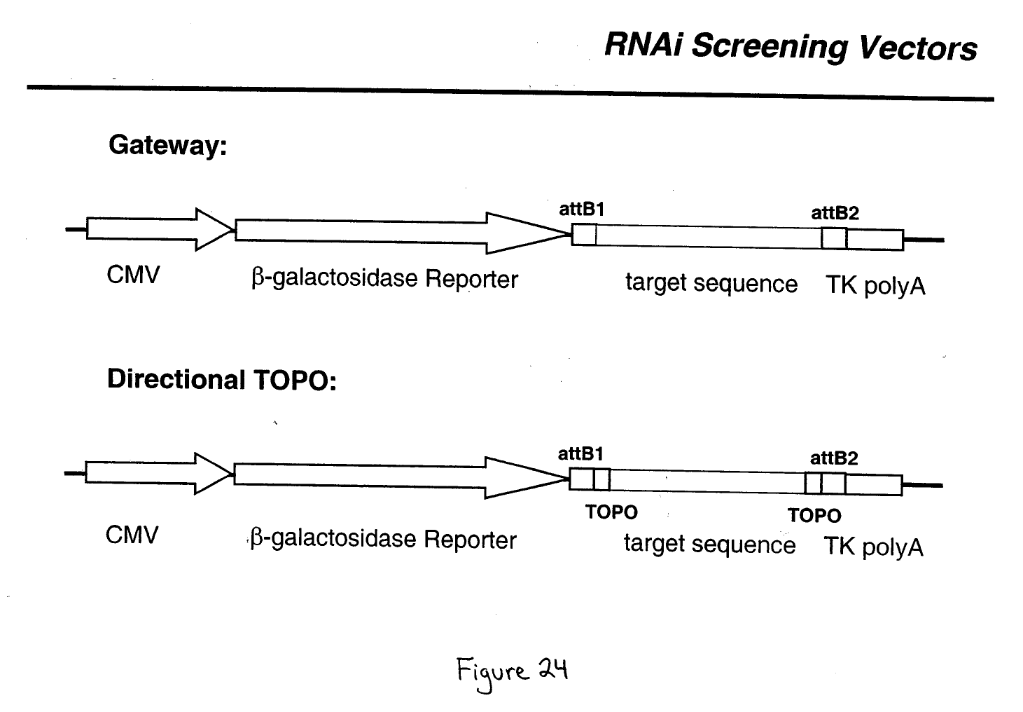

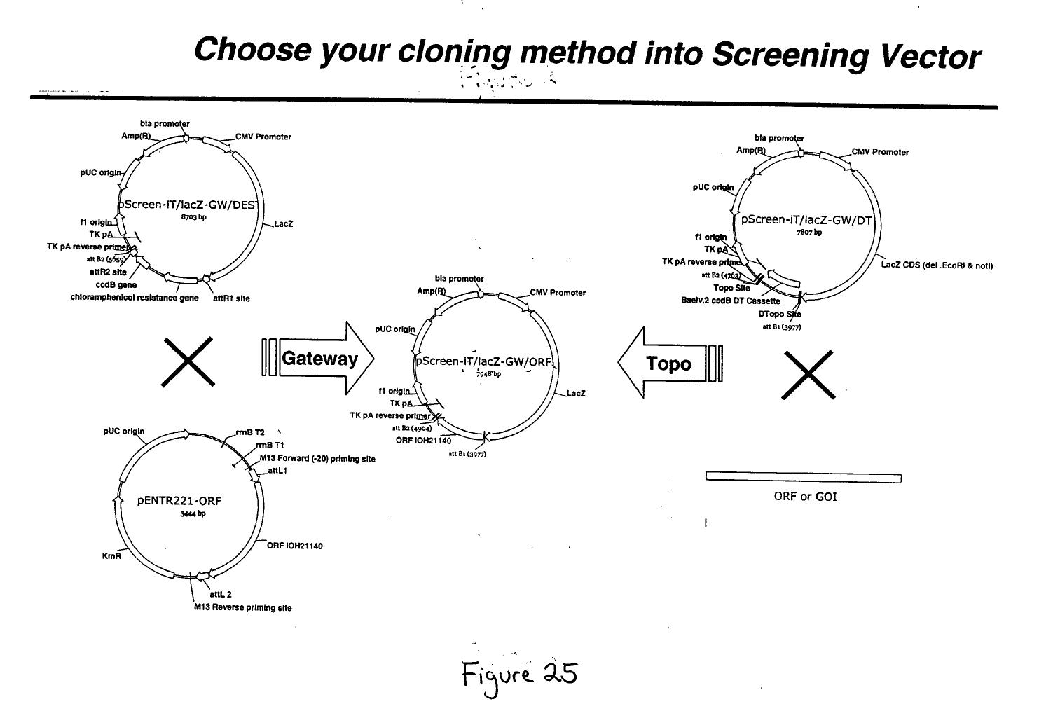

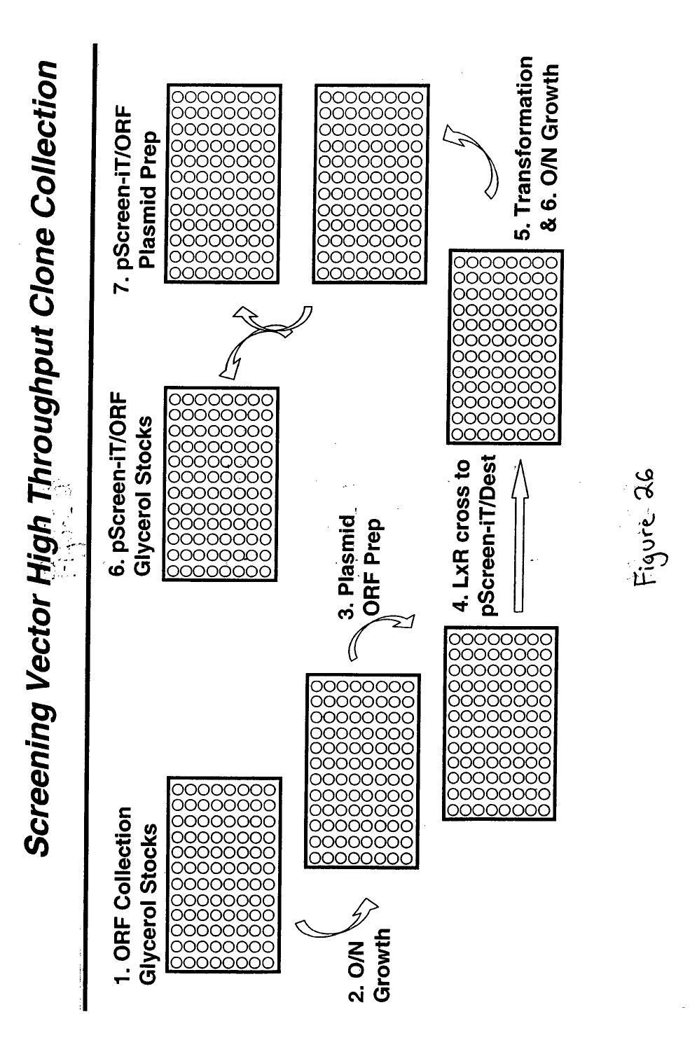

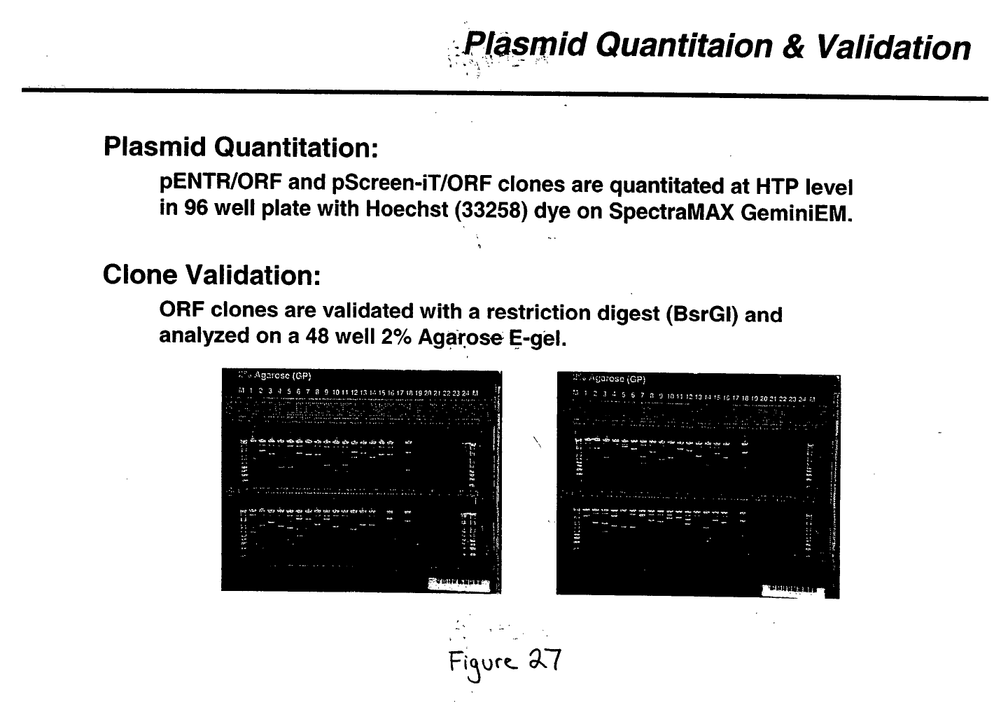

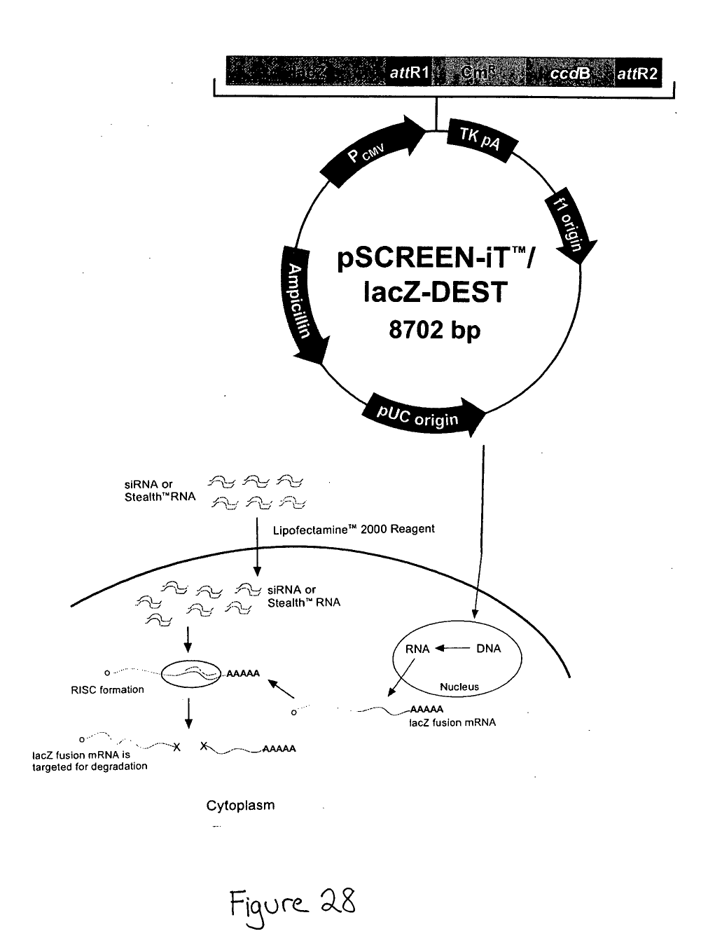

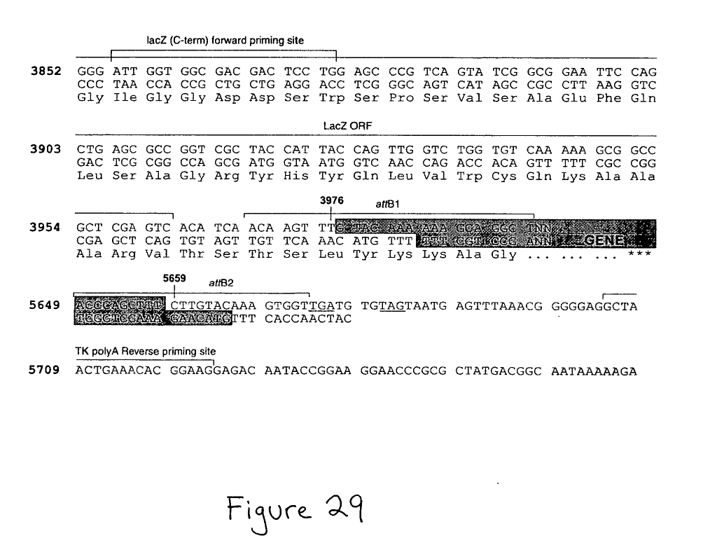

Those skilled in the art will appreciate that any of the β-lactamase enzymes referred to above, in addition to others, may be used in methods and/or compositions of the invention. For additional β-lactamases and a more detailed description of substrate specificities, consult Bush et al. (1995) Antimicrob. Agents Chemother. 39:1211-1233. Those skilled in the art will appreciate that the polypeptides having β-lactamase activity disclosed herein may be altered by for example, mutating, deleting, and/or adding one or more amino acids and may still be used in the practice of the invention so long as the polypeptide retains detectable β-lactamase activity. An example of a suitably altered polypeptide having β-lactamase activity is one from which a signal peptide sequence has been deleted and/or altered such that the polypeptide is retained in the cytosol of prokaryotic and/or eukaryotic cells. The amino acid sequence of one such polypeptide is provided in FIG. 10 (SEQ ID NO: 3).

One skilled in the art will appreciate that the sequence in FIG. 10 (SEQ ID NO: 3) may be modified and still be within the scope of the present invention. For example, the Asp at amino acid position number two of the polypeptide shown in FIG. 10 (SEQ ID NO: 3) may be changed to a Gly-His sequence.

The invention further includes methods for making RNA molecules such as those described in FIG. 2 and DNA molecules (e.g., vectors) which encode such RNA molecules.

As noted above, the invention also includes fusion nucleic acid molecules which encode tags. These tags may be detected by any number of means in methods of the invention. One example of a suitable tag is a LUMIO™ tag. A LUMIO™ tag (also referred to as a FlAsH tag) is a peptide which comprises the amino acid sequences C—C—X—X—C—C. A number of variations of this sequence (e.g., C—C—G—P—C—C) have been shown to bind to biarsenical compounds and become fluorescent in the process. Such peptides, as well as biarsenical compounds themselves, are described in U.S. Pat. No. 6,451,569, the entire disclosure of which is incorporated herein by reference.

When in vivo labeling of cells is employed, it will often be advantageous to add one or more compounds to the cell solution which absorb background light. One example of such a compound is Disperse Blue 3. One example of a method which may be used to label cells which express a protein with a suitable tetracysteine motif with FLASH™-EDT2 is the following. Cells are labeled for 90 minutes at room temperature with 2.5 μM FLASH™-EDT2 in OptiMEM™ (Invitrogen Corp., CA, see, e.g., cat nos. 11058-021, 31985-062, 31985-070, 31985-088, 51985-034). Cells are then gently washed once with OptiMEM™ and visualized in OptiMEM™ containing 20 μM Disperse Blue (Sigma-Aldrich, cat. no. 215651). Cells may then be photographed using a fluoresceine (FITC) filter with excitation wavelength 460-490 nm and emission wavelength 515-550 nm.

Additional tags which may be used in the practice of the invention include those which function as a selection marker. Often, these markers will function as negative selection markers. Examples of such markers include Diphtheria Toxin and Herpes simplex virus thymidine kinase (HSV TK). The choice of selection marker used will vary with the cell type employed. Typically, selection markers will be chosen which are functionally active in the cell type in which they are to be used.

Using HSV TK for purposes of illustration, RNA molecules of the invention may comprise nucleic acid which encodes HSV TK in a translatable format and nucleic acid corresponding to a gene of interest. Such RNA molecules may be used to monitor the progression of RNAi-mediated degradation of the nucleic acid corresponding to the gene of interest. For example, a reaction mixture may be formed in which these fusion RNA molecules are introduced into cells. Either a single dsRNA molecule or a mixed population of dsRNA molecules which correspond to the portion of the target RNA molecules corresponding to the gene of interest may then be added to the reaction mixture, which is then incubated under conditions which allow for RNAi-mediated degradation of the target RNA molecule. After a suitable period of time, an aliquot of cells may be removed and exposed to a compound such as ganciclovir. After a particular period of time, the percentage of cells which remain viable may then be measured. Other aliquots of cells may then be exposed to ganciclovir at timed intervals and the percentages of cells which remain viable may then also be measured. The alterations in the percentage of cells along the incubation time course may then be used as an indicator of the progression of RNAi-mediated degradation of the target RNA molecule.

The invention also includes the use of epitope tags. Expression of epitope tags can be monitored in numerous ways. For example, antigen/antibody reactions (e.g., ELISAs, radio immune assays, slot blots, etc.) may be used to detect the presence of the tag and/or quantification of the amount of tag present. Also, if the tag is localized to the cell surface, then cells (e.g., live cells) which contain the tag may be identified and/or separated using a detectably labeled (e.g., fluorescently labeled) antibody which binds to the tag, followed by a fluorescent activated cell sorter (FACS). Of course, FACS, for example, may also be used with non-epitope tags so long as the cells which contain the tag, or particular concentrations of the tag, can be distinguished from cells which either do not contain the tag or lesser amounts of the tag.

dsRNA Molecules Corresponding to Reporters and Tags

While FIG. 2 shows only the use of dsRNA molecules corresponding to the gene of interest, dsRNA molecules corresponding to reporters or other tags as well as 5′ and 3′ untranslated regions are also useful. One use of such dsRNA molecules is to knock-down expression of a fusion RNA which encodes both (1) a reporter and/or a tag and (2) a gene of interest. Thus, when a reporter and/or tag has been fused to a gene of interest in a manner which, for example, allows for production of the reporter and/or tag polypeptide and a polypeptide encoded by the gene of interest, degradation of the fusion RNA may be measured detecting alterations in reporter activity or tag functions. In other words, when a reporter and/or tag and a gene of interest are transcribed as part of the same transcript, the knock-down of one will correspond with the knock-down of the other. Thus, expression levels of a gene of interest may be measured by detection of reporter activity. This is especially important when one sees a phenotypic effect which results from alterations in expression of the gene of interest. Put another way, reporter expression can be used to measure expression levels of the gene of interest, which may then be correlated with phenotypic changes associated with alterations in expression in the gene of interest. Thus, the invention includes methods for detecting phenotypic expression of a gene of interest by measuring the activity of a reporter.

The invention further relates to individual RNA molecules which correspond to specific reporters, including β-galactosidase and β-lactamase. In many instances, these RNA molecules will have a nucleotide sequence which corresponds to part of the nucleotide sequence shown in FIG. 10 (SEQ ID NO: 4) (e.g., at least 15, 18, 19, 20, 21, 22, 23, 24, 25, 28, 30, 35, 40, 45, 50, etc. nucleotides). In most instances, these RNA molecules will not contain the entire nucleotides sequence shown in FIG. 10 (SEQ ID NO: 4) and further may be lacking at least 15, 18, 19, 20, 21, 22, 23, 24, 25, 28, 30, 35, 40, 45, or 50 of the nucleotides shown therein.

Further, individual RNA molecules of the invention may be single-stranded or double-stranded. As one skilled in the art would recognize, when one or more of these RNA molecules is used for RNA interference, in many instances, these RNA molecules will be double-stranded. Further, single-stranded RNA molecules of the invention may be combined with complementary RNA molecules to produce a double-stranded RNA molecule which may then be used for RNA interference.

As indicated above, RNA molecules of the invention include RNA molecules which are 19, 20, 21, 22, 23, 24, 25, or 26 nucleotides in length and correspond to the nucleotide sequence shown in FIG. 10 (SEQ ID NO: 4). Representative examples of such sequences are shown in the Table below.

TABLE 10

| | |
|---|---|
| RNA Sequence | SEQ ID NO |

| | |
|---|---|
| AUGGACCCAGAAACGCUGGUGA | 26 |
| AAACGCUGGUGAAAGUAAA | 27 |
| CCCCGAAGAACGUUUUCCAAUGAUG | 28 |
| CGUUUUCCAAUGAUGAGCAC | 29 |
| AGCACUUUUAAAGUUCUGCUA | 30 |
| CUCAGAAUGACUUGGUUGAG | 31 |
| UGGGAACCGGAGCUGAAUGA | 32 |
| AGCCAUACCAAACGACGAGCGUGAC | 33 |
| ACUGGCGAACUACUUACUCU | 34 |
| CACUCGCACCCAGAGCGCCA | 35 |
| AGACAGAUCGCUGAGAUAGGUG | 36 |
| CGACGGGGAGUCAGGCAACUA | 37 |
| UGCCUCACUGAUUAAGCAUU | 38 |

Each of the RNA sequences shown in the above Table is a sense sequence but the invention further includes antisense sequences shown in FIG. 10 (SEQ ID NO: 4). In particular embodiments, RNA molecules of the invention will be shorter than 25, 35, 45, 55, 65, 75, 90, 100, 150, 200, 250, 300, 350, 400, 450, 500, 550, 600, 650, 700, 750, or 800 nucleotides in length. In additional embodiments, RNA molecules of the invention will be between 19 and 25, 19 and 30, 19 and 40, 19 and 50, 25 and 30, 25 and 50, 25 and 100, 50 and 100, 50 and 200, 100 and 200, 100 and 300, 200 and 400, 200 and 60, or 300 and 500 nucleotides in length. The lengths referred to above may be the lengths of individual single-stranded RNA molecules of the invention or the lengths of dsRNA molecules of the invention. As already described above, when a dsRNA molecule contains overhangs on one or both ends, the dsRNA molecule may be longer than one or more of the single-stranded RNA molecules of which it is composed. The invention further includes similar RNA molecules corresponding to subportions of open reading frames which encode β-lactamases which differ from the amino acid sequence shown in FIG. 10 (SEQ ID NO: 3). Examples of such β-lactamases include Class A, B, C, and D β-lactamases such as those referred to above. Thus, in one aspect the invention includes dsRNA molecules which correspond to target RNA molecules which encode polypeptides having β-lactamase activity, as well as methods for using these dsRNA molecules in methods described herein and compositions (e.g., reaction mixtures) which contain these dsRNA molecules.

The invention includes RNA molecules in addition to those comprising sequence set out in the above Table. More specifically, the invention includes sense RNA molecules, antisense RNA molecules, and dsRNA molecules which correspond to various subportions (e.g., RNA molecules which are 19, 20, 21, 22, 23, 24, 25, or 26 nucleotides) of the nucleotide sequence shown in FIG. 10 (SEQ ID NO: 4). These subportions include portions of the nucleotide sequence shown in FIG. 10 (SEQ ID NO: 4) comprising nucleotides 1-50, 1-25, 25-45, 25-50, 25-75, 25-50, 50-75, 35-80, 35-55, 55-75, 45-95, 50-100, 50-75, 75-100, 65-120, 65-85, 85-105, 95-115, 80-150, 80-100, 100-120, 110-175, 125-180, 140-200, 160-230, 180-245, 200-250, 220-280, 240-300, 260-340, 300-390, 350-420, 370-430, 370-390, 390-410, 400-420, 400-480, 420-490, 450-500, 460-520, 480-530, 500-540, 520-565, 540-580, 550-600, 570-630, 590-635, 600-675, 630-700, 650-710, 670-750, 700-750, 710-780, 710-730, 730-750, 750-800, 750-770, 770-790, 780-800, 760-804. Examples of such RNA molecules are defined by the nucleotide sequences set out in the table above.

The invention also includes RNA molecules which correspond to RNA which encodes the β-lactamase protein set out in FIG. 10 (SEQ ID NO: 3) but, due to degeneracy of the genetic code, do not have the same nucleotide as that shown in FIG. 10 (SEQ ID NO: 4). The invention further includes subportions of such RNA molecules as described elsewhere herein.

When nucleic acids of the invention are designed, codons may be selected to encode particular amino acids. These codons vary, to some extent, with the translation system of the organism used but one example of a codon usage chart is set out in the table below. Codon selection is one example of a way that nucleic acids of the invention may be designed to have one or more desired properties (e.g., containing particular restriction sites, avoiding rare codons for a particular organism, etc.).

TABLE 11
Codon usage Chart

| | | | | | | | | | | | |
|---|---|---|---|---|---|---|---|---|---|---|---|
| TTT | F | Phe | TCT | S | Ser | TAT | Y | Tyr | TGT | C | Cys |
| TTC | F | Phe | TCC | S | Ser | TAC | Y | Tyr | TGC | C | Cys |
| TTA | L | Leu | TCA | S | Ser | TAA | * | Ter | TGA | * | Ter |
| TTG | L | Leu | TCG | S | Ser | TAG | * | Ter | TGG | W | Trp |
| CTT | L | Leu | CCT | P | Pro | CAT | H | His | CGT | R | Arg |
| CTC | L | Leu | CCC | P | Pro | CAC | H | His | CGC | R | Arg |
| CTA | L | Leu | CCA | P | Pro | CAA | Q | Gln | CGA | R | Arg |
| CTG | L | Leu | CCG | P | Pro | CAG | Q | Gln | CGG | R | Arg |
| ATT | I | Ile | ACT | T | Thr | AAT | N | Asn | AGT | S | Ser |
| ATC | I | Ile | ACC | T | Thr | AAC | N | Asn | AGC | S | Ser |
| ATA | I | Ile | ACA | T | Thr | AAA | K | Lys | AGA | R | Arg |
| ATG | M | Met | ACG | T | Thr | AAG | K | Lys | AGG | R | Arg |
| GTT | V | Val | GCT | A | Ala | GAT | D | Asp | GGT | G | Gly |
| GTC | V | Val | GCC | A | Ala | GAC | D | Asp | GGC | G | Gly |
| GTA | V | Val | GCA | A | Ala | GAA | E | Glu | GGA | G | Gly |
| GTG | V | Val | GCG | A | Ala | GAG | E | Glu | GGG | G | Gly |

For each triplet, the single and three letter abbreviation for the encoded amino acid is shown. Stop codons are represented by *.

The invention thus includes nucleic acid molecules which encode a β-lactamases referred to herein but which have undergone one or more (e.g., one, two, three, four, five, six, seven, etc.) codon alterations. The invention further includes RNA molecules which correspond to altered nucleic acid (e.g., DNA) which encodes these β-lactamases, as well as subportions thereof.

Vectors and Other Nucleic Acid Molecule

Vectors and other nucleic acid molecules of the invention, as well as nucleic acids used in methods of the invention may comprise one or more recombination site. In many instances, these recombination sites will used to generate a nucleic acid molecules which encode a fusion RNA transcript.

A considerable number of recombination systems which are adaptable for recombinational cloning are known in the art. One example of such a system in the Cre/lox system. The recombination site for Cre recombinase is loxP which is a 34 base pair sequence comprised of two 13 base pair inverted repeats (serving as the recombinase binding sites) flanking an 8 base pair core sequence (see FIG. 1 of Sauer, B., Curr. Opin. Biotech. 5:521-527 (1994).) Other examples of recognition sequences include the attB, attP, attL, and attR sequences which are recognized by the recombination protein λ Int. attB is an approximately 25 base pair sequence containing two 9 base pair core-type Int binding sites and a 7 base pair overlap region, while attP is an approximately 240 base pair sequence containing core-type Int binding sites and arm-type Int binding sites as well as sites for auxiliary proteins integration host factor (IHF), FIS and excisionase (Xis). (See Landy, Curr. Opin. Biotech. 3:699-707 (1993).)

Additionally, cloning systems that utilize recombination at defined recombination sites have been previously described in the related applications listed above, and in U.S. application Ser. No. 09/177,387, filed Oct. 23, 1998; U.S. application Ser. No. 09/517,466, filed Mar. 2, 2000; and U.S. Pat. Nos. 5,888,732 and 6,143,557, all of which are specifically incorporated herein by reference. In brief, the GATEWAY™ Cloning System, described in this application and the applications referred to in the related applications section, utilizes vectors that contain at least one recombination site to clone desired nucleic acid molecules in vivo or in vitro. More specifically, the system utilizes vectors that contain at least two different site-specific recombination sites based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are typically cloned and subcloned using the GATEWAY™ system by replacing a selectable marker (for example, ccdb) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

Mutating specific residues in the core region of the att site can generate a large number of different att sites. As with the att1 and att2 sites utilized in GATEWAY™, each additional mutation potentially creates a novel att site with unique specificity that will recombine only with its cognate partner att site bearing the same mutation and will not cross-react with any other mutant or wild-type att site. Novel mutated att sites (e.g., attB 1-10, attP 1-10, attR 1-10 and attL 1-10) are described, for example, in U.S. Patent Publication No. 2002/0007051, which is specifically incorporated herein by reference. Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites; loxP site mutants, variants or derivatives such as loxP511 (see U.S. Pat. No. 5,851,808); frt sites; frt site mutants, variants or derivatives; dif sites; dif site mutants, variants or derivatives; psi sites; psi site mutants, variants or derivatives; cer sites; and cer site mutants, variants or derivatives.

The present invention also involves the use of methods for linking a first and at least a second nucleic acid segment (either or both of which may contain viral sequences and/or sequences of interest) with at least one (e.g., 1, 2, 3, 4, 5, 6, 7, 8, 9, 10, etc.) topoisomerase (e.g., a type IA, type IB, and/or type II topoisomerase) such that either one or both strands of the linked segments are covalently joined at the site where the segments are linked.

A method for generating a double stranded recombinant nucleic acid molecule covalently linked in one strand can be performed by contacting a first nucleic acid molecule which has a site-specific topoisomerase recognition site (e.g. , a type IA or a type II topoisomerase recognition site), or a cleavage product thereof, at a 5′ or 3′ terminus, with a second (or other) nucleic acid molecule, and optionally, a topoisomerase (e.g., a type IA, type ID, and/or type II topoisomerase), such that the second nucleotide sequence can be covalently attached to the first nucleotide sequence. As disclosed herein, the methods of the invention can be performed using any number of nucleotide sequences, typically nucleic acid molecules wherein at least one of the nucleotide sequences has a site-specific topoisomerase recognition site (e.g., a type IA, type IB or type II topoisomerase), or cleavage product thereof, at one or both 5′ and/or 3′ termini

Kits

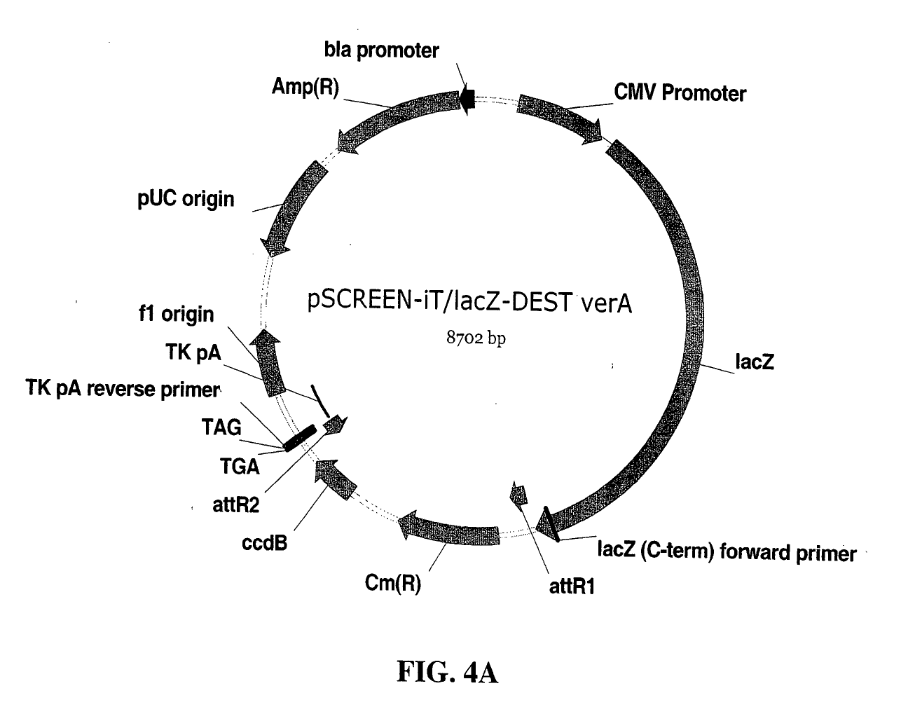

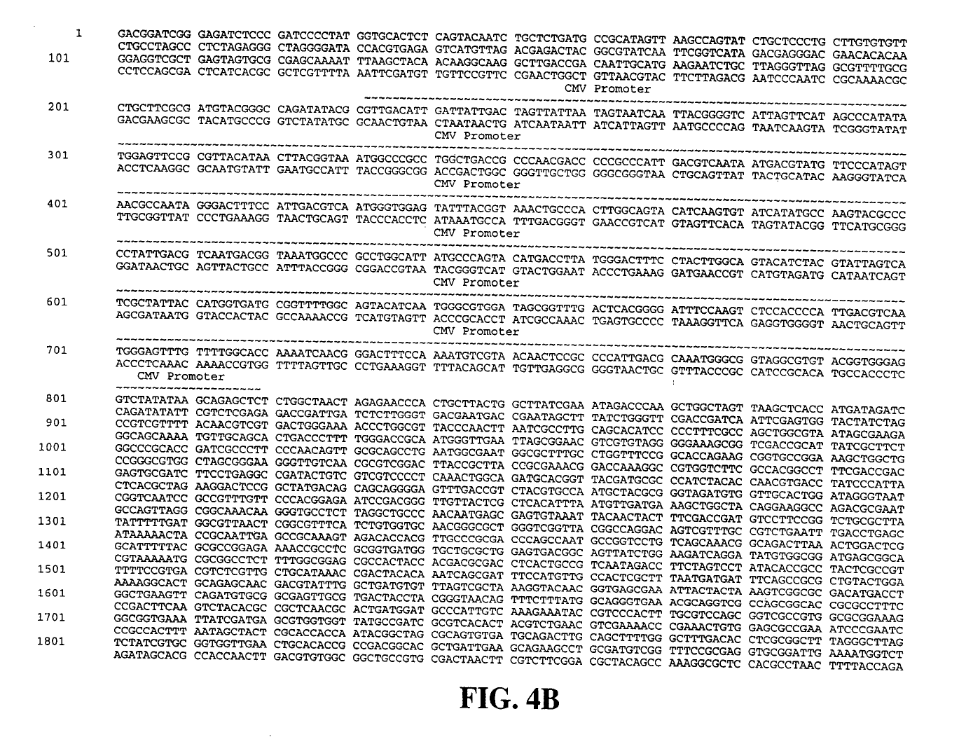

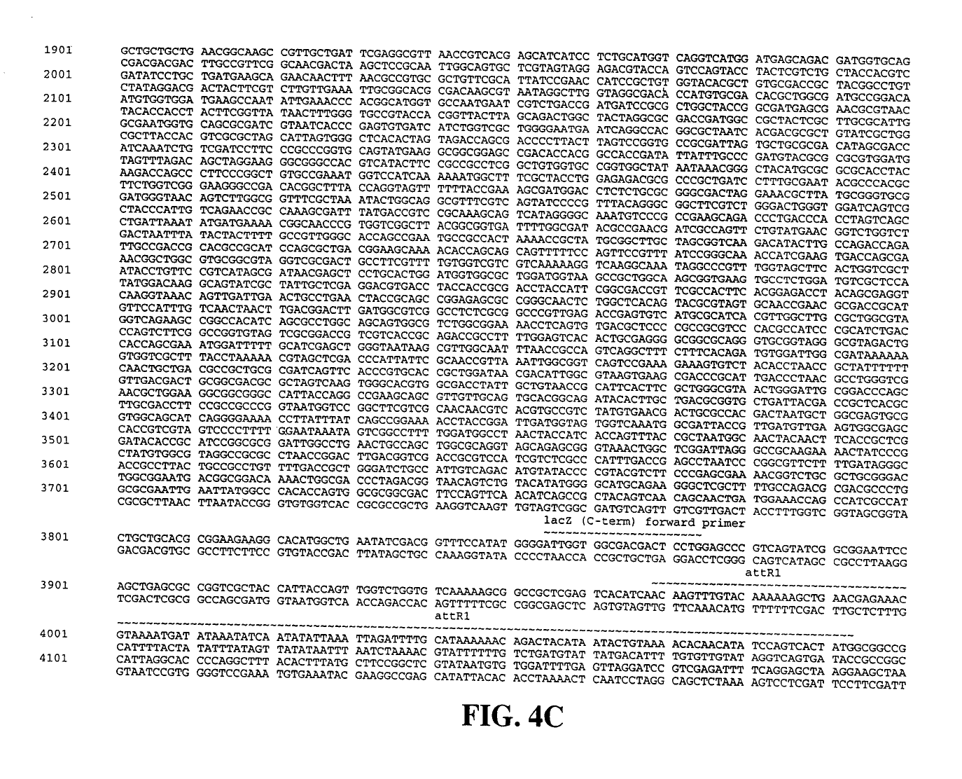

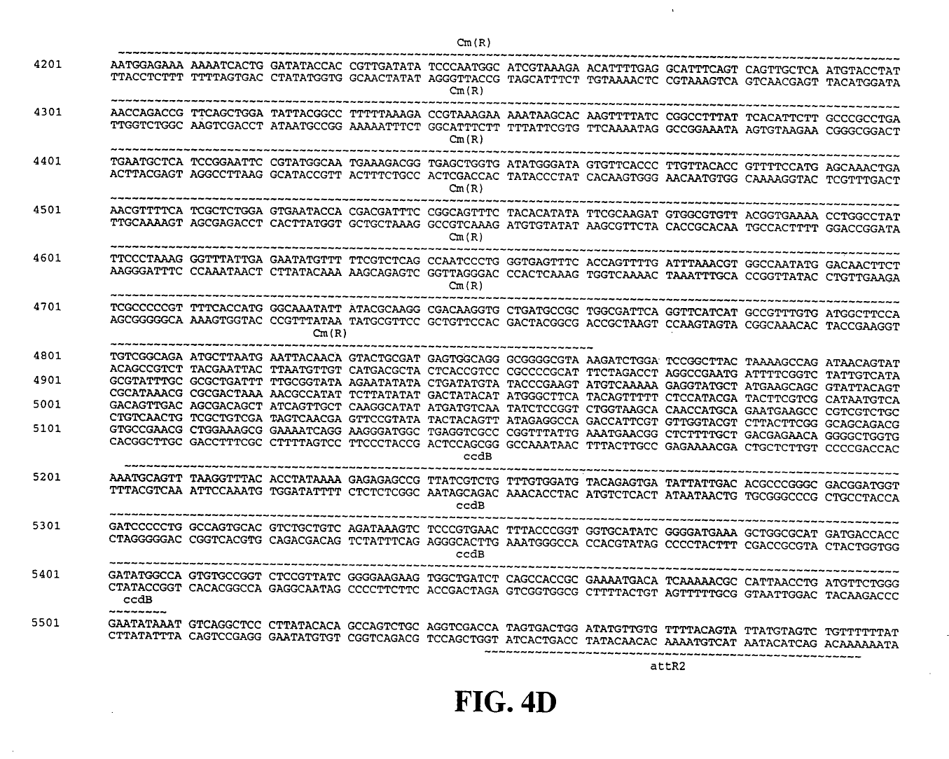

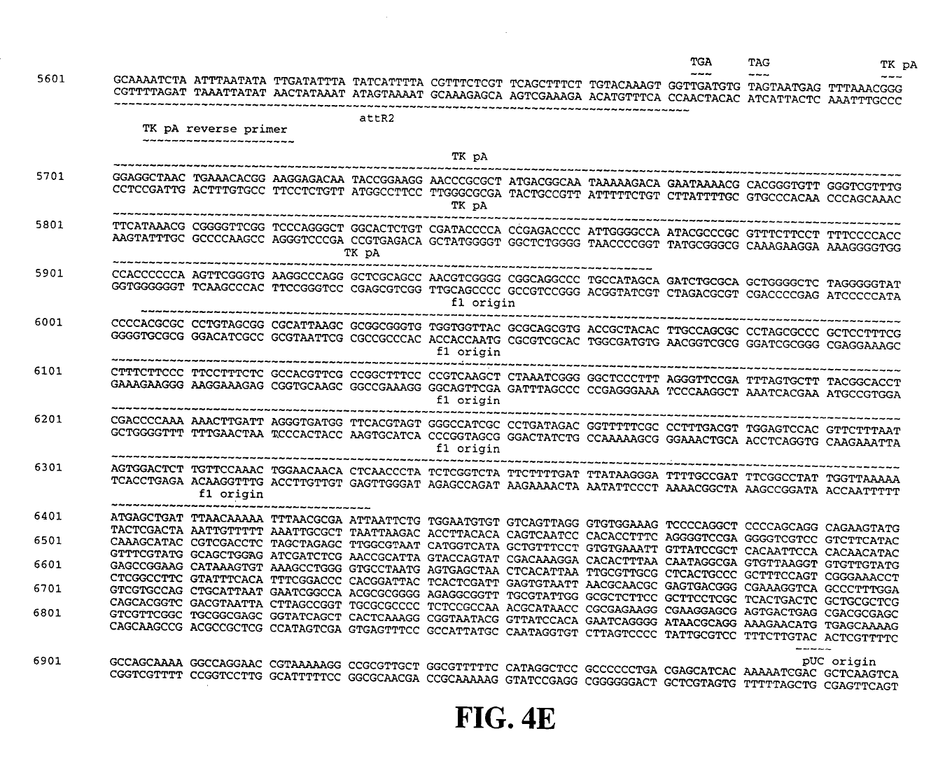

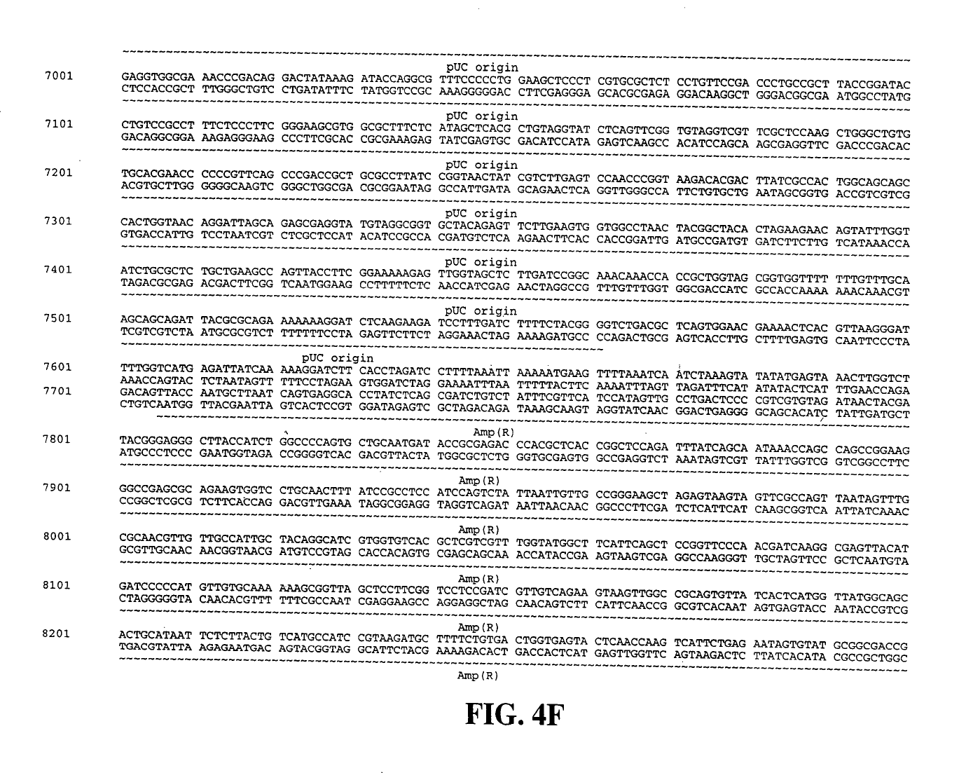

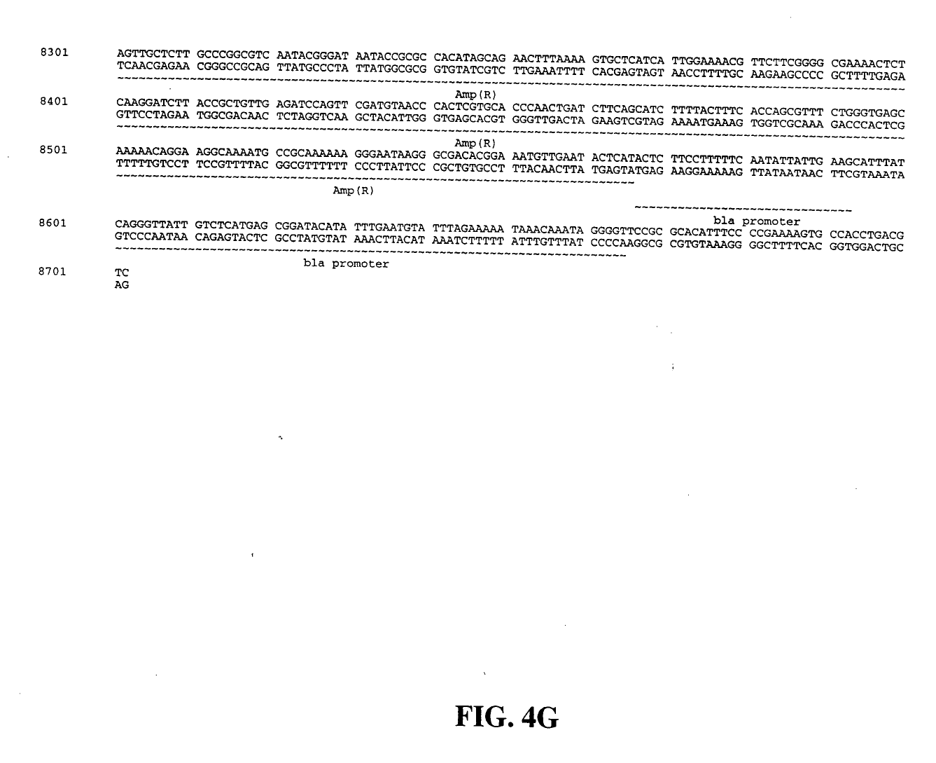

The invention includes kits for identifying one or more RNAi cleavage sites along a target RNA molecule. Kits of the invention may comprise, for example, one or more of the following: (a) one or more dsRNA molecules and/or one or more mixed populations of dsRNA molecules; (b) one or more single-stranded RNA molecules; (c) one or more cells; (d) one or more reagents for introducing nucleic acid molecules into cells (e.g., LIPOFECTAMINE 2000™); (e) one or more enzymes having RNase activity (e.g., a Dicer enzyme); (f) one or more enzymes having RNA polymerase activity; (g) one or more enzymes having DNA polymerase activity; (h) one or more restriction endonucleases; (i) one or more nucleotides; j) one or more enzymes having DNase activity; (k) one or more buffers; (l) one or more plasmids which allows for cloning and/or expression of the nucleic acid of interest (exemplified by the vector shown in FIG. 4), and (m) one or more sets of instructions for performing methods of the invention and/or using kit components.

Kits of the invention may include an instruction set, or the instructions can be provided independently of the kits. Such instructions are characterized, in part, in that they provide a user with information related to performing methods of the invention and/or using kit components. Such instructions may include various details such as suggested reaction times and buffer formulations to be employed.

Instructions may be provided in kits, for example, written on paper or in a computer readable form provided with the kits, or can be made accessible to a user via the internet, for example, on the world wide web at a URL (uniform resources link; i.e., “address”) specified by the provider of the kits or an agent of the provider. Such instructions direct a user of the kits or other party of particular tasks to be performed or of particular ways for performing a task.

The invention further includes product literature which describes methods and compositions of the invention. See Example 4.

The following examples are illustrative, but not limiting, of the method and compositions of the present invention. Other suitable modifications and adaptations of the variety of conditions and parameters normally encountered in the biological and chemical sciences which are obvious to those skilled in the art in view of the present disclosure are within the spirit and scope of the invention.

### EXAMPLES

### Example 1

### Determining the Sites of RNAi Cleavage Along a Target RNA Molecule Using RNAse Generated Mixed Populations of siRNA Preparation of Mixed Populations of SIRNA Molecules

As discussed below, mixed populations of siRNA molecules are prepared by a) chemically synthesizing synthetic populations of siRNAs or b) cleaving two or more dsRNA molecules with enzymes having RNase activity. Typically, the mixed populations of dsRNA molecules used will correspond to one or more target RNA molecules.

Preparing a Mixed Population of Synthetic siRNAS

RNA oligonucleotides of varying length can be synthesized using standard nucleic acid chemistry. Individual ssRNA oligonucleotides can be annealed to a complementary RNA sequence in vitro. Further, a mixed population of dsRNA molecules can be generated by mixing one or more dsRNA molecules. Alternatively, mixed populations of complementary ssRNA oligonucleotides can be annealed in the same reaction to generated a mix population of dsRNA molecules. siRNA sequences used in the invention may correspond to a defined nucleic acid target sequence or may contain random sequences. As indicated above, typically, the mixed populations of dsRNA molecules used will correspond to one or more target RNA molecules.

Preparing dsRNA Molecules Using an RNAse Activity

A target RNA molecule is first selected. The target RNA molecule will often be the transcript of a gene of interest that an investigator is seeking to silence in a particular cell or organism. Once the target RNA molecule is selected, dsRNA molecules are produced that correspond to all or a portion of the target RNA molecule.

Any number of methods may be used to generate target RNA molecules for use in methods of the invention. In many instances such methods will involve generating nucleic acid molecules that encode target RNA molecules in a format which allows for convenient production of target RNA molecules. For example, a gene of interest may be amplified by PCR using a forward primer that contains a T7 promoter and a gene specific reverse primer. In a second reaction, the gene of interest is amplified by PCR using a gene specific forward primer and a reverse primer which contains a T7 promoter. The result of these two amplification reactions is two separate DNA molecules. With one of these DNA molecules, T7 polymerase mediated transcription results in the production of sense RNA corresponding to the gene of interest. With the other DNA molecule, T7 polymerase mediated transcription results in the production of antisense RNA corresponding to the gene of interest. These sense and antisense RNA molecules may then be annealed to each other to generate an intact dsRNA molecules. Thus, after PCR amplification, sense strand and antisense strand RNAs may be generated in separate reactions by in vitro transcription using T7 RNA polymerase. The sense strand and antisense strand RNAs may be purified from the transcription reaction mixtures using standard RNA isolation methods including those set out in the BLOCK-iT™ RNAi purification system (cat. no. K3500-01, Invitrogen Corporation, Carlsbad, Calif.) and allowed to anneal to form dsRNA molecules. Alternatively, the sense and antisense strands may be generated in the same reaction where they can combine to form dsRNA. The dsRNA may then be purified by standard isolation methods including the BLOCK-iT™ RNAi purification system.

In some cases, not all of the gene of interest is amplified. For example, an investigator may wish to focus on the RNAi cleavage sites found within the first one-third of the mRNA transcribed from the gene of interest. Thus, the first one-third of the gene of interest may be amplified with the forward and reverse primers set forth above. Alternatively, a nucleic acid fragment of a gene of interest can amplified by both a forward or reverse primer containing a T7 promoter sequence. Additionally, any nucleic acid fragment from the gene of interest can be ligated to second DNA molecule containing the sequence of the T7 promoter (e.g., TOPO linker or plasmid vector). While this example refers to the generation of RNA molecules using a T7 promoter, any promoter suitable for use in in vitro transcription reactions may be used (e.g., the T3 promoter, the SP6 promoter, etc.).

IN VITRO Dicing of dsRNA Molecules

The dsRNA molecules are then subjected to cleavage by an RNase enzyme to produce a mixed population of ds RNA molecules. Such cleavage reactions may be referred to as “in vitro dicing.” In vitro “dicing” reactions can be carried out as follows: 60 μg of dsRNA is mixed with 60 units of recombinant human Dicer in 300 μl of reaction buffer (250 mM NaCl, 3 mM MgCl₂, 50 mM Tris-HCl (pH 8.5 )). The reactions are incubated for 14 to 16 hrs at 37° C. Detailed methods for in vitro dicing reactions are also set out in the BLOCK-iT™ Dicer RNAi Transfection Kit (Invitrogen Corporation, Carlsbad, Calif., cat. nos. K3600-01 and K3650-01).

siRNA molecules of 21-23 nucleotides in length are recovered from the reaction mixture using a BLOCK-iT™ RNAi purification system The purified siRNAs are eluted in RNase free H₂O and quantified by absorbance at 260 nm. siRNA molecules may also be purified using methods set out in the BLOCK-iT™ Dicer RNAi Transfection Kit (Invitrogen Corporation, Carlsbad, Calif., cat. nos. K3600-01 and K3650-01).

Complex siRNA Mixed Populations

As an alternative to the method described above, mixed populations of dsRNA molecules may be produced which comprise siRNA molecules corresponding to multiple portions of a single target RNA molecules or multiple target RNA molecules. For example, the first one-third (or other portion) of a first gene of interest is amplified by PCR and intact dsRNA molecules are generated as described above. In parallel, the first one-third (or other portion) of a second gene of interest is also amplified by PCR and intact dsRNA molecules are generated as described above.

The first and second sets of intact dsRNA molecules are then combined. Rather than dicing the intact dsRNA molecules separately and then combining the two sets of diced dsRNA molecules, the intact dsRNA molecules are combined before dicing. The combined dsRNA molecules are then subjected to cleavage by an RNase enzyme (as above) to produce a mixed population of dsRNA molecules which correspond to two different target RNA molecules.

Introduction of the Mixed Population of dsRNA Molecules into Cells

The mixed population of ds RNA molecules is then introduced into cells which express the gene of interest (i.e., the cells contain the target RNA molecule). For example, 293 cells that normally express the gene of interest, or are engineered to express the gene of interest, are transfected with 25 nM of the mixed population of dsRNA molecules using the LIPOFECTAMINE 2000 reagent (Invitrogen Corporation, Carlsbad, Calif., cat. no. 11668-019) in accordance with the manufacturer's instructions. Suitable concentrations of mixed population of dsRNA molecules which may be used in these methods vary but may include 1.0 fM to 1.0 μM (e.g., 1.0 fM to 400 nM, 0.04 nM to 1.0 μM, 2 nM to 500 nM, 100 nm to 1.0 μM, etc.).

The transfected cells are incubated at 37° C. for 24 to 72 hours. GRIPTITE™ 293 are cutltuers in DMEM, 10% FBS, 0.1 mM Non-Essential amino acids, and 600 μg/ml Geneticin. FLP-in Luc 293 cells are cultured DMEM, 10% FBS, 0.1 mM Non-Essential amino acids, and 100 μg/ml hygromycin B. The incubation period facilitates the production of target RNA fragments due to siRNA-mediated cleavage of the target RNA molecule using the intracellular RNAi machinery. Suitable incubation times range from as little as 15 minutes to as much as two weeks (e.g., 15 minutes to 96 hours, 1 hour to 96 hours, 2 hours to 48 hours, 3 hours to 48 hours, 3, hours to 24 hours, 5 hours to 24 hours, 6 hours to 24 hours, 8 hours to 24 hours, 8 hours to 48 hours, 8 hours to 72 hours, 12 hours to 24 hours, 12 hours to 48 hours, 24 hours to 48 hours, 36 hours to 72 hours, 36 hours to 96 hours, etc.). This is so because the RNAi mediated degradation process begins shortly after dsRNA molecules are introduced into cells. Thus, short incubation times may be used in the practice of methods of the invention. Longer incubation times (e.g., those greater than 48 hours) may be used in situations where the cleaved target RNA molecule and/or the individual members of the mixed population of dsRNA molecules are not rapidly degraded.

Isolation of Cleaved Target RNA Molecules from Cells

Total RNA or pol(A)⁺ RNA may then be isolated from the transfected cells. An exemplary method that can be used is the Micro-to-Midi Total RNA Purification System (Invitrogen Corp. Carlsbad, Calif., cat. no. 12183-018). Briefly, cells are directly lysed in the culture dish by adding 600 μl of RNA Lysis solution containing 1% β-mercaptoethanol. The cells are frozen at −80° C. in the lysis solution, thawed at 25° C., and passed through a pipet to homogenize the cells. One volume of 80% ethanol is mixed with each cell homogenate. The mixture is centrifuged through a RNA spin cartridge at 20,000×g for 15 minutes at 25° C. in 600 μl aliquots. 700 μl of Wash Buffer I is then applied to the RNA spin cartridge, which is then centrifuged at 20,000×g for 15 minutes at 25° C. The RNA spin cartridge is then washed two consecutive times with 500 μl of Wash Buffer II containing ethanol in a similar manner. The RNA spin cartridge is dried by centrifugation at 20,000×g for 1 minute at 25° C. The RNA is then eluted from the spin cartridge in 20 μl of DEPC treated water. Other preferable methods of RNA isolation include the TRIZOL™ method (Invitrogen Corporation, Carlsbad, Calif., cat. no. 15596-026), FastTrack™ and MICRO-FASTTRACK™ methods (Invitrogen Corporation, Carlsbad, Calif., cat. nos. K1593-02 and K1520-02), Oligo dT cellulose mediated mRNA isolation (Invitrogen Corporation, Carlsbad, Calif., cat. no. R545-01).

It may be advantageous to treat the RNA sample with DNase I to eliminate residual genomic DNA. For example, 1.5 μl amplification grade DNase I is added to a 20 μl reaction containing the RNA sample in reaction buffer (20 mM Tris-HCl (pH 8.4), 50 mM KCl and 2 mM MgCl₂). The reaction is incubated at room temperature for 15 minutes. 1.5 μl of 25 mM EDTA is added, and the reaction is incubated at 10 minutes at 65° C. The remaining RNA is purified by phenol:chloroform extraction and ethanol precipitation. The RNA pellet is resuspended in 7 μl of DEPC treated H₂O.

Obtaining DNA Molecules Complementary to the Target RNA Fragments Using RNA Ligase-Mediated Rapid Amplification of 5′ cDNA Ends Ligation of an RNA Oligonucleotide to the mRNA

A Long RNA Oligonucleotide, (e.g., GENERACER™ RNA Oligo: CGACUGGAGCACGAGGACACUGACAUGGACUGAAGGAGUAGAAA, SEQ ID NO: 39, see the product manual associated with Invitrogen Corporation's cat. no. L1500-01) is first ligated to the 5′ termini of the isolated RNA molecules.

A 10 μl mixture is prepared containing: 0.25 μg of the RNA oligonucleotide, 1-5 μg of isolated total RNA (see above), 33 mM Tris-Acetate, pH 7.8, 66 mM potassium acetate, 10 mM magnesium acetate, 500 μM DTT, 1 mM ATP, 4 U/μl RNaseOut, and 5 U of T4 ligase. This mixture is incubated for 1 hour at 37° C. The mixture is then chilled on ice for 1 minute. The RNA is purified by phenol:chloroform extraction and ethanol precipitation. The RNA pellet is resuspended in 10 μl of DEPC treated H₂O.

Reverse Transcription of Ligated RNA

The population of ligated RNA (11 μl) is then mixed with 1 μl of Oligo dT primer (50 μM) and 1 μl of dNTP Mix (10 mM each) and incubated at 65° C. for 5 minutes to remove secondary structure. The mixture is then chilled on ice for 2 minutes and centrifuged briefly. The following are then added to the mixture: 4 μl of 5× First Strand Buffer (250 mM Tris-HCl (pH 8.3), 375 mM KCl, 15 mM MgCl₂), 2 μl of 0.1 M DTT, 1 μl of RNaseOut (40 U/μl) (Invitrogen Corporation, Carlsbad, Calif., cat. no. 10777-019), and 1 μl of SUPERSCRIPT™ II RT (200 U/μl) (Invitrogen Corporation, Carlsbad, Calif., cat. no. 18064-014). The reaction is incubated at 42° C. for 50 minutes. The reaction is then incubated at 70° C. for 15 minutes to terminate the reaction. The reaction is then chilled in ice for 1 minute and centrifuged briefly. 1 μl of RNase H (2 U) is added, and the resulting mixture is incubated at 37° C. for 20 min. The reaction is collected by brief centrifugation and placed on ice.

Amplification of cDNA Molecules

The cDNA molecules are then PCR amplified using a forward primer that is specific for the GENERACER™ RNA Oligo, GENERACER™ 5′ Primer: CGACTGGAGCACGAGGACACTGA (SEQ ID NO: 40), and a reverse primer that is specific for a sequence within the cDNA. The cDNA-specific primer is complementary to a nucleotide sequence within the target RNA molecule located downstream from the suspected RNAi cleavage sites. PCR amplification is carried out using standard techniques. A second PCR amplification can be performed using a sample of the primary PCR as template to amplify fragments of low abundance in the primary PCR reaction. The nested PCR reaction includes a forward primer that is specific for the GENERACER™ RNA Oligo and 3′ to the GENERACER™ 5′ Primer, GENERACER™ 5′ Nested Primer: GGACACTGACATGGACTGAAGGAGTA (SEQ ID NO: 41), and a gene specific reverse primer that is 5′ to the gene specific primer previously used above.

Obtaining DNA Molecules Complementary to the Target RNA Fragments Using RNA Ligase-Mediated Rapid Amplification of 3′ cDNA Ends

Using a method similar to described above, a 3′ Long RNA oligonucleotide containing a 5′ phosphate and preferably a 3′ blocking group can be ligated to the 3′ end of RNA isolated from cells treated with the dsRNA. An oligonucleotide capable of hybridizing to the 3′ Long RNA oligonucleotide is used to prime the synthesis of the cDNA strand in a reaction containing a reverse transcriptase (e.g., SUPERSCRIPT™ II RT). PCR amplification of the cDNA molecules is performed using forward primers specific a sequence in the cDNA and reverse primers specific to sequences specific to the 3′ Long RNA oligonucleotide.

Cloning and Sequencing the cDNA Molecules

After the complementary DNA molecule are produced and amplified, they are cloned into a suitable vector using methods that are well known in the art. The ends of the complementary DNA molecules are then sequenced using standard DNA sequencing methods. The primer for DNA sequencing can be a primer that is specific for a region of the vector located near or adjacent to the site at which the complementary DNA molecule has been inserted.

Determining the Sites of RNAi Cleavage Based on the Sequence of the RNA Ligase-Mediated Rapid Amplification PCR Products

The nucleotide sequences of the ends of the complementary DNA molecules can be used to determine the sites of RNAi cleavage. The sequences will be the complement of the sequences found at the ends of the target RNA fragments that are produced as described above. The sequences found at the ends of the target RNA fragments are compared to and aligned with the matching sequences within the intact target RNA molecule. The sites along the intact target RNA molecule that correspond to the ends of the target RNA fragments are the sites of RNAi cleavage.

### Example 2

### Obtaining DNA Molecules Complementary to the Target RNA Fragments Using 5′ Race First Strand cDNA Synthesis from Total RNA

A first primer is designed that is specific for a sequence found within the target RNA molecule. The first primer is complementary to a nucleotide sequence within the target RNA molecule located downstream from possible or suspected RNAi cleavage sites.

A mixture is prepared containing: 10 to 25 ng of the first primer, 1-5 μg of isolated total RNA (see above), and DEPC-treated water sufficient to bring the reaction to a final volume of 15.5 μl. This mixture is incubated for 10 minutes at 70° C. to denature the RNA. The mixture is then chilled on ice for 1 minute. The following are then added to the mixture: 2.5 μl of 10× PCR buffer (200 mM Tris-HCl (pH 8.4), 500 mM KCl), 2.5 μl of 25 mM MgCl₂, 1 μl of 10 mM dNTP mix (10 mM each dATP, dCTP, dGTP, dTTP), and 2.5 μl of 0.1 M DTT. The resulting mixture is incubated at 42° C. for 1 minutes. 1 μl of SUPERSCRIPT™ II RT (200 units/l) is added, and the reaction is incubated at 42° C. for 50 minutes. The reaction is then incubated at 70° C. for 15 minutes to terminate the reaction. The reaction is then placed at 37° C. 1 μl of RNase mix (a mixture of RNase H and RNase T1) is added, and the resulting mixture is incubated at 37° C. for 30 minutes. The reaction is collected by brief centrifugation and placed on ice.

Purification of First Strand Products

Excess nucleotides and the first primer are then removed from the first strand products. The first strand products can be purified, for example, using the S.N.A.P. column procedure (Invitrogen Corp. Carlsbad, Calif., cat. no. K1900-01), adapted from the method of Vogelstein and Gillespie, Proc. Natl. Acad. Sci. USA 76:615 (1979).

Homopolymeric Tailing of cDNA Molecules

A homopolymeric tail is then added to the 3′ end of the purified first strand products. A mixture is first prepared containing: 6.5 l of DEPC-treated water, 5 μl of 5× tailing buffer, 2.5 μl of 2 mM dCTP, and 10.0 μl of purified first strand product from above. The mixture is incubated at 94° C. for 2 to 3 minutes. The mixture is then chilled 1 minutes on ice. 1 μl of terminal deoxynucleotidyl transferase (TdT) is added and the reaction is incubated at 37° C. for 10 minutes. The TdT is then heat inactivated at 65° C. for 10 minutes.

Amplification of cDNA Molecules

The “tailed” cDNA molecules are then PCR amplified using a primer that is specific for the tail and a primer that is specific for a sequence within the cDNA molecules. The cDNA-specific primer is complementary to a nucleotide sequence within the target RNA molecule located downstream from the suspected RNAi cleavage sites. PCR amplification is carried out using standard techniques.

Cloning and Sequencing the cDNA Molecules

After the complementary DNA molecule are produced and amplified, they are cloned into a suitable vector using methods that are well known in the art. The ends of the complementary DNA molecules are then sequenced using standard DNA sequencing methods. The primer for DNA sequencing can be a primer that is specific for a region of the vector located near or adjacent to the site at which the complementary DNA molecule has been inserted.

Determining the Sites of RNAi Cleavage Based on the Sequence of the 5′ Race Products

The nucleotide sequences of the ends of the complementary DNA molecules can be used to determine the sites of RNAi cleavage. The sequences will be the complement of the sequences found at the ends of the target RNA fragments that are produced as described above. The sequences found at the ends of the target RNA fragments are compared to and aligned with the matching sequences within the intact target RNA molecule. The sites along the intact target RNA molecule that correspond to the ends of the target RNA fragments are the sites of RNAi cleavage.

### Example 3

RNAi Screening Vector: pSCREEN-iT™/lacZ-DEST and Kits Containing the Same

Abstract. To suppress gene expression using RNA interference, multiple reagents (siRNAs, STEALTH™ molecules, or shRNA vectors) often need to be tested to identify those with sufficient potency. In many cases, the phenotype that will result from knockdown is unknown, so target protein or mRNA levels must be checked; however, this can be difficult and time consuming. This document describes an RNAi screening vector, pSCREEN-iT1™/lacZ-DEST, which enables GATEWAY™ cloning of target RNA sequences behind a lacZ reporter. β-galactosidase activity serves as a simple and accurate readout of target RNA knockdown and correlates with qRT-PCR data from the endogenous transcript. The vector carries no stop codon after the lacZ coding region, a feature that is essential for accurate results with full-length inserts such as those from the Ultimate™ ORF collection, available from Invitrogen Corporation, Carlsbad, Calif.

Introduction

RNA interference (RNAi) is a powerful tool for molecular genetics analysis of gene function in mammalian cells. In RNAi, the primary effector molecules are double-stranded short interfering RNAs (siRNAs, reviewed in Dykxhoorn, D. M., Novina, C. D., and Sharp, P. A. (2003). Killing the Messenger: Short RNAs that Silence Gene Expression. Nat. Rev. Mol. Cell Biol. 4, 457-467). One strand of each siRNA molecule is incorporated into a cytoplasmic, multi-protein RNA-Induced Silencing Complex (RISC) and serves as a guide for locating complementary target RNAs. A RISC nuclease, provisionally identified as a homologue of micrococcal nuclease (Caudy et al., 2003), cleaves the target RNA within the region basepaired to the siRNA guide, between the 10^{th} and 11^{th} nucleotides counting from the 5′ end of the antisense strand (Elbashir et al., EMBO J. 20(23):6877-88 (2001)). The target is then subject to degradation by cytoplasmic exonucleases.

In mammalian cells, RNAi can be induced by direct introduction of siRNAs. These can be chemically synthesized, in vitro transcribed, or generated enzymatically from longer double-stranded RNA (dsRNA) substrates (Elbashir, S. M., Harborth, J., Lendeckel, W., Yalcin, A., Weber, K., and Tuschl, T. (2001). Duplexes of 21-Nucleotide RNAs Mediate RNA Interference in Cultured Mammalian Cells. Nature 411, 494-498, Caplen, N. J., Parrish, S., Imani, F., Fire, A., and Morgan, R. A. (2001). Specific Inhibition of Gene Expression by Small Double-Stranded RNAs in Invertebrates and Vertebrate Systems. Proc. Natl. Acad. Sci. USA 98, 9746-9747, Donze & Picard 2002, Yang et al., 2002, Myers et al., 2003, Kawasaki, H., Suyama, E., Iyo, M., and Taira, K. (2003). siRNAs Generated by Recombinant Human Dicer Induce Specific and Significant But Target Site-Independent Gene Silencing in Human Cells. Nuc. Acids Res. 31, 981-987). SiRNAs can be chemically modified (e.g. STEALTH™ molecules) to increase serum stability and decrease non-specific and off-target effects, such as stimulation of interferon genes or sense-strand directed gene silencing (Woolf and Wiederholt, 2003). In addition, siRNA can be produced within the cell from DNA templates. Typically, an RNA polymerase III (polIII) promoter is employed to express a short hairpin RNA (shRNA) in the nucleus (Brummelkamp, T. R., Bernards, R., and Agami, R. (2002). A System for Stable Expression of Short Interfering RNAs in Mammalian Cells. Science 296, 550-553, McManus et al. , 2002, Paddison, P. J., Caudy, A. A., Bernstein, E., Hannon, G. J., and Conklin, D. S. (2002). Short Hairpin RNAs (shRNAs) Induce Sequence-Specific Silencing in Mammalian Cells. Genes Dev. 16, 948-958, Sui et al., 2002, Yu et al., 2002). The shRNA is actively exported to the cytoplasm by Exportin-5 (Gwizdek et al., 2003; Yi et aL, 2003), where it is recognized and cleaved by the RNase III enzyme Dicer to produce an siRNA (Paddison et al., 2002).

Specific siRNA molecules targeting different regions of a transcript can vary widely in effectiveness at decreasing gene expression (Holen, T., Amarzguioui, M., Wiiger, M., Babaie, E., and Prydz, H. (2002). Positional Effects of Short Interfering RNAs Targeting the Human Coagulation Trigger Tissue Factor. Nuc. Acids Res. 30, 1757-1766, Bohula, E. A., Salisbury, A. J., Sohail, M., Playford, M. P., Riedemann, J., Southern, E. M., and Macaulay, V. M. (2003). The Efficacy of Small Interfering RNAs Targeted to the Type 1 Insulin-Like Growth Factor Receptor (IGF1R) is Influenced by Secondary Structure in the IGF1R Transcript. J. Biol. Chem. 278, 15991-15997, Salisbury, A. J., Sohail, M., Playford, M. P., Riedemann, J., Southern, E. M., and Macaulay, V. M. (2003). The Efficacy of Small Interfering RNAs Targeted to the Type 1 Insulin-Like Growth Factor Receptor (IGF1R) is Influenced by Secondary Structure in the IGF1R Transcript. J. Biol. Chem. 278, 15991-15997; Kawasaki, H., Suyama, E., Iyo, M., and Taira, K. (2003). siRNAs Generated by Recombinant Human Dicer Induce Specific and Significant But Target Site-Independent Gene Silencing in Human Cells. Nuc. Acids Res. 31, 981-987; Vickers, T. A., Koo, S., Bennett, C. F., Crooke, S. T., Dean, N. M., and Baker, B. F. (2003). Efficient Reduction of Target RNAs by Small Interfering RNA and RNase H-Dependent Antisense Agents: A Comparative Analysis. J. Biol. Chem. 278, 7108-7118). Some siRNAs are much more effective at decreasing gene expression than others. Although there have been marked improvements in the design rules to select effective siRNAs (Schwarz, D. S., Hutvagner, G., Du, T., Xu, Z., Aronin, N., and Zamore, P. D. (2003). Asymmetry in the Assembly of the RNAi Enzyme Complex. Cell 115, 199-208), an easy method to empirically compare siRNAs is needed. While the ultimate functional test of an siRNA is its ability to generate a cellular phenotype, in many cases that effect may be unknown and is the object of the investigation. A more direct test is to measure the levels of target gene products. Protein levels are more likely to correspond to phenotypic knockdown but require antibodies to be available. Transcript levels can be assessed by a number of time-consuming methods including Northern blotting and RNase protection assays. Real-time qRT-PCR is generally considered to be the most precise and accurate method to quantitate specific RNAs but requires specialized equipment and validated primer sets.

A fast, simple, and accurate alternative to these techniques is the use of an RNAi screening vector (Lee et al., 2002; Holen, T., Amarzguioui, M., Wiiger, M., Babaie, E., and Prydz, H. (2002). Positional Effects of Short Interfering RNAs Targeting the Human Coagulation Trigger Tissue Factor. Nuc. Acids Res. 30, 1757-1766; Husken et al., 2003, Kumar et al., 2003; Miller et al., 2003; Zeng et al., 2003; Wu et al., 2004). Screening vectors utilize fusion mRNA transcripts between a quantifiable reporter gene and the target RNA of interest. Cleavage of the fusion by siRNAs targeted to the RNA of interest can be measured by the resulting reduction in reporter protein production and activity. An added advantage of screening vectors is that target knockdown can be analyzed in common, easily transfected cell types which need not express the endogenous target. Analysis would typically be carried out within 24 hours of transfection, allowing essential genes to be targeted over a short enough time period to permit cell survival.

In this example, pSCREEN-iT™/lacZ-DEST, a screening vector supporting GATEWAY™-mediated fusion of target RNAs to the lac Z gene, is described. The vector is compatible with the entry clones of the Ultimate™ ORF collection. Alternatively, genes or gene fragments may be first PCR amplified and cloned into pCR®8/GW/TOPO® TA (Invitrogen Corporation, cat. no. K2500-20) prior to recombination into the pSCREEN-iT™ destination vector. Here we demonstrate that the readout of β-galactosidase (β-gal) activity from a number of fusion constructs after cleavage mediated by standard siRNAs and STEALTH™ molecules correlates with qRT-PCR measurements of the endogenous targets. In addition, guidelines for the target RNA size and reading frame are reported. The pSCREEN-iT™ system is shown to be a valuable, easy-to-use tool for the measurement of RNAi-mediated gene knockdown.

Initially, we set out to demonstrate (1) effective discrimination between 3 different siRNAs (i.e., inhibition of 90, 50 and 30%) targeting a control DNA fragment (˜500 bp) in the screening vectors and (2) effective L×R recombination reactions between the DEST vectors and fragments cloned into pCR8.

Components were assembled to form the following kits and system which were to perform various methods:

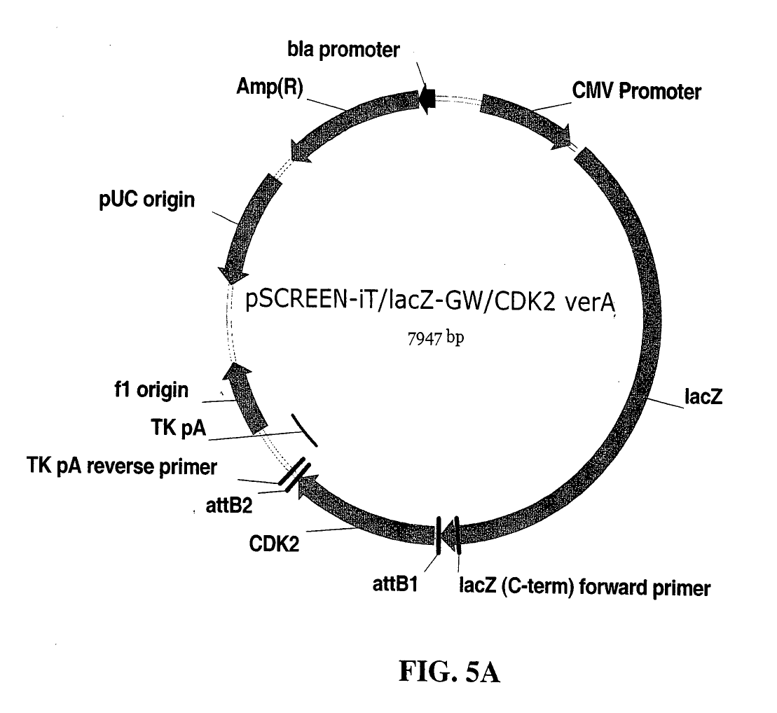

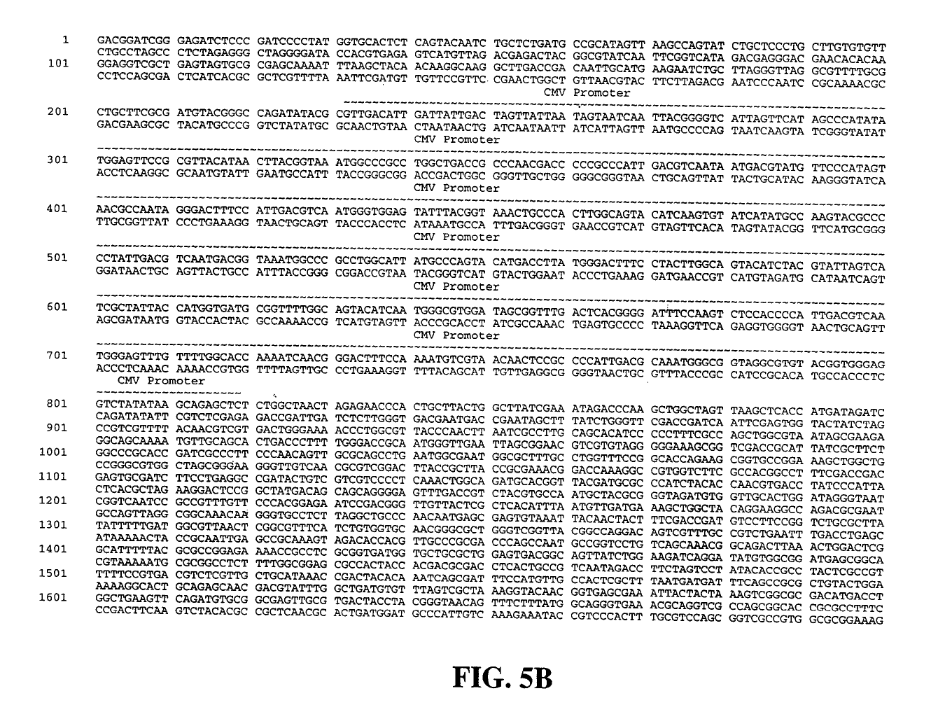

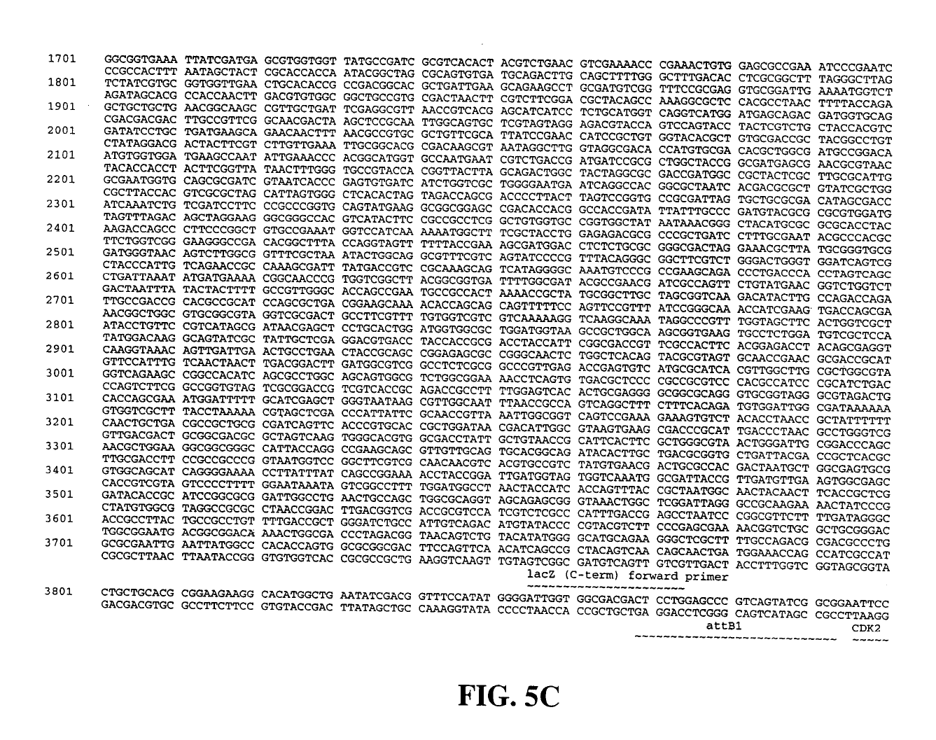

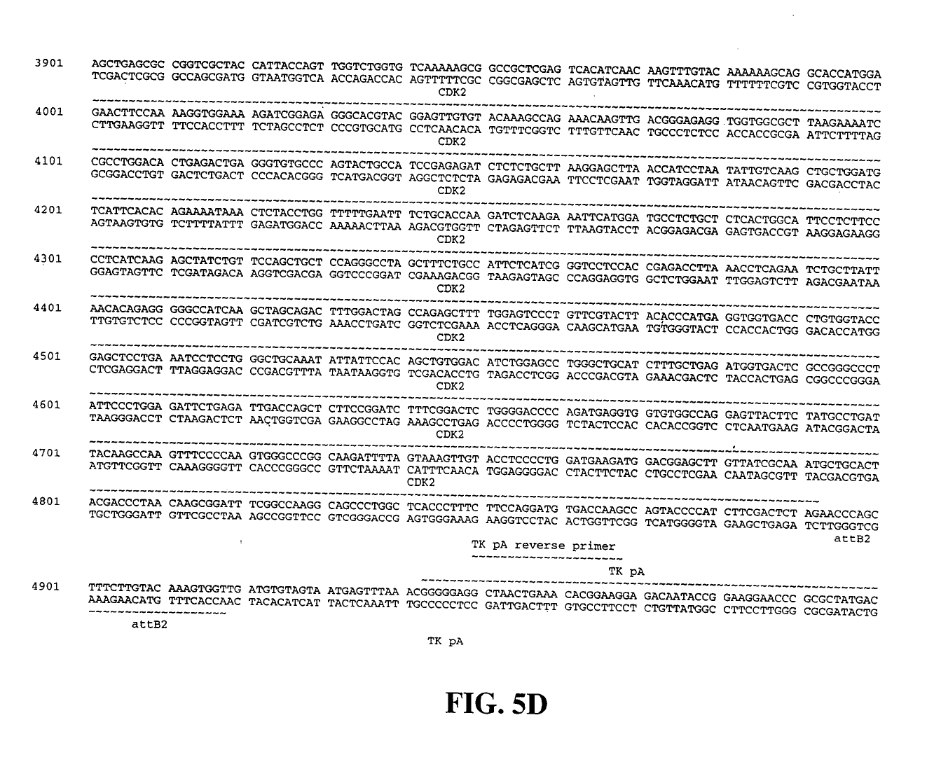

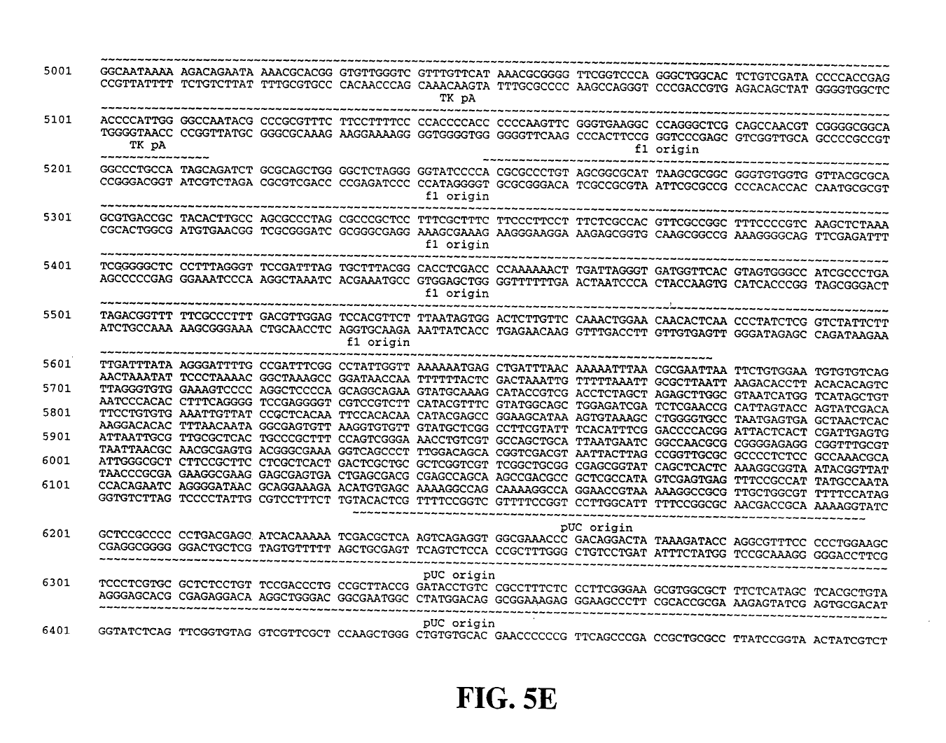

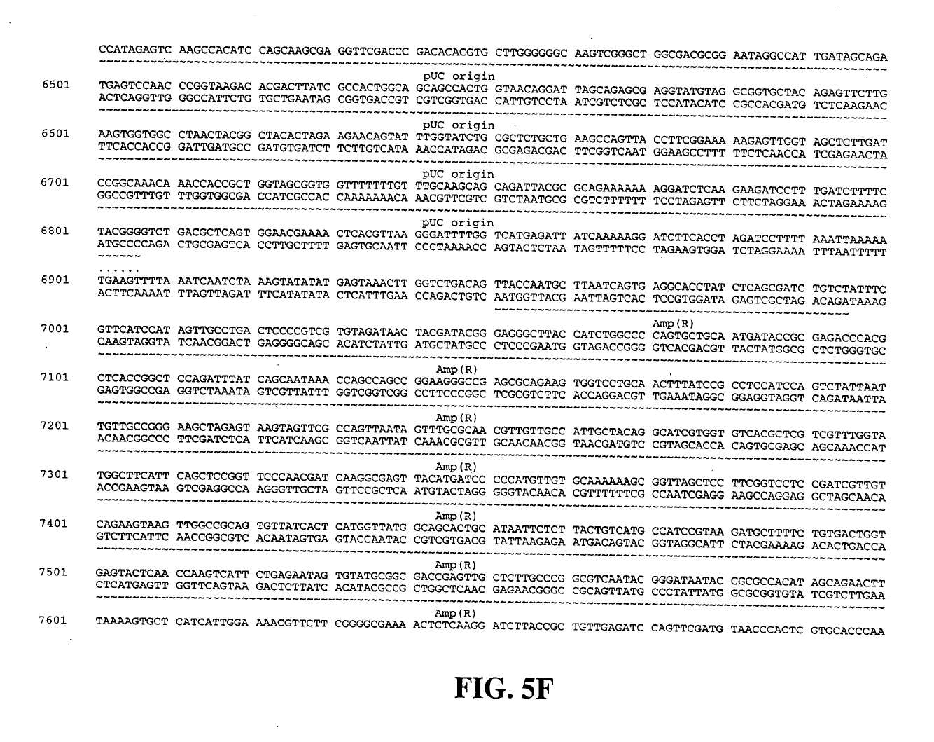

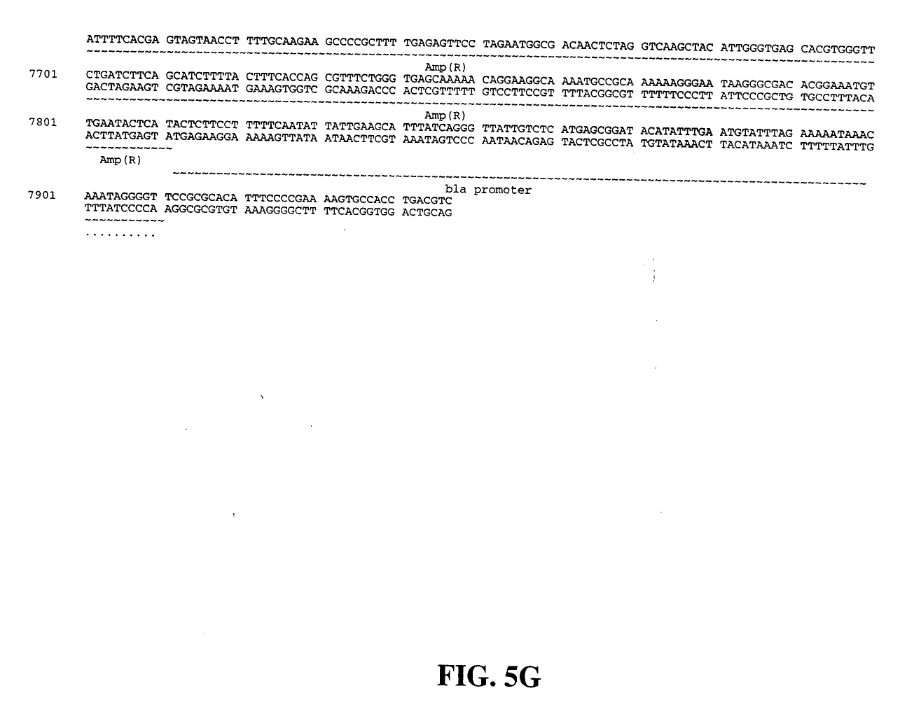

pSCREEN-iT™-DEST GATEWAY™ Vector Kit: (1) pSCREEN-iT™/lacZ-DEST vector (FIG. 4), (2) pSCREEN-iT™/lacZ-GW/CDK2 control vector (FIG. 5), (3) Positive LacZ STEALTH™ Control (sense strand: 5′-CCGUCUGAAUUUGACCUGAGCGCAU, SEQ ID NO: 42; antisense strand: 5′-AUGCGCUCAGGUCAAAUUCAGACGG), (4) Scrambled STEALTH™ Negative Control (sense strand: 5′-GGGAAGACAGAACUUGUACUCAAAA SEQ ID NO: 43; antisense strand: 5′-UUUUGAGUACAAGUUCUGUCUUCCC), and (5) 1× RNA annealing buffer (10 mM Tris pH 8.0, 20 mM NaCl, 1 mM EDTA).

BLOCK-iT™ RNAi Target Screening Kit (w/lac Z reporter): (1) pSCREEN-iT™-DEST GATEWAY® Vector Kit (above), (2) FLUOREPORTER® lac Z/Galactosidase Quantitation Kit (Invitrogen Corporation, cat. no. F-2905), and (3) LIPOFECTAMINE™ 2000, 250 l (Invitrogen Corporation, cat. no. 44-5926).

BLOCK-iT™ RNAi Target Screening System (w/lacZ reporter): (1) BLOCK-iT™ RNAi Target Screening Kit (above), (2) pCR8®/GW/TOPO® TA Entry Vector Kit w/Top10 cells (Invitrogen Corporation, cat. no. K2500-20), and (3) LR Clonase (Invitrogen Corporation, cat. no. 11791-019).

Materials & Methods

Vector Construction

A preliminary vector lacking the T7 promoter sequence, pcDNA™.2-link, was constructed by digestion of pcDNA™6.2/DEST with SacI and PmeI and insertion of the following annealed oligonucleotides: 5′-ctctggctaactagagaacccactgcttactggcttatcgaaatagacccaagctggctagctaagctgagcgttt (SEQ ID NO: 44) and 5′-aaacgctcagcttagctagccagcttgggtctatttcgataagccagtaagcagtgggttctctagttagccagag agct (SEQ ID NO: 45). The lacZ coding region containing a C-terminal stop codon was amplified from pcDNA™.2/n-GeneBLAzer/GW-lacZ (forward primer: gatcgatcactagttaagctcaccatgatagatcccgtcgttttacaacg, SEQ ID NO: 46; reverse primer: gcctcccccgtttaaacaggccttcattactagactcgagcggccgctttttgacacc, SEQ ID NO: 47). A SpeI-PmeI digest of the amplicon was cloned into the NheI and PmeI sites of pcDNA™.2-link to create pcDNA™.2-lacZ. The integrity of the lacZ coding region in three representative clones was functionally tested in a transfection assay and sequenced. A single clone was selected that encoded the expected β-gal polypeptide sequence and was used in subsequent cloning steps. A preliminary functional GATEWAY®-adapted screening vector was created by insertion of a destination cassette (rfc) into the StuI site of pcDNA™.2-lacZ for testing purposes. The SV40 promoter, blasticidin resistance gene, and SV40 polyA sequence were subsequently removed from pcDNA™.2-link by digestion with NsiI and BstZ17, exonuclease digestion with T4 DNA polymerase, and self ligation to create pcDNA™X.2-link. The lacZ coding region was excised from pcDNA™.2-lacZ with SpeI and PmeI and cloned into the same sites in pcDNA™X.2-link to create pcDNA™X.2-lacZ. The pcDNA™X.2-lac Z plasmid was GATEWAY® adapted by insertion of a destination cassette (rfc) into the Stul site to create pcDNA™X.2-lacZ-DEST. The stop codon at the C-terminus of β-gal was removed from pcDNA™X.2-lacZ by digestion with XhoI and PmeI and replaced with annealed oligonucleotides (forward: tcgagtcacgtgtagtaatgagttt, SEQ ID NO: 48; reverse: aaactcattactacacgtgac, SEQ ID NO: 49) to create pcDNA™X.2-lacZ-nostop. The pcDNA™X.2-lacZ-nostop plasmid was GATEWAY® adapted by insertion of a destination cassette (rfb) into the PmlI site to create pcDNA™X.2-lacZ-nostop-DEST (pSCREEN-iT™/lacZ-DEST). The integrity of the attR1 and attR2 site junctions was confirmed by sequencing.

The following ULTIMATE™ ORF entry clones were used in this study: IOH21140 (CDK2), IOH3445 (IKBKG), IOH14527 (PEN2), IOH22884 (PTP4A1), IOH21715 (MAP2K3), IOH3654 (-actin) (available from Invitrogen Corporation, see the ULTIMATE™ ORF Browser). The CDK2 fusion, pSCREEN-iT™/lacZ-GW/CDK2 (FIG. 5), will be used as the positive control; full vector sequencing is ongoing.

For the β-lactamase (β-lac) experiment, a 200 base pair PCR fragment from nt 401-600 of the β-lactamase coding region in pcDNA™.2/nGeneBLAzer-GW/lacZ was amplified with Taq-HiFi (Invitrogen) and cloned into pCR®8/GW/TOPO® TA by standard procedures. The primers used were 5′-atgtaactcgccttgatcgttg (forward, SEQ ID NO: 50) and 5′-ggccgagcgcagaagtggtcct (reverse, SEQ ID NO: 51). SiRNAs β-lac15 through 20 are targeted to this region and were previously tested.

Standard LR CLONASE™ reactions were performed between the pSCREEN-iT™ vectors and the ORF clones or the β-lac PCR fragment subcloned in PCR® 8/GW/TOPO® TA. The plasmids were confirmed by restriction analysis. The pSCREEN-iT™/lacZ-DEST vector passed standard LR and ccdB assays.

Sequencing/Primers

Sequence verification of inserts, such as the CDK2 ORF, was performed with MB108 (forward sequencing primer) 5′-ATTGGTGGCGACGACTCCTG-3′ (SEQ ID NO: 52) (hybridizes to lacZ, 125 bp upstream of attB1), and MB109 (reverse sequencing primer) 5′-ACCCGTGCGTTTTATTCTGTC-3′ (SEQ ID NO: 53)(hybridizes to TK polyA, 85 base pairs downstream of attB2).

Transfection/Cell Culture

GRIPTITE™ 293 MSR cells were cultured in the recommended medium. CMV Bla CHO cells were cultured in DMEM/10% FBS medium. STEALTH™ and siRNA molecules were obtained from Invitrogen Corporation as lyophilized samples and resuspended by the recommended protocols provided with the product. Briefly, the oligo duplexes were resuspended to 20M in the DEPC treated water provided. The resuspension reconstitutes the dry-down buffer to a final concentration of 10 mM Tris-HCl, 1 mM EDTA, 20 mM NaCl. Subsequent dilutions of the oligonucleotides was done in annealing buffer of the same formulation.

For GRIPTITE™ 293 MSR (24-well) transfections, LIPOFECTAMINE™ 2000 (Invitrogen Corporation, cat. no. 11668-027) transfections were performed as recommended for the product with plasmid DNA alone. Briefly, 100-200 ng of the pSCREEN-iT™ vector was transfected using 1.5 l of LIPOFECTAMINE™ 2000/well, with or without the addition of 1 pmol of siRNA or Stealth oligonucleotides in a final volume of 600 l. Medium was changed after 4 hr incubation at 37 C. For CMV Bla CHO (96-well) transfections, 0.7 l of Lipofectamine™ 2000 and 5 pmol of siRNA were transfected per well of containing 10⁵ cells in a final volume of 150 l.

Luciferase and β-gal Assays

Approximately 24 hours after transfection, 500 μl of lysis buffer (25 mM Tris-HCl pH 8.0, 0.1 mM EDTA, 10% glycerol, 0.1% Triton X-100) were added to each well of a 24-well tissue culture plate after the growth medium was removed. Plates were frozen at −70° C. for at least 20 minutes. Samples were thawed and mixed, and aliquots were assayed for luciferase and/or β-gal activity.

Luciferase assays were performed using Luciferase Assay Reagent (Promega Corp., Madison, Wis. 53711) according to the manufacturer's instructions. Luminescence was measured from 50 μl of lysate in each well of a black 96-well plate by a MicroLumat Plus luminometer using Winglow v.1.24 software (EG&G Berthold, Oak Ridge, Tenn.). An equal volume of assay reagent (50 μl) was injected per well and readings were taken for 5 s after a 2 s delay.

β-gal assays were performed using the Molecular Probes FLUOREPORTER® lacZ/Galactosidase Quantitation Kit (Invitrogen Corporation, cat. no. F-2905) according to the Probes protocols. Briefly, 2-10 μl of lysate were combined with the CUG substrate in 100 μl of reaction buffer and incubated for 30 minutes at room temperature before the addition of 50 μl stop buffer (the stop buffer terminates the reaction and causes an increase in the fluorescence of the product). The fluorescence of the resulting solution was measured at 460 nm after excitation at 390 nm. Typically, the reaction solution, sample, and stop solution were all combined in a black walled 96-well plate for direct excitation and emission measurements without transfer.

Results

The goal of the RNAi Screening Vector project was to produce a simple way to screen RNAi reagents (e.g., siRNAs, STEALTH™ molecules, and shRNA-expressing plasmids) for effective knockdown of target genes. A rapid and reliable way to generate test constructs, such as GATEWAY® recombination, needed to be employed. Subsequently, fusions created using the screening vector had to produce data comparable to that obtained from qRT-PCR of the natively expressed target to validate the technique. Specifically, the sensitivity and dynamic range of the readout would need to sufficiently differentiate between RNAi reagents which produce varied levels of knockdown (e.g., 30%, 50%, and 90% reduction in mRNA levels by qRT-PCR).

Features of the pSCREEN-iT™ Plasmids

A map of pSCREEN-iT™/lacZ-DEST is shown in FIG. 4A and the nucleotide sequence of this vector is shown in FIG. 4B. This vector carries the CMV promoter upstream of the lacZ gene followed by an attR1-R2 destination cassette. The lacZ gene was chosen as the reporter because it is easily and accurately quantifiable over a wide dynamic range in cell lysates using the FLUOREPORTER® lacZ/Galactosidase Quantitation Kit from Molecular Probes. Although N-terminal fusions of the gene of interest to lacZ function equally well for screening purposes, the destination cassette was placed downstream of lacZ to make pSCREEN-iT™/lacZ-DEST compatible with the ULTIMATE™ ORF collection. The ULTIMATE™ ORFs carry stop codons upstream of attL2 which, if cloned in front of lacZ, would terminate translation before synthesis of β-gal.

Initially, versions of the plasmid were constructed with and without a stop codon between the lacZ coding region and attRI site. Placing a stop codon between lacZ and the sequence of interest (RNA-only fusion) would offer a number of advantages over no stop (protein fusion). First, the activity of the resulting β-gal protein would be expected to be consistent from construct to construct, as no additional amino acids are added to the reporter's C-terminus. Second, since no part of the target RNA would be translated into protein, there should be no complicating pleiotropic effects from overexpression of the gene of interest. Finally, an RNA-only fusion would obviate the need to position the inserted gene or gene fragment in the correct reading frame.

While the advantages of an RNA-only fusion are clear, there is also a potential drawback. Because RNAi acts at the level of message stability, any transcript containing the target sequence is available for cleavage, whether it encodes a fusion protein or terminates translation after the β-gal coding region. However, the subsequent exonuclease-directed destruction of RISC cleavage fragments appears to be rate-limiting (Javorschi et al., 2004). Since the 5′ fragment of a lacZ-stop-target fusion transcript cleaved in the target RNA region will still contain an uninterrupted lacZ ORF (complete with stop codon), it may continue to be translated, despite the loss of a polyA tail, until sufficiently degraded. This post-cleavage translation could decrease the apparent knockdown. Compounding this effect, a specific degradation pathway for mRNAs lacking a stop codon, such as the 5′ cleavage fragment from a non-stop fusion, has been identified (Maquat et al., 2002). This “non-stop mediated decay” system may accelerate the degradation of RISC cleavage products from transcripts encoding fusion proteins, but not from fusion transcripts with a stop after the reporter.

Comparison of Screening Vectors to qRT-PCR

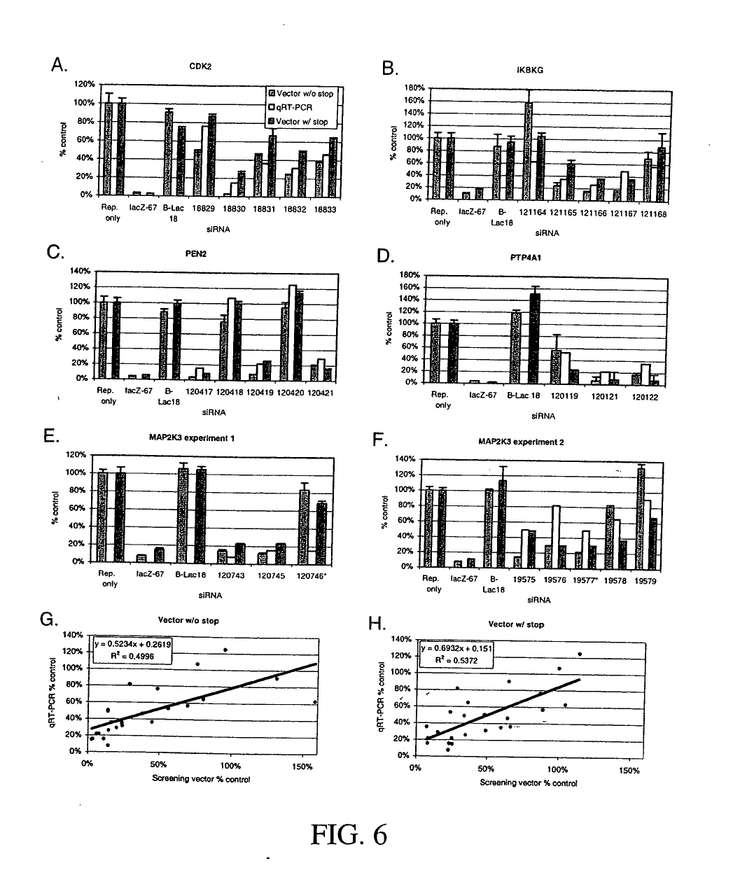

To functionally test pSCREEN-iT™ vectors, targets were chosen for which qRT-PCR data were already available. Fusion transcripts were generated by LR recombination of screening vectors with and without a stop codon following lacZ with ULTIMATE™ ORF entry clones corresponding to the CDK2, IKBKG, PEN-2, PTP4A1, and MAP2K3 genes. The plasmids were cotransfected into GRIPTITE™ 293 MSR cells with only a luciferase (luc) reporter plasmid or with the luc reporter and siRNAs directed against the target gene, against the lacZ coding region (positive control), or against β-lactamase (β-lac, negative control). While there was variation between the measurements made using the stop and no-stop reading vectors and qRT-PCR (FIG. 6A-6F), knockdown effectiveness for each vector tended to correlate with the qRT-PCR measurements of RNA levels (FIG. 6G-6H). The correlation was approximately the same for both versions of the screening vector; however, the readout for the stop codon version was more compressed along its axis, resulting in a higher slope (0.69 vs. 0.52). This means that the distinction between good, moderate, and poor silencers is reduced for the vector with the stop codon.

The screening vector data is expected to represent the activity for each siRNA under somewhat idealized conditions. Unlike qRT-PCR, which measures target gene levels in both siRNA-transfected and untransfected cells, the cotransfection of the pSCREEN-iT™ plasmids with the siRNA delivers the knockdown agent and the target gene to the same cells. Thus, the screening vector approach returns the relative efficacies of the siRNAs without significant influence from the efficiency of delivery. This explains why knockdown of the screening vectors was often greater than that measured by qRT-PCR.

Two of the siRNAs against MAP2K3, 120746 and 19577, were determined to carry single base mismatches to the ORF in the screening vector but not to the endogenous transcript targeted in qRT-PCR studies (FIG. 6E-6F, asterisks). The mismatch in 120746 is in a central region (nucleotide #10 of the antisense strand) and resulted in poor knockdown of the lacZ fusion. In contrast, the mismatch in 19577 was at one end of the siRNA duplex (nt #1 of the antisense strand) and appeared to have no negative effects on its RNAi activity. This is consistent with observations of the consequences of siRNA mismatch location in published reports (Amarzguioui et al., 2003, Czaudema, F., Santel, A., Hinz, M., Fechtner, M., Durieux, B., Fisch, G., Leenders, F., Arnold, W., Giese, K., Klippel, A., and Kaufmann, J. (2003). Inducible shRNA Expression for Application in a Prostate Cancer Mouse Model. Nuc. Acids Res. 31, e127). The mismatched siRNAs were excluded from the scatter plot analysis in FIG. 6G-6H.

Apparent Knockdown From RNA-Fusion Vectors Correlates With Distance From Stop Codon

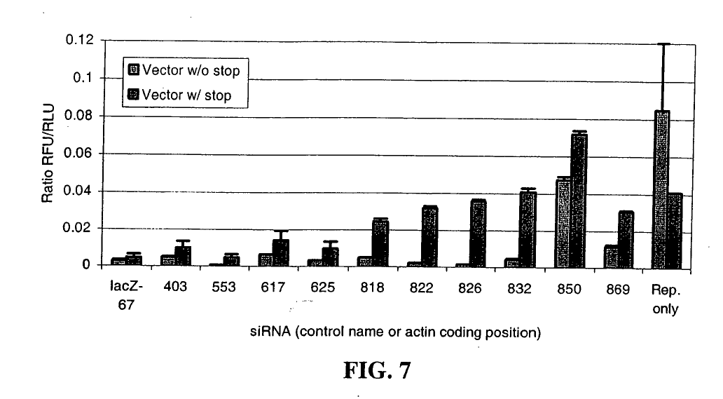

The compressed readout from the stop codon version of pSCREEN-iT™ is a potential pitfall for use of that construct. To more closely investigate the possibility that the compressed readout might be related to the distance between the stop codon and the siRNA target site, a systematic comparison was made using the -actin Ultimate™ ORF. Sequences from positions 403 to 869 in the -actin ORF were targeted for cleavage by ten different siRNAs (FIG. 7). While knockdown of the lacZ-actin no stop fusion was robust for siRNAs 403-832, β-gal activities from the lacZ-stop-actin fusion were consistently higher. Moreover, the discrepancy increased with the distance of the target from the stop codon. Even siRNA 850, which performed poorly for the no stop version, had higher β-gal expression for the stop version. This trend is consistent with our hypothesis that mRNAs cleaved by the RISC in their 3′ untranslated regions can continue to produce protein while being slowly degraded by exonucleases. Under this model, as the distance from the stop codon increases, so does the time it takes for the degradation to reach the protein coding region. This discrepancy between the initial cleavage and the cessation of β-gal synthesis might lead one to rank an effective siRNA (e.g., Actin 832) as poor simply because it is distal to the lacZ stop codon. This would be an obvious hindrance for a product which is meant to discriminate between siRNAs based on their ability to cleave targets. Thus, whenever possible, the target gene should be fused to lacZ in frame and without an intervening stop codon.

PCR Products May Enter the System Through pCR®8/GW/TOPO® TA

When using the system described herein, users will often need to construct an entry vector encoding their RNA of interest. This can be done by amplifying the gene of interest and cloning it into pCR®8/GW/TOPO® TA. The amplicon can then be transferred into the screening vector by an LR recombination reaction. As an example, a 200 base pair region of the β-lac gene was cloned into the pSCREEN-iT™m/lacZ-DEST non-stop version using pCR®8/GW/TOPO® TA as an intermediary. In the fusion construct, the β-lac region lies out of frame with lacZ, creating a stop codon in 5′ β-lac (nucleotides 4-6).

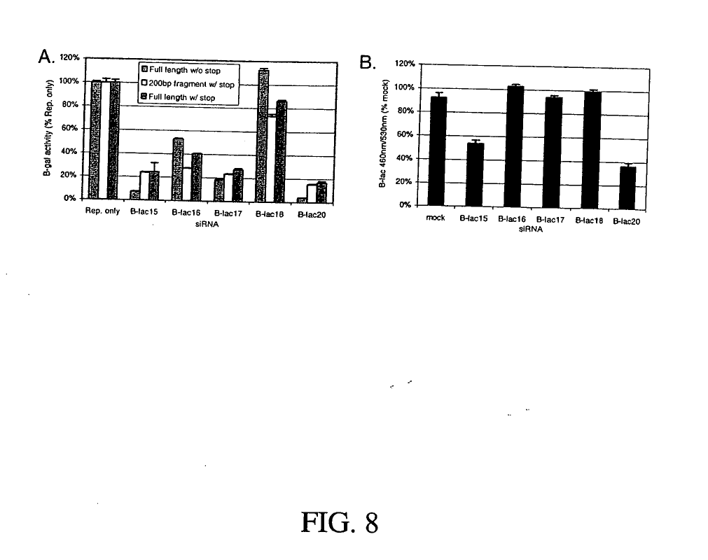

The 200 base pair amplicon includes the target sites of five previously tested β-lac siRNAs in an overlapping cluster (nt 104-131). The target site most distal to the stop codon is positioned only 9 nucleotides downstream of the most proximal; thus, the position effect is expected to be minimal. The screening vector was cotransfected with these siRNAs into GRIPTITE™ 293 MSR cells and assayed for β-gal activity after 24 hours (FIG. 8). The results were normalized to the screening vector only control (FIG. 8, Rep. only) and compared to results from a previous experiment using full length lacZ/β-lac fusions with and without stop codons between the coding regions (pcDNA™.2/cGeneBLAzer-GW/lacZ and pcDNA™.2/LacZ-STOP-GW/BLA). The data from the full-length and 200 base pair fusions carrying stop codons were very similar for each siRNA tested, showing that a small fragment can retain sufficient context to identify good and poor siRNA activities when compared to the entire ORF. However, the level of discrimination between the siRNAs for both of these vectors was lower than that of the full-length fusion without a stop, consistent with our previous observations (FIGS. 6-7). The relative ranking of best three siRNAs for all forms of screening vector (B-lac20>B-lac15>B-lac17) matched the performance of the siRNAs against an unfused β-lac target stably expressed in CMV Bla CHO cells (FIG. 7B). The low apparent knockdown in the CHO experiment likely results from low transfection efficiency and non-linear reporter readout, making the poorer performers difficult to measure. It is because of these kinds of limitations that screening vectors are so useful for identifying the most active siRNAs.

Given the results above and the fact that the PEN-2 ORF (FIG. 6) is only 306 base pairs in length, it appears that fragments in the 200-300 base pair range may be used in the screening vector provided that they are cloned in frame so as to create a protein fusion with lacZ. In those cases in which a protein fusion cannot be made, fragments should be kept small to reduce the stop codon distance bias (FIG. 7). However, under those circumstances, a lower resolution readout should be expected.

Inclusion of STEALTH™ Duplexes as Positive and Negative Controls

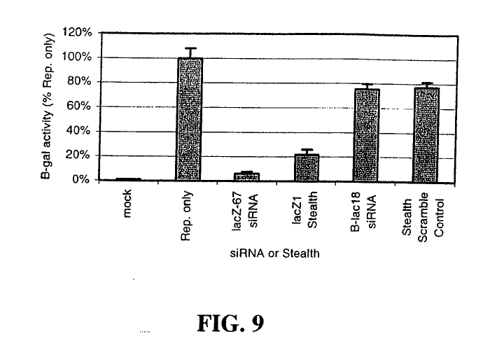

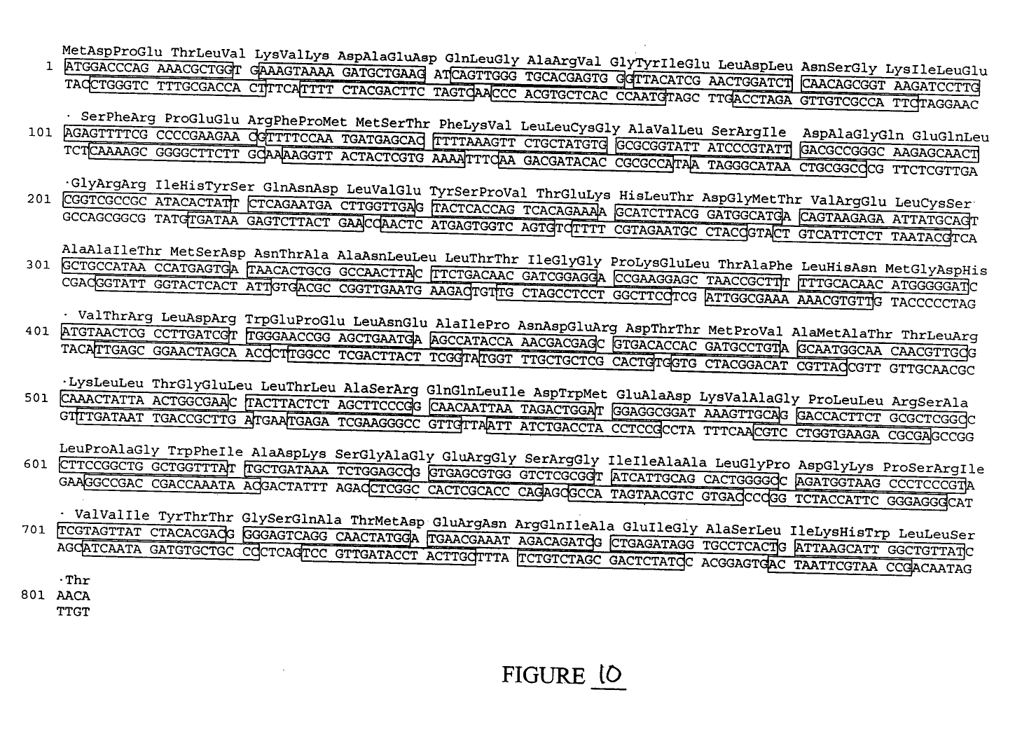

STEALTH™ molecules were tested as the positive and negative controls for the kits. FIG. 9 shows the results of cotransfection of pSCREEN-iT™/lacZ-GW/CDK2 with independent lacZ-directed siRNA and STEALTH™ duplexes, the B-lac18 control siRNA, and a STEALTH™ scrambled control. The negative controls behaved virtually identically. The lacZ1 STEALTH™ has a moderately high effectiveness when compared to lacZ-67 siRNA, which was selected for its potency. In this case, use of a less potent RNAi reagent for the positive control is desirable; it should be more sensitive to nuclease contamination or poor transfections, for example, than a STEALTH™ that can knock down at extremely low concentrations. An additional advantage of a weaker positive control is that it will not produce an unrealistic standard against which STEALTH™ duplexes directed against the target gene will be compared.

Final Kit Configuration

Due to its improved performance for a variety of target sites and lack of position bias, the non-stop plasmid version was chosen as the final pSCREEN-iT™/lacZ-DEST vector. For the kit positive control expression construct, the CDK2 target was chosen (pSCREEN-iT™/lacZ-GW/CDK2, FIGS. 5A-5B and 6A). The lacZ directed STEALTH™ lacZ1 will be the positive control RNAi reagent for the kit, with the STEALTH™ Scramble Control serving as the negative control (FIG. 7).

Conclusions

Screening through RNAi reagents to find sufficient knockdown efficacy is a resource-intensive but necessary step in modern gene suppression experiments. The pSCREEN-iT™/lacZ-DEST vector combines the power of Invitrogen's GATEWAY® technology and lacZ assay capabilities to provide a simple, fast, and reliable means to test RNAi reagents such as BLOCK-iT™ siRNAs or STEALTH™ RNAi molecules, as well as antisense oligonucleotides and ribozymes directed at cleaving their targets, without knowing the knockdown phenotype or requiring specialized cell lines. This gives it major advantages over other techniques. Especially when coupled with the Ultimate™ ORF collection, the SCREEN-iT™ system is a powerful and valuable tool for RNAi research.

Those wishing to express their target as an RNA-only fusion due to toxicity of their gene product or negative effects on β-gal activity from a protein fusion may do so by including a stop codon at the 5′ end of their insert. However, about the position effect and the reduced discrimination between differing RNAi activities in non-stop versions should be kept in mind. In general the untranslated target regions should be kept to 200-500 nucleotides in length to reduce the influence of position but also provide enough context to produce suppression data likely to be valid for the endogenous gene.

Further when making protein fusions to lacZ as described above, users should either include a 3′ stop codon in their target sequence or clone the sequence in frame not only with the upstream lacZ ORF but also with the three stop codons downstream of att B2.

### REFERENCES

1. Amarzguioui et al. (2003). Tolerance for mutations and chemical modifications in a siRNA. Nucleic Acids Res. 31(2):589-95.
2. Bohula et al. (2003). The efficacy of small interfering RNAs targeted to the type 1 insulin-like growth factor receptor (IGF1R) is influenced by secondary structure in the IGF1R transcript. J Biol Chem. 278(18):15991-7.
3. Brummelkamp et al. (2002). A system for stable expression of short interfering RNAs in mammalian cells. Science. 296(5567):550-3.
4. Caplen et al. (2001). Specific inhibition of gene expression by small double-stranded RNAs in invertebrate and vertebrate systems. Proc Natl Acad Sci USA. 98(17):9742-7.
5. Caudy et al. (2003). A micrococcal nuclease homologue in RNAi effector complexes. Nature. 425(6956):411-4.
6. Czauderna et al. (2003). Structural variations and stabilising modifications of synthetic siRNAs in mammalian cells. Nucleic Acids Res. 31(11):2705-16.
7. Donze & Picard (2002). RNA interference in mammalian cells using siRNAs synthesized with T7 RNA polymerase. Nucleic Acids Res. 30(10):e46.
8. Dykxhoorn et al. (2003). Killing the messenger: short RNAs that silence gene expression. Nat Rev Mol Cell Biol. 4(6):457-67.
9. Elbashir et al. (2001a). Duplexes of 21-nucleotide RNAs mediate RNA interference in cultured mammalian cells. Nature. 411(6836):494-8.
10. Elbashir et al. (2001b). Functional anatomy of siRNAs for mediating efficient RNAi in Drosophila melanogaster embryo lysate. EMBO J. 20(23):6877-88.
11. Gwizdek et al. (2003). Exportin-5 mediates nuclear export of minihelix-containing RNAs. J Biol Chem. 278(8):5505-8.
12. Holen et al. (2002). Positional effects of short interfering RNAs targeting the human coagulation trigger Tissue Factor. Nucleic Acids Res. 30(8):1757-66.
13. Husken et al. (2003). mRNA fusion constructs serve in a general cell-based assay to profile oligonucleotide activity Nucleic Acids Res. 31(17):e102.
14. Javorschi et al. (2004). Kinetics of RNA Interference studied by real-time PCR with LUX fluorogenic primers. PharmaGenomics. Feb 4:44-48.
15. Kawasaki et al. (2003). siRNAs generated by recombinant human Dicer induce specific and significant but target site-independent gene silencing in human cells. Nucleic Acids Res. 31(3):981-7.
16. Kumar et al. (2003). High-Throughput Selection of Effective RNAi Probes for Gene Silencing. Genome Res. 13(10):2333-40.
17. Lee et al. (2002). Expression of small interfering RNAs targeted against HIV-1 rev transcripts in human cells. Nat Biotechnol. 20(5):500-5.
18. McManus et al. (2002). Gene silencing using micro-RNA designed hairpins. RNA. 8(6):842-50.
19. Maquat (2002). Nonsense-mediated mRNA decay. Curr Biol. 12(6):R196-7.
20. Miller et al. (2003). Allele-specific silencing of dominant disease genes. Proc Natl Acad Sci USA. 100(12):7195-200.
21. Myers et al. (2003). Recombinant Dicer efficiently converts large dsRNAs into siRNAs suitable for gene silencing. Nat Biotechnol. 21(3):324-8.
22. Paddison et al. (2002). Short hairpin RNAs (shRNAs) induce sequence-specific silencing in mammalian cells. Genes Dev. 16(8):948-58.
23. Schwarz et al. (2003). Asymmetry in the Assembly of the RNAi Enzyme Complex. Cell. 115(2):199-208.
24. Sui et al. (2002). A DNA vector-based RNAi technology to suppress gene expression in mammalian cells. Proc Natl Acad Sci USA. 99(8):5515-20.
25. Vickers et al. (2003). Efficient reduction of target RNAs by small interfering RNA and RNase H-dependent antisense agents. A comparative analysis. J Biol Chem. 278(9):7108-18.
26. Woolf and Wiederholt (2003). U.S. Patent Publication No. 2004/0014956.
27. Wu et al. (2004). A novel approach for evaluating the efficiency of siRNAs on protein levels in cultured cells. Nucleic Acids Res. 32(2):E17.
28. Yang et al. (2002). Short RNA duplexes produced by hydrolysis with Escherichia coli RNase III mediate effective RNA interference in mammalian cells. Proc Natl Acad Sci USA. 99(15):9942-7.
29. Yi et al. (2003). Exportin-5 mediates the nuclear export of pre-microRNAs and short hairpin RNAs. Genes Dev. 17(24):3011-6.
30. Yu et al. (2002). RNA interference by expression of short-interfering RNAs and hairpin RNAs in mammalian cells. Proc Natl Acad Sci USA. 99(9):6047-52.

31. Zeng et al. (2003). MicroRNAs and small interfering RNAs can inhibit mRNA expression by similar mechanisms. Proc Natl Acad Sci USA. 100(17):9779-84.

TABLE 12
LR assay data (Top10)

| | specification | | | | Trans-formation |
|---|---|---|---|---|---|
| | (# of | Actual # | Avg. cfu | Avg. cfu per | efficiency (col./μg |
| Reaction Plate | colonies) | of colonies | per plate | transformation* | DNA) |
| Reaction 1 @ 10⁰(No DNA Control) | 0 | 0 | 0 | 0 | |
| Reaction 2 @ 10⁰ (pSCREEN-iT ™/LacZ- | ≦10 | 0 | 0 | | |
| DEST only) | | | | | |
| Reaction 3 @ 10⁻¹ (pSCREEN-iT ™/LacZ- | ≧15 | 23 | 28 | 1400 | 3(10)^{3†} |
| DEST + pCR7LacZ-as) Plate 1 | | | | | |
| Reaction 3 @ 10⁻¹ (pSCREEN-iT ™/LacZ- | ≧15 | 34 | | | |
| DEST + pCR7LacZ-as) Plate 2 | | | | | |
| Reaction 3 @ 10⁻² (pSCREEN-iT ™/LacZ- | | 3 | 2 | 1000 | |
| DEST + pCR7LacZ-as) Plate 1 | | | | | |
| Reaction 3 @ 10⁻² (pSCREEN-iT ™/LacZ- | | 1 | | | |
| DEST + pCR7LacZ-as)Plate 2 | | | | | |
| pUC19 #1158463 @ 10⁻¹ Plate 1 | | 70 | 73.5 | 3675 | 3.7(10)^{8‡} |
| pUC19 #1158463 @ 10⁻¹ Plate 2 | | 77 | | | |
| pUC19 #1158463 @ 10⁻² Plate 1 | | 7 | 7.5 | 3750 | |
| pUC19 #1158463 @ 10⁻² Plate 2 | | 8 | | | |

*ave. cfu/plate × 10 (dil factor) × 5 (0.1 mL plated)
†(cfu/XF) × (10³ ng/μg)/(400 ng DNA transformed) 1.2(10)³ × (10³ ng/μg)/(400 ng DNA transformed)
‡(cfu/XF) × (10⁶ pg/μg)/(10 pg DNA transformed) 3.7125(10)³ × (10⁶ pg/μg)/(10 pg DNA transformed)

Protocols

Transfection with LIPOFECTAMINE™ 2000

Transfections should be carried out according to Invitrogen's general recommendations for LIPOFECTAMINE™ 2000 (LF2K) mediated plasmid transfection but using the following reagent amounts:

TABLE 13

| | | pSCREEN-iT ™ | RNA/DNA | | LF2K |
|---|---|---|---|---|---|
| Plate Type | Cells per well/density¹ | amount | amount² | | amount |
| 96-well | 3.5 × 10⁴/80% | 50-100 ng | 0.4-1 | pmol/ | 0.2-0.5 μl |
| | | | 150-300 | ng | |
| 24-well | 1.5 × 10⁵/80% | 100-200 ng | 1-5 | pmol/ | 1-1.5 μl |
| | | | 300-600 | ng | |
| 6-well | 7.5 × 10⁵/80% | 500 ng-1 μg | 5-25 | pmol/ | 5-7.5 μl |
| | | | 1.5-3 | μg | |

¹Based on 293 cells. Cell number is the number plated the day before transfection. Density is the relative confluence on the day of transfection.
²RNA = siRNA or Stealth ™ duplex. DNA = shRNA plasmid.

Lysis Protocol

Cell lysates should be made 18-48 hr post transfection. Generally, harvesting on the day after transfection is sufficient.

The following lysis buffer is compatible with the FLUOREPORTER® lacZ/Galactosidase Quantitation Kit (Invitrogen Corporation, cat. no. F-2905), the Tropix β-gal Assay, and the Promega Luciferase Assay Reagent: 25 mM Tris-HCl pH 8.0, 0.1 mM EDTA, 10% glycerol, 0.1% Triton X-100.

The table below gives ranges of acceptable lysis buffer amounts for different tissue culture dishes:

TABLE 14

| Plate Type | Lysis buffer (μl) |
|---|---|
| 96-well | 25-100 |
| 24-well | 125-500 |
| 6-well | 600-2000 |

Medium is removed from each well and lysis buffer is added (an optional wash with 1× Dulbecco's PBS may be performed). Plates should be frozen at −70° C. after collection to enhance lysis. This also creates a convenient stopping point. The lysates may be stored for up to one month if wrapped in parafilm or plastic wrap.

Assay Protocol

Prepare all solutions and enzyme dilutions for the standard curve as described in the FLUOREPORTER® lacZ/Galactosidase Quantitation Kit manual (Invitrogen Corporation, cat. no. F-2905). Thaw lysates in plates at room temperature (approximately 30-45 min). Use 10 μl of lysate per assay, regardless of the starting size of the transfection. It may be necessary to dilute lysates (in lysis buffer) to obtain readings within the linear range of the standard curve. For further details, consult the FLUOREPORTER® lacZ/Galactosidase Quantitation Kit manual (Invitrogen Corporation, cat. no. F-2905)

TABLE 15

RNAi Reagents

| | | | | | |
|---|---|---|---|---|---|
| | | sense | | antisense | |
| RNAi | target | RNA sequence | SEQ ID | RNA sequence | SEQ ID |
| molecule¹ | gene | (5′ to 3′) | NO | (5′ to 3′) | NO |

| | | | | | |
|---|---|---|---|---|---|
| lacZ-67 (si) | lacZ | AUGAAGCAGAACAA | 54 | UAAAGUUGUUCUGC | 98 |
| | | CUUUAAC | | UUCAUCA | |
| B-lac15 (si) | β-lac | ACUAUUAACUGGCG | 55 | UAGUUCGCCAGUUA | 99 |
| | | AACUAUU | | AUAGUUU | |
| B-lac16 (si) | β-lac | AUUAACUGGCGAAC | 56 | AAGUAGUUCGCCAG | 100 |
| | | UACUUUU | | UUAAUUU | |
| B-lac17 (si) | β-lac | UUAACUGGCGAACU | 57 | UAAGUAGUUCGCCA | 101 |
| | | ACUUAUU | | GUUAAUU | |
| B-lac18 (si) | β-lac | UAACUGGCGAACUAC | 58 | GUAAGUAGUUCGCC | 102 |
| | | UUACUU | | AGUUAUU | |
| B-lac20 (si) | β-lac | UGGCGAACUACUUAC | 59 | UAGAGUAAGUAGUU | 103 |
| | | UCUAUU | | CGCCAUU | |
| 18829 (si) | CDK2 | GUUGACGGGAGAGG | 60 | CACCACCUCUCCCGU | 104 |
| | | UGGUGTT | | CAACTT | |
| 18830 (si) | CDK2 | GAUGGACGGAGCUU | 61 | AUAACAAGCUCCGU | 105 |
| | | GUUAUTT | | CCAUCTT | |
| 18831 (si) | CDK2 | GCUAGCAGACUUUG | 62 | UAGUCCAAAGUCUG | 106 |
| | | GACUATT | | CUAGCTT | |
| 18832 (si) | CDK2 | AUCCUCCUGGGCUGC | 63 | AUUUGCAGCCCAGG | 107 |
| | | AAAUTT | | AGGAUTT | |
| 18833 (si) | CDK2 | GUGGGCCCGGCAAGA | 64 | AAAAUCUUGCCGGG | 108 |
| | | UUUUTT | | CCCACTT | |
| 121164 (si) | IKBKG | CAGGAGGUGAUCGA | 65 | GCUUAUCGAUCACC | 109 |
| | | UAAGCTT | | UCCUGTT | |
| 121165 (si) | IKBKG | GCUCGAUCUGAAGA | 66 | CUGCCUCUUCAGAU | 110 |
| | | GGCAGTT | | CGAGCTT | |
| 121166 (si) | IKBKG | GCUCUUCCAAGAAUA | 67 | GUCGUAUUCUUGGA | 111 |
| | | CGACTT | | AGAGCTT | |
| 121167 (si) | IKBKG | GGUGAUCGAUAAGC | 68 | CUUCAGCUUAUCGA | 112 |
| | | UGAAGTT | | UCACCTT | |
| 121168 (si) | IKBKG | UAUCUACAAGGCGG | 69 | GAAGUCCGCCUUGU | 113 |
| | | ACUUCTT | | AGAUATT | |
| 120417 (si) | PEN2 | GUGUCCAAUGAGGA | 70 | AUUUCUCCUCAUUG | 114 |
| | | GAAAUTT | | GACACTT | |
| 120418 (si) | PEN2 | AUCAAAGGCUAUGU | 71 | GCCAGACAUAGCCU | 115 |
| | | CUGGCTT | | UUGAUTT | |
| 120419 (si) | PEN2 | CUACCUCUCCUUCAC | 72 | UAUGGUGAAGGAGA | 116 |
| | | CAUATT | | GGUAGTT | |
| 120420 (si) | PEN2 | AAUGAGGAGAAAUU | 73 | GGUUCAAUUUCUCC | 117 |
| | | GAACCTT | | UCAUUTT | |
| 120421 (si) | PEN2 | UUUCUCUGGUUGGU | 74 | UGUUGACCAACCAG | 118 |
| | | CAACATT | | AGAAATT | |
| 120119 (si) | PTP4A1 | UUGAAGGUGGAAUG | 75 | UAUUUCAUUCCACC | 119 |
| | | AAAUATT | | UUCAATT | |
| 120121 (si) | PTP4A1 | CCAAUGCGACCUUAA | 76 | UUGUUUAAGGUCGC | 120 |
| | | ACAATT | | AUUGGTT | |
| 120122 (si) | PTP4A1 | GCAACUUCUGUAUU | 77 | CUCCAAAUACAGAA | 121 |
| | | UGGAGTT | | GUUGCTT | |
| 120743 (si) | MAP2K3 | CAAGAAGACGGACA | 78 | AGCAAUGUCCGUCU | 122 |
| | | UUGCUTT | | UCUUGTT | |
| 120745 (si) | MAP2K3 | GGACAAGUUCUACCG | 79 | CUUCCGGUAGAACU | 123 |
| | | GAAGTT | | UGUCCTT | |
| 120746² | MAP2K3 | GGUCGACUGCUUCU | 80 | AGUGUAGAAGCAGU | 124 |
| (si) | | ACACUTT | | CGACCTT | |
| 19575 (si) | MAP2K3 | GGGCUACAAUGUCA | 81 | GGACUUGACAUUGU | 125 |
| | | AGUCCTT | | AGCCCTT | |
| 19576 (si) | MAP2K3 | GCCCUCCAAUGUCCU | 82 | GAUAAGGACAUUGG | 126 |
| | | UAUCTT | | AGGGCTT | |
| 19577² (si) | MAP2K3 | CAUGCGCACGGUCGA | 83 | GCAGUCGACCGUGC | 127 |
| | | CUGCTT | | GCAUGTT | |
| 19578 (si) | MAP2K3 | GACGAUGGAUGCCG | 84 | GCAGCCGGCAUCCA | 128 |
| | | GCUGCTT | | UCGUCTT | |
| 19579 (si) | MAP2K3 | GCGGAUCCGGGCCAC | 85 | CACGGUGGCCCGGA | 129 |
| | | CGUGTT | | UCCGCTT | |
| Actin 403 | β-actin | GCUAUCCAGGCUGUG | 86 | AUAGCACAGCCUGG | 130 |
| (si) | | CUAUTT | | AUAGCTT | |
| Actin 553 | β-actin | CUGACUGACUACCUC | 87 | UCAUGAGGUAGUCA | 131 |
| (si) | | AUGATT | | GUCAGTT | |
| Actin 617 | β-actin | GGGAAAUCGUGCGU | 88 | AUGUCACGCACGAU | 132 |
| (si) | | GACAUTT | | UUCCCTT | |
| Actin 625 | β-actin | GUGCGUGACAUUAA | 89 | UCUCCUUAAUGUCA | 133 |
| (si) | | GGAGATT | | CGCACTT | |
| Actin 818 | β-actin | GCAUCCACGAAACUA | 90 | AAGGUAGUUUCGUG | 134 |
| (si) | | CCUUTT | | GAUGCTT | |
| Actin 822 | β-actin | CCACGAAACUACCUU | 91 | GUUGAAGGUAGUUU | 135 |
| (si) | | CAACTT | | CGUGGTT | |
| Actin 826 | β-actin | GAAACUACCUUCAAC | 92 | UGGAGUUGAAGGUA | 136 |
| (si) | | UCCATT | | GUUUCTT | |
| Actin 832 | β-actin | ACCUUCAACUCCAUC | 93 | UCAUGAUGGAGUUG | 137 |
| (si) | | AUGATT | | AAGGUTT | |
| Actin 850 | β-actin | AAGUGUGACGUGGA | 94 | GGAUGUCCACGUCA | 138 |
| (si) | | CAUCCTT | | CACUUTT | |
| Actin 869 | β-actin | GCAAAGACCUGUACG | 95 | UUGGCGUACAGGUC | 139 |
| (si) | | CCAATT | | UUUGCTT | |
| lacZ1 (ST) | lacZ | CCGUCUGAAUUUGAC | 96 | AUGCGCUCAGGUCA | 140 |
| | | CUGAGCGCAU | | AAUUCAGACGG | |
| Scramb. | none | GGGAAGACAGAACU | 97 | UUUUGAGUACAAGU | 141 |
| Ctrl (ST) | | UGUACUCAAAA | | UCUGUCUUCCC | |

¹si = standard siRNA; ST = Stealth ™ modified oligonucleotides.
²Single nucleotide mismatches between the RNAi reagent and the MAP2K3
Ultimate ™ ORF are shown in underlined bold for both strands.

### Example 4

### BLOCK-iT™ RNAi Target Screening System

The following example is intended to illustrate exemplary methods for carrying out the present invention. Variations on the methods set forth herein will be readily appreciated by those skilled in the art. The information set forth in this or any other example should not be construed as limiting the scope of the invention described herein. All catalog numbers mentioned in this example refer to specific products and reagents available from Invitrogen Corporation, Carlsbad, Calif., 92008. The exemplary methods described herein can be carried out using the products and reagents designated by the catalog numbers, or with equivalent products and reagents available from other sources. Methods similar to those set out herein may be found in Invitrogen Corporation's instruction manual 25-0723, version B, dated Sep. 24, 2004.

Kit Components

The BLOCK-iT™ RNAi Target Screening Kits include the following components.

TABLE 16

| | Catalog no. | | |
|---|---|---|---|
| Component | V470-20 | K4915-00 | K4916-00 |
| pSCREEN-iT ™/lacZ-DEST | X | X | X |
| Gateway ® Vector Kit | | | |
| FluoReporter ® lacZ/Galactosidase | | X | X |
| Quantitation Kit | | | |
| Lipofectamine ™ 2000 Reagent | | X | X |
| Gateway ® LR Clonase II Enzyme | | | X |
| Mix | | | |
| pCR ® 8/GW/TOPO ® TA Cloning | | | X |
| Kit | | | |

TABLE 17
Reagents and Storage

| Component | Shipping | Storage |
|---|---|---|
| pSCREEN-iT ™/lacZ-DEST | Dry ice | −20° C. |
| Gateway ® Vector Kit | | |
| FluoReporter ® lacZ/Galactosidase | Dry ice | −20° C., protected |
| Quantitation Kit | | from light |
| Lipofectamine ™ 2000 | Blue ice | +4° C. (do |
| Reagent | | not freeze) |
| Gateway ® LR Clonase ™ II | Dry ice | −20° C. |
| Enzyme Mix | | |
| pCR ® 8/GW/TOPO ® TA | Dry ice | pCR ® 8/GW/ |
| Cloning Kit | | TOPO ® TA Cloning |
| | | Reagents: −20° C. |
| | | One Shot ® TOP10 |
| | | Chemically |
| | | Competent |
| | | E. coli: −80° C. |

TABLE 18

| Reagent | Composition | Amount | |
|---|---|---|---|
| pSCREEN-iT ™/lacZ-DEST | Lyophilized in TE Buffer, pH | 6 | μg |
| | 8.0 | | |
| pSCREEN-iT ™/lacZ-GW/ | Lyophilized in TE Buffer, pH | 10 | μg |
| CDK2 Control Vector | 8.0 | | |
| Positive lacZ Stealth ™ | 20 M Stealth ™ RNAi in: | 125 | μl |
| RNAi Control | 10 mM Tris-HCl, pH 8.0 | | |
| | 20 mM NaCl | | |
| | 1 mM EDTA, pH 8.0 | | |
| Scrambled Negative | 20 M Stealth ™ RNAi in: | 125 | μl |
| Stealth ™ RNAi Control | 10 mM Tris-HCl, pH 8.0 | | |
| | 20 mM NaCl | | |
| | 1 mM EDTA, pH 8.0 | | |
| 1× RNA Annealing/Dilution | 10 mM Tris-HCl, pH 8.0 | 1 | ml |
| Buffer | 20 mM NaCl | | |
| | 1 mM EDTA, pH 8.0 | | |

TABLE 19

| Reagent | Composition | Amount |
|---|---|---|
| Gateway ® LR Clonase ™ II | Proprietary | 40 μl |
| Enzyme Mix | | |
| Proteinase K Solution | 2 μg/μl in: | 40 μl |
| | 10 mM Tris-HCl, pH 7.5 | |
| | 20 mM CaCl₂ | |
| | 50% glycerol | |
| pENTR ™-gus Positive Control | 50 ng/μl in TE Buffer, pH 8.0 | 20 μl |

Accessory Products

The products listed below may be used with the BLOCK-iT™ RNAi Target Screening Kits

TABLE 20

| Product | Amount | Catalog no. |
|---|---|---|
| pCR ® 8/GW/TOPO ® TA Cloning Kit | | |
| with One Shot ® TOP10 Chemically | 20 reactions | K2500-20 |
| Competent E. coli | | |
| with One Shot ® Mach1 ™-T1^{R} | 20 reactions | K2520-20 |
| Chemically Competent E. coli | | |
| Gateway ® LR Clonase ™ II Enzyme Mix | 20 reactions | 11791-020 |
| | 100 reactions | 11791-100 |
| One Shot ® TOP10 Chemically | 20 × 50 μl | C4040-03 |
| Competent | | |
| E. coli | | |
| One Shot ® Mach1 ™-T1^{R} Chemically | 20 × 50 μl | C8620-03 |
| Competent E. coli | | |
| Lipofectamine ™ 2000 Reagent | 0.75 ml | 11668-027 |
| | 1.5 ml | 11668-019 |
| Opti-MEM ® I Reduced Serum Medium | 100 ml | 31985-062 |
| | 500 ml | 31985-070 |
| Dulbecco's Phosphate-Buffered Saline | 500 ml | 14190-144 |
| (D-PBS) | 1 L | 14190-136 |
| BLOCK-iT ™ Fluorescent Oligo | 2 × 125 μl (20 μM) | 2013 |
| | 75 μl (1 mM) | 13750-062 |
| FluoReporter ® LacZ/Galactosidase | 1000 reactions | F-2905 |
| Quantitation Kit | | |
| Blasticidin | 50 mg | R210-01 |
| PureLink ™ HQ Mini Plasmid | 100 reactions | K2100-01 |
| Purification Kit | | |
| S.N.A.P. ™ MidiPrep Kit | 20 reactions | K1910-01 |
| GripTite ™ 293 MSR Cell Line | 3 × 10⁶ cells × 2 vials | R795-07 |

Overview

Introduction

The BLOCK-iT™ RNAi Target Screening System uses a lacZ-based reporter vector that is specifically designed to facilitate accurate and sensitive screening of RNAi molecules targeted towards a gene of interest in mammalian cells. The reporter vector is adapted with Gateway® Technology to allow easy generation of a screening construct containing your target gene or sequence of interest fused to the lacZ reporter gene. The screening construct is then cotransfected with the RNAi molecule into mammalian cells, and target gene knockdown assessed by measuring β-galactosidase reporter readout. The System is suitable for use to screen a variety of RNAi molecules including double-stranded RNA (dsRNA) oligomers (i.e. Stealth™ RNAi or siRNA) or plasmids expressing short hairpin RNA (shRNA).

Advantages of the BLOCK-iT™ RNAi Target Screening System

Use of the BLOCK-iT™ RNAi Target Screening System to facilitate screening of RNAi molecules targeted towards a gene of interest provides the following advantages:

Uses a reporter vector to provide a rapid and efficient way to screen and assess the effectiveness of a wide variety of RNAi molecules including siRNA, Stealth™ RNAi, or shRNA-expressing plasmids targeted towards a gene of interest.

The pSCREEN-iT™/lacZ-DEST reporter vector facilitates fusion of a target gene or sequence of interest to the lacZ reporter, allowing accurate and highly sensitive readout of target RNA knockdown without the need for antibodies to the target protein or prior knowledge of the knockdown phenotype.

The System is sensitive enough to discriminate between highly active (i.e. induces >85% target RNA knockdown) and moderately active (i.e. induces 60-85% target RNA knockdown) RNAi molecules.

Target gene knockdown can be analyzed in common, easily transfected cell types, even those that do not express the target endogenously.

The level of target RNA knockdown observed with an RNAi molecule in the screening system correlates with the level of endogenous mRNA knockdown attained as measured by qRT-PCR, thus eliminating the need for specialized equipment and validated primer sets.

The pSCREEN-iT™/lacZ-DEST reporter vector is Gateway®-adapted for easy recombinational cloning of any target gene or sequence of interest from an entry clone, including Invitrogen's Ultimate™ ORF Clones.

Analysis can be carried out within 24 hours of transfection, allowing essential or toxic genes to be targeted over a short enough time period to permit cell survival.

The Gateway® Technology

Gateway® Technology is a universal cloning method that takes advantage of the site-specific recombination properties of bacteriophage lambda to provide a rapid and highly efficient way to move a DNA sequence of interest into multiple vector systems. The reporter vector in the BLOCK-iT™ RNAi Target Screening System is adapted with Gateway® Technology to facilitate generation of a screening construct. To generate the screening construct, simply:
1. Clone a target gene or sequence of interest into pCR®8/GW/TOPO® or any other suitable Gateway® entry vector to create an entry clone. Alternatively, obtain an Ultimate™ ORF Clone containing a target gene of interest from Invitrogen Corporation (Carlsbad, Calif.).
2. Perform an LR recombination reaction between the entry clone and the pSCREEN-iT™/lacZ-DEST reporter vector to generate the screening construct.
3. Proceed to the screening experiment.
   BLOCK-iT™ RNAi Products

A large selection of BLOCK-iT™ RNAi products is available from Invitrogen Corporation (Carlsbad, Calif.) to facilitate RNAi analysis in mammalian and invertebrate systems including those that:

Facilitate production and expression of shRNA molecules in mammalian cells. These vector-based systems allow constitutive or regulated expression of shRNA molecules in mammalian cells.

Facilitate expression of shRNA molecules in any mammalian cell type. Adenoviral and lentiviral vectors are available to allow transient or stable shRNA expression, respectively, in dividing or non-dividing mammalian cells.

Facilitate production and delivery of synthetic short interfering RNA (siRNA), diced siRNA (d-siRNA), or double-stranded RNA (dsRNA) for RNAi analysis in mammalian cells or invertebrate organisms, as appropriate.

Purpose of this Example

This Example provides an overview of the BLOCK-iT™ RNAi Target Screening System and provides instructions and guidelines to:
1. Perform an LR recombination reaction between the pSCREEN-iT™/lacZ-DEST vector and a suitable entry clone containing the target gene or sequence of interest to generate a screening construct.
2. Co-transfect the pSCREEN-iT™/lacZ-DEST screening construct and an RNAi molecule targeting the gene of interest into mammalian cells.
3. At an appropriate time after transfection, harvest cells and assay for β-galactosidase activity to determine the efficacy of the RNAi molecule in inducing target gene knockdown.
4. The LR Clonase™ II Enzyme Mix and Lipofectamine™ 2000 Reagent included in the BLOCK-iT™ RNAi Target Screening System are available separately from Invitrogen Corporation (Carlsbad, Calif.) and are supplied with individual documentation detailing general use of the product. For instructions to use these products specifically with the BLOCK-iT™ RNAi Target Screening System, follow the recommended protocols in this Example.
5. The BLOCK-iT™ RNAi Target Screening System is designed to help screen and identify effective RNAi molecules targeted to a particular gene of interest.
   The BLOCK-iT™ RNAi Target Screening System

Introduction

Many groups have demonstrated that specific RNAi molecules targeting different regions of a transcript can vary widely in their effectiveness at inducing gene silencing (Bohula et al., 2003; Holen, T., Amarzguioui, M., Wiiger, M., Babaie, E., and Prydz, H. (2002). Positional Effects of Short Interfering RNAs Targeting the Human Coagulation Trigger Tissue Factor. Nuc. Acids Res. 30, 1757-1766; Kawasaki, H., Suyama, E., Iyo, M., and Taira, K. (2003). siRNAs Generated by Recombinant Human Dicer Induce Specific and Significant But Target Site-Independent Gene Silencing in Human Cells. Nuc. Acids Res. 31, 981-987; Vickers, T. A., Koo, S., Bennett, C. F., Crooke, S. T., Dean, N. M., and Baker, B. F. (2003). Efficient Reduction of Target RNAs by Small Interfering RNA and RNase H-Dependent Antisense Agents: A Comparative Analysis. J. Biol. Chem. 278, 7108-7118). Although significant improvements have been made in the design rules used to select effective RNAi molecules (Schwarz, D. S., Hutvagner, G., Du, T., Xu, Z., Aronin, N., and Zamore, P. D. (2003). Asymmetry in the Assembly of the RNAi Enzyme Complex. Cell 115, 199-208), testing the efficacy of each RNAi molecule heretofore has been a tedious and time-consuming process. We have developed the BLOCK-iT™ RNAi Target Screening System to provide a means to easily and empirically compare RNAi molecules for their effectiveness at inducing target gene knockdown. This system is based on transfection and does not require prior knowledge of the cellular knockdown phenotype, antibodies to detect target protein, or specialized equipment as would be needed to perform other types of RNAi analysis.

Components of the System

The BLOCK-iT™ RNAi Target Screening System facilitates rapid and accurate screening of RNAi molecules targeted against a gene of interest for RNAi analysis. The System includes the following major components:
1. The pCR®8/GW/TOPO® TA Cloning Kit containing the pCR®8/GW/TOPO® vector for production of an entry clone. The vector is TOPO®- and Gateway®-adapted to allow rapid, 5-minute TOPO® Cloning of a Taq polymerase-amplified PCR product encoding a target gene or sequence of interest, and easy transfer of the target into the pSCREEN-iT™/lacZ-DEST reporter vector, respectively.
2. The pSCREEN-iT™/lacZ-DEST Gateway® destination vector into which the target gene or sequence of interest will be transferred via LR recombination reaction. The resulting screening construct allows expression of a gene of sequence of interest as a lacZ fusion transcript.
3. LR Clonase™ II Enzyme Mix to facilitate transfer of the target gene of interest into pSCREEN-iT™/lacZ-DEST.
4. Positive and negative control Stealth™ RNAi molecules that may be included in the screening experiment to verify the functionality of the system.
5. Lipofectamine™ 2000 Reagent for highly efficient delivery of the screening construct and the corresponding RNAi molecule to mammalian cells.
6. FluoReporter® lacZ/Galactosidase Quantitation Kit containing an improved fluorogenic substrate for highly sensitive detection of β-galactosidase activity.
7. 1× RNA Annealing/Dilution Buffer for dilution of an RNAi molecule stock solutions, as needed to obtain optimal transfection and screening results.

Once a pSCREEN-iT™/lacZ-DEST screening construct is generated, one may cotransfect the vector with an RNAi molecule of interest into mammalian cells and assay for target gene knockdown by measuring β-galactosidase readout.

How the System Works

In the BLOCK-iT™ RNAi Target Screening System, one can clone a target gene or sequence of interest downstream of the lacZ gene to generate a screening construct. Transfection of the screening construct into mammalian cells allows expression of a fusion lacZ transcript. Simultaneous delivery of an active RNAi molecule to the cells induces cleavage of the lacZ fusion transcript, which is then measured by the resulting reduction in β-galactosidase reporter expression and activity (see FIG. 28). The system utilizes the RNAi machinery in mammalian cells but does not require that the target gene be endogenously expressed. This provides the added benefit that target knockdown can be analyzed in common, easily transfected cell types. Finally, analysis is typically carried out within 24 to 48 hours following transfection, allowing essential or toxic genes to be targeted over a short enough time period to permit cell survival.

RNAi Molecules

The BLOCK-iT™ RNAi Target Screening System may be used to screen various types of RNAi molecules including:

Stealth™ RNAi duplexes

siRNA

Plasmids expressing short hairpin RNA (shRNA)

Sensitivity of the System

When using the BLOCK-iT™ RNAi Target Screening System to screen a panel of RNAi molecules targeting a particular gene of interest, one may assess the potency of each RNAi molecule based on its effectiveness in inducing β-galactosidase knockdown. Efficacy of RNAi molecules is generally categorized as follows:
Highly active RNAi molecule—induces >85% target gene knockdown
Moderately active RNAi molecule—induces 60-85% target gene knockdown
Inactive RNAi molecule—induces <60% target gene knockdown

The sensitivity of the System is such that within a certain class of RNAi molecules, one can identify those that are the most potent in inducing target gene knockdown. For example, among highly active RNAi molecules, this System can distinguish between those that induce 85%, 90%, or 95% target gene knockdown.

Target Sequence Options

When generating a pSCREEN-iT™/lacZ-DEST screening construct for use in screening RNAi molecules, one may fuse any target sequence to the lacZ reporter including:
A sequence encoding the gene of interest (i.e. open reading frame (ORF)) or
5′ or 3′ untranslated region (UTR) of the target gene

If an ORF is being fused to the lacZ gene, one may fuse the target sequence in frame with the reporter so that a β-galactosidase fusion protein will be expressed. See the discussion below.

Size of the Target Gene

One may fuse a target sequence of any size to the lacZ reporter in pSCREEN-iT™/lacZ-DEST; however, addition of amino acids from the target protein to the C-terminus of β-galactosidase can affect the expression levels and activity of the β-galactosidase fusion protein. How much so will depend on the nature and length of the target protein. In some cases, one may not observe any detectable β-galactosidase fusion protein expression from the pSCREEN-iT™/lacZ-DEST screening construct following transfection. If so, one may want to try fusing a shorter region of the target gene (i.e. 200 bp to 1 kb) to lacZ or placing a stop codon between lacZ and the target gene of interest to create an RNA-only fusion.
Note: There are a number of advantages and disadvantages associated with creating an RNA-only fusion (see discussion below).

Advantages to Creating an RNA-Only Fusion

In limited instances (e.g. no β-galactosidase fusion protein expressed when fusing a target gene in frame with the lacZ reporter in pSCREEN-iT™/lacZ-DEST), one may want to generate a screening construct that expresses an RNA-only fusion by placing a stop codon between lacZ and the target gene. Expression of a lacZ fusion transcript offers the following advantages over expression of a protein fusion:

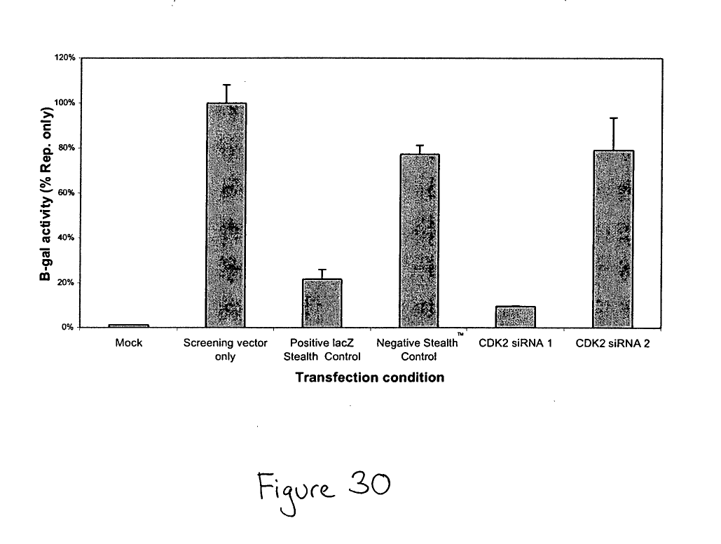

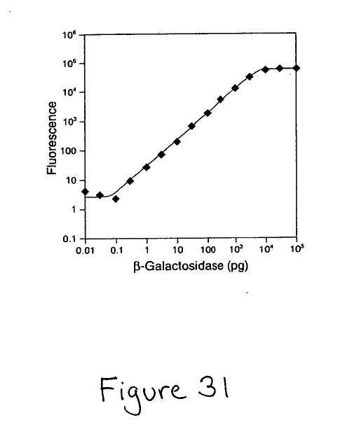

β-galactosidase protein is more likely to be expressed since the only amino acids that are added to the C-terminus of β-galactosidase are those contributed by the attB1 site (see FIG. 29). Because the amount of β-galactosidase expressed depends in part on the stability of the fusion transcript, note that the amount of β-galactosidase protein expressed may still vary from screening construct to screening construct.

Since no part of the target mRNA would be translated into protein, no pleiotropic effects due to overexpression of the target gene should be observed.

Expression of an RNA-only fusion obviates the need to position the inserted gene or gene fragment of interest in frame with lacZ.

Important: While there are a number of advantages to expressing an RNA-only fusion, there is also a disadvantage associated with this option (see below).

Disadvantage to Creating an RNA-Only Fusion

While expression of a lacZ/target gene RNA-only fusion may be desirable for target screening in some cases, this approach also has a disadvantage. It has been observed that the apparent knockdown achieved with a particular RNAi molecule can be negatively affected by the distance between the stop codon and the target site of the RNAi molecule. That is, the farther away the target site from the stop codon, the lower the percentage of β-galactosidase knockdown observed, even with RNAi molecules that are known to be highly active. This phenomenon could result in ranking of effective RNAi molecules as ineffective simply because the target site is distal to the lacZ stop codon. This trend is consistent with the hypothesis that mRNA transcripts cleaved by the RISC in the 3′ UTR can produce functional protein while being slowly degraded by exonucleases. Under this model, as the distance from the stop codon increases, so does the time it takes for the degradation to reach the protein coding region. Note that many other factors can also affect fusion transcript stability. Because of this disadvantage, it is recommended to fuse the target gene in frame with lacZ (to express the fusion protein) whenever possible.
Note: The difference in apparent knockdown achieved with a particular RNAi molecule targeted against a lacZ RNA-only fusion transcript or a fusion protein expressed from the screening construct is minimal when the target sequence is ≦300 bp.

Screening data obtained with RNAi molecules targeted to regions distal to the lacZ junction in a screening construct expressing a lacZ RNA-only fusion transcript does not correlate as well with qRT-PCR analysis (of the endogenous transcript) as does screening vector data obtained with the same RNAi molecules in a screening construct expressing a fusion protein.

Features of the pSCREEN-iT™/lacZ-DEST Vector

The pSCREEN-iT™/lacZ-DEST vector contains the following features:
Human CMV promoter for high-level, constitutive expression of the lacZ/target gene fusion
lacZ gene that is fused to the target gene of interest and functions as a reporter for target gene knockdown following delivery of the screening construct and RNAi molecule to mammalian cells
Two recombination sites, attR1 and attR2, downstream of the lacZ gene for recombinational cloning of the target gene of interest from an entry clone
Chloramphenicol resistance gene (CmR) located between the two attR sites for counterselection
The ccdb gene located between the attR sites for negative selection
pUC origin for high-copy replication of the plasmid in E. coli
Ampicillin resistance gene for selection in E. coli
   Note that the pSCREEN-iT™/lacZ-DEST vector does not contain a selectable marker. The screening construct containing a gene of interest can only be used in transient screening experiments, and not to generate stable cell lines.

Control Stealth™ RNAi Duplexes

The BLOCK-iT™ RNAi Target Screening System includes the Positive lacZ Stealth™ RNAi Control and the Scrambled Negative Stealth™ RNAi Control for use as positive and negative controls for lacZ reporter gene knockdown in mammalian cells. The Positive lacZ Stealth™ RNAi molecule is targeted to and downregulates lacZ mRNA while the Scrambled Negative Stealth™ RNAi molecule does not target any human gene and induces minimal knockdown in mammalian cells. Because it is targeted to lacZ, the Positive lacZ Stealth™ RNAi Control may be used as a positive control for β-galactosidase knockdown in every screening experiment irregardless of the target gene.
Note: In GripTite™ 293 MSR cells, the Positive lacZ Stealth™ RNAi Control is a moderately active RNAi molecule, inducing 70-80% knockdown of β-galactosidase.

Stealth™ RNAi

Stealth™ RNAi is chemically modified dsRNA developed to overcome the limitations of traditional siRNA. Using Stealth™ RNAi for RNAi analysis offers the following advantages:
Produces effective target gene knockdown at levels that are equivalent to or greater than those achieved with traditional siRNA
Reduces non-specific effects caused by induction of cellular stress response pathways
Exhibits enhanced stability for greater flexibility in RNAi analysis.

FluoReporter® lacZ/Galactosidase Quantitation Kit

The BLOCK-iT™ RNAi Target Screening System includes the FluoReporter® lacZ/Galactosidase Quantitation Kit to facilitate highly sensitive measurement of β-galactosidase activity in solution or in cell extracts prepared from cells expressing the lacZ/target gene fusion from a pSCREEN-iT™/lacZ-DEST screening construct. The kit uses an improved fluorogenic substrate, 3-carboxy-umbelliferyl β-D-galactopyranoside (CUG) to allow higher aqueous solubility and increased fluorescence efficiency. This results in a lower threshold of β-galactosidase detection (i.e. 0.5 picograms) over that normally achieved with the more commonly used 4-methylumbelliferyl β-D-galactopyranoside (MUG) substrate.

How the FluoReporter® Kit Works

To use the FluoReporter® Kit, one can add the CUG substrate and an aliquot of cell extract to a well in a 96-well microtiter plate. The β-galactosidase catalyzes the enzymatic cleavage of the CUG substrate to 7-hydroxycoumarin-3-carboxylic acid, a highly fluorescent product (λex=386 nm, λem=448 nm). The fluorescence of the sample can be quantitated in a fluorescence microplate reader equipped with an excitation filter centered at 390 nm and an emission filter centered at 460 nm.

Experimental Outline

The table below describes the general steps required to generate a pSCREEN-iT™/lacZ-DEST screening construct, and to use the screening construct to screen a set of RNAi molecules for target gene knockdown.

TABLE 21

| Step | Action |
|---|---|
| 1 | Generate or obtain a Gateway ® entry clone |
| | containing a target gene or sequence of interest. |
| 2 | Perform an LR recombination reaction between |
| | pSCREEN-iT ™/lacZ-DEST and the entry clone |
| | containing the target gene or sequence of interest |
| | to generate a screening construct. |
| 3 | Purify plasmid DNA from the pSCREEN- |
| | iT ™/lacZ-DEST screening construct. |
| 4 | Cotransfect the pSCREEN-iT ™/lacZ-DEST |
| | plasmid and the RNAi molecule into mammalian |
| | cells. |
| 5 | Harvest cells 24 to 48 hours following transfection |
| | and prepare a cell lysate. |
| 6 | Assay the cell lysate for β-galactosidase activity. |

Methods
Generating an Entry Clone

Introduction

To recombine a gene of interest into pSCREEN-iT™/lacZ-DEST, one may first generate an entry clone containing the target gene or sequence of interest using one of the options discussed below.

Options to Generate an Entry Clone

A number of options exist to generate an entry clone containing a target gene or sequence of interest.

TABLE 22

| Option | Procedure |
|---|---|
| 1 | Use an existing Gateway ® entry clone containing the target |
| | gene of interest or one of Invitrogen's Ultimate ™ ORF Clones. |
| | Note: Entry clones containing an RNAi cassette that is generated |
| | in the BLOCK-iT ™ pENTR ™/U6 or pENTR ™/H1/TO vector |
| | are not suitable for use in this application. However, these |
| | shRNA-expressing plasmids may be used as RNAi knockdown |
| | reagents. |
| 2 | Use the pCR ® 8/GW/TOPO ® TA Cloning Kit to generate the |
| | entry clone. The pCR ® 8/GW/TOPO ® vector facilitates |
| | simple generation of an entry clone using a 5-minute TOPO ® |
| | Cloning reaction with a Taq polymerase-amplified PCR product. |
| 3 | Use another suitable Gateway ® entry vector to generate the |
| | entry clone. |

Ultimate™ ORF Clones

If it is desired to target a human or murine gene of interest, it is recommended that an Ultimate™ Human ORF (hORF) or Mouse ORF (mORF) Clone be used, respectively, available from Invitrogen Corporation (Carlsbad, Calif.). Each Ultimate™ ORF Clone is a fully-sequenced clone provided in a Gateway® entry vector that is ready-to-use in a Gateway® LR recombination reaction with pSCREEN-iT™/lacZ-DEST.
Note: If an Ultimate™ ORF Clone is used in an LR recombination reaction with pSCREEN-iT™/lacZ-DEST, the gene of interest will be cloned in frame with the lacZ reporter gene.

Insert Requirements

For compatibility with the BLOCK-iT™ RNAi Target Screening System, the following factors may be considered when generating an insert to clone into an appropriate entry vector:
The gene of interest should be in frame with the N-terminal lacZ ORF after recombination with the pSCREEN-iT™/lacZ-DEST vector.
Tip: If a PCR product is being produced to clone into an entry vector (e.g. pCR®8/GW/TOPO®), the forward PCR primer can be designed such that the translation reading frame of the PCR product is in the same frame as the -AAA-AAA- triplets in the attL1 site of the entry vector. Note that the first three base pairs of the PCR product should constitute a functional codon.

If it is desired to express an RNA-only fusion after recombination with the pSCREEN-iT™/lacZ-DEST vector, a stop codon may be added to the beginning of the insert.

Although the protein may be fused to the N-terminal lacZ ORF after recombination with the pSCREEN-iT™/lacZ-DEST vector, one may include the ATG initiation codon for the protein in the insert. Inclusion of a Kozak consensus sequence is not necessary.

It should be confirmed that the gene of interest contains a stop codon for proper translation termination of the β-galactosidase fusion protein.
Note: If a stop codon is not included in the insert, note that stop codons in two reading frames are present in the pSCREEN-iT™/lacZ-DEST vector downstream of the attR2 site. Use of these stop codons will result in addition of amino acids to the end of the fusion protein.

Using pCR®8/GW/TOPO®

To generate an entry clone in pCR®8/GW/TOPO®, one may:
Amplify the target gene or sequence of interest using Taq polymerase and the appropriate PCR primers
TOPO® Clone the PCR product into pCR®8/GW/TOPO® in a 5-minute TOPO® Cloning reaction
Transform the TOPO® reaction into competent E. coli and select for entry clones.
   Creating Expression Clones

Introduction

After an entry clone is generated, the LR recombination reaction is performed to transfer the gene of interest into the pSCREEN-iT™/lacZ-DEST vector to create an expression clone.

Experimental Outline

To generate an expression clone, one may:
1. Perform an LR recombination reaction using the attL-containing entry clone (or any Ultimate™ ORF Clone) and the attR-containing pSCREEN-iT™/lacZ-DEST vector. Note: Both the entry clone and the destination vector can be supercoiled.
2. Transform the reaction mixture into a suitable E. coli host.
3. Select for expression clones (see FIG. 29 for a diagram of the recombination region of expression clones in pSCREEN-iT™/lacZ-DEST).

The pSCREEN-iT™/lacZ-DEST vector is supplied as a supercoiled plasmid.

Propagating the Destination Vector

If it is desired to propagate and maintain the pSCREEN-iT™/lacZ-DEST vector, it is recommended that One Shot® ccdB Survival T1R Chemically Competent E. coli from Invitrogen Corporation (Carlsbad, Calif.) (Catalog no. C7510-03) be used for transformation. The ccdB Survival T1R E. coli strain is resistant to CcdB effects and can support the propagation of plasmids containing the ccdB gene. To maintain the integrity of the vector, select for transformants in media containing 100 microgram/ml ampicillin and 15-30 microgram/ml chloramphenicol.
Note: general E. coli cloning strains including TOP10 or DH5α should not be used for propagation and maintenance as these strains are sensitive to CcdB effects.

Recombination Region of pSCREEN-iT™/lacZ-DEST

The recombination region of the expression clone resulting from pSCREEN-iT™/lacZ-DEST x entry clone is shown in FIG. 29.

Features of the Recombination Region:

Shaded regions correspond to those DNA sequences transferred from the entry clone into the pSCREEN-iT™/lacZ-DEST vector by recombination. Non-shaded regions are derived from the pSCREEN-iT™/lacZ-DEST vector.

Bases 3976 and 5659 of the pSCREEN-iT™/lacZ-DEST sequence are indicated.

Potential stop codons that are located downstream of the attB2 site are underlined.

Performing the LR Recombination Reaction

Introduction

E. coli Host

One may use any recA, endA E. coli strain including TOP10, Mach1™-T1R, or DH5α™ for transformation. The LR recombination reaction should not be transformed into E. coli strains that contain the F′ episome (e.g. TOP10F′). These strains contain the ccdA gene and will prevent negative selection with the ccdB gene.

LR Clonase™ II Enzyme Mix

LR Clonase™ II enzyme mix is available from Invitrogen Corporation (Carlsbad, Calif.) to catalyze the LR recombination reaction. The LR Clonase™ II enzyme mix combines the proprietary enzyme formulation and 5× LR Clonase Reaction Buffer previously supplied as separate components in LR Clonase™ enzyme mix into an optimized single-tube format for easier set-up of the LR recombination reaction.
Note: One may perform the LR recombination reaction using LR Clonase™ enzyme mix, if desired.
   Positive Control for LR Reaction

The pENTR™-gus plasmid may be used in an LR recombination reaction to verify the efficiency of the LR reaction. The resulting expression clone may be used to express a lacZ/gus fusion, if desired. For a map of pENTR™-gus, see FIG. 32.

Materials

Purified plasmid DNA of the entry clone (50-150 ng/microliter in TE Buffer, pH 8.0)

pSCREEN-iT™/lacZ-DEST vector (resuspend in water to 150 ng/microliter)

pENTR™-gus control

LR Clonase™ II enzyme mix

2 microgram/microliter Proteinase K solution

TE Buffer, pH 8.0 (10 mM Tris-HCl, pH 8.0, 1 mM EDTA)

Sterile 0.5 ml microcentrifuge tubes

Appropriate competent E. coli host and growth media for expression

S.O.C. Medium

LB agar plates containing 100 microgam/ml ampicillin .

Setting Up the LR Recombination Reaction
1. Add the following components to 0.5 ml microcentrifuge tubes at room temperature and mix.
2. Remove the LR Clonase™ II enzyme mix from −20° C. and thaw on ice (˜2 minutes).
3. Vortex the LR Clonase™ II enzyme mix briefly twice (2 seconds each time).
4. To the sample above, add 2 microliter of LR Clonase™ II enzyme mix. Mix well by pipetting up and down.
Reminder: Return LR Clonase™ II enzyme mix to −20° C. immediately after use.
5. Incubate the reaction at 25° C. for 1 hour.
Note: Extending the incubation time to 18 hours typically yields more colonies.
   6. Add 1 microliter of the Proteinase K solution to each reaction. Incubate for 10 minutes at 37° C.
   7. Transform 1 microliter of the LR recombination reaction into a suitable competent

E. coli host (follow the manufacturer's instructions) and select for expression clones.
Note: The LR reaction may be stored at −20° C. for up to 1 week before transformation, if desired.

If E. coli cells with a transformation efficiency of 1×10⁸ cfu/microgram are used, the LR recombination reaction should result in greater than 5,000 colonies if the entire LR reaction is transformed and plated.

Confirming the Expression Clone

The ccdB gene mutates at a very low frequency, resulting in a very low number of false positives. True expression clones will be chloramphenicol-sensitive and ampicillin-resistant. Transformants containing a plasmid with a mutated ccdB gene will be chloramphenicol- and ampicillin-resistant. To check the putative expression clone, growth on LB plates containing 30 microgram/ml chloramphenicol is tested. A true expression clone should not grow in the presence of chloramphenicol.

Sequencing

Sequencing the expression construct is not required as transfer of the target gene of interest from the entry vector into the pSCREEN-iT™/lacZ-DEST vector preserves the orientation and reading frame of the gene. However, if it is desired to confirm that the gene of interest in pSCREEN-iT™/lacZ-DEST is in the correct orientation and in frame with the lacZ ORF, one may sequence the expression construct.

General Guidelines for Screening

Introduction

Once a pSCREEN-iT™/lacZ-DEST expression construct is generated containing a target sequence fused to the lacZ reporter, this screening construct may be used to screen any type of RNAi molecule targeted towards the gene including:
Stealth™ RNAi
siRNA
shRNA-expressing plasmids

If there are multiple RNAi molecules, the pSCREEN-iT™/lacZ-DEST screening construct can be used to measure the effectiveness of each molecule in inducing target gene knockdown. To screen the RNAi molecules, the pSCREEN-iT™/lacZ-DEST expression construct is cotransfected with the RNAi molecule into a dividing mammalian cell line and knockdown of β-galactosidase reporter activity is assayed. This section provides general guidelines for transfection and discusses factors that can affect the success of the screening experiment.

Factors Affecting Screening Success

A number of factors can influence the degree of success achieved with a screening experiment including:
The mammalian cell line used for screening
Method of transfection and the transfection reagent used
Amount of RNAi molecule transfected
Amount of pSCREEN-iT™/lacZ-DEST plasmid transfected
Transfection format and number of transfections per RNAi molecule
The size of the target gene
The location of the sequence targeted by the RNAi molecule
Each of these factors is discussed in greater detail in this section.
   Selecting a Cell Line

The RNAi molecules may be screened using any dividing mammalian cell line of choice, even one that does not endogenously express the target gene of interest. When choosing a cell line to use for the screening experiments, one with the following characteristics can be chosen:

Transfects efficiently (i.e. easy-to-transfect)

Grows as an adherent cell line

Easy to handle

Exhibits a doubling time in the range of 18-25 hours

Non-migratory

The GripTite™ 293 MSR cell line (Invitrogen Corporation (Carlsbad, Calif.), Catalog no. R795-07) can be used, but the parental HEK293 cell line or other 293 derivatives are also suitable.

Culturing Cells

The health of the cells at the time of transfection can affect the success of the screening experiment. Use of “unhealthy” cells can negatively affect the transfection efficiency, resulting in variability and low-to-moderate target gene knockdown. For optimal results, follow the guidelines below to culture mammalian cells before use in transfection:
Make sure that cells are healthy and greater than 90% viable.
Subculture and maintain cells as recommended by the supplier of the cell line. Cells should not be allowed to overgrow before passaging.
Cells that have been subcultured for less than 20 passages should be used.
   Methods of Transfection

Methods for transfection include calcium phosphate (Chen, C., and Okayama, H. (1987) High-Efficiency Transformation of Mammalian Cells by Plasmid DNA. Mol. Cell. Biol. 7, 2745-2752; Wigler, M., Silverstein, S., Lee, L.-S., Pellicer, A., Cheng, Y.-C., and Axel, R. (1977). Transfer of Purified Herpes Virus Thymidine Kinase Gene to Cultured Mouse Cells. Cell 11, 223-232), lipid-mediated (Felgner, P. L. a., and Ringold, G. M. (1989) Cationic Liposome-Mediated Transfection. Nature 337, 387-388. Cationic Liposome-Mediated Transfection. Nature 337, 387-388), and electroporation (Chu, G., Hayakawa, H., and Berg, P. (1987). Electroporation for the Efficient Transfection of Mammalian Cells with DNA. Nucleic Acids Res. 15, 1311-1326; Shigekawa, K., and Dower, W. J. (1988). Electroporation of Eukaryotes and Prokaryotes: A General Approach to the Introduction of Macromolecules into Cells. BioTechniques 6, 742-751. Electroporation of Eukaryotes and Prokaryotes: A General Approach to the Introduction of Macromolecules into Cells. BioTechniques 6, 742-751).

If Stealth™ RNAi molecules or siRNA are being screened, it should be acknowledged that plasmid DNA (pSCREEN-iT™/lacZ-DEST construct) and double-stranded RNA (dsRNA) will be transfected. The transfection reagent should be one that provides highly efficient delivery of both DNA and RNA to mammalian cells.

For high-efficiency transfection of DNA and dsRNA in a broad range of mammalian cell lines, the cationic lipid-based Lipofectamine™ 2000 Reagent available from Invitrogen Corporation (Carlsbad, Calif.) (Ciccarone, V., Chu, Y., Schifferli, K., Pichet, J.-P., Hawley-Nelson, P., Evans, K., Roy, L., and Bennett, S. (1999). Lipofectamine™ 2000 Reagent for Rapid, Efficient Transfection of Eukaryotic Cells. Focus 21, 54-55) can be used. Using Lipofectamine™ 2000 for transfection offers the following advantages:

Provides the highest transfection efficiency in many mammalian cell types.

DNA- (and/or dsRNA)-Lipofectamine™ 2000 complexes can be added directly to cells in culture medium in the presence of serum.

Removal of complexes, medium change, or medium addition following transfection is not required, although complexes can be removed after 4-6 hours without loss of activity.

Lipofectamine™ 2000 Reagent is available from Invitrogen Corporation (Carlsbad, Calif.).

Opti-MEM® I

To facilitate optimal formation of DNA- (and dsRNA)-Lipofectamine™ 2000 complexes, Opti-MEM® I Reduced Serum Medium available from Invitrogen Corporation (Carlsbad, Calif.) can be used.

Amount of DNA and RNAi Molecule to Use for Transfection

When performing the screening experiment, target gene knockdown is measured using an artificial system rather than knockdown of the endogenous target transcript. Because the pSCREEN-iT™/lacZ-DEST screening construct is simultaneously transfected with the RNAi molecule into mammalian cells, and because it is not necessary to deliver the RNAi molecule to all cells to achieve an RNAi response, the level of sensitivity of target gene knockdown achieved with this system (as measured by β-galactosidase readout) is greater than that achieved with endogenous target gene knockdown. Because of the sensitivity of the system, a lower amount of RNAi molecule is required to elicit an RNAi response. Indeed, transfecting dsRNA or shRNA-containing plasmid DNA at amounts typically used in RNAi analysis (e.g. 50 pmoles of siRNA or 600 ng of shRNA plasmid in a 24-well format) in the context of this system can swamp the system, resulting in significant knockdown of β-galactosidase expression even from RNAi molecules with low to moderate activity. The following factors may be considered when setting up a transfection:

Use 2 to 20-fold less RNAi molecule (i.e. Stealth™ RNAi, siRNA, or shRNA plasmid DNA) in the cotransfection with the screening construct. Optimize as necessary for the mammalian cell line.

To maximize transfection efficiency and prevent cell toxicity, the total amount of nucleic acid transfected (i.e. screening vector construct+RNAi molecule) should not exceed the amount recommended by the manufacturer of the transfection reagent used.

pSCREEN-iT™/lacZ-GW/CDK2 Control

The pSCREEN-iT™/lacZ-GW/CDK2 plasmid (FIG. 5) is a positive control to help optimize transfection conditions in the mammalian cell line. The pSCREEN-iT™/lacZ-GW/CDK2 plasmid expresses the human CDK2 gene as a C-terminal fusion with the lacZ gene. Transfecting the plasmid alone into the mammalian cell line of interest helps to establish a baseline measurement of the amount of β-galactosidase fusion protein expressed in the cells.

To facilitate optimization of transfection conditions for the mammalian cell line, the BLOCK-iT™ Fluorescent Oligo (Catalog no. 2013) available from Invitrogen Corporation (Carlsbad, Calif.) can be used. The BLOCK-iT™ Fluorescent Oligo allows strong, easy fluorescence-based assessment of dsRNA oligomer uptake into mammalian cells, and is ideal for use as an indicator of transfection efficiency.

The effective concentration of RNAi molecule required to induce an RNAi response (assuming the RNAi molecule is active) depends in part on the transfection efficiency of the mammalian cell line and may vary from cell line to cell line. After transfection conditions are optimized for the mammalian cell line and an appropriate amount of RNAi molecule to transfect to obtain an RNAi response is determined, this same amount should be used when screening other RNAi molecules in the same cell line. That is, to accurately compare the effectiveness of an RNAi molecule relative to other RNAi molecules targeted to the same gene in a particular cell line, the same amount of each RNAi molecule should be delivered to the cells.

Transfection Format

The screening experiment may be performed in any tissue culture format. For example:

Transfect Cells in 24-Well Format

For each sample, transfect cells in triplicate. This increases the accuracy of results obtained and accounts for variability associated with transfection.
Note: The screening experiment may be performed in 96-well format, but transfection in this format typically requires more optimization as results obtained are more sensitive to assay variability.
   Plasmid Preparation

Once the pSCREEN-iT™/lacZ-DEST expression clone is generated, plasmid DNA is isolated for transfection. This also applies to shRNA-containing plasmids. Plasmid DNA for transfection into eukaryotic cells should be very clean and free from contamination with phenol and sodium chloride. Contaminants will kill the cells, and salt will interfere with lipid complexing, decreasing transfection efficiency. It is recommended to isolate plasmid DNA using the PureLink™ HQ Mini Plasmid Purification Kit (Catalog no. K2100-01) or S.N.A.P.™ MidiPrep Kit (Catalog no. K1910-01) available from Invitrogen Corporation (Carlsbad, Calif.) or CsCl gradient centrifugation.

Resuspend the purified plasmid DNA in sterile water or TE Buffer, pH 8.0 to a final concentration ranging from 0.1-3.0 microgram/microliter.

Recommended Positive and Negative Controls

The following positive and negative controls may be included in each screening experiment to help interpret the results. The screening vector construct is the pSCREEN-iT™/lacZ-DEST vector containing the target gene or sequence of interest.

Mock transfection (i.e. no screening vector, no RNAi molecule): This control assesses the effects of the transfection reagent on the mammalian cells.

Screening vector construct only: This control provides a baseline measurement of the amount of the β-galactosidase fusion protein expressed in mammalian cells after transfection.
Reminder: If the mammalian cell line is being transfected for the first time and it is desired to optimize transfection conditions, the pSCREEN-iT™/lacZ-GW/CDK2 vector can be used.

Screening vector construct+positive control RNAi molecule: The positive control RNAi molecule can be an active RNAi molecule targeted to the gene of interest or the Positive lacZ Stealth™ RNAi Control. Use of the Positive lacZ Stealth™ RNAi Control effectively targets the lacZ reporter gene, resulting in >70% knockdown of β-galactosidase expression.

Screening vector construct +negative control RNAi molecule: The negative control RNAi molecule can be an inactive RNAi molecule targeted to the gene of interest or the Scrambled Negative Stealth™ RNAi Control. Use of the Scrambled Negative Stealth™ RNAi control does not target any human gene and should induce minimal knockdown of β-galactosidase expression when transfected into mammalian cells at concentrations less than 50 nM.

Transfecting Cells Using Lipofectamine™ 2000

Introduction

This section provides a protocol to cotransfect the pSCREEN-iT™/lacZ-DEST screening construct and a corresponding RNAi molecule (i.e. Stealth™ RNAi, siRNA, or shRNA plasmid) into mammalian cells using Lipofectamine™ 2000 Reagent.

Experimental Outline

To perform a screening experiment:
Co-transfect the pSCREEN-iT™/lacZ-DEST screening construct and the RNAi molecule into mammalian cells using Lipofectamine™ 2000.
Harvest cells and prepare a cell lysate 24-48 hours after transfection.
Assay the cell lysates for β-galactosidase activity.
   Note that the guidelines provided in this section regarding the time period in which to harvest cells are optimized for transfection with Lipofectamine™ 2000. If another transfection reagent is used, the optimal transfection conditions to use and when to harvest cells to obtain the best screening results should be determined.

Materials

Mammalian cell line cultured in the appropriate growth medium

pSCREEN-iT™/lacZ-DEST screening construct (0.1-3.0 microgram/microliter in sterile water or TE Buffer, pH 8.0)

Stealth™ RNAi or siRNA of interest (20 μM stock in 1× RNA Annealing/Dilution Buffer) or shRNA expression plasmids of interest (0.1-3.0 μg/μl in sterile water or TE Buffer, pH 8.0)

20 μM Positive lacZ Stealth™ RNAi control

20 μM Scrambled Negative Stealth™ RNAi control

1× RNA Annealing/Dilution Buffer

pSCREEN-iT™/lacZ-GW/CDK2 control plasmid

Lipofectamine™ 2000 Reagent

Opti-MEM® I Reduced Serum Medium (pre-warmed)

Appropriate tissue culture plates and supplies

Dulbecco's Phosphate-Buffered Saline (D-PBS; Catalog no. 14190-144)

Cell Lysis Buffer (25 mM Tris-HCl, pH 8.0, 0.1 mM EDTA, pH 8.0, 10% glycerol, 0.1% Triton-X-100)

General Guidelines for Transfection

Use low-passage cells, and make sure that cells are healthy and greater than 90% viable before transfection.

Transfect cells at 80-90% confluence.

Do not add antibiotics to the medium during transfection as this reduces transfection efficiency and causes cell death.

For optimal results, use Opti-MEM® I Reduced Serum Medium to dilute Lipofectamine™ 2000, DNA, and dsRNA oligomers prior to complex formation.

Stealth™ RNAi duplexes or siRNA are generally supplied as a 20 micromolar stock solution. If transfection is performed in a format smaller than a 6-well dish (e.g. 24-well format), the 20 micromolar stock solution should be diluted 10- to 20-fold in 1× RNA Annealing/Dilution Buffer to prepare a 1-2 micromolar stock solution, as appropriate. The 1-2 micromolar stock solution is used for transfection. Store the 2 micromolar stock solution at −20° C.
Example: To prepare a 2 micromolar stock solution, dilute 2 microliters of the 20 micromolar siRNA or StealthTm RNAi stock solution in 18 microliters of 1× RNA Annealing/Dilution Buffer).

To increase accuracy and reduce assay variability, triplicate transfections for each sample condition can be performed.

Transfection Procedure

This procedure may be used to cotransfect the pSCREEN-iT™/lacZ-DEST screening construct containing the target gene or sequence of interest and the RNAi molecule into mammalian cells using Lipofectamine™ 2000.
1. One day before transfection, plate cells in the appropriate amount of growth medium without antibiotics such that they will be 80-90% confluent at the time of transfection.
2. For each transfection sample, prepare DNA-RNAi molecule-Lipofectamine™ 2000 complexes as follows.
   a. Dilute the DNA and RNAi molecule in the appropriate amount of Opti-MEM® I Medium without serum. Mix gently.
   b. Mix Lipofectamine™ 2000 gently before use, then dilute the appropriate amount in Opti-MEM® I Medium without serum. Mix gently and incubate for 5 minutes at room temperature.
   c. After the 5 minute incubation, combine the diluted DNA and RNAi molecule with the diluted Lipofectamine™ 2000. Mix gently and incubate for 20 minutes at room temperature to allow complex formation to occur. The solution may appear cloudy, but this will not impede the transfection.
3. Add the DNA-RNAi molecule-Lipofectamine™ 2000 complexes to each well containing cells and medium. Mix gently by rocking the plate back and forth.
4. Incubate the cells at 37° C. in a CO₂ incubator until you are ready to harvest cells and assay for β-galactosidase activity. Removal of complexes or media change is not required; however, growth medium may be replaced after 4-6 hours without loss of transfection activity.
Tip: Cells can be harvested 24-48 hours after transfection.

Suggested Reagent Amounts and Volumes

The table below lists the range of recommended reagent amounts and volumes to use to transfect cells in various tissue culture formats. As a starting point, use an amount of pSCREEN-iT™/lacZ-DEST DNA (see column 4), dsRNA or shRNA plasmid DNA (see column 5), and Lipofectamine™ 2000 (see column 7) that falls around the mid-point of the recommended range, then optimize conditions for the cell line by varying reagent amounts within the recommended range. If it is desired to perform transfection in 96-well format, see the additional guidelines in Guidelines for Transfection in 96-Well Format, below.
Example: Use 150 ng of screening vector DNA, 5 pmol of Stealth™ RNAi, and 1 microliter of Lipofectamine™ 2000 to transfect GripTite™ 293 MSR cells in 24-well format.

Tip: 20 micromolar dsRNA (i.e. siRNA or Stealth™ RNAi)=20 pmol/microliter.

TABLE 23

| | Relative | | pSCREEN- | | | Lipid (I) |
|---|---|---|---|---|---|---|
| | Surface | Volume of | iT ™/ | dsRNA (pmol)/ | DNA/RNA | and |
| Culture | Area (vs. | Plating | lacZ-DEST | shRNA DNA (ng) | Dilution | Dilution |
| Vessel | 24-well) | Medium | DNA (ng) Amt | Amt¹ | Volume (I)² | Volume (I) |
| 96-well | 0.2 | 100 μl | 10-100 ng | 0.1-1 pmol/ | 25 μl | 0.2-0.5 μl |
| | | | | 150-300 ng | | in 25 μl |
| 48-well | 0.4 | 200 μl | 50-100 ng | 0.5-5 pmol/ | 25 μl | 0.3-0.8 μl |
| | | | | 150-300 ng | | in 25 μl |
| 24-well | 1 | 500 μl | 100-200 ng | 1-10 pmol/ | 50 μl | 0.5-1.5 μl |
| | | | | 300-600 ng | | in 50 μl |
| 6-well | 5 | 2 ml | 500-1000 ng | 5-50 pmol/ | 250 μl | 2.5-6 μl in |
| | | | | 1.5-3 μg | | 250 μl |
| ¹dsRNA = siRNA or Stealth ™ RNAi; shRNA DNA = shRNA-containing plasmid | | | | | | |
| ²Dilute the pSCREEN-iT ™/lacZ-DEST DNA and the dsRNA or shRNA DNA into this volume of Opti-MEM ® I. | | | | | | |
| Note that for highly potent RNAi molecules (i.e. RNAi molecules inducing >90% target knockdown), the amount of dsRNA or shRNA DNA required to | | | | | | |
| # obtain effective knockdown may be less than the amounts specified in the table above (see column 5). This needs to be determined empirically for each cell line. | | | | | | |

Guidelines for Transfection in 96-Well Format

The screening experiment may be performed in 96-well format, if desired. Note that in this format, the results obtained from the screening experiment are much more sensitive to well-to-well variability caused by differences in cell density, transfection efficiency, and reagent amounts used. If cells are transfected in 96-well format, significant optimization of transfection conditions may be required. Follow the guidelines below to cotransfect mammalian cells in 96-well format:

To address potential problems caused by well-to-well variability, more replicates should be performed for each sample condition; e.g., transfect each sample into 6-7 individual wells.

When plating cells, cells should be evenly distributed over the surface of each well. As with the other tissue culture formats, transfect cells at 80-90% confluence.

Use the following range of recommended reagent amounts and volumes listed in the table above and optimize accordingly.

Cells can be harvested and assayed for β-galactosidase activity 24 hours after transfection.

Preparing Cell Lysates

This procedure can be used to prepare cell lysates from untransfected and transfected cells. The amount of Cell Lysis Buffer recommended in column 2 of the table below can be used as a starting point. The β-galactosidase assay can be optimized by varying the amount of Cell Lysis Buffer used within the recommended range (see column 3).
1. Remove the growth medium from each well of the tissue culture dish and wash the cells once with D-PBS.

2. Add the appropriate amount of Cell Lysis Buffer to each well containing cells.

TABLE 24

| Tissue-Culture | Amt of Cell Lysis Buffer | Cell Lysis Buffer Range to |
|---|---|---|
| Format | (μl) | Optimize (μl) |
| 96-well | 100 μl | 25-100 |
| 48-well | 250 μl | 100-250 |
| 24-well | 500 μl | 125-500 |
| 6-well | 2000 μl | 600-2000 |

3. Transfer the plate containing cells and Cell Lysis Buffer to −80° C. for at least 20 minutes until samples are frozen.
   Note: Samples may be stored for up to one month at this stage by wrapping the plate with parafilm or plastic wrap and storing at −80° C.
4. Proceed to assay for β-galactosidase activity.
   Guidelines to Perform the β-galactosidase Assay

Introduction

Once cell lysates of the untransfected and transfected cells are prepared, each sample can be assayed for β-galactosidase activity using, e.g., the FluoReporter® lacZ/Galactosidase Quantitation Kit (Invitrogen Corporation, Carlsbad, Calif.) (Catalog nos. K4915-00 and K4916-00 only). The kit uses a fluorogenic substrate to allow highly sensitive measurement of β-galactosidase activity in cell extracts using a fluorescence microplate reader equipped with the proper filter set.
Note: The FluoReporter® lacZ/Galactosidase Quantitation Kit is available separately from Invitrogen Corporation (Carlsbad, Calif.) (Catalog no. F-2905). Other methods or commercial kits may also be used to assay for β-galactosidase activity.
   Assay Format

The β-galactosidase assay may be performed in a 96-well format. This allows rapid analysis of multiple samples and minimizes the amount of cell lysate required for each assay.

Fluorescence Plate Readers and Filter Sets

Any fluorescence plate reader may be used to detect the fluorescence signal after performing the β-galactosidase assay.

For optimal sensitivity, a bottom-read fluorescence plate reader (e.g. Gemini-EM Fluorescence Microtiter Plate Reader, Molecular Devices, CytoFluor® 4000 Fluorescence Plate Reader, PerSeptive Biosystems, or Safire Microplate Reader, Tecan) is recommended. Top-read fluorescence plate readers (e.g. Gemini-XS Fluorescence Microtiter Plate Reader, Molecular Devices) can be used.

To detect the blue fluorescence signal, a fluorescence microplate reader equipped with an excitation filter centered at ˜390 nm and an emission filter centered at ˜460 nm can be used.

The following filter set from Chroma Technologies (Catalog no. 31047) can be used:
Excitation filter: D405/10x
Dichroic mirror: 425DCLP
Emission filter: D460/50m.
   General Recommendations

The β-galactosidase assay can preferably be performed in a black-walled, clear-bottom microtiter plate with low autofluorescence (Costar, Catalog nos. 3603 or 3631). Using a black-walled microtiter plate blocks any signal from adjoining wells during quantitation by the fluorescence microplate reader.

Some plates/plate readers exhibit edge effects that may affect data. If edge effects are noticed, consider the plate layout when setting up the assay.

The bottom of the microtiter plate should not be touched; dust should not be allowed to cover the tissue culture surface. Fingerprints and dust can autofluoresce, introducing well-to-well variability in replicate wells.

Include the Reference Standard and the appropriate controls (mock transfection, screening construct only transfection) in the experiment.

Reference Standard

The Reference Standard (7-hydroxycoumarin-3-carboxylic acid) may serve as an instrument-independent control, and can be used to normalize fluorescence. This allows a single standard curve to be used for assays performed at different times, even if performed on different instruments or with different instrument settings. The reference standard can also be used to convert the fluorescence signal into moles of product.

Generating a Standard Curve

When using the FluoReporter® lacZ/Galactosidase Quantitation Kit, a standard curve can be generated using purified β-galactosidase solutions of known concentration. Generating a standard curve allows one to:
Determine the linear detection range of β-galactosidase based on the reagents, buffers, and fluorescence microplate reader;
Convert the fluorescence readings for your samples into picograms of β-galactosidase.
   Performing the β-galactosidase Assay

Introduction

This section provides exemplary instructions to perform a galactosidase assay.

Experimental Outline

To assay samples for β-galactosidase activity:
1. Add an aliquot of cell extract and the CUG substrate to wells in a 96-well microtiter plate.
Recommendation: For increased accuracy, the assay can be performed in triplicate.
2. Incubate the sample(s) at room temperature for 30 minutes.
3. Add a stop buffer to terminate the reaction.
4. Measure fluorescence signal using a fluorescence microplate reader equipped with the appropriate filter set.
   Amount of Cell Extract to Assay

The β-galactosidase assay is generally performed using 10 μl of cell extract. If the sample contains high levels of β-galactosidase activity, the fluorescence signal may exceed the linear range of detection. In this case, it may be necessary to dilute the cell extracts in Cell Lysis Buffer prior to performing the assay.

Materials Needed

Cell extracts of interest (in Cell Lysis Buffer);
40 mM CUG Substrate Reagent;
10 mM Reference Standard (optional);
Reaction Buffer (0.1 M sodium phosphate, pH 7.3, 1 mM MgCl₂, 45 mM β-mercaptoethanol).
Note: Approximately 10 ml of Reaction Buffer is needed for every 96-well plate. If a standard curve will be generated or the Reference Standard is used, additional Reaction Buffer may be needed to prepare the enzyme dilution buffer and dilute the Reference Standard.
   Stop Buffer (0.2 M Na₂CO₃);
Note: You approximately 5 ml of Stop Buffer is needed for every 96-well plate.
   Enzyme Dilution Buffer (if generating a standard curve; Reaction Buffer containing 1 mg/ml BSA)
   1 μg/ml β-galactosidase solution in Enzyme Dilution Buffer (if generating a standard curve).
      Handling the Reagents

The CUG Substrate Reagent may be supplied as a 40 mM stock solution in 100 mM sodium phosphate buffer (pH 7.0), 1 mM MgCl₂, and 110 mM β-mercaptoethanol while the Reference Standard may be supplied as a 10 mM stock solution in dimethylformamide.

The CUG Substrate Reagent and the Reference Standard are light sensitive. Store the CUG Substrate Reagent at −20° C., protected from light. Store the Reference Standard at −20° C. or +4° C. The stock solutions are stable for at least 6 months if stored properly.

When using, thaw the CUG substrate stock solution at room temperature, protected from light. Thaw immediately before use. Do not expose to room temperature for an extended period of time as spontaneous hydrolysis will occur. After use, return stock solution to −20° C. storage.

Note: The Reference Standard does not freeze.

The CUG Substrate Reagent stock solution may be frozen and thawed multiple times without loss of fluorescence signal if handled properly.

Before Beginning

Prepare a 1.1 mM working solution of the CUG Substrate Reagent by diluting 275 μl of the 40 mM stock solution with 9.73 ml of Reaction Buffer. Approximately 10 ml of CUG working solution is needed for each 96-well microtiter plate. Scale up the volume needed accordingly. Do not leave the CUG Substrate Reagent at room temperature for an extended period of time (see handling instructions above).
Note: Store the working solution at −20° C. for at least six months.

If the Reference Standard is used, dilute the 10 mM Reference Standard 100-fold into 200 μl of Reaction Buffer to prepare a 0.1 mM working solution (i.e. add 5 μl of 10 mM Reference Standard to 495 μl of Reaction Buffer).

β-galactosidase Assay Procedure

1. Remove the plate containing cell lysates from the freezer and thaw the cell lysates at room temperature for 30-45 minutes.
2. Rock the plate gently to mix the solution, then pipette 10 μl of cell lysate into individual wells of a black-walled, 96-well microtiter plate. Take the clear solution; do not pipette any insoluble material into the 96-well plate. Wrap the plate containing unused cell lysate with parafilm or plastic wrap and store at −80° C.
Tip: For more accurate results, it is recommended to assay each sample in triplicate.
3. Pipet 10 μl of Reaction Buffer into a well to serve as a blank.
4. Add 100 μl of the 1.1 mM CUG substrate working solution to each well containing 10 μl of cell lysate.
5. Optional: Pipet 100 μl of the 0.1 mM Reference Standard into an empty well.
6. Incubate the samples at room temperature for 30 minutes.
Important: If results are compared to a previously generated standard curve, incubation time may be critical. The same incubation time and temperature should be used to ensure accurate quantitation.
7. Add 50 μl of Stop Buffer to each well to terminate the reaction. In addition to terminating the reaction, the Stop Buffer causes an increase in the fluorescence of the product.
8. Measure the fluorescence signal in each well using a fluorescence microplate reader equipped with the appropriate filter set.
Important: Measure fluorescence signal within 15 minutes of adding the Stop Buffer. If comparing results to a previously generated standard curve, use the same time interval between stopping the reaction and reading the fluorescence signal.
9. Analyze results (see below).
   Analyzing Results

Analyze the fluorescence of the samples by subtracting the fluorescence of the blank from that of each sample. If the Reference Standard is used, divide the corrected fluorescence by the background-subtracted fluorescence of the Reference Standard. Use the standard curve to determine the amount of β-galactosidase in each well, if desired.

Example of Expected Results

Screening siRNA Targeting the Human CDK2 Gene

In this experiment, we wish to screen several synthetic siRNA targeting the human CDK2 gene (i.e. CDK2 siRNA 1 and CDK2 siRNA 2). An Ultimate™ hORF entry clone containing the human CDK2 gene (Invitrogen Corporation (Carlsbad, Calif.), ORF no. IOH21140) was transferred into pSCREEN-iT™/lacZ-DEST using the LR recombination reaction to generate the pSCREEN-iT™/lacZ-GW/CDK2 screening construct.

GripTite™ 293 MSR cells (Catalog no. R795-07) plated in a 24-well plate were transfected using Lipofectamine™ 2000 with either the pSCREEN-iT™/lacZ-GW/CDK2 screening vector alone or together with a Stealth™ RNAi control or one of the CDK2 siRNA. Twenty-four hours after transfection, cell lysates were prepared and assayed in triplicate for β-galactosidase activity using the FluoReporter® lacZ/Galactosidase Quantitation Kit reagents. The β-galactosidase activity reported is normalized to the % activity obtained from the screening vector (i.e. reporter) alone.

Results

The results indicate that CDK2 siRNA 1 is a highly active siRNA for human CDK2 as measured by >85% knockdown of lacZ reporter activity. In contrast, CDK2 siRNA 2 is not an active siRNA, with only 20% knockdown of lacZ reporter activity achieved.

The results obtained from the screening experiment correlate with real-time quantitative RT-PCR (qRT-PCR) analysis of the endogenous CDK2 transcript.

Troubleshooting

Introduction

LR Reaction and Transformation

TABLE 25

| Problem | Reason | Solution |
|---|---|---|
| Few or no colonies | LR recombination reaction not | Treat reaction with proteinase K |
| obtained after | treated with proteinase K | before transformation. |
| transformation of LR | | |
| reaction | | |
| | Did not use the suggested amount of | Make sure to store the LR |
| | LR Clonase ™ II enzyme mix or LR | Clonase ™ II enzyme mix at −20° C. |
| | Clonase ™ II enzyme mix was | Do not freeze/thaw the LR |
| | inactive | Clonase ™ II enzyme mix more than |
| | | 10 times. |
| | | Use the recommended amount of |
| | | LR Clonase ™ II enzyme mix. |
| | | Test another aliquot of the LR |
| | | Clonase ™ II enzyme mix. |
| | Not enough LR reaction transformed | Transform 2-3 μl of the LR reaction |
| | | into a suitable chemically competent |
| | | E. coli strain. |
| | Not enough transformation mixture | Increase the amount of E. coli |
| | plated | plated. |
| | Did not perform the 1 hour grow-out | After the heat-shock step, add |
| | period before plating the | S.O.C. Medium and incubate the |
| | transformation mixture | transformation mixture for 1 hour at |
| | | 37° C. with shaking before plating. |
| | Too much entry clone DNA used in | Use 50-150 ng of the entry clone in |
| | the LR reaction | the LR reaction. |
| | Used low efficiency competent cells | Use competent E. coli with a |
| | | transformation efficiency 1 × 10⁸ cfu/μg. |

Screening Experiment

TABLE 26

| Problem | Reason | Solution |
|---|---|---|
| Kockdown | Too much RNAi molecule | Reduce the amount of RNAi |
| observed when | transfected | molecule transfected. |
| cotransfecting | | |
| screening construct | | |
| and negative control | | |
| (i.e. inactive) RNAi | | |
| molecule | | |
| Low levels of | Low transfection efficiency: | Use the PureLink ™ HQ Mini |
| β-galactosidase | Used poor quality pSCREEN-iT ™/ | Plasmid Purification Kit (Catalog |
| activity obtained | lacZ-DEST screening construct | no., K2100-01), S.N.A.P. ™ |
| when screening | plasmid DNA (e.g. DNA contaminated | MidiPrep Kit (Catalog no. K1910- |
| construct alone is | with phenol) | 01) or CsC1 gradient centrifugation |
| transfected | Transfected unhealthy mammalian | to prepare DNA. |
| Note: Assumes that | cells; cells exhibit low viability | Use healthy mammalian cells under |
| Lipofectamine ™ | Cells transfected in media | passage 20. Do not overgrow; make |
| 2000 used for | containing antibiotics (e.g. | sure cells are >90% viable before |
| transfection | penicillin/streptomycin) | transfection. |
| | Did not transfect enough screening | Do not add antibiotics to media |
| | construct plasmid DNA | during transfection; this reduces |
| | Mammalian cells plated too sparsely | transfection efficiency and causes |
| | Used a cell line that does not | cell death. |
| | transfect efficiently | Use an amount of plasmid DNA that |
| | Plasmid DNA: transfection reagent | falls within the range recommended. |
| | ratio used not optimal | Plate cells such that they are 80-90% |
| | | confluent at the time of |
| | | transfection. |
| | | Use a different mammalian cell line |
| | | for transfection (e.g. GripTite ™ 293 |
| | | MSR). |
| | | Use an amount of plasmid DNA and |
| | | lipid that falls within the range |
| | | recommended. |
| | C-terminal fusion of your target | Reduce the amount of Cell Lysis |
| | gene to lacZ interferes with | Buffer used to lyse cells. |
| | β-galactosidase activity or | Test pSCREEN-iT ™/lacZ- |
| | expression | GW/CDK2 for β-galactosidase |
| | | fusion protein expression. |
| | | Reduce the size of the target |
| | | sequence fused to lacZ; use a DNA |
| | | fragment ranging from 200 bp to 1 kb. |
| | | Place a stop codon before the |
| | | beginning of the target sequence. |
| | Lipofectamine ™ 2000 Reagent | Store at +4° C. Do not freeze. |
| | handled incorrectly | Mix gently by inversion before use. |
| | | Do not vortex. |

TABLE 27

| Problem | Reason | Solution |
|---|---|---|
| Poor knockdown or | Insufficient amount of RNAi | Increase the amount of RNAi |
| no knockdown | molecule transfected | molecule transfected. |
| observed when | | Optimize cotransfection conditions |
| cotransfecting | | for the cell line by varying screening |
| screening construct | | construct plasmid DNA, RNAi |
| and positive control | | molecule, and lipid amounts used. |
| (i.e. highly potent) | | |
| RNAi molecule | | |
| | Cell lysate assayed contained too | Dilute the cell lysate in Cell Lysis |
| | much β-galactosidase | Buffer and repeat the β- |
| | | galactosidase detection assay. Make |
| | | sure that the amount of β- |
| | | galactosidase in the sample is within |
| | | the linear range of detection. |
| Significant | Too much Lipofectamine ™ 2000 | Reduce the amount of |
| cytotoxicity | used | Lipofectamine ™ 2000 used. |
| observed | | |
| Note: Assumes that | | |
| Lipofectamine ™ | | |
| 2000 used for | | |
| transfection | | |
| | Mammalian cells plated too sparsely | Plate cells such that they are 80-90% |
| | | confluent at the time of transfection. |
| | Too much nucleic acid (i.e. | Reduce the total amount of nucleic |
| | screening construct DNA + RNAi | acid transfected. |
| | molecule) transfected | |
| No fluorescence | CUG substrate stock solution | Store the CUG substrate stock |
| signal (i.e. no β- | exposed to light during storage | solution at |
| galactosidase activity | | −20° C., protected from light. |
| in all samples) | | |
| | Used the incorrect filter set | Measure fluorescence using a |
| | | fuorescence microplate reader |
| | | equipped with an excitation filter |
| | | centered at 390 nm and an emission |
| | | fliter centered at 460 nm. |
| CUG substrate | CUG substrate has spontaneously | Do not leave the CUG substrate |
| exhibits fluorescence | hydrolyzed | stock solution at room temperature |
| signal in the absence | | for extended periods of time. |
| of β-galactosidase | | Store the CUG substrate stock |
| | | solution at −20° C., protected from |
| | | light. |
| Observe well-to-well | Bubbles are present in the cell | Carefully transfer cell lysates to a |
| variability in | lysates | new tissue culture plate, taking care |
| replicate wells (most | | not to introduce bubbles. Read |
| notable when using | | fluorescence signal. |
| top-read | | |
| fluorescence plate | | |
| readers) | | |
| | Touched the bottom of the | Do not touch the bottom of the |
| | microtiter plate | microtiter plate as fingerprints can |
| | | autofluoresce. |
| | Microtiter plate covered with dust or | Dust can autofluoresce. Keep the |
| | lint | bottom and top surface of the |
| | | microtiter plate free of dust. |

Appendix
Recipes

Cell Lysis Buffer
25 mM Tris-HCl, pH 8.0
0.1 mM EDTA, pH 8.0
10% glycerol
0.1% Triton X-100

1. In a sterile beaker, combine the following:
1 M Tris-HCl, pH 8.0: 12.5 ml
0.5 M EDTA, pH 8.0: 100 ml
Glycerol: 50 ml
Triton X-100: 5 ml
Sterile deionized water: 332.5 ml
Total volume: 500 ml

2. Stir to mix thoroughly.

3. Filter-sterilize and store at +4° C.

Reaction Buffer

Follow this procedure to prepare 10 ml of Reaction Buffer for use with the reagents supplied in the FluoReporter® lacZ/Galactosidase Quantitation Kit. To prepare a larger volume of Reaction Buffer, scale up the amounts of each reagent accordingly.

Composition:

0.1 M Sodium Phosphate, pH 7.3
1 mM MgCl₂
45 mM β-mercaptoethanol.
   Recipe:

1. In a 15 ml sterile, conical tube, combine the following:
1 M Sodium Phosphate, pH 7.3: 1 ml
1 M MgCl₂: 10 μl
β-mercaptoethanol: 31.5 μl
Sterile deionized water: 8.96 ml
Total volume: 10 ml

2. Mix thoroughly.

3. Store at room temperature until use.
1 M Sodium Phosphate, pH 7.3.
   Materials Needed

Sodium phosphate monobasic monohydrate (H₂NaPO₄.H₂O; Sigma, Catalog no. S-9638)

Sodium phosphate dibasic (HNa₂PO₄; Sigma, Catalog no. S-7907).

Recipe:

1. Prepare 2 M stock solutions of each reagent:
a. 2 M H₂NaPO₄.H₂O: Dissolve 55.2 g in 200 ml sterile deionized water.
b. 2 M HNa₂PO₄: Dissolve 56.8 g in 200 ml sterile deionized water.

2. In a beaker, combine the following:
2 M H₂NaPO₄.H₂O: 23 ml
2 M HNa₂PO₄: 77 ml
Sterile deionized water: 100 ml
Total volume: 200 ml

3. Stir to mix thoroughly. This is the 1 M Sodium Phosphate, pH 7.3 solution.

4. Filter-sterilize and store at room temperature.

Stop Buffer
Stop Buffer=0.2 M Na₂CO₃ (Sigma, Catalog no. 71350)
   1. To prepare a 2 M stock solution of Na₂CO₃, add 10.6 g of Na₂CO₃ to 45 ml of sterile deionized water. Stir to mix and bring the volume up to 50 ml with sterile deionized water. Filter-sterilize.
   2. Dilute an aliquot of the 2 M Na₂CO₃ stock solution 10-fold in sterile deionized water (e.g. add 1 ml of 2 M Na₂CO₃ to 9 ml of sterile deionized water) to prepare a 0.2 M working solution.
   3. Store at room temperature until use.
      Generating a β-galactosidase Standard Curve

Introduction

Follow the guidelines provided in this section to generate a standard curve using purified β-galactosidase solutions and reagents supplied in the FluoReporter® lacZ/Galactosidase Quantitation Kit.

Materials Needed:

Bovine Serum Albumin (BSA; Invitrogen Corporation (Carlsbad, Calif.), Catalog no. 15561-020)

1 μg/ml β-galactosidase (Sigma, Catalog no. G4155) in Enzyme Dilution Buffer (see below)

1.1 mM working solution of CUG Substrate Reagent

Reaction Buffer

0.1 mM working solution of Reference Standard

96-well black-walled, microtiter plate.

Before Beginning

1. Prepare Enzyme Dilution Buffer by adding BSA to a final concentration of 1 mg/ml in 1 ml of Reaction Buffer.
2. Prepare a fresh 1 μg/ml solution of β-galactosidase in Enzyme Dilution Buffer. Keep at room temperature until use.
3. Prepare 10-fold serial dilutions of the β-galactosidase solution ranging from 10-1 to 10-4 in Enzyme Dilution Buffer. For each dilution, dilute the β-galacto-sidase solution into Enzyme Dilution Buffer to a final volume of 100 μl (i.e. dilute 10 μl of β-galactosidase solution into 90 μl of Enzyme Dilution Buffer). Keep at room temperature until use.
4. If using the Reference Standard, dilute the 10 mM Reference Standard 100-fold into 200 μl of Reaction Buffer to prepare a 0.1 mM working solution (i.e. add 5 μl of 10 mM Reference Standard to 495 μl of Reaction Buffer).
   Performing the β-galactosidase Assay

Follow the procedure below to perform the β-galactosidase assay.
1. Into individual wells in a 96-well black-walled microtiter plate, pipet 10 μl of each of the purified β-galactosidase dilutions (100 to 10⁴ dilutions), yielding 10 ng, 1 ng, 100 pg, 10 pg, and 1 pg standards. For more accurate results, assay each sample in triplicate.
2. Pipet 10 μl of Reaction Buffer into a well to serve as a blank.
3. Pipet 100 μl of the 0.1 mM Reference Standard into an empty well (if desired).
4. Add 100 μl of the 1.1 mM CUG substrate working solution to each well containing β-galactosidase.
5. Follow Steps 6-8 of the β-galactosidase Assay Procedure.
   Generating the Standard Curve

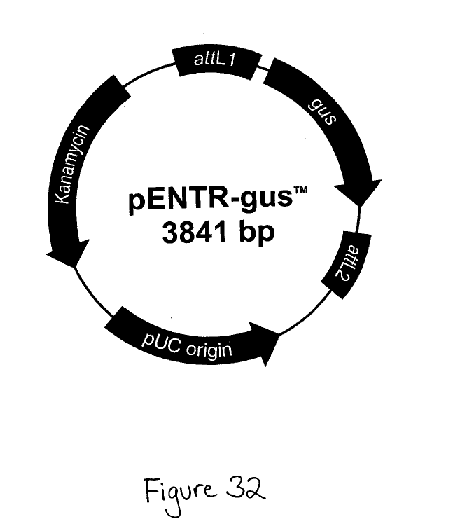

To generate a standard curve, first subtract the fluorescence of the blank from that of each of the samples containing the purified β-galactosidase solutions. If the standard curve will be used for comparison with assays performed at a later date, divide the background-subtracted fluorescence of the β-galactosidase standards by the background-subtracted fluorescence of the reference standard. Plot the resulting corrected fluorescence intensities versus enzyme amount on a log-log scale. Adjust the values for enzyme amount to compensate for the purity of the enzyme preparation. Alternatively, plot fluorescence versus units of β-galactosidase activity. A standard curve (without reference standard normalization) should resemble the sample curve shown in FIG. 31.

Note that the assay has a linear detection range of about 0.5 to over 1000 pg β-galactosidase, and that fluorescence units ranging from about 10 to 10⁵ fall within the linear range of the assay. The lower detection limit corresponds to about ten lacZ-positive NIH3T3 cells per well.

Map and Features of pSCREEN-iT™/lacZ-DEST

The map shown in FIG. 4 shows the elements of pSCREEN-iT™/lacZ-DEST. DNA from the entry clone replaces the region between the attR sites at bases 3976 and 5659. The complete sequence for pSCREEN-iT™/lacZ-DEST is available from Invitrogen Corporation (Carlsbad, Calif.).

Features of the Vector

The pSCREEN-iT™/lacZ-DEST vector (8702 bp) contains the following elements. All features have been functionally tested and the vector fully sequenced.

Map of pSCREEN-iT™/lacZ-GW/CDK2

Description

pSCREEN-iT™/lacZ-GW/CDK2 is a 7947 bp control vector containing the human CDK2 gene (Elledge, S. J., and Spottswood, M. R. (1991). A New Human p34 Protein Kinase, CDK2, Identified by Complementation of a cdc28 Mutation in Saccharomyces cerevisiae, is a Homolog of Xenopus Egl. EMBO J. 10, 2653-2659; Ninomiya-Tsuji, J., Nomoto, S., Yasuda, H., Reed, S. I., and Matsumoto, K. (1991). Cloning of a Human cDNA Encoding a CDC2-Related Kinase by Complementation of a Budding Yeast cdc28 Mutation. Proc. Natl. Acad. Sci. USA 88, 9006-9010; Tsai, L. H., Harlow, E., and Meyerson, M. (1991). Isolation of the Human cdk2 Gene that Encodes the Cyclin A- and Adenovirus E1A-Associated p33 Kinase. Nature 353, 174-177) fused to the lacZ reporter gene, and was generated by performing an LR recombination with the pSCREEN-iT™/lacZ-DEST vector and an Ultimate™ hORF Clone containing the human CDK2 gene (Invitrogen Corporation (Carlsbad, Calif.) Clone ID No. IOH21140; Genbank Accession No. NM_{—}001798).

Map of pSCREEN-iT™/lacZ-GW/CDK2

The map shown in FIG. 5 shows the elements of pSCREEN-iT™/lacZ-GW/CDK2. The complete sequence of the vector is shown in FIG. 5B-5G and is available from Invitrogen Corporation (Carlsbad, Calif.).

CDK2 CDK2 is a member of the serine/threonine protein kinase family, and is a catalytic subunit of the cyclin-dependent protein kinase complex whose activity is restricted to the G1-S phase and essential for cell cycle G1/S phase transition. The protein associates with and is regulated by the regulatory subunits of the complex including cyclin A or E, CDK inhibitor p21Cip1 (CDKN1A) and p27Kip1 (CDKN1B). Its activity is also regulated by its protein phosphorylation.

Map of pENTR™-gus

Description

pENTR™-gus is a 3841 bp entry clone containing the Arabidopsis thaliana gene for β-glucuronidase (gus) (Kertbundit, S., Greve, H. d., Deboeck, F., Montagu, M. V., and Hemalsteens, J. P. (1991). In vivo Random b-glucuronidase Gene Fusions in Arabidopsis thaliana. Proc. Natl. Acad. Sci. USA 88, 5212-5216). The gus gene was amplified using PCR primers containing attB recombination sites. The amplified PCR product was then used in a BP recombination reaction with pDONR201™ to generate the entry clone. For more information about the BP recombination reaction, refer to the Gateway® Technology with Clonase™ II manual which is available from Invitrogen Corporation (Carlsbad, Calif.).

Map of Control Vector

FIG. 32 summarizes the features of the pENTR™-gus vector. The complete sequence for pENTR™-gus is available from Invitrogen Corporation (Carlsbad, Calif.).

Although the foregoing invention has been described in some detail by way of illustration and example for purposes of clarity of understanding, this invention is not limited to the particular embodiments disclosed, but is intended to cover all changes and modifications that are within the spirit and scope of the invention as defined by the appended claims.

All publications and patents mentioned in this specification are indicative of the level of skill of those skilled in the art to which this invention pertains. All publications and patents are herein incorporated by reference to the same extent as if each individual publication or patent application were specifically and individually indicated to be incorporated by reference.

1. A method for identifying one or more RNAi cleavage sites along a target RNA molecule, the method comprising:
introducing one or more double stranded RNA (dsRNA) molecules into one or more cells comprising the target RNA molecule, wherein the nucleotide sequence of at least one of the strands of the one or more dsRNA molecules is identical to a nucleotide sequence found within the target RNA molecule;
incubating the one or more cells under conditions which allow for cleavage of the target RNA molecule, thereby producing two or more target RNA fragments;
releasing RNA from the cells;
determining the nucleotide sequence of (i) one or more of the target RNA fragments, or (ii) one or more terminal portions of one or more of the target RNA fragments; and
comparing the sequence data obtained in (d) to the sequence of the target RNA molecule.

2. The method of claim 1, wherein the comparison in step (e) is used to identify one or more RNAi cleavages in the target RNA molecule.

3. A method for identifying one or more RNAi cleavage sites along a target RNA molecule, the method comprising:
introducing a mixed population of double stranded RNA (dsRNA) molecules into one or more cells comprising the target RNA molecule, wherein the nucleotide sequence of at least one of the strands of each member of the mixed population of dsRNA molecules is identical to a nucleotide sequence found within the target RNA molecule;
incubating the one or more cells under conditions which allow for cleavage of the target RNA molecule, thereby producing two or more target RNA fragments;
releasing RNA from the cells;
determining the nucleotide sequence of (i) one or more of the target RNA fragments, or (ii) one or more terminal portions of one or more of the target RNA fragments; and
comparing the sequence data obtained in (d) to the sequence of the intact target RNA molecule.

4. The method of claim 3, wherein the comparison in step (e) is used to identify one or more RNAi cleavages in the target RNA molecule.

5. The method of claim 3, wherein the mixed population comprises 2 to 200 non-identical dsRNA molecules.

6. The method of claim 3, wherein the mixed population comprises 5 to 50 non-identical dsRNA molecules.

7. The method of claim 3, wherein the mixed population comprises 10 to 20 non-identical dsRNA molecules.

8. The method of claim 3, wherein the dsRNA molecules are synthetic RNA molecules.

9. The method of claim 3, wherein the dsRNA molecules are produced by cleavage of one or more dsRNA molecules with an enzyme having RNase activity.

10. The method of claim 3, wherein one or both strands of the dsRNA molecules are 15 to 30 nucleotides in length.

11. The method of claim 3, wherein one or both strands of the dsRNA molecules are 21 to 23 nucleotides in length.

12. The method of claim 3, wherein some or all of the members of the mixed population of dsRNA molecules have two 5′ overhangs.

13. The method of claim 3, wherein some or all of the members of the mixed population of dsRNA molecules has two 3′ overhangs.

14. The method of claim 3, wherein some or all of the members of the mixed population of dsRNA molecules has a blunt 5′ end or a blunt 3′ end.

15. The method of claim 14, wherein some or all of the members of the mixed population of dsRNA molecules has a blunt 5′ and 3′ ends.

16. The method of claim 3, wherein some or all of the members of the mixed population of dsRNA molecules are siRNA molecules.

17. The method of claim 3, wherein the one or more cells in step (a) are contacted with a lipophilic reagent.

18. The method of claim 3, wherein the dsRNA molecules are introduced into the one or more cells by electroporation.

19. The method of claim 3, wherein the nucleotide sequence of (i) one or more of the target RNA fragments, or (ii) one or more terminal portions of one or more of the target RNA fragments, is determined by a method comprising:
synthesizing one or more DNA molecules complementary to the one or more target RNA fragments or to a terminal portion of the one or more target RNA fragments; and
sequencing all or part of the complementary DNA molecules.

20. The method of claim 3, wherein the nucleotide sequence of (i) one or more of the target RNA fragments, or (ii) one or more terminal portions of one or more of the target RNA fragments, is determined by a method comprising:
hybridizing one or more of the target RNA fragments to at least a portion of a labeled single stranded nucleic acid molecule, wherein the labeled single stranded nucleic acid molecule comprises a nucleotide sequence that is complementary to one or more of the target RNA fragments;
digesting portions of the labeled single stranded nucleic acid molecule that are not bound to one or more of the target RNA fragments through base-pair interactions, thereby producing one or more labeled complementary nucleic acid molecules having a nucleotide sequence complementary to the one or more target RNA fragments; and
sequencing the labeled complementary nucleic acid molecules or a terminal portion thereof;
wherein the sequence of the complementary nucleic acid molecule is the complement of the sequence of the target RNA fragments or a terminal portion thereof.

21. (canceled)

22. (canceled)

23. A method for producing a mixed population of double stranded RNA (dsRNA) fragments, the method comprising:
incubating a first intact dsRNA molecule with an enzyme having RNase activity, thereby producing a first set of two or more dsRNA fragments;
incubating a second intact dsRNA molecule with an enzyme having RNase activity, thereby producing a second set of two or more dsRNA fragments; and
combining the first set of two or more dsRNA fragments with the second set of two or more dsRNA fragments, thereby producing a mixed population of dsRNA fragments;
wherein the first intact dsRNA molecule and the second intact dsRNA molecule are non-identical.

24. A method for producing a mixed population of double stranded RNA (dsRNA) fragments, the method comprising:
combining a first intact dsRNA molecule and a second intact dsRNA molecule to form a mixture of intact dsRNA molecules;
incubating the mixture of intact dsRNA molecules with an enzyme having RNase activity, thereby producing a mixed population of dsRNA fragments;
wherein the first intact dsRNA molecule and the second intact dsRNA molecule are non-identical.

25. The method of claim 24, wherein the enzyme having RNase activity is an enzyme selected from the group consisting of Dicer and E. coli RNase III.

26. (canceled)

27. The method of claim 24, wherein the enzyme having RNase activity is recombinant human dicer.

28. The method of claim 24, wherein the nucleotide sequence of at least one of the strands of the first intact dsRNA molecule is at least 90% identical to the nucleotide sequence encoded by a first gene or a portion thereof,
and wherein the nucleotide sequence of at least one of the strands of the second intact dsRNA molecule is at least 90% identical to the nucleotide sequence encoded by a second gene or a portion thereof.

29. (canceled)

30. (canceled)

31. (canceled)

32. (canceled)

33. The method of claim 24, wherein one or both strands of one or more of the dsRNA fragments are 15 to 30 nucleotides in length.

34. The method of claim 24, wherein one or both strands of one or more of the dsRNA fragments are 21 to 23 nucleotides in length.

35. The method of claim 24, wherein one or more of the dsRNA fragments have 5′ overhangs.

36. The method of claim 24, wherein one or more of the dsRNA fragments have 3′ overhangs.

37. The method of claim 24, wherein one or more of the dsRNA fragments have 5′ or 3′ blunt ends.

38. The method of claim 24, wherein one or more of the dsRNA fragments have 5′ and 3′ blunt ends.

39. The method of claim 24, wherein the dsRNA fragments are siRNA molecules.

40. A mixed population of dsRNA molecules produced by the method of claim 24.

41. A mixed population of double stranded RNA (dsRNA) molecules, the mixed population comprising at least one first dsRNA molecule and at least one second dsRNA molecule,
wherein the nucleotide sequence of at least one of the strands of the first dsRNA molecule is at least 90% identical to the nucleotide sequence encoded by a first gene or a portion thereof,
wherein the nucleotide sequence of at least one of the strands of the second dsRNA molecule is at least 90% identical to the nucleotide sequence encoded by a second gene or a portion thereof, and
wherein the first and the second dsRNA molecules are non-identical.

42. The mixed population of claim 41, wherein one or both strands of the first and second dsRNA molecules are 15 to 30 nucleotides in length.

43. The mixed population of claim 41, wherein one or both strands of the first and second dsRNA molecules are 21 to 23 nucleotides in length.

44. (canceled)

45. (canceled)

46. (canceled)

47. (canceled)

48. (canceled)

49. An isolated dsRNA molecule comprising a nucleotide sequence, at least one strand of which is identical to at least 10 nucleotides of a messenger RNA which encodes a polypeptide with β-lactamase activity.

50. (canceled)

51. (canceled)


SEQUENCE LISTING (151 sequences)

SEQ ID NO: 1 - DNA, 8702 bp, artificial sequence
  pSCREEN-iT/lacZ-DEST vector
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatagacccaagctggctagttaagctcaccatgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccggccacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgacgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaagcggccgctcgagtcacatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatgtgtagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 8047 bp, artificial sequence
  pSCREEN-iT/lacZ GW/CDK2 vector
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatagacccaagctggctagttaagctcaccatgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccggccacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgacgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaagcggccgctcgagtcacatcaacaagtttgtacaaaaaagcaggcaccatggagaacttccaaaaggtggaaaagatcggagagggcacgtacggagttgtgtacaaagccagaaacaagttgacgggagaggtggtggcgcttaagaaaatccgcctggacactgagactgagggtgtgcccagtactgccatccgagagatctctctgcttaaggagcttaaccatcctaatattgtcaagctgctggatgtcattcacacagaaaataaactctacctggtttttgaatttctgcaccaagatctcaagaaattcatggatgcctctgctctcactggcattcctcttcccctcatcaagagctatctgttccagctgctccagggcctagctttctgccattctcatcgggtcctccaccgagaccttaaacctcagaatctgcttattaacacagagggggccatcaagctagcagactttggactagccagagcttttggagtccctgttcgtacttacacccatgaggtggtgaccctgtggtaccgagctcctgaaatcctcctgggctgcaaatattattccacagctgtggacatctggagcctgggctgcatctttgctgagatggtgactcgccgggccctattccctggagattctgagattgaccagctcttccggatctttcggactctggggaccccagatgaggtggtgtggccaggagttacttctatgcctgattacaagccaagtttccccaagtgggcccggcaagattttagtaaagttgtacctcccctggatgaagatggacggagcttgttatcgcaaatgctgcactacgaccctaacaagcggatttcggccaaggcagccctggctcaccctttcttccaggatgtgaccaagccagtaccccatcttcgactctagaacccagctgctgggattgttcgcctaaagccggttccgtcgggaccgagtgggaaagaaggtcctacactggttcggtcatggggtagaagctgagatcttgggtcgtttcttgtacaaagtggttgatgtgtagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - protein, 268 aa, artificial sequence
  altered polypeptide with B-lactomase activity
MDPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWLLST

SEQ ID NO: 4 - DNA, 804 bp, artificial sequence
  nucleotide sequence that encodes aa sequence of altered polypeptide
atggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggctgttatcaaca

SEQ ID NO: 5 - RNA, 35 nt, artificial sequence
  siRNA target sequence identification (top nucleotide)
gugaacaucacguacgcggaauacuucgaaauguc

SEQ ID NO: 6 - RNA, 21 nt, artificial sequence
  siRNA target sequence idenitification (middle nucleotide)
cguacgcggaauacuucgauu

SEQ ID NO: 7 - RNA, 21 nt, artificial sequence
  siRNA target sequence identification (bottom nucleotide)
ucgaaguauuccgcguacguu

SEQ ID NO: 8 - DNA, 700 bp, artificial sequence
  RISC cleavage sites in Luciferase
tagagaacccactgcttactggcttatcgaaattaatacgactcactatagggagacccaagctggctagcgtttaaacttaagcttggtaccgagctcggatccactagtccagtgtggtggaattctgcagatccacaaccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctt

SEQ ID NO: 9 - RNA, 21 nt, artificial sequence
  emperically identified siRNA (top sequence ID1)
gaacaucacguacgcggaaua

SEQ ID NO: 10 - RNA, 21 nt, artificial sequence
  emperically identified siRNA (bottom sequence ID1)
cacuuguagugcaugcgccuu

SEQ ID NO: 11 - RNA, 21 nt, artificial sequence
  emperically identified siRNA (top sequence ID2)
aaacgauaugggcugaauaca

SEQ ID NO: 12 - RNA, 21 nt, artificial sequence
  emperically identified siRNA (bottom sequence ID2)
acuuugcuauacccgacuuau

SEQ ID NO: 13 - RNA, 21 nt, artificial sequence
  emperically identified siRNA (top sequence ID3)
aaaucacagaaucgucguaug

SEQ ID NO: 14 - RNA, 21 nt, artificial sequence
  emperically identified siRNA (bottom  sequence ID3)
uguuuagugucuuagcagcau

SEQ ID NO: 15 - DNA, 1600 bp, artificial sequence
  RISC cleavage sites in lacZ following transfection of i-siRNA
ccatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgccc

SEQ ID NO: 16 - RNA, 19 nt, artificial sequence
  GL2
cguacgcggaauacuucga

SEQ ID NO: 17 - RNA, 19 nt, artificial sequence
  GL2-22-AS
gcaugcgccuuaugaagcu

SEQ ID NO: 18 - DNA, 264 bp, artificial sequence
  nucleotide sequence of recombination region of an expression clone
gggattggtggcgacgactcctggagcccgtcagtatcggcggaattccagctgagcgccggtcgctaccattaccagttggtctggtgtcaaaaagcggccgctcgagtcacatcaacaagtttgtacaaaaaagcaggctnnacccagctttcttgtacaaagtggttgatgtgtagtaatgagtttaaacgggggaggctaactgaaacacggaaggagacaataccggaaggaacccgcgctatgacggcaataaaaaga

SEQ ID NO: 19 - protein, 47 aa, artificial sequence
  aa sequence encoded by recombination region of an expression clone
GIGGDDSWSPSVSAEFQLSAGRYHYQLVWCQKAAARVTSTSLYKKAG

SEQ ID NO: 20 - RNA, 21 nt, artificial sequence
  targeted oligonucleotide
cccuucugucuugaacaugag

SEQ ID NO: 21 - DNA, 21 bp, artificial sequence
  targeted oligonucleotide
ctgatgttcaagacagaacgg

SEQ ID NO: 22 - RNA, 21 nt, artificial sequence
  targeted oligonucleotide
cucauguucaagacagaaggg

SEQ ID NO: 23 - DNA, 21 bp, artificial sequence
  non-targeted oligonucleotide
gagtacaagttctgtcttccc

SEQ ID NO: 24 - DNA, 21 bp, artificial sequence
  non-targeted oligonucleotide
ggcaagacagaacttgtagtc

SEQ ID NO: 25 - DNA, 21 bp, artificial sequence
  non-targeted oligonucleotide
gggaagacagaacttgtactc

SEQ ID NO: 26 - RNA, 22 nt, artificial sequence
  example RNA molecule
auggacccagaaacgcugguga

SEQ ID NO: 27 - RNA, 19 nt, artificial sequence
  example RNA molecule
aaacgcuggugaaaguaaa

SEQ ID NO: 28 - RNA, 25 nt, artificial sequence
  example RNA molecule
ccccgaagaacguuuuccaaugaug

SEQ ID NO: 29 - RNA, 20 nt, artificial sequence
  example RNA molecule
cguuuuccaaugaugagcac

SEQ ID NO: 30 - RNA, 21 nt, artificial sequence
  example RNA molecule
agcacuuuuaaaguucugcua

SEQ ID NO: 31 - RNA, 20 nt, artificial sequence
  example RNA molecule
cucagaaugacuugguugag

SEQ ID NO: 32 - RNA, 20 nt, artificial sequence
  example RNA molecule
ugggaaccggagcugaauga

SEQ ID NO: 33 - RNA, 25 nt, artificial sequence
  example RNA molecule
agccauaccaaacgacgagcgugac

SEQ ID NO: 34 - RNA, 20 nt, artificial sequence
  example RNA molecule
acuggcgaacuacuuacucu

SEQ ID NO: 35 - RNA, 20 nt, artificial sequence
  example RNA molecule
cacucgcacccagagcgcca

SEQ ID NO: 36 - RNA, 22 nt, artificial sequence
  example RNA molecule
agacagaucgcugagauaggug

SEQ ID NO: 37 - RNA, 21 nt, artificial sequence
  example RNA molecule
cgacggggagucaggcaacua

SEQ ID NO: 38 - RNA, 20 nt, artificial sequence
  example RNA molecule
ugccucacugauuaagcauu

SEQ ID NO: 39 - RNA, 44 nt, artificial sequence
  GeneRacer RNA Oligo
cgacuggagcacgaggacacugacauggacugaaggaguagaaa

SEQ ID NO: 40 - DNA, 23 bp, artificial sequence
  GeneRacer 5′ Primer
cgactggagcacgaggacactga

SEQ ID NO: 41 - DNA, 26 bp, artificial sequence
  GeneRacer 5′ Nested Primer
ggacactgacatggactgaaggagta

SEQ ID NO: 42 - RNA, 25 nt, artificial sequence
  Positive LacZ Stealth Control
ccgucugaauuugaccugagcgcau

SEQ ID NO: 43 - RNA, 25 nt, artificial sequence
  Scrambled Stealth Negative Control
gggaagacagaacuuguacucaaaa

SEQ ID NO: 44 - DNA, 76 bp, artificial sequence
  annealed oligonucleotide
ctctggctaactagagaacccactgcttactggcttatcgaaatagacccaagctggctagctaagctgagcgttt

SEQ ID NO: 45 - DNA, 80 bp, artificial sequence
  annealed oligonucleotide
aaacgctcagcttagctagccagcttgggtctatttcgataagccagtaagcagtgggttctctagttagccagagagct

SEQ ID NO: 46 - DNA, 50 bp, artificial sequence
  pcDNA.2/n-GeneBLAzer/GW-lacZ, forward primer
gatcgatcactagttaagctcaccatgatagatcccgtcgttttacaacg

SEQ ID NO: 47 - DNA, 58 bp, artificial sequence
  pcDNA.2/n-GeneBLAzer/GW-lacZ, reverse primer
gcctcccccgtttaaacaggccttcattactagactcgagcggccgctttttgacacc

SEQ ID NO: 48 - DNA, 25 bp, artificial sequence
  forward annealed oligonucleotide
tcgagtcacgtgtagtaatgagttt

SEQ ID NO: 49 - DNA, 21 bp, artificial sequence
  reverse annealed oligonucleotide
aaactcattactacacgtgac

SEQ ID NO: 50 - DNA, 22 bp, artificial sequence
  forward primer
atgtaactcgccttgatcgttg

SEQ ID NO: 51 - DNA, 22 bp, artificial sequence
  reverse primer
ggccgagcgcagaagtggtcct

SEQ ID NO: 52 - DNA, 20 bp, artificial sequence
  forward sequencing primer
attggtggcgacgactcctg

SEQ ID NO: 53 - DNA, 21 bp, artificial sequence
  reverse sequencing primer
acccgtgcgttttattctgtc

SEQ ID NO: 54 - RNA, 21 nt, artificial sequence
  sense RNA sequence
augaagcagaacaacuuuaac

SEQ ID NO: 55 - RNA, 21 nt, artificial sequence
  sense RNA sequence
acuauuaacuggcgaacuauu

SEQ ID NO: 56 - RNA, 21 nt, artificial sequence
  sense RNA sequence
auuaacuggcgaacuacuuuu

SEQ ID NO: 57 - RNA, 21 nt, artificial sequence
  sense RNA sequence
uuaacuggcgaacuacuuauu

SEQ ID NO: 58 - RNA, 21 nt, artificial sequence
  sense RNA sequence
uaacuggcgaacuacuuacuu

SEQ ID NO: 59 - RNA, 21 nt, artificial sequence
  sense RNA sequence
uggcgaacuacuuacucuauu

SEQ ID NO: 60 - RNA, 19 nt, artificial sequence
  sense RNA sequence
guugacgggagagguggug

SEQ ID NO: 61 - RNA, 19 nt, artificial sequence
  sense RNA sequence
gauggacggagcuuguuau

SEQ ID NO: 62 - RNA, 19 nt, artificial sequence
  sense RNA sequence
gcuagcagacuuuggacua

SEQ ID NO: 63 - RNA, 19 nt, artificial sequence
  sense RNA sequence
auccuccugggcugcaaau

SEQ ID NO: 64 - RNA, 19 nt, artificial sequence
  sense RNA sequence
gugggcccggcaagauuuu

SEQ ID NO: 65 - RNA, 19 nt, artificial sequence
  sense RNA sequence
caggaggugaucgauaagc

SEQ ID NO: 66 - RNA, 19 nt, artificial sequence
  sense RNA sequence
gcucgaucugaagaggcag

SEQ ID NO: 67 - RNA, 19 nt, artificial sequence
  sense RNA sequence
gcucuuccaagaauacgac

SEQ ID NO: 68 - RNA, 19 nt, artificial sequence
  sense RNA sequence
ggugaucgauaagcugaag

SEQ ID NO: 69 - RNA, 19 nt, artificial sequence
  sense RNA sequence
uaucuacaaggcggacuuc

SEQ ID NO: 70 - RNA, 19 nt, artificial sequence
  sense RNA sequence
guguccaaugaggagaaau

SEQ ID NO: 71 - RNA, 19 nt, artificial sequence
  sense RNA sequence
aucaaaggcuaugucuggc

SEQ ID NO: 72 - RNA, 19 nt, artificial sequence
  sense RNA sequence
cuaccucuccuucaccaua

SEQ ID NO: 73 - RNA, 19 nt, artificial sequence
  sense RNA sequence
aaugaggagaaauugaacc

SEQ ID NO: 74 - RNA, 19 nt, artificial sequence
  sense RNA sequence
uuucucugguuggucaaca

SEQ ID NO: 75 - RNA, 19 nt, artificial sequence
  sense RNA sequence
uugaagguggaaugaaaua

SEQ ID NO: 76 - RNA, 19 nt, artificial sequence
  sense RNA sequence
ccaaugcgaccuuaaacaa

SEQ ID NO: 77 - RNA, 19 nt, artificial sequence
  sense RNA sequence
gcaacuucuguauuuggag

SEQ ID NO: 78 - RNA, 19 nt, artificial sequence
  sense RNA sequence
caagaagacggacauugcu

SEQ ID NO: 79 - RNA, 19 nt, artificial sequence
  sense RNA sequence
ggacaaguucuaccggaag

SEQ ID NO: 80 - RNA, 19 nt, artificial sequence
  sense RNA sequence
ggucgacugcuucuacacu

SEQ ID NO: 81 - RNA, 19 nt, artificial sequence
  sense RNA sequence
gggcuacaaugucaagucc

SEQ ID NO: 82 - RNA, 19 nt, artificial sequence
  sense RNA sequence
gcccuccaauguccuuauc

SEQ ID NO: 83 - RNA, 19 nt, artificial sequence
  sense RNA sequence
caugcgcacggucgacugc

SEQ ID NO: 84 - RNA, 19 nt, artificial sequence
  sense RNA sequence
gacgauggaugccggcugc

SEQ ID NO: 85 - RNA, 19 nt, artificial sequence
  sense RNA sequence
gcggauccgggccaccgug

SEQ ID NO: 86 - RNA, 19 nt, artificial sequence
  sense RNA sequence
gcuauccaggcugugcuau

SEQ ID NO: 87 - RNA, 19 nt, artificial sequence
  sense RNA sequence
cugacugacuaccucauga

SEQ ID NO: 88 - RNA, 19 nt, artificial sequence
  sense RNA sequence
gggaaaucgugcgugacau

SEQ ID NO: 89 - RNA, 19 nt, artificial sequence
  sense RNA sequence
gugcgugacauuaaggaga

SEQ ID NO: 90 - RNA, 19 nt, artificial sequence
  sense RNA sequence
gcauccacgaaacuaccuu

SEQ ID NO: 91 - RNA, 19 nt, artificial sequence
  sense RNA sequence
ccacgaaacuaccuucaac

SEQ ID NO: 92 - RNA, 19 nt, artificial sequence
  sense RNA sequence
gaaacuaccuucaacucca

SEQ ID NO: 93 - RNA, 19 nt, artificial sequence
  sense RNA sequence
accuucaacuccaucauga

SEQ ID NO: 94 - RNA, 19 nt, artificial sequence
  sense RNA sequence
aagugugacguggacaucc

SEQ ID NO: 95 - RNA, 19 nt, artificial sequence
  sense RNA sequence
gcaaagaccuguacgccaa

SEQ ID NO: 96 - RNA, 25 nt, artificial sequence
  sense RNA sequence
ccgucugaauuugaccugagcgcau

SEQ ID NO: 97 - RNA, 25 nt, artificial sequence
  sense RNA sequence
gggaagacagaacuuguacucaaaa

SEQ ID NO: 98 - RNA, 21 nt, artificial sequence
  antisense RNA sequence
uaaaguuguucugcuucauca

SEQ ID NO: 99 - RNA, 21 nt, artificial sequence
  antisense RNA sequence
uaguucgccaguuaauaguuu

SEQ ID NO: 100 - RNA, 21 nt, artificial sequence
  antisense RNA sequence
aaguaguucgccaguuaauuu

SEQ ID NO: 101 - RNA, 21 nt, artificial sequence
  antisense RNA sequence
uaaguaguucgccaguuaauu

SEQ ID NO: 102 - RNA, 21 nt, artificial sequence
  antisense RNA sequence
guaaguaguucgccaguuauu

SEQ ID NO: 103 - RNA, 21 nt, artificial sequence
  antisense RNA sequence
uagaguaaguaguucgccauu

SEQ ID NO: 104 - RNA, 19 nt, artificial sequence
  antisense RNA sequence
caccaccucucccgucaac

SEQ ID NO: 105 - RNA, 19 nt, artificial sequence
  antisense RNA sequence
auaacaagcuccguccauc

SEQ ID NO: 106 - RNA, 19 nt, artificial sequence
  antisense RNA sequence
uaguccaaagucugcuagc

SEQ ID NO: 107 - RNA, 19 nt, artificial sequence
  antisense RNA sequence
auuugcagcccaggaggau

SEQ ID NO: 108 - RNA, 19 nt, artificial sequence
  antisense RNA sequence
aaaaucuugccgggcccac

SEQ ID NO: 109 - RNA, 19 nt, artificial sequence
  antisense RNA sequence
gcuuaucgaucaccuccug

SEQ ID NO: 110 - RNA, 19 nt, artificial sequence
  antisense RNA sequence
cugccucuucagaucgagc

SEQ ID NO: 111 - RNA, 19 nt, artificial sequence
  antisense RNA sequence
gucguauucuuggaagagc

SEQ ID NO: 112 - RNA, 19 nt, artificial sequence
  antisense RNA sequence
cuucagcuuaucgaucacc

SEQ ID NO: 113 - RNA, 19 nt, artificial sequence
  antisense RNA sequence
gaaguccgccuuguagaua

SEQ ID NO: 114 - RNA, 19 nt, artificial sequence
  antisense RNA sequence
auuucuccucauuggacac

SEQ ID NO: 115 - RNA, 19 nt, artificial sequence
  antisense RNA sequence
gccagacauagccuuugau

SEQ ID NO: 116 - RNA, 19 nt, artificial sequence
  antisense RNA sequence
uauggugaaggagagguag

SEQ ID NO: 117 - RNA, 19 nt, artificial sequence
  antisense RNA sequence
gguucaauuucuccucauu

SEQ ID NO: 118 - RNA, 19 nt, artificial sequence
  antisense RNA sequence
uguugaccaaccagagaaa

SEQ ID NO: 119 - RNA, 19 nt, artificial sequence
  antisense RNA sequence
uauuucauuccaccuucaa

SEQ ID NO: 120 - RNA, 19 nt, artificial sequence
  antisense RNA sequence
uuguuuaaggucgcauugg

SEQ ID NO: 121 - RNA, 19 nt, artificial sequence
  antisense RNA sequence
cuccaaauacagaaguugc

SEQ ID NO: 122 - RNA, 19 nt, artificial sequence
  antisense RNA sequence
agcaauguccgucuucuug

SEQ ID NO: 123 - RNA, 19 nt, artificial sequence
  antisense RNA sequence
cuuccgguagaacuugucc

SEQ ID NO: 124 - RNA, 19 nt, artificial sequence
  antisense RNA sequence
aguguagaagcagucgacc

SEQ ID NO: 125 - RNA, 19 nt, artificial sequence
  antisense RNA sequence
ggacuugacauuguagccc

SEQ ID NO: 126 - RNA, 19 nt, artificial sequence
  antisense RNA sequence
gauaaggacauuggagggc

SEQ ID NO: 127 - RNA, 19 nt, artificial sequence
  antisense RNA sequence
gcagucgaccgugcgcaug

SEQ ID NO: 128 - RNA, 19 nt, artificial sequence
  antisense RNA sequence
gcagccggcauccaucguc

SEQ ID NO: 129 - RNA, 19 nt, artificial sequence
  antisense RNA sequence
cacgguggcccggauccgc

SEQ ID NO: 130 - RNA, 19 nt, artificial sequence
  antisense RNA sequence
auagcacagccuggauagc

SEQ ID NO: 131 - RNA, 19 nt, artificial sequence
  antisense RNA sequence
ucaugagguagucagucag

SEQ ID NO: 132 - RNA, 19 nt, artificial sequence
  antisense RNA sequence
augucacgcacgauuuccc

SEQ ID NO: 133 - RNA, 19 nt, artificial sequence
  antisense RNA sequence
ucuccuuaaugucacgcac

SEQ ID NO: 134 - RNA, 19 nt, artificial sequence
  antisense RNA sequence
aagguaguuucguggaugc

SEQ ID NO: 135 - RNA, 19 nt, artificial sequence
  antisense RNA sequence
guugaagguaguuucgugg

SEQ ID NO: 136 - RNA, 19 nt, artificial sequence
  antisense RNA sequence
uggaguugaagguaguuuc

SEQ ID NO: 137 - RNA, 19 nt, artificial sequence
  antisense RNA sequence
ucaugauggaguugaaggu

SEQ ID NO: 138 - RNA, 19 nt, artificial sequence
  antisense RNA sequence
ggauguccacgucacacuu

SEQ ID NO: 139 - RNA, 19 nt, artificial sequence
  antisense RNA sequence
uuggcguacaggucuuugc

SEQ ID NO: 140 - RNA, 25 nt, artificial sequence
  antisense RNA sequence
augcgcucaggucaaauucagacgg

SEQ ID NO: 141 - RNA, 25 nt, artificial sequence
  antisense RNA sequence
uuuugaguacaaguucugucuuccc

SEQ ID NO: 142 - RNA, 15 nt, artificial sequence
  dsRNA
auacacuauucucag

SEQ ID NO: 143 - RNA, 15 nt, artificial sequence
  dsRNA
ccaugagugauaaca

SEQ ID NO: 144 - RNA, 15 nt, artificial sequence
  dsRNA
ccuugaucguuggga

SEQ ID NO: 145 - RNA, 15 nt, artificial sequence
  dsRNA
acuggcgaacuacuu

SEQ ID NO: 146 - RNA, 15 nt, artificial sequence
  dsRNA
gauggcaugacagua

SEQ ID NO: 147 - RNA, 15 nt, artificial sequence
  dsRNA
gugacaccacgaugc

SEQ ID NO: 148 - RNA, 15 nt, artificial sequence
  dsRNA
ggaggcggauaaagu

SEQ ID NO: 149 - RNA, 15 nt, artificial sequence
  dsRNA
gucucgcgguaucau

SEQ ID NO: 150 - RNA, 15 nt, artificial sequence
  dsRNA
caacuauggaugaac

SEQ ID NO: 151 - RNA, 15 nt, artificial sequence
  dsRNA
cacuggggccagaug